SEQUENCE LISTING (299 sequences)

SEQ ID NO: 1 - protein, 5 aa, synthetic construct
  REGION 1..5: note = TCR (T-cell receptor) amino acid sequence, Donor HD1
KALYS

SEQ ID NO: 2 - protein, 7 aa, synthetic construct
  REGION 1..7: note = TCR amino acid sequence, Donor HD1
LLKGGEQ

SEQ ID NO: 3 - protein, 13 aa, synthetic construct
  REGION 1..13: note = TCR amino acid sequence, Donor HD1
CGTAWINDYKLSF

SEQ ID NO: 4 - protein, 132 aa, synthetic construct
  REGION 1..132: note = TCR amino acid sequence, Donor HD1
METLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTAWINDYKLSFGAGTTVTVRAN

SEQ ID NO: 5 - protein, 272 aa, synthetic construct
  REGION 1..272: note = TCR amino acid sequence, Donor HD1
METLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTAWINDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 6 - protein, 5 aa, synthetic construct
  REGION 1..5: note = TCR amino acid sequence, Donor HD1
SGHDY

SEQ ID NO: 7 - protein, 6 aa, synthetic construct
  REGION 1..6: note = TCR amino acid sequence, Donor HD1
FNNNVP

SEQ ID NO: 8 - protein, 16 aa, synthetic construct
  REGION 1..16: note = TCR amino acid sequence, Donor HD1
CASRKTGGYSNQPQHF

SEQ ID NO: 9 - protein, 136 aa, synthetic construct
  REGION 1..136: note = TCR amino acid sequence, Donor HD1
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRKTGGYSNQPQHFGDGTRLSILE

SEQ ID NO: 10 - protein, 312 aa, synthetic construct
  REGION 1..312: note = TCR amino acid sequence, Donor HD1
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRKTGGYSNQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 11 - protein, 314 aa, synthetic construct
  REGION 1..314: note = TCR amino acid sequence, Donor HD1
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRKTGGYSNQPQHFGDGTRLSILEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 12 - protein, 6 aa, synthetic construct
  REGION 1..6: note = TCR amino acid sequence, Donor HD2
SSVPPY

SEQ ID NO: 13 - protein, 8 aa, synthetic construct
  REGION 1..8: note = TCR amino acid sequence, Donor HD2
YTSAATLV

SEQ ID NO: 14 - protein, 14 aa, synthetic construct
  REGION 1..14: note = TCR amino acid sequence, Donor HD2
CAVRLSGSARQLTF

SEQ ID NO: 15 - protein, 134 aa, synthetic construct
  REGION 1..134: note = TCR amino acid sequence, Donor HD2
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVRLSGSARQLTFGSGTQLTVLPD

SEQ ID NO: 16 - protein, 274 aa, synthetic construct
  REGION 1..274: note = TCR amino acid sequence, Donor HD2
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVRLSGSARQLTFGSGTQLTVLPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 17 - protein, 7 aa, synthetic construct
  REGION 1..7: note = TCR amino acid sequence, Donor HD2
TSESDYY

SEQ ID NO: 18 - protein, 8 aa, synthetic construct
  REGION 1..8: note = TCR amino acid sequence, Donor HD2
QEAYKQQN

SEQ ID NO: 19 - protein, 14 aa, synthetic construct
  REGION 1..14: note = TCR amino acid sequence, Donor HD2
CAYRSLKYGNKLVF

SEQ ID NO: 20 - protein, 136 aa, synthetic construct
  REGION 1..136: note = TCR amino acid sequence, Donor HD2
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSLKYGNKLVFGAGTILRVKSY

SEQ ID NO: 21 - protein, 276 aa, synthetic construct
  REGION 1..276: note = TCR amino acid sequence, Donor HD2
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSLKYGNKLVFGAGTILRVKSYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 22 - protein, 5 aa, synthetic construct
  REGION 1..5: note = TCR amino acid sequence, Donor HD2
SGHAT

SEQ ID NO: 23 - protein, 6 aa, synthetic construct
  REGION 1..6: note = TCR amino acid sequence, Donor HD2
FQNNGV

SEQ ID NO: 24 - protein, 12 aa, synthetic construct
  REGION 1..12: note = TCR amino acid sequence, Donor HD2
CASSLLGDEQYF

SEQ ID NO: 25 - protein, 132 aa, synthetic construct
  REGION 1..132: note = TCR amino acid sequence, Donor HD2
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLLGDEQYFGPGTRLTVTE

SEQ ID NO: 26 - protein, 308 aa, synthetic construct
  REGION 1..308: note = TCR amino acid sequence, Donor HD2
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLLGDEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 27 - protein, 310 aa, synthetic construct
  REGION 1..310: note = TCR amino acid sequence, Donor HD2
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLLGDEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 28 - protein, 5 aa, synthetic construct
  REGION 1..5: note = TCR amino acid sequence, Donor HD2
SGHTA

SEQ ID NO: 29 - protein, 6 aa, synthetic construct
  REGION 1..6: note = TCR amino acid sequence, Donor HD2
FQGNSA

SEQ ID NO: 30 - protein, 16 aa, synthetic construct
  REGION 1..16: note = TCR amino acid sequence, Donor HD2
CASSLVALQGAGEQYF

SEQ ID NO: 31 - protein, 136 aa, synthetic construct
  REGION 1..136: note = TCR amino acid sequence, Donor HD2
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLVALQGAGEQYFGPGTRLTVTE

SEQ ID NO: 32 - protein, 312 aa, synthetic construct
  REGION 1..312: note = TCR amino acid sequence, Donor HD2
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLVALQGAGEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 33 - protein, 314 aa, synthetic construct
  REGION 1..314: note = TCR amino acid sequence, Donor HD2
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSLVALQGAGEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 34 - protein, 6 aa, synthetic construct
  REGION 1..6: note = TCR amino acid sequence, Donor HD3
NSASQS

SEQ ID NO: 35 - protein, 6 aa, synthetic construct
  REGION 1..6: note = TCR amino acid sequence, Donor HD3
VYSSGN

SEQ ID NO: 36 - protein, 15 aa, synthetic construct
  REGION 1..15: note = TCR amino acid sequence, Donor HD3
CVVNLLSNQGGKLIF

SEQ ID NO: 37 - protein, 134 aa, synthetic construct
  REGION 1..134: note = TCR amino acid sequence, Donor HD3
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNLLSNQGGKLIFGQGTELSVKPN

SEQ ID NO: 38 - protein, 274 aa, synthetic construct
  REGION 1..274: note = TCR amino acid sequence, Donor HD3
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNLLSNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 39 - protein, 5 aa, synthetic construct
  REGION 1..5: note = TCR amino acid sequence, Donor HD3
LGHNA

SEQ ID NO: 40 - protein, 6 aa, synthetic construct
  REGION 1..6: note = TCR amino acid sequence, Donor HD3
YSLEER

SEQ ID NO: 41 - protein, 16 aa, synthetic construct
  REGION 1..16: note = TCR amino acid sequence, Donor HD3
CASSQDYLVSNEKLFF

SEQ ID NO: 42 - protein, 138 aa, synthetic construct
  REGION 1..138: note = TCR amino acid sequence, Donor HD3
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDYLVSNEKLFFGSGTQLSVLE

SEQ ID NO: 43 - protein, 314 aa, synthetic construct
  REGION 1..314: note = TCR amino acid sequence, Donor HD3
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDYLVSNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 44 - protein, 316 aa, synthetic construct
  REGION 1..316: note = TCR amino acid sequence, Donor HD3
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDYLVSNEKLFFGSGTQLSVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 45 - protein, 5 aa, synthetic construct
  REGION 1..5: note = TCR amino acid sequence, Donor HD4
TSINN

SEQ ID NO: 46 - protein, 7 aa, synthetic construct
  REGION 1..7: note = TCR amino acid sequence, Donor HD4
IRSNERE

SEQ ID NO: 47 - protein, 14 aa, synthetic construct
  REGION 1..14: note = TCR amino acid sequence, Donor HD4
CATDAYSGNTPLVF

SEQ ID NO: 48 - protein, 133 aa, synthetic construct
  REGION 1..133: note = TCR amino acid sequence, Donor HD4
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAYSGNTPLVFGKGTRLSVIAN

SEQ ID NO: 49 - protein, 273 aa, synthetic construct
  REGION 1..273: note = TCR amino acid sequence, Donor HD4
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAYSGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 50 - protein, 6 aa, synthetic construct
  REGION 1..6: note = TCR amino acid sequence, Donor HD4
DSAIYN

SEQ ID NO: 51 - protein, 7 aa, synthetic construct
  REGION 1..7: note = TCR amino acid sequence, Donor HD4
IQSSQRE

SEQ ID NO: 52 - protein, 16 aa, synthetic construct
  REGION 1..16: note = TCR amino acid sequence, Donor HD4
CAVRAEIYNQGGKLIF

SEQ ID NO: 53 - protein, 135 aa, synthetic construct
  REGION 1..135: note = TCR amino acid sequence, Donor HD4
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRAEIYNQGGKLIFGQGTELSVKPN

SEQ ID NO: 54 - protein, 275 aa, synthetic construct
  REGION 1..275: note = TCR amino acid sequence, Donor HD4
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRAEIYNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 55 - protein, 5 aa, synthetic construct
  REGION 1..5: note = TCR amino acid sequence, Donor HD4
MNHNS

SEQ ID NO: 56 - protein, 6 aa, synthetic construct
  REGION 1..6: note = TCR amino acid sequence, Donor HD4
SASEGT

SEQ ID NO: 57 - protein, 14 aa, synthetic construct
  REGION 1..14: note = TCR amino acid sequence, Donor HD4
CASRAAGLDTEAFF

SEQ ID NO: 58 - protein, 133 aa, synthetic construct
  REGION 1..133: note = TCR amino acid sequence, Donor HD4
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASRAAGLDTEAFFGQGTRLTVVE

SEQ ID NO: 59 - protein, 309 aa, synthetic construct
  REGION 1..309: note = TCR amino acid sequence, Donor HD4
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASRAAGLDTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 60 - protein, 311 aa, synthetic construct
  REGION 1..311: note = TCR amino acid sequence, Donor HD4
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASRAAGLDTEAFFGQGTRLTVVEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 61 - protein, 5 aa, synthetic construct
  REGION 1..5: note = TCR amino acid sequence, Donor HD4
MNHNY

SEQ ID NO: 62 - protein, 6 aa, synthetic construct
  REGION 1..6: note = TCR amino acid sequence, Donor HD4
SVGAGI

SEQ ID NO: 63 - protein, 12 aa, synthetic construct
  REGION 1..12: note = TCR amino acid sequence, Donor HD4
CASTQTPYEQYF

SEQ ID NO: 64 - protein, 131 aa, synthetic construct
  REGION 1..131: note = TCR amino acid sequence, Donor HD4
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASTQTPYEQYFGPGTRLTVTE

SEQ ID NO: 65 - protein, 307 aa, synthetic construct
  REGION 1..307: note = TCR amino acid sequence, Donor HD4
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASTQTPYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 66 - protein, 309 aa, synthetic construct
  REGION 1..309: note = TCR amino acid sequence, Donor HD4
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASTQTPYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 67 - protein, 5 aa, synthetic construct
  REGION 1..5: note = TCR amino acid sequence, Donor HD4
SGHNS

SEQ ID NO: 68 - protein, 6 aa, synthetic construct
  REGION 1..6: note = TCR amino acid sequence, Donor HD4
FNNNVP

SEQ ID NO: 69 - protein, 15 aa, synthetic construct
  REGION 1..15: note = TCR amino acid sequence, Donor HD4
CASSTVGGEDYGYTF

SEQ ID NO: 70 - protein, 135 aa, synthetic construct
  REGION 1..135: note = TCR amino acid sequence, Donor HD4
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSTVGGEDYGYTFGSGTRLTVVE

SEQ ID NO: 71 - protein, 311 aa, synthetic construct
  REGION 1..311: note = TCR amino acid sequence, Donor HD4
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSTVGGEDYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 72 - protein, 313 aa, synthetic construct
  REGION 1..313: note = TCR amino acid sequence, Donor HD4
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSTVGGEDYGYTFGSGTRLTVVEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 73 - protein, 6 aa, synthetic construct
  REGION 1..6: note = TCR amino acid sequence, Donor HD5
DSASNY

SEQ ID NO: 74 - protein, 7 aa, synthetic construct
  REGION 1..7: note = TCR amino acid sequence, Donor HD5
IRSNVGE

SEQ ID NO: 75 - protein, 15 aa, synthetic construct
  REGION 1..15: note = TCR amino acid sequence, Donor HD5
CAASMAGAGSYQLTF

SEQ ID NO: 76 - protein, 134 aa, synthetic construct
  REGION 1..134: note = TCR amino acid sequence, Donor HD5
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASMAGAGSYQLTFGKGTKLSVIPN

SEQ ID NO: 77 - protein, 274 aa, synthetic construct
  REGION 1..274: note = TCR amino acid sequence, Donor HD5
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASMAGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 78 - protein, 5 aa, synthetic construct
  REGION 1..5: note = TCR amino acid sequence, Donor HD5
ENHRY

SEQ ID NO: 79 - protein, 6 aa, synthetic construct
  REGION 1..6: note = TCR amino acid sequence, Donor HD5
SYGVKD

SEQ ID NO: 80 - protein, 15 aa, synthetic construct
  REGION 1..15: note = TCR amino acid sequence, Donor HD5
CAISVGQGALYEQYF

SEQ ID NO: 81 - protein, 134 aa, synthetic construct
  REGION 1..134: note = TCR amino acid sequence, Donor HD5
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISVGQGALYEQYFGPGTRLTVTE

SEQ ID NO: 82 - protein, 310 aa, synthetic construct
  REGION 1..310: note = TCR amino acid sequence, Donor HD5
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISVGQGALYEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 83 - protein, 312 aa, synthetic construct
  REGION 1..312: note = TCR amino acid sequence, Donor HD5
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISVGQGALYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 84 - protein, 5 aa, synthetic construct
  REGION 1..5: note = TCR amino acid sequence, Donor HD5
SGDLS

SEQ ID NO: 85 - protein, 6 aa, synthetic construct
  REGION 1..6: note = TCR amino acid sequence, Donor HD5
YYNGEE

SEQ ID NO: 86 - protein, 16 aa, synthetic construct
  REGION 1..16: note = TCR amino acid sequence, Donor HD5
CASSVARDRRNYGYTF

SEQ ID NO: 87 - protein, 135 aa, synthetic construct
  REGION 1..135: note = TCR amino acid sequence, Donor HD5
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVARDRRNYGYTFGSGTRLTVVE

SEQ ID NO: 88 - protein, 311 aa, synthetic construct
  REGION 1..311: note = TCR amino acid sequence, Donor HD5
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVARDRRNYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 89 - protein, 313 aa, synthetic construct
  REGION 1..313: note = TCR amino acid sequence, Donor HD5
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVARDRRNYGYTFGSGTRLTVVEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 90 - protein, 6 aa, synthetic construct
  REGION 1..6: note = TCR amino acid sequence, Donor HD6
NSMFDY

SEQ ID NO: 91 - protein, 7 aa, synthetic construct
  REGION 1..7: note = TCR amino acid sequence, Donor HD6
ISSIKDK

SEQ ID NO: 92 - protein, 10 aa, synthetic construct
  REGION 1..10: note = TCR amino acid sequence, Donor HD6
CAANNARLMF

SEQ ID NO: 93 - protein, 136 aa, synthetic construct
  REGION 1..136: note = TCR amino acid sequence, Donor HD6
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAANNARLMFGDGTQLVVKPN

SEQ ID NO: 94 - protein, 276 aa, synthetic construct
  REGION 1..276: note = TCR amino acid sequence, Donor HD6
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAANNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 95 - protein, 5 aa, synthetic construct
  REGION 1..5: note = TCR amino acid sequence, Donor HD6
SGHNS

SEQ ID NO: 96 - protein, 6 aa, synthetic construct
  REGION 1..6: note = TCR amino acid sequence, Donor HD6
FNNNVP

SEQ ID NO: 97 - protein, 13 aa, synthetic construct
  REGION 1..13: note = TCR amino acid sequence, Donor HD6
CASSDTRAREQFF

SEQ ID NO: 98 - protein, 133 aa, synthetic construct
  REGION 1..133: note = TCR amino acid sequence, Donor HD6
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSDTRAREQFFGPGTRLTVLE

SEQ ID NO: 99 - protein, 309 aa, synthetic construct
  REGION 1..309: note = TCR amino acid sequence, Donor HD6
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSDTRAREQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 100 - protein, 311 aa, synthetic construct
  REGION 1..311: note = TCR amino acid sequence, Donor HD6
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSDTRAREQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 101 - protein, 6 aa, synthetic construct
  REGION 1..6: note = TCR amino acid sequence, Donor HD7
DSSSTY

SEQ ID NO: 102 - protein, 7 aa, synthetic construct
  REGION 1..7: note = TCR amino acid sequence, Donor HD7
IFSNMDM

SEQ ID NO: 103 - protein, 12 aa, synthetic construct
  REGION 1..12: note = TCR amino acid sequence, Donor HD7
CAERLNTDKLIF

SEQ ID NO: 104 - protein, 132 aa, synthetic construct
  REGION 1..132: note = TCR amino acid sequence, Donor HD7
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAERLNTDKLIFGTGTRLQVFPN

SEQ ID NO: 105 - protein, 272 aa, synthetic construct
  REGION 1..272: note = TCR amino acid sequence, Donor HD7
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAERLNTDKLIFGTGTRLQVFPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 106 - protein, 5 aa, synthetic construct
  REGION 1..5: note = TCR amino acid sequence, Donor HD7
DSVNN

SEQ ID NO: 107 - protein, 5 aa, synthetic construct
  REGION 1..5: note = TCR amino acid sequence, Donor HD7
IPSGT

SEQ ID NO: 108 - protein, 14 aa, synthetic construct
  REGION 1..14: note = TCR amino acid sequence, Donor HD7
CAVEATDSWGKLQF

SEQ ID NO: 109 - protein, 131 aa, synthetic construct
  REGION 1..131: note = TCR amino acid sequence, Donor HD7
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVEATDSWGKLQFGAGTQVVVTPD

SEQ ID NO: 110 - protein, 271 aa, synthetic construct
  REGION 1..271: note = TCR amino acid sequence, Donor HD7
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVEATDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 111 - protein, 6 aa, synthetic construct
  REGION 1..6: note = TCR amino acid sequence, Donor HD7
DSASNY

SEQ ID NO: 112 - protein, 7 aa, synthetic construct
  REGION 1..7: note = TCR amino acid sequence, Donor HD7
IRSNVGE

SEQ ID NO: 113 - protein, 12 aa, synthetic construct
  REGION 1..12: note = TCR amino acid sequence, Donor HD7
CAVRTSYDKVIF

SEQ ID NO: 114 - protein, 131 aa, synthetic construct
  REGION 1..131: note = TCR amino acid sequence, Donor HD7
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAVRTSYDKVIFGPGTSLSVIPN

SEQ ID NO: 115 - protein, 15 aa, synthetic construct
  REGION 1..15: note = Wilms tumour 1 (WT1) peptide 40
AAQWAPVLDFAPPGA

SEQ ID NO: 116 - protein, 15 aa, synthetic construct
  REGION 1..15: note = WT1 peptide 41
APVLDFAPPGASAYG

SEQ ID NO: 117 - protein, 11 aa, synthetic construct
  REGION 1..11: note = WT1 peptide Overlapping sequence
APVLDFAPPGA

SEQ ID NO: 118 - protein, 15 aa, synthetic construct
  REGION 1..15: note = WT1 peptide 54
QCLSAFTVHFSGQFT

SEQ ID NO: 119 - protein, 15 aa, synthetic construct
  REGION 1..15: note = WT1 peptide 77
EDPMGQQGSLGEQQY

SEQ ID NO: 120 - protein, 15 aa, synthetic construct
  REGION 1..15: note = WT1 peptide 90
SQLECMTWNQMNLGA

SEQ ID NO: 121 - protein, 15 aa, synthetic construct
  REGION 1..15: note = WT1 peptide 17
TCVPEPASQHTLRSG

SEQ ID NO: 122 - protein, 15 aa, synthetic construct
  REGION 1..15: note = WT1 peptide 18
EPASQHTLRSGPGCL

SEQ ID NO: 123 - protein, 11 aa, synthetic construct
  REGION 1..11: note = WT1 peptide Overlapping sequence
EPASQHTLRSG

SEQ ID NO: 124 - protein, 15 aa, synthetic construct
  REGION 1..15: note = WT1 peptide 99
HSTGYESDNHTTPIL

SEQ ID NO: 125 - protein, 15 aa, synthetic construct
  REGION 1..15: note = WT1 peptide 100
YESDNHTTPILCGAQ

SEQ ID NO: 126 - protein, 11 aa, synthetic construct
  REGION 1..11: note = WT1 peptide Overlapping sequence
YESDNHTTPIL

SEQ ID NO: 127 - protein, 15 aa, synthetic construct
  REGION 1..15: note = WT1 peptide 101
NHTTPILCGAQYRIH

SEQ ID NO: 128 - protein, 140 aa, synthetic construct
  REGION 1..140: note = sequence of an alpha chain constant domain encoded by a TRAC gene
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 129 - protein, 176 aa, synthetic construct
  REGION 1..176: note = sequence of a beta chain constant domain encoded by a TRBC1 gene
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 130 - protein, 178 aa, synthetic construct
  REGION 1..178: note = sequence of a beta chain constant domain encoded by a TRBC2 gene
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 131 - protein, 575 aa, synthetic construct
  REGION 1..575: note = WT1 protein sequence
SRQRPHPGALRNPTACPLPHFPPSLPPTHSPTHPPRAGTAAQAPGPRRLLAAILDFLLLQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 132 - DNA, 816 bp, synthetic construct
  misc_feature 1..816: note = Nucleotide sequence encoding TCR, Donor HD1
  source 1..816: mol_type = other DNA
atggagactctcctgaaagtgctttcaggcaccttgttgtggcagttgacctgggtgagaagccaacaaccagtgcagagtcctcaagccgtgatcctccgagaaggggaagatgctgtcatcaactgcagttcctccaaggctttatattctgtacactggtacaggcagaagcatggtgaagcacccgtcttcctgatgatattactgaagggtggagaacagaagggtcatgaaaaaatatctgcttcatttaatgaaaaaaagcagcaaagctccctgtaccttacggcctcccagctcagttactcaggaacctacttctgcggcacagcttggattaacgactacaagctcagctttggagccggaaccacagtaactgtaagagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 133 - DNA, 936 bp, synthetic construct
  misc_feature 1..936: note = Nucleotide sequence encoding TCR, Donor HD1
  source 1..936: mol_type = other DNA
atgggctcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagaaaaaccgggggatatagcaatcagccccagcattttggtgatgggactcgactctccatcctagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 134 - DNA, 942 bp, synthetic construct
  misc_feature 1..942: note = Nucleotide sequence encoding TCR, Donor HD1
  source 1..942: mol_type = other DNA
atgggctcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagaaaaaccgggggatatagcaatcagccccagcattttggtgatgggactcgactctccatcctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 135 - DNA, 822 bp, synthetic construct
  misc_feature 1..822: note = Nucleotide sequence encoding TCR, Donor HD2-1
  source 1..822: mol_type = other DNA
atgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgctgtgagattatctggttctgcaaggcaactgacctttggatctgggacacaattgactgttttacctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 136 - DNA, 924 bp, synthetic construct
  misc_feature 1..924: note = Nucleotide sequence encoding TCR, Donor HD2-1
  source 1..924: mol_type = other DNA
atgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctctgtgccagcagcttactgggagacgagcagtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 137 - DNA, 930 bp, synthetic construct
  misc_feature 1..930: note = Nucleotide sequence encoding TCR, Donor HD2-1
  source 1..930: mol_type = other DNA
atgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctctgtgccagcagcttactgggagacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 138 - DNA, 828 bp, synthetic construct
  misc_feature 1..828: note = Nucleotide sequence encoding TCR, Donor HD2-2
  source 1..828: mol_type = other DNA
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagtctaaaatatggaaacaaactggtctttggcgcaggaaccattctgagagtcaagtcctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 139 - DNA, 936 bp, synthetic construct
  misc_feature 1..936: note = Nucleotide sequence encoding TCR, Donor HD2-2
  source 1..936: mol_type = other DNA
atgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctctgtgccagcagcttggtagctttacagggtgcgggcgagcagtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 140 - DNA, 942 bp, synthetic construct
  misc_feature 1..942: note = Nucleotide sequence encoding TCR, Donor HD2-2
  source 1..942: mol_type = other DNA
atgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctctgtgccagcagcttggtagctttacagggtgcgggcgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 141 - DNA, 822 bp, synthetic construct
  misc_feature 1..822: note = Nucleotide sequence encoding TCR, Donor HD3
  source 1..822: mol_type = other DNA
atgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgaacctcctgtctaaccagggaggaaagcttatcttcggacagggaacggagttatctgtgaaacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 142 - DNA, 942 bp, synthetic construct
  misc_feature 1..942: note = Nucleotide sequence encoding TCR, Donor HD3
  source 1..942: mol_type = other DNA
atgggctgcaggctgctctgctgtgcggttctctgtctcctgggagcgggtgagttggtccccatggaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagccaagattacttggtttctaatgaaaaactgttttttggcagtggaacccagctctctgtcttggaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 143 - DNA, 948 bp, synthetic construct
  misc_feature 1..948: note = Nucleotide sequence encoding TCR, Donor HD3
  source 1..948: mol_type = other DNA
atgggctgcaggctgctctgctgtgcggttctctgtctcctgggagcgggtgagttggtccccatggaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagccaagattacttggtttctaatgaaaaactgttttttggcagtggaacccagctctctgtcttggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 144 - DNA, 819 bp, synthetic construct
  misc_feature 1..819: note = Nucleotide sequence encoding TCR, Donor HD4-1
  source 1..819: mol_type = other DNA
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggacgcgtattcaggaaacacacctcttgtctttggaaagggcacaagactttctgtgattgcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 145 - DNA, 927 bp, synthetic construct
  misc_feature 1..927: note = Nucleotide sequence encoding TCR, Donor HD4-1
  source 1..927: mol_type = other DNA
atgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagggcagcagggttggacactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 146 - DNA, 933 bp, synthetic construct
  misc_feature 1..933: note = Nucleotide sequence encoding TCR, Donor HD4-1
  source 1..933: mol_type = other DNA
atgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagggcagcagggttggacactgaagctttctttggacaaggcaccagactcacagttgtagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 147 - DNA, 825 bp, synthetic construct
  misc_feature 1..825: note = Nucleotide sequence encoding TCR, Donor HD4-2
  source 1..825: mol_type = other DNA
atggagaccctcttgggcctgcttatcctttggctgcagctgcaatgggtgagcagcaaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtccgggcagagatttataaccagggaggaaagcttatcttcggacagggaacggagttatctgtgaaacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 148 - DNA, 921 bp, synthetic construct
  misc_feature 1..921: note = Nucleotide sequence encoding TCR, Donor HD4-2
  source 1..921: mol_type = other DNA
atgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagtacccaaactccctacgagcagtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 149 - DNA, 927 bp, synthetic construct
  misc_feature 1..927: note = Nucleotide sequence encoding TCR, Donor HD4-2
  source 1..927: mol_type = other DNA
atgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagtacccaaactccctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 150 - DNA, 933 bp, synthetic construct
  misc_feature 1..933: note = Nucleotide sequence encoding TCR, Donor HD4-2
  source 1..933: mol_type = other DNA
atggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagcacagtgggaggggaggattatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 151 - DNA, 939 bp, synthetic construct
  misc_feature 1..939: note = Nucleotide sequence encoding TCR, Donor HD4-2
  source 1..939: mol_type = other DNA
atggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagcacagtgggaggggaggattatggctacaccttcggttcggggaccaggttaaccgttgtagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 152 - DNA, 822 bp, synthetic construct
  misc_feature 1..822: note = Nucleotide sequence encoding TCR, Donor HD5
  source 1..822: mol_type = other DNA
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtatggctggggctgggagttaccaactcactttcgggaaggggaccaaactctcggtcataccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 153 - DNA, 930 bp, synthetic construct
  misc_feature 1..930: note = Nucleotide sequence encoding TCR, Donor HD5
  source 1..930: mol_type = other DNA
atgggcacaaggttgttcttctatgtggccctttgtctcctgtggacaggacacatggatgctggaatcacccagagcccaagacacaaggtcacagagacaggaacaccagtgactctgagatgtcaccagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatctcggtgggacagggggccctctacgagcagtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 154 - DNA, 936 bp, synthetic construct
  misc_feature 1..936: note = Nucleotide sequence encoding TCR, Donor HD5
  source 1..936: mol_type = other DNA
atgggcacaaggttgttcttctatgtggccctttgtctcctgtggacaggacacatggatgctggaatcacccagagcccaagacacaaggtcacagagacaggaacaccagtgactctgagatgtcaccagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatctcggtgggacagggggccctctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 155 - DNA, 933 bp, synthetic construct
  misc_feature 1..933: note = Nucleotide sequence encoding TCR, Donor HD5
  source 1..933: mol_type = other DNA
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtagctcgggacaggcggaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 156 - DNA, 939 bp, synthetic construct
  misc_feature 1..939: note = Nucleotide sequence encoding TCR, Donor HD5
  source 1..939: mol_type = other DNA
atgggcttcaggctcctctgctgtgtggccttttgtctcctgggagcaggcccagtggattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtagctcgggacaggcggaactatggctacaccttcggttcggggaccaggttaaccgttgtagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 157 - protein, 9 aa, synthetic construct
  REGION 1..9: note = nonamer peptide VLD
VLDFAPPGA

SEQ ID NO: 158 - protein, 9 aa, synthetic construct
  REGION 1..9: note = nonamer peptide PVL
PVLDFAPPG

SEQ ID NO: 159 - protein, 9 aa, synthetic construct
  REGION 1..9: note = nonamer peptide LDF
LDFAPPGAS

SEQ ID NO: 160 - protein, 271 aa, synthetic construct
  REGION 1..271: note = TCR amino acid sequence, Donor HD7
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAVRTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 161 - protein, 6 aa, synthetic construct
  REGION 1..6: note = TCR amino acid sequence, Donor HD7
DFQATT

SEQ ID NO: 162 - protein, 7 aa, synthetic construct
  REGION 1..7: note = TCR amino acid sequence, Donor HD7
SNEGSKA

SEQ ID NO: 163 - protein, 14 aa, synthetic construct
  REGION 1..14: note = TCR amino acid sequence, Donor HD7
CSARDSVSGNTIYF

SEQ ID NO: 164 - protein, 142 aa, synthetic construct
  REGION 1..142: note = TCR amino acid sequence, Donor HD7
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDSVSGNTIYFGEGSWLTVVE

SEQ ID NO: 165 - protein, 318 aa, synthetic construct
  REGION 1..318: note = TCR amino acid sequence, Donor HD7
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDSVSGNTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 166 - protein, 320 aa, synthetic construct
  REGION 1..320: note = TCR amino acid sequence, Donor HD7
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDSVSGNTIYFGEGSWLTVVEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 167 - protein, 5 aa, synthetic construct
  REGION 1..5: note = TCR amino acid sequence, Donor HD7
SQVTM

SEQ ID NO: 168 - protein, 7 aa, synthetic construct
  REGION 1..7: note = TCR amino acid sequence, Donor HD7
ANQGSEA

SEQ ID NO: 169 - protein, 14 aa, synthetic construct
  REGION 1..14: note = TCR amino acid sequence, Donor HD7
CSVGGSGSYNEQFF

SEQ ID NO: 170 - protein, 131 aa, synthetic construct
  REGION 1..131: note = TCR amino acid sequence, Donor HD7
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVGGSGSYNEQFFGPGTRLTVLE

SEQ ID NO: 171 - protein, 307 aa, synthetic construct
  REGION 1..307: note = TCR amino acid sequence, Donor HD7
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVGGSGSYNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 172 - protein, 309 aa, synthetic construct
  REGION 1..309: note = TCR amino acid sequence, Donor HD7
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVGGSGSYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 173 - protein, 5 aa, synthetic construct
  REGION 1..5: note = TCR amino acid sequence, Donor HD8
VGISA

SEQ ID NO: 174 - protein, 5 aa, synthetic construct
  REGION 1..5: note = TCR amino acid sequence, Donor HD8
LSSGK

SEQ ID NO: 175 - protein, 11 aa, synthetic construct
  REGION 1..11: note = TCR amino acid sequence, Donor HD8
CAVTVGNKLVF

SEQ ID NO: 176 - protein, 130 aa, synthetic construct
  REGION 1..130: note = TCR amino acid sequence, Donor HD8
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAVTVGNKLVFGAGTILRVKSY

SEQ ID NO: 177 - protein, 270 aa, synthetic construct
  REGION 1..270: note = TCR amino acid sequence, Donor HD8
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAVTVGNKLVFGAGTILRVKSYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 178 - protein, 5 aa, synthetic construct
  REGION 1..5: note = TCR amino acid sequence, Donor HD8
MNHNS

SEQ ID NO: 179 - protein, 6 aa, synthetic construct
  REGION 1..6: note = TCR amino acid sequence, Donor HD8
SASEGT

SEQ ID NO: 180 - protein, 11 aa, synthetic construct
  REGION 1..11: note = TCR amino acid sequence, Donor HD8
CASRGWREQFF

SEQ ID NO: 181 - protein, 130 aa, synthetic construct
  REGION 1..130: note = TCR amino acid sequence, Donor HD8
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASRGWREQFFGPGTRLTVLE

SEQ ID NO: 182 - protein, 306 aa, synthetic construct
  REGION 1..306: note = TCR amino acid sequence, Donor HD8
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASRGWREQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 183 - protein, 308 aa, synthetic construct
  REGION 1..308: note = TCR amino acid sequence, Donor HD8
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASRGWREQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 184 - protein, 5 aa, synthetic construct
  REGION 1..5: note = TCR amino acid sequence, Donor HD9
VGISA

SEQ ID NO: 185 - protein, 5 aa, synthetic construct
  REGION 1..5: note = TCR amino acid sequence, Donor HD9
LSSGK

SEQ ID NO: 186 - protein, 13 aa, synthetic construct
  REGION 1..13: note = TCR amino acid sequence, Donor HD9
CAARSYNTDKLIF

SEQ ID NO: 187 - protein, 132 aa, synthetic construct
  REGION 1..132: note = TCR amino acid sequence, Donor HD9
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAARSYNTDKLIFGTGTRLQVFPN

SEQ ID NO: 188 - protein, 272 aa, synthetic construct
  REGION 1..272: note = TCR amino acid sequence, Donor HD9
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAARSYNTDKLIFGTGTRLQVFPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 189 - protein, 6 aa, synthetic construct
  REGION 1..6: note = TCR amino acid sequence, Donor HD9
NSMFDY

SEQ ID NO: 190 - protein, 7 aa, synthetic construct
  REGION 1..7: note = TCR amino acid sequence, Donor HD9
ISSIKDK

SEQ ID NO: 191 - protein, 12 aa, synthetic construct
  REGION 1..12: note = TCR amino acid sequence, Donor HD9
CAASYNNARLMF

SEQ ID NO: 192 - protein, 138 aa, synthetic construct
  REGION 1..138: note = TCR amino acid sequence, Donor HD9
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASYNNARLMFGDGTQLVVKPN

SEQ ID NO: 193 - protein, 278 aa, synthetic construct
  REGION 1..278: note = TCR amino acid sequence, Donor HD9
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASYNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 194 - protein, 5 aa, synthetic construct
  REGION 1..5: note = TCR amino acid sequence, Donor HD9
SGHTS

SEQ ID NO: 195 - protein, 6 aa, synthetic construct
  REGION 1..6: note = TCR amino acid sequence, Donor HD9
YDEGEE

SEQ ID NO: 196 - protein, 13 aa, synthetic construct
  REGION 1..13: note = TCR amino acid sequence, Donor HD9
CASSWGYQETQYF

SEQ ID NO: 197 - protein, 132 aa, synthetic construct
  REGION 1..132: note = TCR amino acid sequence, Donor HD9
MGPRLLFWALLCLLGTGPVEAGVTQSPTHLIKTRGQQATLRCSPISGHTSVYWYQQALGLGLQFLLWYDEGEERNRGNFPPRFSGRQFPNYSSELNVNALELEDSALYLCASSWGYQETQYFGPGTRLLVLE

SEQ ID NO: 198 - protein, 308 aa, synthetic construct
  REGION 1..308: note = TCR amino acid sequence, Donor HD9
MGPRLLFWALLCLLGTGPVEAGVTQSPTHLIKTRGQQATLRCSPISGHTSVYWYQQALGLGLQFLLWYDEGEERNRGNFPPRFSGRQFPNYSSELNVNALELEDSALYLCASSWGYQETQYFGPGTRLLVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 199 - protein, 310 aa, synthetic construct
  REGION 1..310: note = TCR amino acid sequence, Donor HD9
MGPRLLFWALLCLLGTGPVEAGVTQSPTHLIKTRGQQATLRCSPISGHTSVYWYQQALGLGLQFLLWYDEGEERNRGNFPPRFSGRQFPNYSSELNVNALELEDSALYLCASSWGYQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 200 - protein, 5 aa, synthetic construct
  REGION 1..5: note = TCR amino acid sequence, Donor HD9
KGHSH

SEQ ID NO: 201 - protein, 6 aa, synthetic construct
  REGION 1..6: note = TCR amino acid sequence, Donor HD9
LQKENI

SEQ ID NO: 202 - protein, 15 aa, synthetic construct
  REGION 1..15: note = TCR amino acid sequence, Donor HD9
CASSPTGGEYYGYTF

SEQ ID NO: 203 - protein, 135 aa, synthetic construct
  REGION 1..135: note = TCR amino acid sequence, Donor HD9
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPTGGEYYGYTFGSGTRLTVVE

SEQ ID NO: 204 - protein, 311 aa, synthetic construct
  REGION 1..311: note = TCR amino acid sequence, Donor HD9
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPTGGEYYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 205 - protein, 313 aa, synthetic construct
  REGION 1..313: note = TCR amino acid sequence, Donor HD9
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPTGGEYYGYTFGSGTRLTVVEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 206 - protein, 5 aa, synthetic construct
  REGION 1..5: note = TCR amino acid sequence, Donor HD9
MNHEY

SEQ ID NO: 207 - protein, 6 aa, synthetic construct
  REGION 1..6: note = TCR amino acid sequence, Donor HD9
SVGAGI

SEQ ID NO: 208 - protein, 22 aa, synthetic construct
  REGION 1..22: note = TCR amino acid sequence, Donor HD9
CASSSYPLRTGRYNSYNSPLHF

SEQ ID NO: 209 - protein, 141 aa, synthetic construct
  REGION 1..141: note = TCR amino acid sequence, Donor HD9
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSSYPLRTGRYNSYNSPLHFGNGTRLTVTE

SEQ ID NO: 210 - protein, 317 aa, synthetic construct
  REGION 1..317: note = TCR amino acid sequence, Donor HD9
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSSYPLRTGRYNSYNSPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 211 - protein, 319 aa, synthetic construct
  REGION 1..319: note = TCR amino acid sequence, Donor HD9
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSSYPLRTGRYNSYNSPLHFGNGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 212 - protein, 6 aa, synthetic construct
  REGION 1..6: note = TCR amino acid sequence, Donor HD10
NSMFDY

SEQ ID NO: 213 - protein, 7 aa, synthetic construct
  REGION 1..7: note = TCR amino acid sequence, Donor HD10
ISSIKDK

SEQ ID NO: 214 - protein, 13 aa, synthetic construct
  REGION 1..13: note = TCR amino acid sequence, Donor HD10
CAASGGRDDKIIF

SEQ ID NO: 215 - protein, 139 aa, synthetic construct
  REGION 1..139: note = TCR amino acid sequence, Donor HD10
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGGRDDKIIFGKGTRLHILPN

SEQ ID NO: 216 - protein, 279 aa, synthetic construct
  REGION 1..279: note = TCR amino acid sequence, Donor HD10
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGGRDDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 217 - protein, 5 aa, synthetic construct
  REGION 1..5: note = TCR amino acid sequence, Donor HD10
MNHEY

SEQ ID NO: 218 - protein, 6 aa, synthetic construct
  REGION 1..6: note = TCR amino acid sequence, Donor HD10
SVGAGI

SEQ ID NO: 219 - protein, 16 aa, synthetic construct
  REGION 1..16: note = TCR amino acid sequence, Donor HD10
CASSYSRTESTDTQYF

SEQ ID NO: 220 - protein, 135 aa, synthetic construct
  REGION 1..135: note = TCR amino acid sequence, Donor HD10
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSRTESTDTQYFGPGTRLTVLE

SEQ ID NO: 221 - protein, 311 aa, synthetic construct
  REGION 1..311: note = TCR amino acid sequence, Donor HD10
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSRTESTDTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 222 - protein, 313 aa, synthetic construct
  REGION 1..313: note = TCR amino acid sequence, Donor HD10
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSRTESTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 223 - DNA, 828 bp, synthetic construct
  misc_feature 1..828: note = Nucleotide sequence encoding TCR, Donor HD6
  source 1..828: mol_type = other DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaaacaatgccagactcatgtttggagatggaactcagctggtggtgaagcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 224 - DNA, 927 bp, synthetic construct
  misc_feature 1..927: note = Nucleotide sequence encoding TCR, Donor HD6
  source 1..927: mol_type = other DNA
atggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtgataccagggcccgggagcagttcttcgggccagggacacggctcaccgtgctagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 225 - DNA, 933 bp, synthetic construct
  misc_feature 1..933: note = Nucleotide sequence encoding TCR, Donor HD6
  source 1..933: mol_type = other DNA
atggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtgataccagggcccgggagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 226 - DNA, 816 bp, synthetic construct
  misc_feature 1..816: note = Nucleotide sequence encoding TCR, Donor HD7
  source 1..816: mol_type = other DNA
atgaagacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagagaggcttaacaccgacaagctcatctttgggactgggaccagattacaagtctttccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 227 - DNA, 813 bp, synthetic construct
  misc_feature 1..813: note = Nucleotide sequence encoding TCR, Donor HD7
  source 1..813: mol_type = other DNA
atgaagaggatattgggagctctgctggggctcttgagtgcccaggtttgctgtgtgagaggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtggaggcaactgacagctgggggaaattgcagtttggagcagggacccaggttgtggtcaccccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 228 - DNA, 813 bp, synthetic construct
  misc_feature 1..813: note = Nucleotide sequence encoding TCR, Donor HD7
  source 1..813: mol_type = other DNA
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagtacgaacctcctacgacaaggtgatatttgggccagggacaagcttatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 229 - DNA, 954 bp, synthetic construct
  misc_feature 1..954: note = Nucleotide sequence encoding TCR, Donor HD7
  source 1..954: mol_type = other DNA
atgctgctgcttctgctgcttctggggccaggtataagcctccttctacctgggagcttggcaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagggacagtgtgtctggaaacaccatatattttggagagggaagttggctcactgttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 230 - DNA, 960 bp, synthetic construct
  misc_feature 1..960: note = Nucleotide sequence encoding TCR, Donor HD7
  source 1..960: mol_type = other DNA
atgctgctgcttctgctgcttctggggccaggtataagcctccttctacctgggagcttggcaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagggacagtgtgtctggaaacaccatatattttggagagggaagttggctcactgttgtagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 231 - DNA, 921 bp, synthetic construct
  misc_feature 1..921: note = Nucleotide sequence encoding TCR, Donor HD7
  source 1..921: mol_type = other DNA
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgggggtagcgggagttacaatgagcagttcttcgggccagggacacggctcaccgtgctagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 232 - DNA, 927 bp, synthetic construct
  misc_feature 1..927: note = Nucleotide sequence encoding TCR, Donor HD7
  source 1..927: mol_type = other DNA
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgggggtagcgggagttacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 233 - DNA, 810 bp, synthetic construct
  misc_feature 1..810: note = Nucleotide sequence encoding TCR, Donor HD8
  source 1..810: mol_type = other DNA
atggtgaagatccggcaatttttgttggctattttgtggcttcagctaagctgtgtaagtgccgccaaaaatgaagtggagcagagtcctcagaacctgactgcccaggaaggagaatttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgtgctgtcacagtcggaaacaaactggtctttggcgcaggaaccattctgagagtcaagtcctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 234 - DNA, 918 bp, synthetic construct
  misc_feature 1..918: note = Nucleotide sequence encoding TCR, Donor HD8
  source 1..918: mol_type = other DNA
atgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagggggtggcgtgagcagttcttcgggccagggacacggctcaccgtgctagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 235 - DNA, 924 bp, synthetic construct
  misc_feature 1..924: note = Nucleotide sequence encoding TCR, Donor HD8
  source 1..924: mol_type = other DNA
atgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagggggtggcgtgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 236 - DNA, 816 bp, synthetic construct
  misc_feature 1..816: note = Nucleotide sequence encoding TCR, Donor HD9
  source 1..816: mol_type = other DNA
atggtgaagatccggcaatttttgttggctattttgtggcttcagctaagctgtgtaagtgccgccaaaaatgaagtggagcagagtcctcagaacctgactgcccaggaaggagaatttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgtgctgcccgatcttataacaccgacaagctcatctttgggactgggaccagattacaagtctttccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 237 - DNA, 834 bp, synthetic construct
  misc_feature 1..834: note = Nucleotide sequence encoding TCR, Donor HD9
  source 1..834: mol_type = other DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagttacaacaatgccagactcatgtttggagatggaactcagctggtggtgaagcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 238 - DNA, 834 bp, synthetic construct; identical to SEQ ID NO: 237
  misc_feature 1..834: note = Nucleotide sequence encoding TCR, Donor HD9
  source 1..834: mol_type = other DNA

SEQ ID NO: 239 - DNA, 924 bp, synthetic construct
  misc_feature 1..924: note = Nucleotide sequence encoding TCR, Donor HD9
  source 1..924: mol_type = other DNA
atgggacccaggctcctcttctgggcactgctttgtctcctcggaacaggcccagtggaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctctgtgccagcagctgggggtaccaagagacccagtacttcgggccaggcacgcggctcctggtgctcgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 240 - DNA, 930 bp, synthetic construct
  misc_feature 1..930: note = Nucleotide sequence encoding TCR, Donor HD9
  source 1..930: mol_type = other DNA
atgggacccaggctcctcttctgggcactgctttgtctcctcggaacaggcccagtggaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctctgtgccagcagctgggggtaccaagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 241 - DNA, 933 bp, synthetic construct
  misc_feature 1..933: note = Nucleotide sequence encoding TCR, Donor HD9
  source 1..933: mol_type = other DNA
atggacaccagagtactctgctgtgcggtcatctgtcttctgggggcaggtctctcaaatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttatttctgtgccagctcaccgacaggtggcgagtactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 242 - DNA, 939 bp, synthetic construct
  misc_feature 1..939: note = Nucleotide sequence encoding TCR, Donor HD9
  source 1..939: mol_type = other DNA
atggacaccagagtactctgctgtgcggtcatctgtcttctgggggcaggtctctcaaatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttatttctgtgccagctcaccgacaggtggcgagtactatggctacaccttcggttcggggaccaggttaaccgttgtagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 243 - DNA, 954 bp, synthetic construct
  misc_feature 1..954: note = Nucleotide sequence encoding TCR, Donor HD9
  source 1..954: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttcatacccccttcggacagggcgatacaactcctataattcacccctccactttgggaacgggaccaggctcactgtgacagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 244 - DNA, 957 bp, synthetic construct
  misc_feature 1..957: note = Nucleotide sequence encoding TCR, Donor HD9
  source 1..957: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttcatacccccttcggacagggcgatacaactcctataattcacccctccactttgggaacgggaccaggctcactgtgacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 245 - DNA, 837 bp, synthetic construct
  misc_feature 1..837: note = Nucleotide sequence encoding TCR, Donor HD10
  source 1..837: mol_type = other DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagcggaggaagagatgacaagatcatctttggaaaagggacacgacttcatattctccccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 246 - DNA, 933 bp, synthetic construct
  misc_feature 1..933: note = Nucleotide sequence encoding TCR, Donor HD10
  source 1..933: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagctactcccggacagagagcacagatacgcagtattttggcccaggcacccggctgacagtgctcgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 247 - DNA, 939 bp, synthetic construct
  misc_feature 1..939: note = Nucleotide sequence encoding TCR, Donor HD10
  source 1..939: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagctactcccggacagagagcacagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 248 - protein, 15 aa, synthetic construct
  REGION 1..15: note = WT1 peptide 91
CMTWNQMNLGATLKG

SEQ ID NO: 249 - protein, 15 aa, synthetic construct
  REGION 1..15: note = WT1 peptide 92
NQMNLGATLKGVAAG

SEQ ID NO: 250 - protein, 11 aa, synthetic construct
  REGION 1..11: note = WT1 peptide Overlapping sequence
NQMNLGATLKG

SEQ ID NO: 251 - protein, 15 aa, synthetic construct
  REGION 1..15: note = WT1 peptide 24
DPGGIWAKLGAAEAS

SEQ ID NO: 252 - protein, 15 aa, synthetic construct
  REGION 1..15: note = WT1 peptide 101
NHTTPILCGAQYRIH

SEQ ID NO: 253 - protein, 15 aa, synthetic construct
  REGION 1..15: note = WT1 peptide 125
KRHQRRHTGVKPFQC

SEQ ID NO: 254 - protein, 15 aa, synthetic construct
  REGION 1..15: note = WT1 peptide 137
PSCQKKFARSDELVR

SEQ ID NO: 255 - protein, 9 aa, synthetic construct
  REGION 1..9: note = WT1 126-134 epitope
RMFPNAPYL

SEQ ID NO: 256 - DNA, 816 bp, synthetic construct
  misc_feature 1..816: note = HD1a TCR sequence
  source 1..816: mol_type = other DNA
atggaaaccctgctgaaggtgctgagcggcacactgctgtggcagctgacatgggtccgatctcagcagcctgtgcagtctcctcaggccgtgattctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgtggcaccgcctggatcaacgactacaagctgtctttcggagccggcaccacagtgacagtgcgggccaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttcagaatcctgctgctcaaggtggccggcttcaacctgctgatgaccctgagactgtggtccagc

SEQ ID NO: 257 - protein, 272 aa, synthetic construct
  REGION 1..272: note = HD1a TCR sequence
METLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTAWINDYKLSFGAGTTVTVRANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 258 - DNA, 942 bp, synthetic construct
  misc_feature 1..942: note = HD1b TCR sequence
  source 1..942: mol_type = other DNA
atgggatcttggacactgtgttgcgtgtccctgtgcatcctggtggccaagcacacagatgccggcgtgatccagtctcctagacacgaagtgaccgagatgggccaagaagtgaccctgcgctgcaagcctatcagcggccacgattacctgttctggtacagacagaccatgatgagaggcctggaactgctgatctacttcaacaacaacgtgcccatcgacgacagcggcatgcccgaggatagattcagcgccaagatgcccaacgccagcttcagcaccctgaagatccagcctagcgagcccagagatagcgccgtgtacttctgcgccagcagaaagacaggcggctacagcaatcagccccagcactttggagatggcacccggctgagcatcctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggc

SEQ ID NO: 259 - protein, 314 aa, synthetic construct
  REGION 1..314: note = HD1b TCR sequence
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRKTGGYSNQPQHFGDGTRLSILEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 260 - DNA, 822 bp, synthetic construct
  misc_feature 1..822: note = HD3a TCR sequence
  source 1..822: mol_type = other DNA
atgatcagcctgagagtgctgctggtcatcctgtggctgcagctgtcttgggtctggtcccagcggaaagaggtggaacaggaccccggacctttcaatgtgcctgaaggcgccaccgtggccttcaactgcacctacagcaatagcgccagccagagcttcttctggtacagacaggactgccggaaagaacccaagctgctgatgagcgtgtacagcagcggcaacgaggacggcagattcacagcccagctgaacagagccagccagtacatcagcctgctgatccgggatagcaagctgagcgatagcgccacctacctgtgcgtggtcaacctgctgtctaatcaaggcggcaagctgatcttcggccagggcacagagctgagcgtgaagcccaacattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccggatcctgctgctgaaagtggccggcttcaacctcctgatgaccctgagactgtggtccagc

SEQ ID NO: 261 - protein, 274 aa, synthetic construct
  REGION 1..274: note = HD3a TCR sequence
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNLLSNQGGKLIFGQGTELSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 262 - DNA, 948 bp, synthetic construct
  misc_feature 1..948: note = HD3b TCR sequence
  source 1..948: mol_type = other DNA
atgggatgtagacttctgtgttgcgccgtgctgtgtctgcttggagctggcgaactggtgcctatggaaaccggcgtgacccagacacctagacacctggtcatgggcatgacaaacaagaaaagcctgaagtgcgagcagcacctgggccacaatgccatgtactggtacaagcagagcgccaagaaacccctggaactgatgttcgtgtacagcctggaagagagggtcgagaacaacagcgtgcccagcagattcagccctgagtgccctaatagcagccacctgtttctgcatctgcacaccctgcagcctgaggactctgccctgtatctgtgtgccagcagccaggactacctggtgtccaacgagaagctgttcttcggcagcggcacacagctgagcgtgctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggaaaagccactctgtacgctgtgctggtgtccgctctggtgctgatggccatggtcaagcggaaggatagcaggggc

SEQ ID NO: 263 - protein, 316 aa, synthetic construct
  REGION 1..316: note = HD3b TCR sequence
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDYLVSNEKLFFGSGTQLSVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 264 - protein, 6 aa, synthetic construct
  REGION 1..6: note = TCR amino acid sequence, Donor HD6
NSMFDY

SEQ ID NO: 265 - protein, 7 aa, synthetic construct
  REGION 1..7: note = TCR amino acid sequence, Donor HD6
ISSIKDK

SEQ ID NO: 266 - protein, 12 aa, synthetic construct
  REGION 1..12: note = TCR amino acid sequence, Donor HD6
CAASATGNQFYF

SEQ ID NO: 267 - protein, 138 aa, synthetic construct
  REGION 1..138: note = TCR amino acid sequence, Donor HD6
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASATGNQFYFGTGTSLTVIPN

SEQ ID NO: 268 - protein, 278 aa, synthetic construct
  REGION 1..278: note = TCR amino acid sequence, Donor HD6
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASATGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 269 - protein, 5 aa, synthetic construct
  REGION 1..5: note = TCR amino acid sequence, Donor HD6
SGHRS

SEQ ID NO: 270 - protein, 6 aa, synthetic construct
  REGION 1..6: note = TCR amino acid sequence, Donor HD6
YFSETQ

SEQ ID NO: 271 - protein, 13 aa, synthetic construct
  REGION 1..13: note = TCR amino acid sequence, Donor HD6
CASSPGQHGELFF

SEQ ID NO: 272 - protein, 132 aa, synthetic construct
  REGION 1..132: note = TCR amino acid sequence, Donor HD6
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPGQHGELFFGEGSRLTVLE

SEQ ID NO: 273 - protein, 308 aa, synthetic construct
  REGION 1..308: note = TCR amino acid sequence, Donor HD6
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPGQHGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 274 - protein, 310 aa, synthetic construct
  REGION 1..310: note = TCR amino acid sequence, Donor HD6
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPGQHGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 275 - protein, 5 aa, synthetic construct
  REGION 1..5: note = TCR amino acid sequence, Donor HD7
TSINN

SEQ ID NO: 276 - protein, 7 aa, synthetic construct
  REGION 1..7: note = TCR amino acid sequence, Donor HD7
IRSNERE

SEQ ID NO: 277 - protein, 13 aa, synthetic construct
  REGION 1..13: note = TCR amino acid sequence, Donor HD7
CATDGDSSYKLIF

SEQ ID NO: 278 - protein, 132 aa, synthetic construct
  REGION 1..132: note = TCR amino acid sequence, Donor HD7
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDGDSSYKLIFGSGTRLLVRPD

SEQ ID NO: 279 - protein, 272 aa, synthetic construct
  REGION 1..272: note = TCR amino acid sequence, Donor HD7
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDGDSSYKLIFGSGTRLLVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 280 - protein, 6 aa, synthetic construct
  REGION 1..6: note = TCR amino acid sequence, Donor HD7
DFQATT

SEQ ID NO: 281 - protein, 7 aa, synthetic construct
  REGION 1..7: note = TCR amino acid sequence, Donor HD7
SNEGSKA

SEQ ID NO: 282 - protein, 15 aa, synthetic construct
  REGION 1..15: note = TCR amino acid sequence, Donor HD7
CSARDVLTGDYGYTF

SEQ ID NO: 283 - protein, 142 aa, synthetic construct
  REGION 1..142: note = TCR amino acid sequence, Donor HD7
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDVLTGDYGYTFGSGTRLTVV

SEQ ID NO: 284 - protein, 319 aa, synthetic construct
  REGION 1..319: note = TCR amino acid sequence, Donor HD7
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDVLTGDYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 285 - protein, 321 aa, synthetic construct
  REGION 1..321: note = TCR amino acid sequence, Donor HD7
MLLLLLLLGPGISLLLPGSLAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDVLTGDYGYTFGSGTRLTVVEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 286 - protein, 5 aa, synthetic construct
  REGION 1..5: note = TCR amino acid sequence, Donor HD7
SGHDY

SEQ ID NO: 287 - protein, 6 aa, synthetic construct
  REGION 1..6: note = TCR amino acid sequence, Donor HD7
FNNNVP

SEQ ID NO: 288 - protein, 16 aa, synthetic construct
  REGION 1..16: note = TCR amino acid sequence, Donor HD7
CASSLGLSISQETQYF

SEQ ID NO: 289 - protein, 136 aa, synthetic construct
  REGION 1..136: note = TCR amino acid sequence, Donor HD7
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLGLSISQETQYFGPGTRLLVLE

SEQ ID NO: 290 - protein, 312 aa, synthetic construct
  REGION 1..312: note = TCR amino acid sequence, Donor HD7
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLGLSISQETQYFGPGTRLLVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 291 - protein, 314 aa, synthetic construct
  REGION 1..314: note = TCR amino acid sequence, Donor HD7
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLGLSISQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 292 - DNA, 837 bp, synthetic construct
  misc_feature 1..837: note = Nucleotide sequence encoding TCR, Donor HD6
  source 1..837: mol_type = other DNA
atggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagcgctaccggtaaccagttctattttgggacagggacaagtttgacggtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 293 - DNA, 924 bp, synthetic construct
  misc_feature 1..924: note = Nucleotide sequence encoding TCR, Donor HD6
  source 1..924: mol_type = other DNA
atgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagccctggacagcacggggagctgttttttggagaaggctctaggctgaccgtactggaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 294 - DNA, 930 bp, synthetic construct
  misc_feature 1..930: note = Nucleotide sequence encoding TCR, Donor HD6
  source 1..930: mol_type = other DNA
atgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagccctggacagcacggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 295 - DNA, 816 bp, synthetic construct
  misc_feature 1..816: note = Nucleotide sequence encoding TCR, Donor HD7
  source 1..816: mol_type = other DNA
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggacggggatagcagctataaattgatcttcgggagtgggaccagactgctggtcaggcctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 296 - DNA, 957 bp, synthetic construct
  misc_feature 1..957: note = Nucleotide sequence encoding TCR, Donor HD7
  source 1..957: mol_type = other DNA
atgctgctgcttctgctgcttctggggccaggtataagcctccttctacctgggagcttggcaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagacgtactgacaggggactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 297 - DNA, 963 bp, synthetic construct
  misc_feature 1..963: note = Nucleotide sequence encoding TCR, Donor HD7
  source 1..963: mol_type = other DNA
atgctgctgcttctgctgcttctggggccaggtataagcctccttctacctgggagcttggcaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagacgtactgacaggggactatggctacaccttcggttcggggaccaggttaaccgttgtagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 298 - DNA, 936 bp, synthetic construct
  misc_feature 1..936: note = Nucleotide sequence encoding TCR, Donor HD7
  source 1..936: mol_type = other DNA
atgggctcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttaggactgagcatttcccaagagacccagtacttcgggccaggcacgcggctcctggtgctcgaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 299 - DNA, 942 bp, synthetic construct
  misc_feature 1..942: note = Nucleotide sequence encoding TCR, Donor HD7
  source 1..942: mol_type = other DNA
atgggctcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttaggactgagcatttcccaagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

(19) United States
(12) Patent Application Publication
(10) Pub. No.: US 2024/0132569 A1
(43) Pub. Date: Apr. 25, 2024

(54) TCR AND PEPTIDES

(71) Applicants: Ospedale San Raffaele S.r.I., Milan (IT); Fondazione Centro San Raffaele, Milan (IT)

(72) Inventors: Maria Chiara Bonini, Milan (IT); Eliana Ruggiero, Milan (IT); Zulma Irene Magnani, Milan (IT); Luca Aldo Edoardo Vago, Milan (IT); Attilio Bondanza, Milan (IT); Fabio Ciceri, Milan (IT)

(21) Appl. No.: 18/157,632

(22) Filed: Jan. 20, 2023

Related U.S. Application Data
(63) Continuation of application No. 16/605,561, filed on Oct. 16, 2019, now Pat. No. 11,597,755.
(60) Provisional application No. 62/489,226, filed on Apr. 24, 2017.

(51) Int. Cl. C07K 14/725; A61K 35/17; A61P 35/00; C07K 14/47; C12N 5/0783

(52) U.S. Cl. CPC C07K 14/7051; A61K 35/17; A61P 35/00; C07K 14/4748; C12N 5/0636; A61K 38/00

(57) ABSTRACT

A T-cell receptor (TCR), which binds to a Wilms tumour 1 protein (WT1) peptide when presented by a major histocompatibility complex (MHC).

### CROSS-REFERENCE TO RELATED APPLICATIONS

This application is a continuation of U.S. patent application Ser. No. 16/605,561, filed (§ 371(c)) on Oct. 16, 2019, incorporated herein by reference in its entirety; which is a U.S. National Phase of International Patent Application No. PCT/EP2018/060477, filed on Apr. 24, 2018; which claims the benefit under 35 USC § 119 of U.S. Provisional Application No. 62/489,226, filed on Apr. 24, 2017.

### INCORPORATION BY REFERENCE OF MATERIAL SUBMITTED ELECTRONICALLY

This application incorporates by reference in its entirety a computer-readable nucleotide/amino acid sequence listing identified as 51974B_Seqlisting.XML; Size: 457,343 bytes; Created: Jan. 18, 2023.

### FIELD OF THE INVENTION

The present invention relates to T-cell receptors (TCRs) which bind to peptides derived from Wilms tumour 1 protein (WT1) when presented by a major histocompatibility complex. In this regard, the present invention relates to complementarity determining regions (CDRs) which specifically recognise WT1 peptides. The present invention further relates to immunogenic peptides derived from WT1.

### BACKGROUND TO THE INVENTION

T cell receptor (TCR) gene therapy is based on the genetic transfer of high-avidity tumour-specific TCR genes into T lymphocytes, thus enabling the specific targeting of the desired tumour-associated antigens and leading to a less toxic and more specific and effective therapy. This approach has shown promise in clinical trials. One of the main barriers limiting the exploitation of TCR gene therapy for clinical treatment of cancers is the lack of tumour-specific T-cells and corresponding TCRs. Thus, the low availability of tumour-specific TCRs still remains an open issue limiting the broad exploitation of TCR-based immunotherapeutic approaches.

The majority of tumour-associated antigens (TAAs) are self antigens, thus T-cells specific for such molecules are either destroyed or anergized due to central and peripheral tolerance. Despite this, naturally occurring tumour-specific T-cells have been observed in healthy donors and patients, particularly in patients affected by hematological malignancies, after allogeneic hematopoietic stem cell transplantation (allo-HSCT) where frequencies of tumor-specific lymphocytes have been correlated with disease regression (Kapp, M. et al. Bone Marrow Transplantation 43, 399-410 (2009); and Tyler, E. M. et al. Blood 121, 308-317 (2013)).

The choice of a tumor antigen to be targeted by immunotherapeutic approaches is still a matter of debate. Ideal TAAs are highly expressed on tumor cells while being minimally expressed in healthy tissue.

Wilms tumor 1 (WT1) is an intracellular protein encoding a zinc finger transcription factor that plays an important role in cell growth and differentiation (Yang, L. et al. Leukemia 21, 868-876 (2007)). WT1 is widely expressed on a variety of hematological and solid tumors, while showing limited expression on various healthy tissues (e.g. gonads, uterus, kidney, mesothelium, progenitor cells in different tissues). Recent evidence suggests a role for WT1 in leukemogenesis and tumorigenesis.

Several ongoing clinical trials rely on the generation of cytotoxic T lymphocyte (CTL) responses upon vaccination with WT1 peptides. However, despite the recognition that WT1 is useful for immunotherapy, a small number of WT1 epitopes, which are restricted to a limited number of HLA alleles, are presently used for vaccination purposes (Di Stasi, A. et al. Front. Immunol. (2015)). One such epitope is the WT1 126-134 epitope (RMFPNAPYL; SEQ ID NO: 255), which is presented by MHC encoded by the HLA-A*0201 allele (i.e. the epitope is HLA-A*0201 restricted).

HLA-A*0201 restricted epitopes and corresponding TCRs are of interest since major histocompatibility complex (MHC) having the HLA-A*0201 haplotype are expressed in the vast majority (60%) of the Caucasian population. Accordingly, TCRs that target HLA-A*0201-restricted WT1 epitopes are particularly advantageous since an immunotherapy making use of such TCRs may be widely applied.

The WT1 126-134 epitope has been widely studied in several trials, alone or in combination with additional tumor antigens. However, recent reports have highlighted a major concern regarding the processing of this particular epitope, which may impair its use for immunotherapy purposes. Notably, the WT1 126-134 epitope is more efficiently processed by the immunoproteasome compared with standard proteasomes (Jaigirdar, A. et al. J Immunother. 39(3):105-16 (2016)), which leads to poor recognition of many HLA-A*0201 tumour cell lines or primary leukemia cells that endogenously express WT1.

Thus, there remains a need for new WT1 epitopes, particularly those presented by MHC with prevalent HLA haplotypes (e.g. HLA-A*0201).

One naturally processed HLA-A*0201 restricted epitope that has been identified is WT1 37-45, which has the amino acid sequence VLDFAPPGA (SEQ ID NO: 157, see e.g. Smithgall et al 2001; Blood 98 (11 Part 1): 121a). However, few TCR amino acid sequences, particularly CDR sequences, specific for this peptide sequence have been reported (Schmitt, T. M. et al. (2017) Nat Biotechnol 35: 1188-1195).

Accordingly, there remains a need for new WT1 epitopes, particularly those restricted to common HLA alleles and a need for new TCRs capable of binding to WT1 epitopes.

### SUMMARY OF THE INVENTION

We have identified novel TCRs that bind to WT1 peptides when presented by an MHC. Further, we have determined the amino acid sequences of the TCRs, including the amino acid sequences of their CDR regions, which are responsible for binding specificity for WT1. Moreover, we have demonstrated that T-cells expressing TCRs according to the present invention specifically target and kill cells that overexpress the WT1 protein. In addition, it has been shown that the TCRs of the present invention are restricted to MHC encoded by HLA class 1 and 2 alleles common in the Caucasian population, such as HLA-A*0201 and HLA-B*3501 or HLA-B*3502.

Accordingly, in a first aspect, the present invention provides a T-cell receptor (TCR), which binds to a Wilms tumour 1 protein (WT1) peptide when presented by a major histocompatibility complex (MHC), wherein the TCR:
(i) comprises a CDR3α comprising the amino acid sequence of CGTAWINDYKLSF (SEQ ID NO: 3) or a variant thereof having up to three amino acid substitutions, additions or deletions, and a CDR3β comprising the amino acid sequence of CASRKTGGYSNQPQHF (SEQ ID NO: 8) or a variant thereof having up to three amino acid substitutions, additions or deletions;
(ii) comprises a CDR3α comprising the amino acid sequence of CVVNLLSNQGGKLIF (SEQ ID NO: 36) or a variant thereof having up to three amino acid substitutions, additions or deletions, and a CDR3β comprising the amino acid sequence of CASSQDYLVSNEKLFF (SEQ ID NO: 41) or a variant thereof having up to three amino acid substitutions, additions or deletions;
(iii) comprises a CDR3α comprising the amino acid sequence of CAVRLSGSARQLTF (SEQ ID NO: 14) or a variant thereof having up to three amino acid substitutions, additions or deletions, and a CDR3β comprising the amino acid sequence of CASSLLGDEQYF (SEQ ID NO: 24) or a variant thereof having up to three amino acid substitutions, additions or deletions;
(iv) comprises a CDR3α comprising the amino acid sequence of CAVRLSGSARQLTF (SEQ ID NO: 14) or a variant thereof having up to three amino acid substitutions, additions or deletions, and a CDR3β comprising the amino acid sequence of CASSLVALQGAGEQYF (SEQ ID NO: 30) or a variant thereof having up to three amino acid substitutions, additions or deletions;
(v) comprises a CDR3α comprising the amino acid sequence of CAYRSLKYGNKLVF (SEQ ID NO: 19) or a variant thereof having up to three amino acid substitutions, additions or deletions, and a CDR3β comprising the amino acid sequence of CASSLLGDEQYF (SEQ ID NO: 24) or a variant thereof having up to three amino acid substitutions, additions or deletions;
(vi) comprises a CDR3α comprising the amino acid sequence of CAYRSLKYGNKLVF (SEQ ID NO: 19) or a variant thereof having up to three amino acid substitutions, additions or deletions, and a CDR3β comprising the amino acid sequence of CASSLVALQGAGEQYF (SEQ ID NO: 30) or a variant thereof having up to three amino acid substitutions, additions or deletions;
(vii) comprises a CDR3α comprising the amino acid sequence of CATDAYSGNTPLVF (SEQ ID NO: 47) or a variant thereof having up to three amino acid substitutions, additions or deletions, and a CDR3β comprising the amino acid sequence of CASRAAGLDTEAFF (SEQ ID NO: 57) or a variant thereof having up to three amino acid substitutions, additions or deletions;
(viii) comprises a CDR3α comprising the amino acid sequence of CATDAYSGNTPLVF (SEQ ID NO: 47) or a variant thereof having up to three amino acid substitutions, additions or deletions, and a CDR3β comprising the amino acid sequence of CASTQTPYEQYF (SEQ ID NO: 63) or a variant thereof having up to three amino acid substitutions, additions or deletions;
(ix) comprises a CDR3α comprising the amino acid sequence of CATDAYSGNTPLVF (SEQ ID NO: 47) or a variant thereof having up to three amino acid substitutions, additions or deletions, and a CDR3β comprising the amino acid sequence of CASSTVGGEDYGYTF (SEQ ID NO: 69) or a variant thereof having up to three amino acid substitutions, additions or deletions;
(x) comprises a CDR3α comprising the amino acid sequence of CAVRAEIYNQGGKLIF (SEQ ID NO: 52) or a variant thereof having up to three amino acid substitutions, additions or deletions, and a CDR3β comprising the amino acid sequence of CASRAAGLDTEAFF (SEQ ID NO:57) or a variant thereof having up to three amino acid substitutions, additions or deletions;
(xi) comprises a CDR3α comprising the amino acid sequence of CAVRAEIYNQGGKLIF (SEQ ID NO: 52) or a variant thereof having up to three amino acid substitutions, additions or deletions, and a CDR3β comprising the amino acid sequence of CASTQTPYEQYF (SEQ ID NO: 63) or a variant thereof having up to three amino acid substitutions, additions or deletions;
(xii) comprises a CDR3α comprising the amino acid sequence of CAVRAEIYNQGGKLIF (SEQ ID NO: 52) or a variant thereof having up to three amino acid substitutions, additions or deletions, and a CDR3β comprising the amino acid sequence of CASSTVGGEDYGYTF (SEQ ID NO: 69) or a variant thereof having up to three amino acid substitutions, additions or deletions;
(xiii) comprises a CDR3α comprising the amino acid sequence of CAASMAGAGSYQLTF (SEQ ID NO: 75) or a variant thereof having up to three amino acid substitutions, additions or deletions, and a CDR3β comprising the amino acid sequence of CAISVGQGALYEQYF (SEQ ID NO: 80) or a variant thereof having up to three amino acid substitutions, additions or deletions;
(xiv) comprises a CDR3α comprising the amino acid sequence of CAASMAGAGSYQLTF (SEQ ID NO: 75) or a variant thereof having up to three amino acid substitutions, additions or deletions, and a CDR3β comprising the amino acid sequence of CASSVARDRRNYGYTF (SEQ ID NO: 86) or a variant thereof having up to three amino acid substitutions, additions or deletions;
(xv) comprises a CDR3α comprising the amino acid sequence of CAANNARLMF (SEQ ID NO: 92) or a variant thereof having up to three amino acid substitutions, additions or deletions, and a CDR3β comprising the amino acid sequence of CASSDTRAREQFF (SEQ ID NO: 97) or a variant thereof having up to three amino acid substitutions, additions or deletions;
(xvi) comprises a CDR3α comprising the amino acid sequence of CAERLNTDKLIF (SEQ ID NO: 103) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions;
(xvii) comprises a CDR3α comprising the amino acid sequence of CAERLNTDKLIF (SEQ ID NO: 103) or a variant thereof having up to three amino acid substitutions, additions or deletions, and a CDR3β comprising the amino acid sequence of CSVGGSGSYNEQFF (SEQ ID NO: 169) or a variant thereof having up to three amino acid substitutions, additions or deletions;
(xviii) comprises a CDR3α comprising the amino acid sequence of CAVEATDSWGKLQF (SEQ ID NO: 108) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions;
(xix) comprises a CDR3α comprising the amino acid sequence of CAVEATDSWGKLQF (SEQ ID NO: 108) or a variant thereof having up to three amino acid substitutions, additions or deletions, and a CDR3β comprising the amino acid sequence of CSVGGSGSYNEQFF (SEQ ID NO: 169) or a variant thereof having up to three amino acid substitutions, additions or deletions;
(xx) comprises a CDR3α comprising the amino acid sequence of CAVRTSYDKVIF (SEQ ID NO: 113) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions;
(xxi) comprises a CDR3α comprising the amino acid sequence of CAVRTSYDKVIF (SEQ ID NO: 113) or a variant thereof having up to three amino acid substitutions, additions or deletions, and a CDR3β comprising the amino acid sequence of CSVGGSGSYNEQFF (SEQ ID NO: 169) or a variant thereof having up to three amino acid substitutions, additions or deletions;
(xxii) comprises a CDR3α comprising the amino acid sequence of CAVTVGNKLVF (SEQ ID NO: 175) or a variant thereof having up to three amino acid substitutions, additions or deletions, and a CDR3β comprising the amino acid sequence of CASRGWREQFF (SEQ ID NO: 180) or a variant thereof having up to three amino acid substitutions, additions or deletions;
(xxiii) comprises a CDR3α comprising the amino acid sequence of CAARSYNTDKLIF (SEQ ID NO: 186) or a variant thereof having up to three amino acid substitutions, additions or deletions, and a CDR3f3 comprising the amino acid sequence of CASSWGYQETQYF (SEQ ID NO: 196) or a variant thereof having up to three amino acid substitutions, additions or deletions;
(xxiv) comprises a CDR3α comprising the amino acid sequence of CAARSYNTDKLIF (SEQ ID NO: 186) or a variant thereof having up to three amino acid substitutions, additions or deletions, and a CDR3β comprising the amino acid sequence of CASSPTGGEYYGYTF (SEQ ID NO: 202) or a variant thereof having up to three amino acid substitutions, additions or deletions;
(xxv) comprises a CDR3α comprising the amino acid sequence of CAARSYNTDKLIF (SEQ ID NO: 186) or a variant thereof having up to three amino acid substitutions, additions or deletions, and a CDR3β comprising the amino acid sequence of CASSSYPLRTGRYNSYNSPLHF (SEQ ID NO: 208) or a variant thereof having up to three amino acid substitutions, additions or deletions;
(xxvi) comprises a CDR3α comprising the amino acid sequence of CAASYNNARLMF (SEQ ID NO: 191) or a variant thereof having up to three amino acid substitutions, additions or deletions, and a CDR3β comprising the amino acid sequence of CASSWGYQETQYF (SEQ ID NO: 196) or a variant thereof having up to three amino acid substitutions, additions or deletions;
(xxvii) comprises a CDR3α comprising the amino acid sequence of CAASYNNARLMF (SEQ ID NO: 191) or a variant thereof having up to three amino acid substitutions, additions or deletions, and a CDR3β comprising the amino acid sequence of CASSPTGGEYYGYTF (SEQ ID NO: 202) or a variant thereof having up to three amino acid substitutions, additions or deletions;
(xxviii) comprises a CDR3α comprising the amino acid sequence of CAASYNNARLMF (SEQ ID NO: 191) or a variant thereof having up to three amino acid substitutions, additions or deletions, and a CDR3β comprising the amino acid sequence of CASSSYPLRTGRYNSYNSPLHF (SEQ ID NO: 208) or a variant thereof having up to three amino acid substitutions, additions or deletions;
(xxix) comprises a CDR3α comprising the amino acid sequence of CAASGGRDDKIIF (SEQ ID NO: 214) or a variant thereof having up to three amino acid substitutions, additions or deletions, and a CDR3β comprising the amino acid sequence of CASSYSRTESTDTQYF (SEQ ID NO: 219) or a variant thereof having up to three amino acid substitutions, additions or deletions;
(xxx) comprises a CDR3α comprising the amino acid sequence of CAANNARLMF (SEQ ID NO: 92) or a variant thereof having up to three amino acid substitutions, additions or deletions, and a CDR3β comprising the amino acid sequence of CASSPGQHGELFF (SEQ ID NO: 271) or a variant thereof having up to three amino acid substitutions, additions or deletions;
(xxxi) comprises a CDR3α comprising the amino acid sequence of CAASATGNQFYF (SEQ ID NO: 266) or a variant thereof having up to three amino acid substitutions, additions or deletions, and a CDR3β comprising the amino acid sequence of CASSDTRAREQFF (SEQ ID NO: 97) or a variant thereof having up to three amino acid substitutions, additions or deletions;
(xxxii) comprises a CDR3α comprising the amino acid sequence of CAASATGNQFYF (SEQ ID NO: 266) or a variant thereof having up to three amino acid substitutions, additions or deletions, and a CDR3β comprising the amino acid sequence of CASSPGQHGELFF (SEQ ID NO: 271) or a variant thereof having up to three amino acid substitutions, additions or deletions;
(xxxiii) comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions;
(xxxiv) comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSVGGSGSYNEQFF (SEQ ID NO: 169) or a variant thereof having up to three amino acid substitutions, additions or deletions;
(xxxv) comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions;
(xxxvi) comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CASSLGLSISQETQYF (SEQ ID NO: 288) or a variant thereof having up to three amino acid substitutions, additions or deletions;
(xxxvii) comprises a CDR3α comprising the amino acid sequence of CAERLNTDKLIF (SEQ ID NO: 103) or a variant thereof having up to three amino acid substitutions, additions or deletions, and a CDR3β comprising the amino acid sequence of CASSLGLSISQETQYF (SEQ ID NO: 288) or a variant thereof having up to three amino acid substitutions, additions or deletions;
(xxxviii) comprises a CDR3α comprising the amino acid sequence of CAVEATDSWGKLQF (SEQ ID NO: 108) or a variant thereof having up to three amino acid substitutions, additions or deletions, and a CDR3β comprising the amino acid sequence of CASSLGLSISQETQYF (SEQ ID NO: 288) or a variant thereof having up to three amino acid substitutions, additions or deletions;
(xxxix) comprises a CDR3α comprising the amino acid sequence of CAVRTSYDKVIF (SEQ ID NO: 113) or a variant thereof having up to three amino acid substitutions, additions or deletions, and a CDR3β comprising the amino acid sequence of CASSLGLSISQETQYF (SEQ ID NO: 288) or a variant thereof having up to three amino acid substitutions, additions or deletions;
(xxxx) comprises a CDR3α comprising the amino acid sequence of CAERLNTDKLIF (SEQ ID NO: 103) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions;
(xxxxi) comprises a CDR3α comprising the amino acid sequence of CAVEATDSWGKLQF (SEQ ID NO: 108) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions; or
(xxxxii) comprises a CDR3α comprising the amino acid sequence of CAVRTSYDKVIF (SEQ ID NO: 113) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions.

In one embodiment, the present invention provides a TCR of the present invention comprising the following CDR sequences:

(i)
(SEQ ID NO: 1)
CDR1α - KALYS,
(SEQ ID NO: 2)
CDR2α - LLKGGEQ,
(SEQ ID NO: 3)
CDR3α - CGTAWINDYKLSF,
(SEQ ID NO: 6)
CDR1β - SGHDY,
(SEQ ID NO: 7)
CDR2β - FNNNVP,
and
(SEQ ID NO: 8)
CDR3β - CASRKTGGYSNQPQHF,

or variants thereof each having up to three amino acid substitutions, additions or deletions;

(ii)
(SEQ ID NO: 34)
CDR1α - NSASQS,
(SEQ ID NO: 35)
CDR2α - VYSSGN,
(SEQ ID NO: 36)
CDR3α - CVVNLLSNQGGKLIF,
(SEQ ID NO: 39)
CDR1β - LGHNA,
(SEQ ID NO: 40)
CDR2β - YSLEER,
and
(SEQ ID NO: 41)
CDR3β - CASSQDYLVSNEKLFF,

or variants thereof each having up to three amino acid substitutions, additions or deletions;

(iii)
CDR1α-
(SEQ ID NO: 12)
SSVPPY,
CDR2α-
(SEQ ID NO: 13)
YTSAATLV,
CDR3α-
(SEQ ID NO: 14)
CAVRLSGSARQLTF,
CDR1β-
(SEQ ID NO: 22)
SGHAT,
CDR2β-
(SEQ ID NO: 23)
FQNNGV,
and
CDR3β-
(SEQ ID NO: 24)
CASSLLGDEQYF,

or variants thereof each having up to three amino acid substitutions, additions or deletions;

(iv)
CDR1α -
(SEQ ID NO: 12)
SSVPPY,
CDR2α -
(SEQ ID NO: 13)
YTSAATLV,
CDR3α -
(SEQ ID NO: 14)
CAVRLSGSARQLTF,
CDR1β -
(SEQ ID NO: 28)
SGHTA,
CDR2β -
(SEQ ID NO: 29)
FQGNSA,
and
CDR3β -
(SEQ ID NO: 30)
CASSLVALQGAGEQYF,

or variants thereof each having up to three amino acid substitutions, additions or deletions;

(v)
CDR1α -
(SEQ ID NO: 17)
TSESDYY,
CDR2α -
(SEQ ID NO: 18)
QEAYKQQN,
CDR3α -
(SEQ ID NO: 19)
CAYRSLKYGNKLVF,
CDR1β -
(SEQ ID NO: 22)
SGHAT,
CDR2β -
(SEQ ID NO: 23)
FQNNGV,
and
CDR3β -
(SEQ ID NO: 24)
CASSLLGDEQYF,

or variants thereof each having up to three amino acid substitutions, additions or deletions;

(vi)
CDR1α -
(SEQ ID NO: 17)
TSESDYY,
CDR2α -
(SEQ ID NO: 18)
QEAYKQQN,
CDR3α -
(SEQ ID NO: 19)
CAYRSLKYGNKLVF,
CDR1β -
(SEQ ID NO: 28)
SGHTA,
CDR2β -
(SEQ ID NO: 29)
FQGNSA,
and
CDR3β -
(SEQ ID NO: 30)
CASSLVALQGAGEQYF,

or variants thereof each having up to three amino acid substitutions, additions or deletions;

(vii)
CDR1α -
(SEQ ID NO: 45)
TSINN,
CDR2α -
(SEQ ID NO: 46)
IRSNERE,
CDR3α -
(SEQ ID NO: 47)
CATDAYSGNTPLVF,
CDR1β -
(SEQ ID NO: 55)
MNHNS,
CDR2β -
(SEQ ID NO: 56)
SASEGT,
and
CDR3β -
(SEQ ID NO: 57)
CASRAAGLDTEAFF,

or variants thereof each having up to three amino acid substitutions, additions or deletions;

(viii)
CDR1α -
(SEQ ID NO: 45)
TSINN,
CDR2α -
(SEQ ID NO: 46)
IRSNERE,
CDR3α -
(SEQ ID NO: 47)
CATDAYSGNTPLVF,
CDR1β -
(SEQ ID NO: 61)
MNHNY,
CDR2β -
(SEQ ID NO: 62)
SVGAGI,
and
CDR3β -
(SEQ ID NO: 63)
CASTQTPYEQYF,

or variants thereof each having up to three amino acid substitutions, additions or deletions;

(ix)
CDR1α -
(SEQ ID NO: 45)
TSINN,
CDR2α -
(SEQ ID NO: 46)
IRSNERE,
CDR3α -
(SEQ ID NO: 47)
CATDAYSGNTPLVF,
CDR1β -
(SEQ ID NO: 67)
SGHNS,
CDR2β -
(SEQ ID NO: 68)
FNNNVP,
and
CDR3β -
(SEQ ID NO: 69)
CASSTVGGEDYGYTF,

or variants thereof each having up to three amino acid substitutions, additions or deletions;

(x)
CDR1α -
(SEQ ID NO: 50)
DSAIYN,
CDR2α -
(SEQ ID NO: 51)
IQSSQRE,
CDR3α -
(SEQ ID NO: 52)
CAVRAEIYNQGGKLIF,
CDR1β -
(SEQ ID NO: 55)
MNHNS,
CDR2β -
(SEQ ID NO: 56)
SASEGT,
and
CDR3β -
(SEQ ID NO: 57)
CASRAAGLDTEAFF,

or variants thereof each having up to three amino acid substitutions, additions or deletions;

(xi)
CDR1α -
(SEQ ID NO: 50)
DSAIYN,
CDR2α -
(SEQ ID NO: 51)
IQSSQRE,
CDR3α -
(SEQ ID NO: 52)
CAVRAEIYNQGGKLIF,
CDR1β -
(SEQ ID NO: 61)
MNHNY,
CDR2β -
(SEQ ID NO: 62)
SVGAGI,
and
CDR3β -
(SEQ ID NO: 63)
CASTQTPYEQYF,

or variants thereof each having up to three amino acid substitutions, additions or deletions;

(xii)
CDR1α -
(SEQ ID NO: 50)
DSAIYN,
CDR2α -
(SEQ ID NO: 51)
IQSSQRE,
CDR3α -
(SEQ ID NO: 52)
CAVRAEIYNQGGKLIF,
CDR1β -
(SEQ ID NO: 67)
SGHNS,
CDR2β -
(SEQ ID NO: 68)
FNNNVP,
and
CDR3β -
(SEQ ID NO: 69)
CASSTVGGEDYGYTF,

or variants thereof each having up to three amino acid substitutions, additions or deletions;

(xiii)
CDR1α -
(SEQ ID NO: 73)
DSASNY,
CDR2α -
(SEQ ID NO: 74)
IRSNVGE,
CDR3α -
(SEQ ID NO: 75)
CAASMAGAGSYQLTF,
CDR1β -
(SEQ ID NO: 78)
ENHRY,
CDR2β -
(SEQ ID NO: 79)
SYGVKD,
and
CDR3β -
(SEQ ID NO: 80)
CAISVGQGALYEQYF,

or variants thereof each having up to three amino acid substitutions, additions or deletions;

(xiv)
CDR1α -
(SEQ ID NO: 73)
DSASNY,
CDR2α -
(SEQ ID NO: 74)
IRSNVGE,
CDR3α -
(SEQ ID NO: 75)
CAASMAGAGSYQLTF,
CDR1β -
(SEQ ID NO: 84)
SGDLS,
CDR2β -
(SEQ ID NO: 85)
YYNGEE,
and
CDR3β -
(SEQ ID NO: 86)
CASSVARDRRNYGYTF,

or variants thereof each having up to three amino acid substitutions, additions or deletions;

(xv)
CDR1α -
(SEQ ID NO: 90)
NSMFDY,
CDR2α -
(SEQ ID NO: 91)
ISSIKDK,
CDR3α -
(SEQ ID NO: 92)
CAANNARLMF,
CDR1β -
(SEQ ID NO: 95)
SGHNS,
CDR2β -
(SEQ ID NO: 96)
FNNNVP,
and
CDR3β -
(SEQ ID NO: 97)
CASSDTRAREQFF,

or variants thereof each having up to three amino acid substitutions, additions or deletions;

(xvi)
CDR1α -
(SEQ ID NO: 101)
DSSSTY,
CDR2α -
(SEQ ID NO: 102)
IFSNMDM,
CDR3α -
(SEQ ID NO: 103)
CAERLNTDKLIF,
CDR1β -
(SEQ ID NO: 161)
DFQATT,
CDR2β -
(SEQ ID NO: 162)
SNEGSKA,
and
CDR3β -
(SEQ ID NO: 163)
CSARDSVSGNTIYF,

or variants thereof each having up to three amino acid substitutions, additions or deletions;

(xvii)
CDR1α -
(SEQ ID NO: 101)
DSSSTY,
CDR2α -
(SEQ ID NO: 102)
IFSNMDM,
CDR3α -
(SEQ ID NO: 103)
CAERLNTDKLIF,
CDR1β -
(SEQ ID NO: 167)
SQVTM,
CDR2β -
(SEQ ID NO: 168)
ANQGSEA,
and
CDR3β -
(SEQ ID NO: 169)
CSVGGSGSYNEQFF,

or variants thereof each having up to three amino acid substitutions, additions or deletions;

(xviii)
CDR1α -
(SEQ ID NO: 106)
DSVNN,
CDR2α -
(SEQ ID NO: 107)
IPSGT,
CDR3α -
(SEQ ID NO: 108)
CAVEATDSWGKLQF,
CDR1β -
(SEQ ID NO: 161)
DFQATT,
CDR2β -
(SEQ ID NO: 162)
SNEGSKA,
and
CDR3β -
(SEQ ID NO: 163)
CSARDSVSGNTIYF,

or variants thereof each having up to three amino acid substitutions, additions or deletions;

(xix)
(SEQ ID NO: 106)
CDR1α - DSVNN,
(SEQ ID NO: 107)
CDR2α - IPSGT,
(SEQ ID NO: 108)
CDR3α - CAVEATDSWGKLQF,
(SEQ ID NO: 167)
CDR1β - SQVTM,
(SEQ ID NO: 168)
CDR2β - ANQGSEA,
and
(SEQ ID NO: 169)
CDR3β - CSVGGSGSYNEQFF,

or variants thereof each having up to three amino acid substitutions, additions or deletions;

(xx)
(SEQ ID NO: 111)
CDR1α - DSASNY,
(SEQ ID NO: 112)
CDR2α - IRSNVGE,
(SEQ ID NO: 113)
CDR3α - CAVRTSYDKVIF,
(SEQ ID NO: 161)
CDR1β - DFQATT,
(SEQ ID NO: 162)
CDR2β - SNEGSKA,
and
(SEQ ID NO: 163)
CDR3β - CSARDSVSGNTIYF,

or variants thereof each having up to three amino acid substitutions, additions or deletions;

(xxi)
(SEQ ID NO: 111)
CDR1α - DSASNY,
(SEQ ID NO: 112)
CDR2α - IRSNVGE,
(SEQ ID NO: 113)
CDR3α - CAVRTSYDKVIF,
(SEQ ID NO: 167)
CDR1β - SQVTM,
(SEQ ID NO: 168)
CDR2β - ANQGSEA,
and
(SEQ ID NO: 169)
CDR3β - CSVGGSGSYNEQFF,

or variants thereof each having up to three amino acid substitutions, additions or deletions;

(xxii)
(SEQ ID NO: 173)
CDR1α - VGISA,
(SEQ ID NO: 174)
CDR2α - LSSGK,
(SEQ ID NO: 175)
CDR3α - CAVTVGNKLVF,
(SEQ ID NO: 178)
CDR1β - MNHNS,
(SEQ ID NO: 179)
CDR2β - SASEGT,
and
(SEQ ID NO: 180)
CDR3β - CASRGWREQFF,

or variants thereof each having up to three amino acid substitutions, additions or deletions;

(xxiii)
(SEQ ID NO: 184)
CDR1α - VGISA,
(SEQ ID NO: 185)
CDR2α - LSSGK,
(SEQ ID NO: 186)
CDR3α - CAARSYNTDKLIF,
(SEQ ID NO: 194)
CDR1β - SGHTS,
(SEQ ID NO: 195)
CDR2β - YDEGEE,
and
(SEQ ID NO: 196)
CDR3β - CASSWGYQETQYF,

or variants thereof each having up to three amino acid substitutions, additions or deletions;

(xxiv)
(SEQ ID NO: 184)
CDR1α - VGISA,
(SEQ ID NO: 185)
CDR2α - LSSGK,
(SEQ ID NO: 186)
CDR3α - CAARSYNTDKLIF,
(SEQ ID NO: 200)
CDR1β - KGHSH,
(SEQ ID NO: 201)
CDR2β - LQKENI,
and
(SEQ ID NO: 202)
CDR3β - CASSPTGGEYYGYTF,

or variants thereof each having up to three amino acid substitutions, additions or deletions;

(xxv)
(SEQ ID NO: 184)
CDR1α - VGISA,
(SEQ ID NO: 185)
CDR2α - LSSGK,
(SEQ ID NO: 186)
CDR3α - CAARSYNTDKLIF,
(SEQ ID NO: 206)
CDR1β - MNHEY,
(SEQ ID NO: 207)
CDR2β - SVGAGI,
and
(SEQ ID NO: 208)
CDR3β - CASSSYPLRTGRYNSYNSPLHF,

or variants thereof each having up to three amino acid substitutions, additions or deletions;

(xxvi)
(SEQ ID NO: 189)
CDR1α - NSMFDY,
(SEQ ID NO: 190)
CDR2α - ISSIKDK,
(SEQ ID NO: 191)
CDR3α - CAASYNNARLMF,
(SEQ ID NO: 194)
CDR1β - SGHTS,
(SEQ ID NO: 195)
CDR2β - YDEGEE,
and
(SEQ ID NO: 196)
CDR3β - CASSWGYQETQYF,

or variants thereof each having up to three amino acid substitutions, additions or deletions;

(xxvii)
(SEQ ID NO: 189)
CDR1α - NSMFDY,
(SEQ ID NO: 190)
CDR2α - ISSIKDK,
(SEQ ID NO: 191)
CDR3α - CAASYNNARLMF,
(SEQ ID NO: 200)
CDR1β - KGHSH,
(SEQ ID NO: 201)
CDR2β - LQKENI,
and
(SEQ ID NO: 202)
CDR3β - CASSPTGGEYYGYTF,

or variants thereof each having up to three amino acid substitutions, additions or deletions;

(xxviii)
(SEQ ID NO: 189)
CDR1α - NSMFDY,
(SEQ ID NO: 190)
CDR2α - ISSIKDK,
(SEQ ID NO: 191)
CDR3α - CAASYNNARLMF,
(SEQ ID NO: 206)
CDR1β - MNHEY,
(SEQ ID NO: 207)
CDR2β - SVGAGI,
and
(SEQ ID NO: 208)
CDR3β - CASSSYPLRTGRYNSYNSPLHF,

or variants thereof each having up to three amino acid substitutions, additions or deletions;

(xxix)
(SEQ ID NO: 212)
CDR1α - NSMFDY,
(SEQ ID NO: 213)
CDR2α - ISSIKDK,
(SEQ ID NO: 214)
CDR3α - CAASGGRDDKIIF,
(SEQ ID NO: 217)
CDR1β - MNHEY,
(SEQ ID NO: 218)
CDR2β - SVGAGI,
and
(SEQ ID NO: 219)
CDR3β - CASSYSRTESTDTQYF,

or variants thereof each having up to three amino acid substitutions, additions or deletions;

(xxx)
(SEQ ID NO: 90)
CDR1α - NSMFDY,
(SEQ ID NO: 91)
CDR2α - ISSIKDK,
(SEQ ID NO: 92)
CDR3α - CAANNARLMF,
(SEQ ID NO: 269)
CDR1β - SGHRS,
(SEQ ID NO: 270)
CDR2β - YFSETQ,
and
(SEQ ID NO: 271)
CDR3β - CASSPGQHGELFF,

or variants thereof each having up to three amino acid substitutions, additions or deletions;

(xxxi)
(SEQ ID NO: 264)
CDRlα - NSMFDY,
(SEQ ID NO: 265)
CDR2α - ISSIKDK,
(SEQ ID NO: 266)
CDR3α - CAASATGNQFYF,
(SEQ ID NO: 95)
CDR1β - SGHNS,
(SEQ ID NO: 96)
CDR2β - FNNNVP,
and
(SEQ ID NO: 97)
CDR3β - CASSDTRAREQFF,

or variants thereof each having up to three amino acid substitutions, additions or deletions;

(xxxii)
(SEQ ID NO: 264)
CDRlα - NSMFDY,
(SEQ ID NO: 265)
CDR2α - ISSIKDK,
(SEQ ID NO: 266)
CDR3α - CAASATGNQFYF,
(SEQ ID NO: 269)
CDR1β - SGHRS,
(SEQ ID NO: 270)
CDR2β - YFSETQ,
and
(SEQ ID NO: 271)
CDR3β - CASSPGQHGELFF,

or variants thereof each having up to three amino acid substitutions, additions or deletions;

(xxxiii)
(SEQ ID NO: 275)
CDR1α - TSINN,
(SEQ ID NO: 276)
CDR2α - IRSNERE,
(SEQ ID NO: 277)
CDR3α - CATDGDSSYKLIF,
(SEQ ID NO: 161)
CDR1β - DFQATT,
(SEQ ID NO: 162)
CDR2β - SNEGSKA,
and
(SEQ ID NO: 163)
CDR3β - CSARDSVSGNTIYF,

or variants thereof each having up to three amino acid substitutions, additions or deletions;

(xxxiv)
(SEQ ID NO: 275)
CDR1α - TSINN,
(SEQ ID NO: 276)
CDR2α - IRSNERE,
(SEQ ID NO: 277)
CDR3α - CATDGDSSYKLIF,
(SEQ ID NO: 167)
CDR1β - SQVTM,
(SEQ ID NO: 168)
CDR2β - ANQGSEA,
and
(SEQ ID NO: 169)
CDR3β - CSVGGSGSYNEQFF,

or variants thereof each having up to three amino acid substitutions, additions or deletions;

(xxxv)
(SEQ ID NO: 275)
CDR1α - TSINN,
(SEQ ID NO: 276)
CDR2α - IRSNERE,
(SEQ ID NO: 277)
CDR3α - CATDGDSSYKLIF,
(SEQ ID NO: 280)
CDR1β - DFQATT,
(SEQ ID NO: 281)
CDR2β - SNEGSKA,
and
(SEQ ID NO: 282)
CDR3β - CSARDVLTGDYGYTF,

or variants thereof each having up to three amino acid substitutions, additions or deletions;

(xxxvi)
(SEQ ID NO: 275)
CDR1α - TSINN,
(SEQ ID NO: 276)
CDR2α - IRSNERE,
(SEQ ID NO: 277)
CDR3α - CATDGDSSYKLIF,
(SEQ ID NO: 286)
CDR1β - SGHDY,
(SEQ ID NO: 287)
CDR2β - FNNNVP,
and
(SEQ ID NO: 288)
CDR3β - CASSLGLSISQETQYF,

or variants thereof each having up to three amino acid substitutions, additions or deletions;

(xxxvii)
(SEQ ID NO: 101)
CDR1α - DSSSTY,
(SEQ ID NO: 102)
CDR2α - IFSNMDM,
(SEQ ID NO: 103)
CDR3α - CAERLNTDKLIF,
(SEQ ID NO: 286)
CDR1β - SGHDY,
(SEQ ID NO: 287)
CDR2β - FNNNVP,
and
(SEQ ID NO: 288)
CDR3β - CASSLGLSISQETQYF,

or variants thereof each having up to three amino acid substitutions, additions or deletions;

(xxxviii)
(SEQ ID NO: 106)
CDR1α - DSVNN,
(SEQ ID NO: 107)
CDR2α - IPSGT,
(SEQ ID NO: 108)
CDR3α - CAVEATDSWGKLQF,
(SEQ ID NO: 286)
CDR1β - SGHDY,
(SEQ ID NO: 287)
CDR2β - FNNNVP,
and
(SEQ ID NO: 288)
CDR3β - CASSLGLSISQETQYF,

or variants thereof each having up to three amino acid substitutions, additions or deletions;

(xxxix)
(SEQ ID NO: 111)
CDR1α - DSASNY,
(SEQ ID NO: 112)
CDR2α - IRSNVGE,
(SEQ ID NO: 113)
CDR3α - CAVRTSYDKVIF,
(SEQ ID NO: 286)
CDR1β - SGHDY,
(SEQ ID NO: 287)
CDR2β - FNNNVP,
and
(SEQ ID NO: 288)
CDR3β - CASSLGLSISQETQYF,

or variants thereof each having up to three amino acid substitutions, additions or deletions;

(xxxx)
(SEQ ID NO: 101)
CDR1α - DSSSTY,
(SEQ ID NO: 102)
CDR2α - IFSNMDM,
(SEQ ID NO: 103)
CDR3α - CAERLNTDKLIF,
(SEQ ID NO: 280)
CDR1β - DFQATT,
(SEQ ID NO: 281)
CDR2β - SNEGSKA,
and
(SEQ ID NO: 282)
CDR3β - CSARDVLTGDYGYTF,

or variants thereof each having up to three amino acid substitutions, additions or deletions;

(xxxxi)
(SEQ ID NO: 106)
CDR1α - DSVNN,
(SEQ ID NO: 107)
CDR2α - IPSGT,
(SEQ ID NO: 108)
CDR3α - CAVEATDSWGKLQF,
(SEQ ID NO: 280)
CDR1β - DFQATT,
(SEQ ID NO: 281)
CDR2β - SNEGSKA,
and
(SEQ ID NO: 282)
CDR3β - CSARDVLTGDYGYTF,

or variants thereof each having up to three amino acid substitutions, additions or deletions; or

(xxxxii)
(SEQ ID NO: 111)
CDR1α - DSASNY,
(SEQ ID NO: 112)
CDR2α - IRSNVGE,
(SEQ ID NO: 113)
CDR3α - CAVRTSYDKVIF,
(SEQ ID NO: 280)
CDR1β - DFQATT,
(SEQ ID NO: 281)
CDR2β - SNEGSKA,
and
(SEQ ID NO: 282)
CDR3β - CSARDVLTGDYGYTF,

or variants thereof each having up to three amino acid substitutions, additions or deletions.

In one embodiment, the present invention provides a TCR of the present invention comprising:
(i) an α chain variable domain comprising the amino acid sequence of SEQ ID NO: 4 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 9 or a variant thereof having at least 75%, at least 80%, at least 85%, at least 90%, at least 95%, at least 96%, at least 97%, at least 98%, or at least 99%, preferably at least 75%, sequence identity thereto;
(ii) an α chain variable domain comprising the amino acid sequence of SEQ ID NO: 37 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 42 or a variant thereof having at least 75%, at least 80%, at least 85%, at least 90%, at least 95%, at least 96%, at least 97%, at least 98%, or at least 99%, preferably at least 75%, sequence identity thereto;
(iii) an α chain variable domain comprising the amino acid sequence of SEQ ID NO: 15 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 25 or a variant thereof having at least 75%, at least 80%, at least 85%, at least 90%, at least 95%, at least 96%, at least 97%, at least 98%, or at least 99%, preferably at least 75%, sequence identity thereto;
(iv) an α chain variable domain comprising the amino acid sequence of SEQ ID NO: 15 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 31 or a variant thereof having at least 75%, at least 80%, at least 85%, at least 90%, at least 95%, at least 96%, at least 97%, at least 98%, or at least 99%, preferably at least 75%, sequence identity thereto;
(v) an α chain variable domain comprising the amino acid sequence of SEQ ID NO: 20 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 25 or a variant thereof having at least 75%, at least 80%, at least 85%, at least 90%, at least 95%, at least 96%, at least 97%, at least 98%, or at least 99%, preferably at least 75%, sequence identity thereto;
(vi) an α chain variable domain comprising the amino acid sequence of SEQ ID NO: 20 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 31 or a variant thereof having at least 75%, at least 80%, at least 85%, at least 90%, at least 95%, at least 96%, at least 97%, at least 98%, or at least 99%, preferably at least 75%, sequence identity thereto;
(vii) an α chain variable domain comprising the amino acid sequence of SEQ ID NO: 48 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 58 or a variant thereof having at least 75%, at least 80%, at least 85%, at least 90%, at least 95%, at least 96%, at least 97%, at least 98%, or at least 99%, preferably at least 75%, sequence identity thereto;
(viii) an α chain variable domain comprising the amino acid sequence of SEQ ID NO: 48 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 64 or a variant thereof having at least 75%, at least 80%, at least 85%, at least 90%, at least 95%, at least 96%, at least 97%, at least 98%, or at least 99%, preferably at least 75%, sequence identity thereto;
(ix) an α chain variable domain comprising the amino acid sequence of SEQ ID NO: 48 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 70 or a variant thereof having at least 75%, at least 80%, at least 85%, at least 90%, at least 95%, at least 96%, at least 97%, at least 98%, or at least 99%, preferably at least 75%, sequence identity thereto;
(x) an α chain variable domain comprising the amino acid sequence of SEQ ID NO: 53 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 58 or a variant thereof having at least 75%, at least 80%, at least 85%, at least 90%, at least 95%, at least 96%, at least 97%, at least 98%, or at least 99%, preferably at least 75%, sequence identity thereto;
(xi) an α chain variable domain comprising the amino acid sequence of SEQ ID NO: 53 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 64 or a variant thereof having at least 75%, at least 80%, at least 85%, at least 90%, at least 95%, at least 96%, at least 97%, at least 98%, or at least 99%, preferably at least 75%, sequence identity thereto;
(xii) an α chain variable domain comprising the amino acid sequence of SEQ ID NO: 53 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 70 or a variant thereof having at least 75%, at least 80%, at least 85%, at least 90%, at least 95%, at least 96%, at least 97%, at least 98%, or at least 99%, preferably at least 75%, sequence identity thereto;
(xiii) an α chain variable domain comprising the amino acid sequence of SEQ ID NO: 76 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 81 or a variant thereof having at least 75%, at least 80%, at least 85%, at least 90%, at least 95%, at least 96%, at least 97%, at least 98%, or at least 99%, preferably at least 75%, sequence identity thereto;
(xiv) an α chain variable domain comprising the amino acid sequence of SEQ ID NO: 76 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 87 or a variant thereof at least 75%, at least 80%, at least 85%, at least 90%, at least 95%, at least 96%, at least 97%, at least 98%, or at least 99%, preferably at least 75%, sequence identity thereto;
(xv) an α chain variable domain comprising the amino acid sequence of SEQ ID NO: 93 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 98 or a variant thereof having at least 75%, at least 80%, at least 85%, at least 90%, at least 95%, at least 96%, at least 97%, at least 98%, or at least 99%, preferably at least 75%, sequence identity thereto;
(xvi) an α chain variable domain comprising the amino acid sequence of SEQ ID NO: 104 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 164 or a variant thereof having at least 75%, at least 80%, at least 85%, at least 90%, at least 95%, at least 96%, at least 97%, at least 98%, or at least 99%, preferably at least 75%, sequence identity thereto;
(xvii) an α chain variable domain comprising the amino acid sequence of SEQ ID NO: 104 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 170 or a variant thereof having at least 75%, at least 80%, at least 85%, at least 90%, at least 95%, at least 96%, at least 97%, at least 98%, or at least 99%, preferably at least 75%, sequence identity thereto;
(xviii) an α chain variable domain comprising the amino acid sequence of SEQ ID NO: 109 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 164 or a variant thereof having at least 75%, at least 80%, at least 85%, at least 90%, at least 95%, at least 96%, at least 97%, at least 98%, or at least 99%, preferably at least 75%, sequence identity thereto;
(xix) an α chain variable domain comprising the amino acid sequence of SEQ ID NO: 109 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 170 or a variant thereof having at least 75%, at least 80%, at least 85%, at least 90%, at least 95%, at least 96%, at least 97%, at least 98%, or at least 99%, preferably at least 75%, sequence identity thereto;
(xx) an α chain variable domain comprising the amino acid sequence of SEQ ID NO: 114 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 164 or a variant thereof having at least 75%, at least 80%, at least 85%, at least 90%, at least 95%, at least 96%, at least 97%, at least 98%, or at least 99%, preferably at least 75%, sequence identity thereto;
(xxi) an α chain variable domain comprising the amino acid sequence of SEQ ID NO: 114 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 170 or a variant thereof having at least 75%, at least 80%, at least 85%, at least 90%, at least 95%, at least 96%, at least 97%, at least 98%, or at least 99%, preferably at least 75%, sequence identity thereto;
(xxii) an α chain variable domain comprising the amino acid sequence of SEQ ID NO: 176 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 181 or a variant thereof having at least 75%, at least 80%, at least 85%, at least 90%, at least 95%, at least 96%, at least 97%, at least 98%, or at least 99%, preferably at least 75%, sequence identity thereto;
(xxiii) an α chain variable domain comprising the amino acid sequence of SEQ ID NO: 187 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 197 or a variant thereof having at least 75%, at least 80%, at least 85%, at least 90%, at least 95%, at least 96%, at least 97%, at least 98%, or at least 99%, preferably at least 75%, sequence identity thereto;
(xxiv) an α chain variable domain comprising the amino acid sequence of SEQ ID NO: 187 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 203 or a variant thereof having at least 75%, at least 80%, at least 85%, at least 90%, at least 95%, at least 96%, at least 97%, at least 98%, or at least 99%, preferably at least 75%, sequence identity thereto;
(xxv) an α chain variable domain comprising the amino acid sequence of SEQ ID NO: 187 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 209 or a variant thereof having at least 75%, at least 80%, at least 85%, at least 90%, at least 95%, at least 96%, at least 97%, at least 98%, or at least 99%, preferably at least 75%, sequence identity thereto;
(xxvi) an α chain variable domain comprising the amino acid sequence of SEQ ID NO: 192 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 197 or a variant thereof having at least 75%, at least 80%, at least 85%, at least 90%, at least 95%, at least 96%, at least 97%, at least 98%, or at least 99%, preferably at least 75%, sequence identity thereto;
(xxvii) an α chain variable domain comprising the amino acid sequence of SEQ ID NO: 192 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 203 or a variant thereof having at least 75%, at least 80%, at least 85%, at least 90%, at least 95%, at least 96%, at least 97%, at least 98%, or at least 99%, preferably at least 75%, sequence identity thereto;
(xxviii) an α chain variable domain comprising the amino acid sequence of SEQ ID NO: 192 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 209 or a variant thereof having at least 75%, at least 80%, at least 85%, at least 90%, at least 95%, at least 96%, at least 97%, at least 98%, or at least 99%, preferably at least 75%, sequence identity thereto; or
(xxix) an α chain variable domain comprising the amino acid sequence of SEQ ID NO: 215 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 220 or a variant thereof having at least 75%, at least 80%, at least 85%, at least 90%, at least 95%, at least 96%, at least 97%, at least 98%, or at least 99%, preferably at least 75%, sequence identity thereto;
(xxx) an α chain variable domain comprising the amino acid sequence of SEQ ID NO: 93 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 272 or a variant thereof having at least 75%, at least 80%, at least 85%, at least 90%, at least 95%, at least 96%, at least 97%, at least 98%, or at least 99%, preferably at least 75%, sequence identity thereto;
(xxxi) an α chain variable domain comprising the amino acid sequence of SEQ ID NO: 267 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 98 or a variant thereof having at least 75%, at least 80%, at least 85%, at least 90%, at least 95%, at least 96%, at least 97%, at least 98%, or at least 99%, preferably at least 75%, sequence identity thereto;
(xxxii) an α chain variable domain comprising the amino acid sequence of SEQ ID NO: 267 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 272 or a variant thereof having at least 75%, at least 80%, at least 85%, at least 90%, at least 95%, at least 96%, at least 97%, at least 98%, or at least 99%, preferably at least 75%, sequence identity thereto;
(xxxiii) an α chain variable domain comprising the amino acid sequence of SEQ ID NO: 278 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 164 or a variant thereof having at least 75%, at least 80%, at least 85%, at least 90%, at least 95%, at least 96%, at least 97%, at least 98%, or at least 99%, preferably at least 75%, sequence identity thereto;
(xxxiv) an α chain variable domain comprising the amino acid sequence of SEQ ID NO: 278 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 170 or a variant thereof having at least 75%, at least 80%, at least 85%, at least 90%, at least 95%, at least 96%, at least 97%, at least 98%, or at least 99%, preferably at least 75%, sequence identity thereto;
(xxxv) an α chain variable domain comprising the amino acid sequence of SEQ ID NO: 278 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 283 or a variant thereof having at least 75%, at least 80%, at least 85%, at least 90%, at least 95%, at least 96%, at least 97%, at least 98%, or at least 99%, preferably at least 75%, sequence identity thereto;
(xxxvi) an α chain variable domain comprising the amino acid sequence of SEQ ID NO: 278 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 289 or a variant thereof having at least 75%, at least 80%, at least 85%, at least 90%, at least 95%, at least 96%, at least 97%, at least 98%, or at least 99%, preferably at least 75%, sequence identity thereto;
(xxxvii) an α chain variable domain comprising the amino acid sequence of SEQ ID NO: 104 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 289 or a variant thereof having at least 75%, at least 80%, at least 85%, at least 90%, at least 95%, at least 96%, at least 97%, at least 98%, or at least 99%, preferably at least 75%, sequence identity thereto;
(xxxviii) an α chain variable domain comprising the amino acid sequence of SEQ ID NO: 109 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 289 or a variant thereof having at least 75%, at least 80%, at least 85%, at least 90%, at least 95%, at least 96%, at least 97%, at least 98%, or at least 99%, preferably at least 75%, sequence identity thereto;
(xxxix) an α chain variable domain comprising the amino acid sequence of SEQ ID NO: 114 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 289 or a variant thereof having at least 75%, at least 80%, at least 85%, at least 90%, at least 95%, at least 96%, at least 97%, at least 98%, or at least 99%, preferably at least 75%, sequence identity thereto;
(xxxx) an α chain variable domain comprising the amino acid sequence of SEQ ID NO: 104 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 283 or a variant thereof having at least 75%, at least 80%, at least 85%, at least 90%, at least 95%, at least 96%, at least 97%, at least 98%, or at least 99%, preferably at least 75%, sequence identity thereto;
(xxxxi) an α chain variable domain comprising the amino acid sequence of SEQ ID NO: 109 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 283 or a variant thereof having at least 75%, at least 80%, at least 85%, at least 90%, at least 95%, at least 96%, at least 97%, at least 98%, or at least 99%, preferably at least 75%, sequence identity thereto; or
(xxxxii) an α chain variable domain comprising the amino acid sequence of SEQ ID NO: 114 or a variant thereof having at least 75%, at least 80%, at least 85%, at least 90%, at least 95%, at least 96%, at least 97%, at least 98%, or at least 99%, preferably at least 75%, sequence identity thereto; and a β chain variable domain comprising the amino acid sequence of SEQ ID NO: 283 or a variant thereof having at least 75%, at least 80%, at least 85%, at least 90%, at least 95%, at least 96%, at least 97%, at least 98%, or at least 99%, preferably at least 75%, sequence identity thereto.

In one embodiment, the present invention provides a TCR of the present invention comprising:
(i) an α chain comprising the amino acid sequence of SEQ ID NO: 5 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 10, SEQ ID NO: 11 and variants of SEQ ID NOs: 10 and 11 having at least 75%, at least 80%, at least 85%, at least 90%, at least 95%, at least 96%, at least 97%, at least 98%, or at least 99%, preferably at least 75%, sequence identity thereto;
(ii) an α chain comprising the amino acid sequence of SEQ ID NO: 38 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 43, SEQ ID NO: 44 and variants of SEQ ID NOs: 43 and 44 having at least 75%, at least 80%, at least 85%, at least 90%, at least 95%, at least 96%, at least 97%, at least 98%, or at least 99%, preferably at least 75%, sequence identity thereto;
(iii) an α chain comprising the amino acid sequence of SEQ ID NO: 16 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 26, SEQ ID NO: 27 and variants of SEQ ID NOs: 26 and 27 having at least 75%, at least 80%, at least 85%, at least 90%, at least 95%, at least 96%, at least 97%, at least 98%, or at least 99%, preferably at least 75%, sequence identity thereto;
(iv) an α chain comprising the amino acid sequence of SEQ ID NO: 16 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 32, SEQ ID NO: 33 and variants of SEQ ID NOs: 32 and 33 having at least 75%, at least 80%, at least 85%, at least 90%, at least 95%, at least 96%, at least 97%, at least 98%, or at least 99%, preferably at least 75%, sequence identity thereto;
(v) an α chain comprising the amino acid sequence of SEQ ID NO: 21 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 26, SEQ ID NO: 27 and variants of SEQ ID NOs: 26 and 27 having at least 75%, at least 80%, at least 85%, at least 90%, at least 95%, at least 96%, at least 97%, at least 98%, or at least 99%, preferably at least 75%, sequence identity thereto;
(vi) an α chain comprising the amino acid sequence of SEQ ID NO: 21 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 32, SEQ ID NO: 33 and variants of SEQ ID NOs: 32 and 33 having at least 75%, at least 80%, at least 85%, at least 90%, at least 95%, at least 96%, at least 97%, at least 98%, or at least 99%, preferably at least 75%, sequence identity thereto;
(vii) an α chain comprising the amino acid sequence of SEQ ID NO: 49 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 59, SEQ ID NO: 60 and variants of SEQ ID NOs: 59 and 60 having at least 75%, at least 80%, at least 85%, at least 90%, at least 95%, at least 96%, at least 97%, at least 98%, or at least 99%, preferably at least 75%, sequence identity thereto;
(viii) an α chain comprising the amino acid sequence of SEQ ID NO: 49 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 65, SEQ ID NO: 66 and variants of SEQ ID NOs: 65 and 66 having at least 75%, at least 80%, at least 85%, at least 90%, at least 95%, at least 96%, at least 97%, at least 98%, or at least 99%, preferably at least 75%, sequence identity thereto;
(ix) an α chain comprising the amino acid sequence of SEQ ID NO: 49 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 71, SEQ ID NO: 72 and variants of SEQ ID NOs: 71 and 72 having at least 75%, at least 80%, at least 85%, at least 90%, at least 95%, at least 96%, at least 97%, at least 98%, or at least 99%, preferably at least 75%, sequence identity thereto;
(x) an α chain comprising the amino acid sequence of SEQ ID NO: 54 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 59, SEQ ID NO: 60 and variants of SEQ ID NOs: 59 and 60 having at least 75%, at least 80%, at least 85%, at least 90%, at least 95%, at least 96%, at least 97%, at least 98%, or at least 99%, preferably at least 75%, sequence identity thereto;
(xi) an α chain comprising the amino acid sequence of SEQ ID NO: 54 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 65, SEQ ID NO: 66 and variants of SEQ ID NOs: 65 and 66 having at least 75%, at least 80%, at least 85%, at least 90%, at least 95%, at least 96%, at least 97%, at least 98%, or at least 99%, preferably at least 75%, sequence identity thereto;
(xii) an α chain comprising the amino acid sequence of SEQ ID NO: 54 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 71, SEQ ID NO: 72 and variants of SEQ ID NOs: 71 and 72 having at least 75%, at least 80%, at least 85%, at least 90%, at least 95%, at least 96%, at least 97%, at least 98%, or at least 99%, preferably at least 75%, sequence identity thereto;
(xiii) an α chain comprising the amino acid sequence of SEQ ID NO: 77 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 82, SEQ ID NO: 83 and variants of SEQ ID NOs: 82 and 83 having at least 75%, at least 80%, at least 85%, at least 90%, at least 95%, at least 96%, at least 97%, at least 98%, or at least 99%, preferably at least 75%, sequence identity thereto;
(xiv) an α chain comprising the amino acid sequence of SEQ ID NO: 77 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 88, SEQ ID NO: 89 and variants of SEQ ID NOs: 88 and 89 having at least 75%, at least 80%, at least 85%, at least 90%, at least 95%, at least 96%, at least 97%, at least 98%, or at least 99%, preferably at least 75%, sequence identity thereto;
(xv) an α chain comprising the amino acid sequence of SEQ ID NO: 94 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 99, SEQ ID NO: 100 and variants of SEQ ID NO: 99, SEQ ID NO: 100 having at least 75%, at least 80%, at least 85%, at least 90%, at least 95%, at least 96%, at least 97%, at least 98%, or at least 99%, preferably at least 75%, sequence identity thereto;
(xvi) an α chain comprising the amino acid sequence of SEQ ID NO: 105 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 165, SEQ ID NO: 166 and variants of SEQ ID NOs: 165 and 166 having at least 75%, at least 80%, at least 85%, at least 90%, at least 95%, at least 96%, at least 97%, at least 98%, or at least 99%, preferably at least 75%, sequence identity thereto;
(xvii) an α chain comprising the amino acid sequence of SEQ ID NO: 105 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 171, SEQ ID NO: 172 and variants of SEQ ID NOs: 171 and 172 having at least 75%, at least 80%, at least 85%, at least 90%, at least 95%, at least 96%, at least 97%, at least 98%, or at least 99%, preferably at least 75%, sequence identity thereto;
(xviii) an α chain comprising the amino acid sequence of SEQ ID NO: 110 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 165, SEQ ID NO: 166 and variants of SEQ ID NOs: 165 and 166 having at least 75%, at least 80%, at least 85%, at least 90%, at least 95%, at least 96%, at least 97%, at least 98%, or at least 99%, preferably at least 75%, sequence identity thereto;
(xix) an α chain comprising the amino acid sequence of SEQ ID NO: 110 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 171, SEQ ID NO: 172 and variants of SEQ ID NOs: 171 and 172 having at least 75%, at least 80%, at least 85%, at least 90%, at least 95%, at least 96%, at least 97%, at least 98%, or at least 99%, preferably at least 75%, sequence identity thereto;
(xx) an α chain comprising the amino acid sequence of SEQ ID NO: 160 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 165, SEQ ID NO: 166 and variants of SEQ ID NOs: 165 and 166 having at least 75%, at least 80%, at least 85%, at least 90%, at least 95%, at least 96%, at least 97%, at least 98%, or at least 99%, preferably at least 75%, sequence identity thereto;
(xxi) an α chain comprising the amino acid sequence of SEQ ID NO: 160 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 171, SEQ ID NO: 172 and variants of SEQ ID NOs: 171 and 172 having at least 75%, at least 80%, at least 85%, at least 90%, at least 95%, at least 96%, at least 97%, at least 98%, or at least 99%, preferably at least 75%, sequence identity thereto;
(xxii) an α chain comprising the amino acid sequence of SEQ ID NO: 177 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 182, SEQ ID NO: 183 and variants of SEQ ID NOs: 182 and 183 having at least 75%, at least 80%, at least 85%, at least 90%, at least 95%, at least 96%, at least 97%, at least 98%, or at least 99%, preferably at least 75%, sequence identity thereto;
(xxiii) an α chain comprising the amino acid sequence of SEQ ID NO: 188 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 198, SEQ ID NO: 199 and variants of SEQ ID NOs: 198 and 199 having at least 75%, at least 80%, at least 85%, at least 90%, at least 95%, at least 96%, at least 97%, at least 98%, or at least 99%, preferably at least 75%, sequence identity thereto;
(xxiv) an α chain comprising the amino acid sequence of SEQ ID NO: 188 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 204, SEQ ID NO: 205 and variants of SEQ ID NOs: 204 and 205 having at least 75%, at least 80%, at least 85%, at least 90%, at least 95%, at least 96%, at least 97%, at least 98%, or at least 99%, preferably at least 75%, sequence identity thereto;
(xxv) an α chain comprising the amino acid sequence of SEQ ID NO: 188 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 210, SEQ ID NO: 211 and variants of SEQ ID NOs: 210 and 211 having at least 75%, at least 80%, at least 85%, at least 90%, at least 95%, at least 96%, at least 97%, at least 98%, or at least 99%, preferably at least 75%, sequence identity thereto;
(xxvi) an α chain comprising the amino acid sequence of SEQ ID NO: 193 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 198, SEQ ID NO: 199 and variants of SEQ ID NOs: 198 and 199 having at least 75%, at least 80%, at least 85%, at least 90%, at least 95%, at least 96%, at least 97%, at least 98%, or at least 99%, preferably at least 75%, sequence identity thereto;
(xxvii) an α chain comprising the amino acid sequence of SEQ ID NO: 193 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 204, SEQ ID NO: 205 and variants of SEQ ID NOs: 204 and 205 having at least 75%, at least 80%, at least 85%, at least 90%, at least 95%, at least 96%, at least 97%, at least 98%, or at least 99%, preferably at least 75%, sequence identity thereto;
(xxviii) an α chain comprising the amino acid sequence of SEQ ID NO: 193 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 210, SEQ ID NO: 211 and variants of SEQ ID NOs: 210 and 211 having at least 75%, at least 80%, at least 85%, at least 90%, at least 95%, at least 96%, at least 97%, at least 98%, or at least 99%, preferably at least 75%, sequence identity thereto;
(xxix) an α chain comprising the amino acid sequence of SEQ ID NO: 216 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 221, SEQ ID NO: 222 and variants of SEQ ID NOs: 221 and 222 having at least 75%, at least 80%, at least 85%, at least 90%, at least 95%, at least 96%, at least 97%, at least 98%, or at least 99%, preferably at least 75%, sequence identity thereto;
(xxx) an α chain comprising the amino acid sequence of SEQ ID NO: 94 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 273, SEQ ID NO: 274 and variants of SEQ ID NOs: 273 and 274 having at least 75%, at least 80%, at least 85%, at least 90%, at least 95%, at least 96%, at least 97%, at least 98%, or at least 99%, preferably at least 75%, sequence identity thereto;
(xxxi) an α chain comprising the amino acid sequence of SEQ ID NO: 268 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 99, SEQ ID NO: 100 and variants of SEQ ID NOs: 99 and 100 having at least 75%, at least 80%, at least 85%, at least 90%, at least 95%, at least 96%, at least 97%, at least 98%, or at least 99%, preferably at least 75%, sequence identity thereto;
(xxxii) an α chain comprising the amino acid sequence of SEQ ID NO: 268 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 273, SEQ ID NO: 274 and variants of SEQ ID NOs: 273 and 274 having at least 75%, at least 80%, at least 85%, at least 90%, at least 95%, at least 96%, at least 97%, at least 98%, or at least 99%, preferably at least 75%, sequence identity thereto;
(xxxiii) an α chain comprising the amino acid sequence of SEQ ID NO: 279 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 165, SEQ ID NO: 166 and variants of SEQ ID NOs: 165 and 166 having at least 75%, at least 80%, at least 85%, at least 90%, at least 95%, at least 96%, at least 97%, at least 98%, or at least 99%, preferably at least 75%, sequence identity thereto;
(xxxiv) an α chain comprising the amino acid sequence of SEQ ID NO: 279 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 171, SEQ ID NO: 172 and variants of SEQ ID NOs: 171 and 172 having at least 75%, at least 80%, at least 85%, at least 90%, at least 95%, at least 96%, at least 97%, at least 98%, or at least 99%, preferably at least 75%, sequence identity thereto;
(xxxv) an α chain comprising the amino acid sequence of SEQ ID NO: 279 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 284, SEQ ID NO: 285 and variants of SEQ ID NOs: 284 and 285 having at least 75%, at least 80%, at least 85%, at least 90%, at least 95%, at least 96%, at least 97%, at least 98%, or at least 99%, preferably at least 75%, sequence identity thereto;
(xxxvi) an α chain comprising the amino acid sequence of SEQ ID NO: 279 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 290, SEQ ID NO: 291 and variants of SEQ ID NOs: 290 and 291 having at least 75%, at least 80%, at least 85%, at least 90%, at least 95%, at least 96%, at least 97%, at least 98%, or at least 99%, preferably at least 75%, sequence identity thereto;
(xxxvii) an α chain comprising the amino acid sequence of SEQ ID NO: 105 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 290, SEQ ID NO: 291 and variants of SEQ ID NOs: 290 and 291 having at least 75%, at least 80%, at least 85%, at least 90%, at least 95%, at least 96%, at least 97%, at least 98%, or at least 99%, preferably at least 75%, sequence identity thereto;
(xxxviii) an α chain comprising the amino acid sequence of SEQ ID NO: 110 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 290, SEQ ID NO: 291 and variants of SEQ ID NOs: 290 and 291 having at least 75%, at least 80%, at least 85%, at least 90%, at least 95%, at least 96%, at least 97%, at least 98%, or at least 99%, preferably at least 75%, sequence identity thereto;
(xxxix) an α chain comprising the amino acid sequence of SEQ ID NO: 160 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 290, SEQ ID NO: 291 and variants of SEQ ID NOs: 290 and 291 having at least 75%, at least 80%, at least 85%, at least 90%, at least 95%, at least 96%, at least 97%, at least 98%, or at least 99%, preferably at least 75%, sequence identity thereto;
(xxxx) an α chain comprising the amino acid sequence of SEQ ID NO: 105 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 284, SEQ ID NO: 285 and variants of SEQ ID NOs: 284 and 285 having at least 75%, at least 80%, at least 85%, at least 90%, at least 95%, at least 96%, at least 97%, at least 98%, or at least 99%, preferably at least 75%, sequence identity thereto;
(xxxxi) an α chain comprising the amino acid sequence of SEQ ID NO: 110 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 284, SEQ ID NO: 285 and variants of SEQ ID NOs: 284 and 285 having at least 75%, at least 80%, at least 85%, at least 90%, at least 95%, at least 96%, at least 97%, at least 98%, or at least 99%, preferably at least 75%, sequence identity thereto; or
(xxxxii) an α chain comprising the amino acid sequence of SEQ ID NO: 160 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence selected from the group consisting of SEQ ID NO: 284, SEQ ID NO: 285 and variants of SEQ ID NOs: 284 and 285 having at least 75%, at least 80%, at least 85%, at least 90%, at least 95%, at least 96%, at least 97%, at least 98%, or at least 99%, preferably at least 75%, sequence identity thereto.

In one embodiment, the present invention provides a TCR of the present invention comprising an α chain comprising the amino acid sequence of SEQ ID NO: 257 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence of SEQ ID NO: 259 or a variant thereof having at least 75%, at least 80%, at least 85%, at least 90%, at least 95%, at least 96%, at least 97%, at least 98%, or at least 99%, preferably at least 75%, sequence identity thereto.

In one embodiment, the present invention provides a TCR of the present invention comprising an α chain comprising the amino acid sequence of SEQ ID NO: 261 or a variant thereof having at least 75%, at least 80%, at least 85%, at least 90%, at least 95%, at least 96%, at least 97%, at least 98%, or at least 99%, preferably at least 75%, sequence identity thereto; and a β chain comprising an amino acid sequence of SEQ ID NO: 263 or a variant thereof having at least 75%, at least 80%, at least 85%, at least 90%, at least 95%, at least 96%, at least 97%, at least 98%, or at least 99%, preferably at least 75%, sequence identity thereto.

A TCR of the present invention may bind to a WT1 peptide comprising an amino acid sequence selected from the group consisting of EPASQHTLRSG (SEQ ID NO: 123), YESDNHTTPIL (SEQ ID NO: 126), NHTTPILCGAQYRIH (SEQ ID NO: 127), QCLSAFTVHFSGQFT (SEQ ID NO: 118), EDPMGQQGSLGEQQY (SEQ ID NO: 119), SQLECMTWNQMNLGA (SEQ ID NO: 120), APVLDFAPPGA (SEQ ID NO: 117) NQMNLGATLKG (SEQ ID NO: 250), DPGGIWAKLGAAEAS (SEQ ID NO: 251), NHTTPILCGAQYRIH (SEQ ID NO: 252), KRHQRRHTGVKPFQC (SEQ ID NO: 253), PSCQKKFARSDELVR (SEQ ID NO: 254) and variants thereof each having up to three amino acid substitutions, additions or deletions.

In another aspect, the present invention provides a T-cell receptor (TCR), which binds to a Wilms tumour 1 protein (WT1) peptide when presented by a major histocompatibility complex (MHC), wherein the WT1 peptide comprises an amino acid sequence selected from the group consisting of EPASQHTLRSG (SEQ ID NO: 123), YESDNHTTPIL (SEQ ID NO: 126), NHTTPILCGAQYRIH (SEQ ID NO: 127), QCLSAFTVHFSGQFT (SEQ ID NO: 118), EDPMGQQGSLGEQQY (SEQ ID NO: 119), SQLECMTWNQMNLGA (SEQ ID NO: 120), APVLDFAPPGA (SEQ ID NO: 117), NQMNLGATLKG (SEQ ID NO: 250), DPGGIWAKLGAAEAS (SEQ ID NO: 251), NHTTPILCGAQYRIH (SEQ ID NO: 252), KRHQRRHTGVKPFQC (SEQ ID NO: 253), PSCQKKFARSDELVR (SEQ ID NO: 254) and variants thereof each having up to three amino acid substitutions, additions or deletions.

In one embodiment, a TCR of the present invention binds to an MHC I and/or MHC II peptide complex.

In one embodiment, a TCR of the present invention is restricted to a human leukocyte antigen (HLA) allele. In one embodiment, a TCR of the present invention is restricted to a HLA-A or a HLA-B allele. In one embodiment, a TCR of the present invention is restricted to a HLA-A allele selected from the group consisting of HLA-A*0201, HLA-A*0101, HLA-A*2402 and HLA-A*0301 or a HLA-B allele selected from the group consisting of HLA-B*0702, HLA-B*3501 and HLA-B*3502.

In one embodiment, a TCR of the present invention is restricted to HLA-A*0201.

In one embodiment, a TCR of the present invention is restricted to HLA-B*3502.

In one embodiment, a TCR of the present invention is restricted to HLA-B*3501.

In one embodiment, a TCR of the present invention is restricted to a HLA-C allele. In one embodiment, a TCR of the present invention is restricted to a HLA-C allele selected from the group consisting of HLA-C*07:01, HLA-C*03:04, HLA-C*04:01, HLA-C*05:01, HLA-C*06:02 and HLA-C*07:02.

In one embodiment, a TCR of the present invention comprises one or more mutations at the α chain/β chain interface, such that when the α chain and the β chain are expressed in a T-cell, the frequency of mispairing between said chains and endogenous TCR α and β chains is reduced.

In one embodiment, a TCR of the present invention comprises one or more mutations at the α chain/β chain interface, such that when the α chain and the β chain are expressed in a T-cell, the level of expression of the TCR α and β chains is increased.

In one embodiment, the one or more mutations introduce a cysteine residue into the constant region domain of each of the α chain and the β chain, wherein the cysteine residues are capable of forming a disulphide bond between the α chain and the β chain.

A TCR of the present invention may comprise a murinized constant region.

In one embodiment, the TCR of the invention is a soluble TCR.

In another aspect, the present invention provides an isolated polynucleotide encoding the α chain of a T-cell receptor (TCR) of the present invention, and/or the β chain of a TCR of the present invention.

In another aspect, the present invention provides an isolated polynucleotide comprising a nucleotide sequence of SEQ ID NO: 256 and/or a nucleotide sequence of SEQ ID NO: 258, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In another aspect, the present invention provides an isolated polynucleotide comprising a nucleotide sequence of SEQ ID NO: 260 and/or a nucleotide sequence of SEQ ID NO: 262, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

In one embodiment, the isolated polynucleotide encodes the α chain linked to the β chain. In one embodiment, the isolated polynucleotide encodes one or more short interfering RNA (siRNA) sequences and/or one or more other agents capable of reducing or preventing expression of one or more endogenous TCR genes.

In another aspect, the present invention provides a vector comprising a polynucleotide of the present invention. In one embodiment, the vector comprises a polynucleotide which encodes one or more CD3 chains, CD8, a suicide gene, and/or a selectable marker.

In another aspect, the present invention provides a cell comprising a TCR of the present invention, a polynucleotide of the present invention, or a vector of the present invention.

In one embodiment, the cell further comprises a vector which encodes one or more CD3 chains, CD8, a suicide gene and/or a selectable marker.

In one embodiment, the cell is a T-cell, a lymphocyte or a stem cell, such as hematopoietic stem cells or induced pluripotent stem cells (iPS). The T-cell, the lymphocyte, or the stem cell may be selected from the group consisting of CD4 cells, CD8 cells, Th0 cells, Tc0 cells, Th1 cells, Tc1 cells, Th2 cells, Tc2 cells, Th17 cells, Th22 cells, gamma/delta T-cells, natural killer (NK) cells, natural killer T (NKT) cells, double negative T-cells, naive T-cells, memory stem T-cells, central memory T-cells, effector memory T-cells, effector T cells, hematopoeitic stem cells and pluripotent stem cells.

In one embodiment, the cell is a T-cell which has been isolated from a subject.

In one embodiment, an endogenous gene encoding a TCR α chain and/or an endogenous gene encoding a TCR β chain in the cell is disrupted, preferably such that the endogenous gene encoding a TCR α chain and/or the endogenous gene encoding a TCR β chain is not expressed. In one embodiment, the endogenous gene encoding a TCR α chain and/or the endogenous gene encoding a TCR β chain is disrupted by insertion of an expression cassette comprising a polynucleotide sequence encoding a TCR of the present invention. In one embodiment, one or more endogenous genes encoding an MHC in the cell is disrupted, preferably wherein the cell is a non-alloreactive universal T-cell. In one embodiment, an endogenous gene involved in persistence, expansion, activity, resistance to exhaustion/senescence/inhibitory signals, homing capacity, or other T-cell functions in the cell is disrupted, preferably wherein the endogenous gene involved in persistence, expansion, activity, resistance to exhaustion/senescence/inhibitory signals, homing capacity, or other T-cell functions is selected from the group consisting of PD1, TIM3, LAG3, 2B4, KLRG1, TGFbR, CD160 and CTLA4. In one embodiment, the endogenous gene involved in persistence, expansion, activity, resistance to exhaustion/senescence/inhibitory signals, homing capacity, or other T-cell functions is disrupted by integration of an expression cassette, wherein the expression cassette comprises a polynucleotide sequence encoding a TCR of the present invention.

In another aspect, the present invention provides a method of preparing a cell, which comprises the step of introducing a vector of the invention into a cell in vitro, ex vivo or in vivo, for example by transfection or transduction.

In another aspect, the present invention provides a method of preparing a cell, which comprises the step of transducing a cell in vitro, ex vivo or in vivo with one or more vectors of the present invention.

In one embodiment, the cell to be transduced with the one or more vectors is selected from the group consisting of T-cells, lymphocytes or stem cells, such as hematopoietic stem cells or induced pluripotent stem cells (iPS), optionally the T-cell, the lymphocyte or the stem cell may be selected from the group consisting of CD4 cells, CD8 cells, Th0 cells, Tc0 cells, Th1 cells, Tc1 cells, Th2 cells, Tc2 cells, Th17 cells, Th22 cells, gamma/delta T-cells, natural killer (NK) cells, natural killer T (NKT) cells, double negative T-cells, naive T-cells, memory stem T-cells, central memory T-cells, effector memory T-cells, effector T cells, hematopoeitic stem cells and pluripotent stem cells.

In one embodiment, the method comprises the step of T-cell editing, which comprises disrupting an endogenous gene, for example an endogenous gene encoding a TCR α chain and/or an endogenous gene encoding a TCR β chain with an artificial nuclease, preferably wherein the artificial nuclease is selected from the group consisting of zinc finger nucleases (ZFN), transcription activator-like effector nucleases (TALEN) and CRISPR/Cas system.

In one embodiment, the method comprises the step of T-cell editing, which comprises disrupting an endogenous gene encoding a TCR α chain and/or an endogenous gene encoding a TCR β chain with an artificial nuclease, preferably wherein the artificial nuclease is selected from the group consisting of zinc finger nucleases (ZFN), transcription activator-like effector nucleases (TALEN) and CRISPR/Cas system.

In one embodiment, the method comprises the step of targeted integration of an expression cassette into the endogenous gene encoding the TCR α chain gene and/or the endogenous gene encoding the TCR β chain disrupted by the artificial nuclease, wherein the expression cassette comprises a polynucleotide encoding a TCR of the present invention or a polynucleotide sequence of the present invention.

In one embodiment, the method comprises the step of disrupting one or more endogenous genes encoding an MHC, preferably wherein the cell prepared by the method is a non-alloreactive universal T-cell.

In one embodiment, the method comprises the step of disrupting one or more endogenous MHC genes, preferably wherein the cell prepared by the method is a non-alloreactive universal T-cell.

In one embodiment, the method comprises the step of disrupting one or more endogenous genes to modify the persistence, expansion, activity, resistance to exhaustion/senescence/inhibitory signals, homing capacity, or other T-cell functions, preferably wherein the method comprises the step of targeted integration of an expression cassette into an endogenous gene involved in persistence, expansion, activity, resistance to exhaustion/senescence/inhibitory signals, homing capacity, or other T-cell functions disrupted by an artificial nuclease, wherein the expression cassette comprises a polynucleotide sequence encoding a TCR of the present invention, preferably wherein the endogenous gene is selected from the group consisting of PD1, TIM3, LAG3, 2B4, KLRG1, TGFbR, CD160 and CTLA4.

In another aspect, the present invention provides a cell of the present invention or a cell prepared by a method of the present invention for use in adoptive cell transfer, preferably adoptive T-cell transfer, optionally the adoptive T-cell transfer may be allogenic adoptive T-cell transfer, universal non-alloreactive T-cell transfer, or autologous adoptive T-cell transfer.

In another aspect, the present invention provides a TCR of the present invention, an isolated polynucleotide of the present invention, a vector of the present invention, a cell of the present invention, a cell prepared by a method of the present invention, or a chimeric molecule of the present invention for use in therapy.

In another aspect, the present invention provides a TCR of the present invention, an isolated polynucleotide of the present invention, a vector of the present invention, a cell of the present invention, a cell prepared by a method the present invention for use in treating and/or preventing a disease associated with expression of WT1.

In another aspect, the present invention provides a T-cell genetically engineered (genetically edited) to modify the persistence, expansion, activity, resistance to exaustion/senescence/inhibitory signals, homing capacity, or other T cell functions, wherein the T-cell expresses a TCR α chain of the present invention and/or a TCR β chain of the present invention.

In another aspect, the present invention provides a T cell genetically engineered (genetically edited) by a protocol which comprises the step of targeted integration of an expression cassette into an endogenous gene involved in persistence, expansion, activity, resistance to exhaustion/senescence/inhibitory signals, homing capacity, or other T-cell functions disrupted by an artificial nuclease, wherein the expression cassette comprises a polynucleotide sequence encoding TCR α chain of the present invention and/or a TCR β chain of the present invention.

In another aspect, the present invention provides a method for treating and/or preventing a disease associated with expression of WT1, which comprises the step of administering a TCR of the present invention, an isolated polynucleotide of the present invention, a vector of the present invention, a cell of the present invention, a cell prepared by a method of the present invention, or a chimeric molecule of the present invention to a subject in need thereof.

The disease associated with expression of WT1 may be a proliferative disorder. Preferably the proliferative disorder may be selected from the group consisting of hematological malignancies, such as acute myeloid leukemia (AML), chronic myeloid leukemia (CML), lymphoblastic leukemia, myelodisplastic syndromes, multiple myeloma, non Hodgkin lymphoma, Hodgkin lymphoma. The proliferative disorder may be selected from the group of solid tumors, such as lung cancer, breast cancer, oesophageal cancer, gastric cancer, colon cancer, cholangiocarcinoma, pancreatic cancer, ovarian cancer, head and neck cancers, synovial sarcoma, angiosarcoma, osteosarcoma, thyroid cancer, endometrial cancer, neuroblastoma, rabdomyosarcoma, liver cancer, melanoma, prostate cancer, renal cancer, soft tissue sarcoma, urothelial cancer, biliary cancer, glioblastoma, mesothelioma, cervical cancer, and colorectal cancer.

In a preferred embodiment, the disease associated with expression of WT1 is acute myeloid leukemia (AML).

In another preferred embodiment, the disease associated with expression of WT1 is chronic myeloid leukemia (CML).

In another aspect, the present invention provides an isolated immunogenic WT1 peptide comprising an amino acid sequence selected from the group consisting of EPASQHTLRSG (SEQ ID NO: 123), YESDNHTTPIL (SEQ ID NO: 126), NHTTPILCGAQYRIH (SEQ ID NO: 127), QCLSAFTVHFSGQFT (SEQ ID NO: 118), EDPMGQQGSLGEQQY (SEQ ID NO: 119), SQLECMTWNQMNLGA (SEQ ID NO: 120), APVLDFAPPGA (SEQ ID NO: 117), NQMNLGATLKG (SEQ ID NO: 250), DPGGIWAKLGAAEAS (SEQ ID NO: 251), NHTTPILCGAQYRIH (SEQ ID NO: 252), KRHQRRHTGVKPFQC (SEQ ID NO: 253), PSCQKKFARSDELVR (SEQ ID NO: 254) and variants thereof each having up to three amino acid substitutions, additions or deletions.

### DESCRIPTION OF THE DRAWINGS

FIGS. 1A-1J. Plots showing the results of in vitro expansion of functional WT-1 specific T-cells from peripheral blood of ten healthy donors

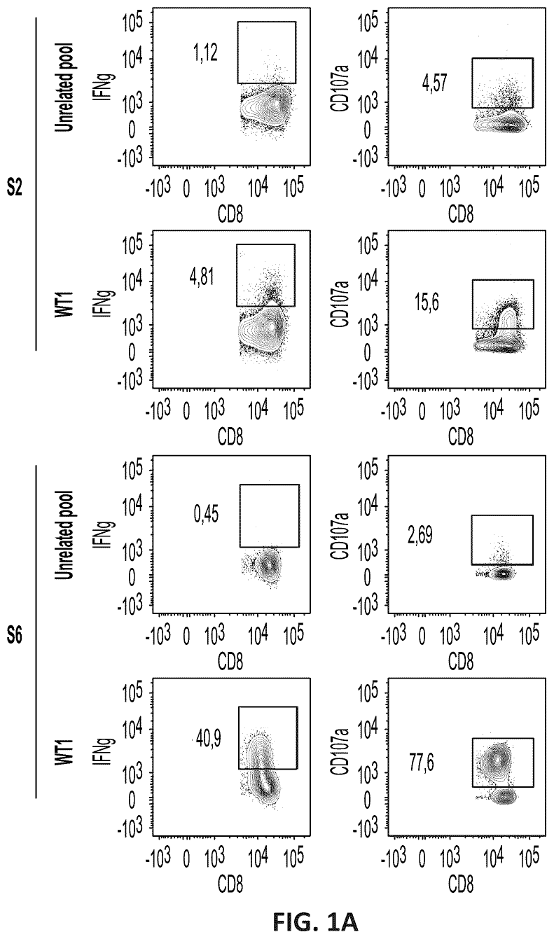

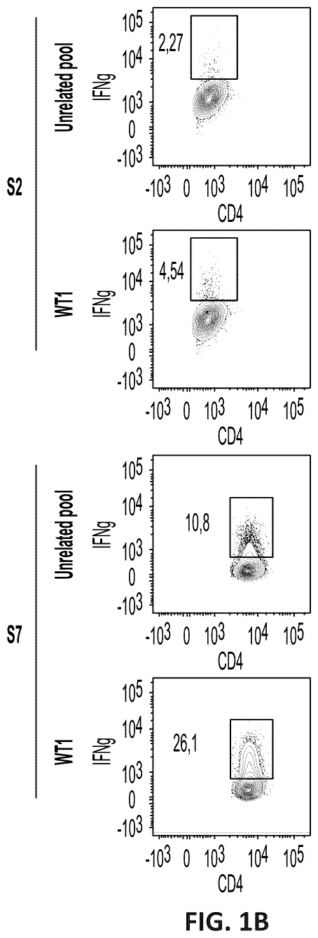

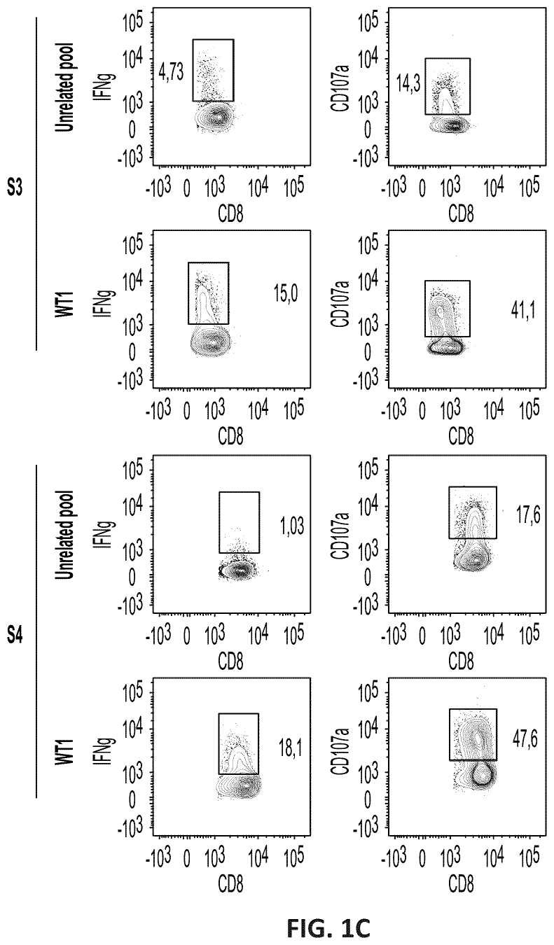

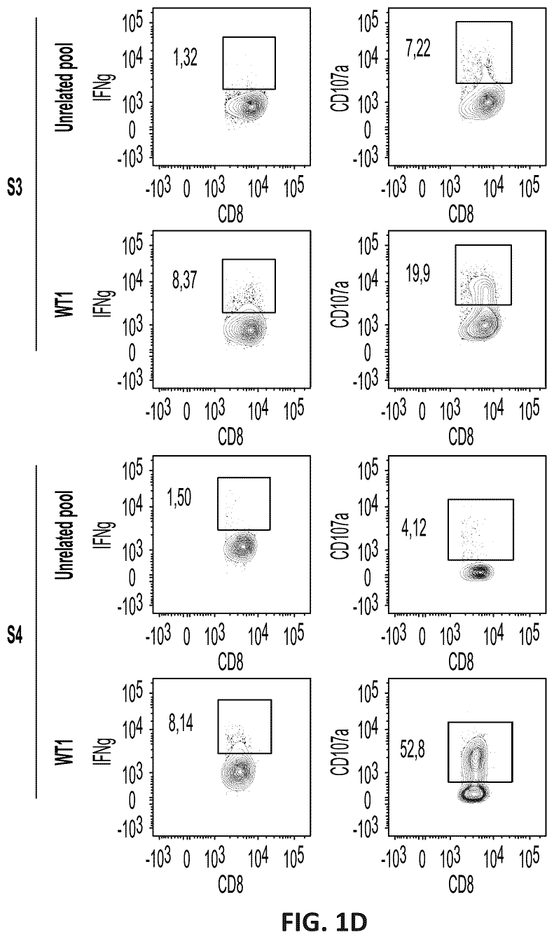

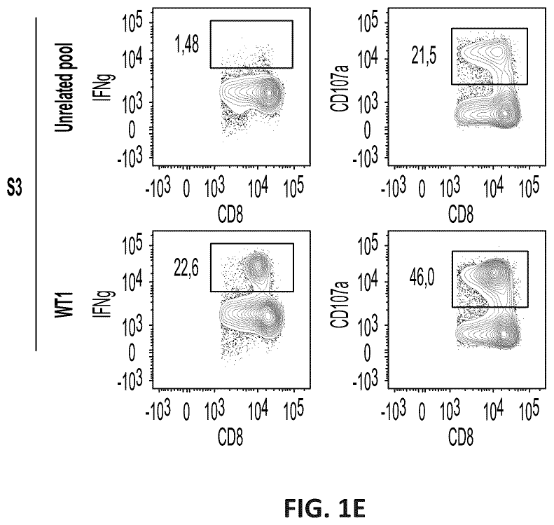

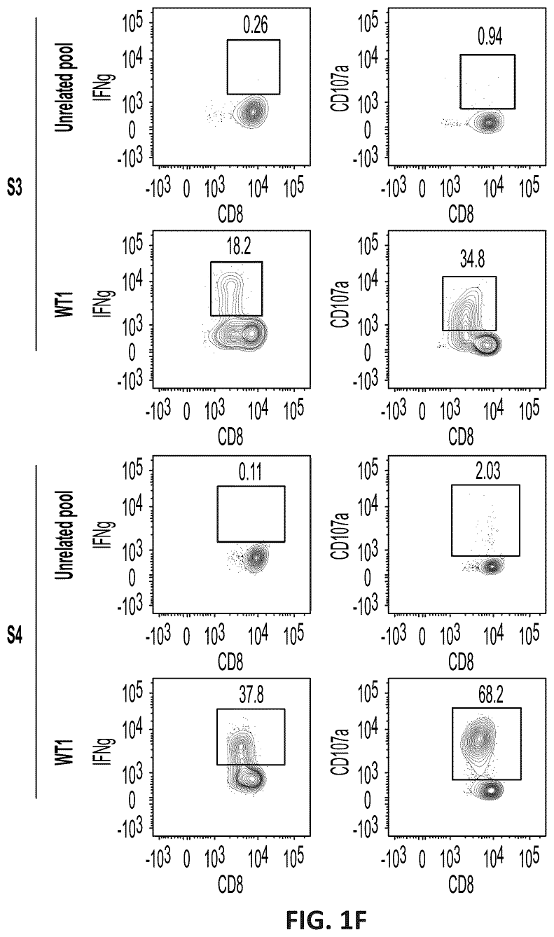

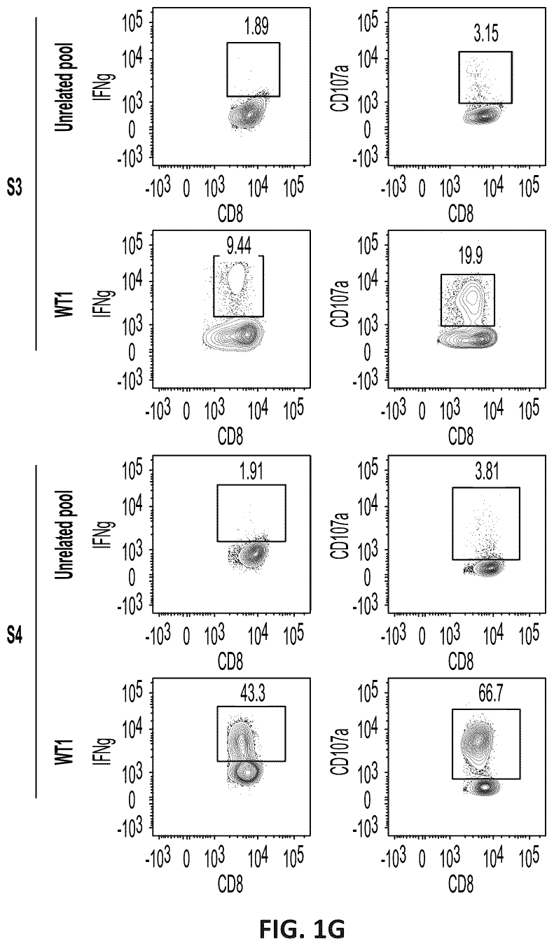

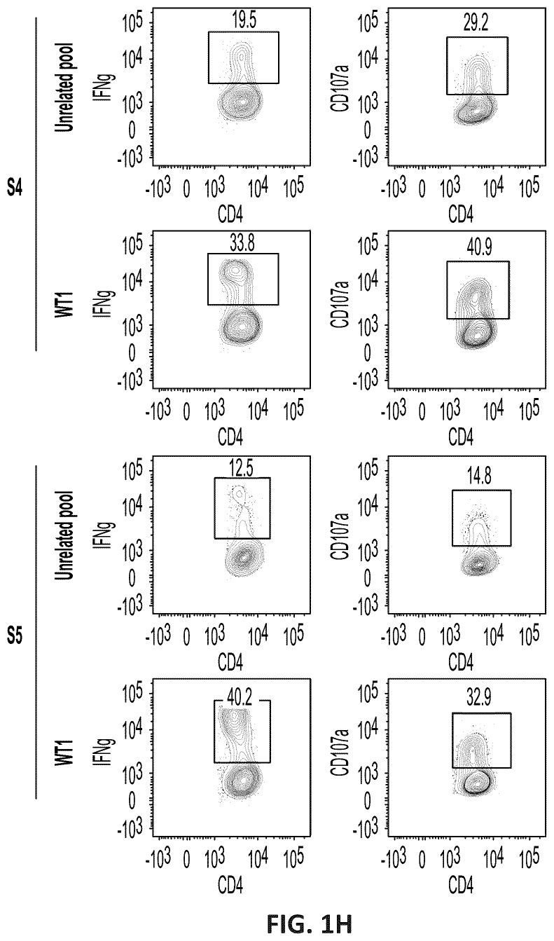

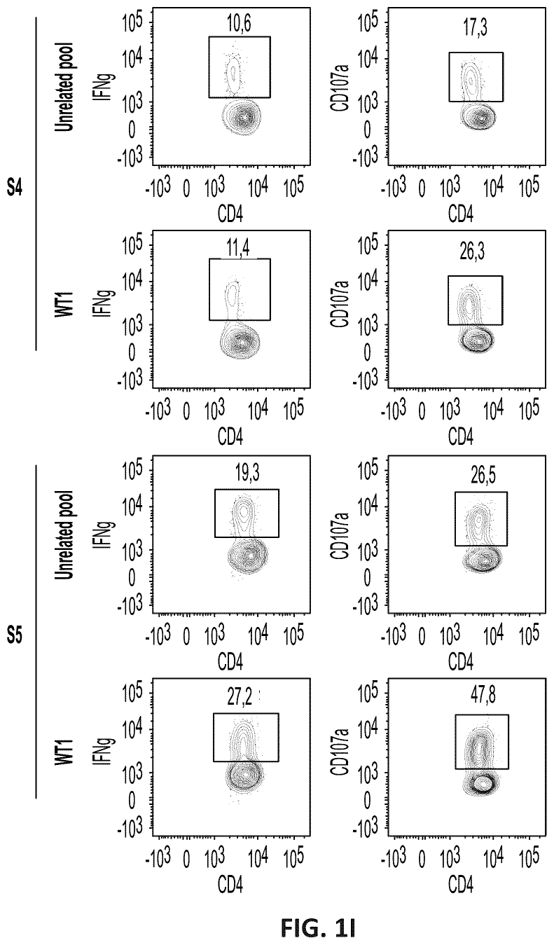

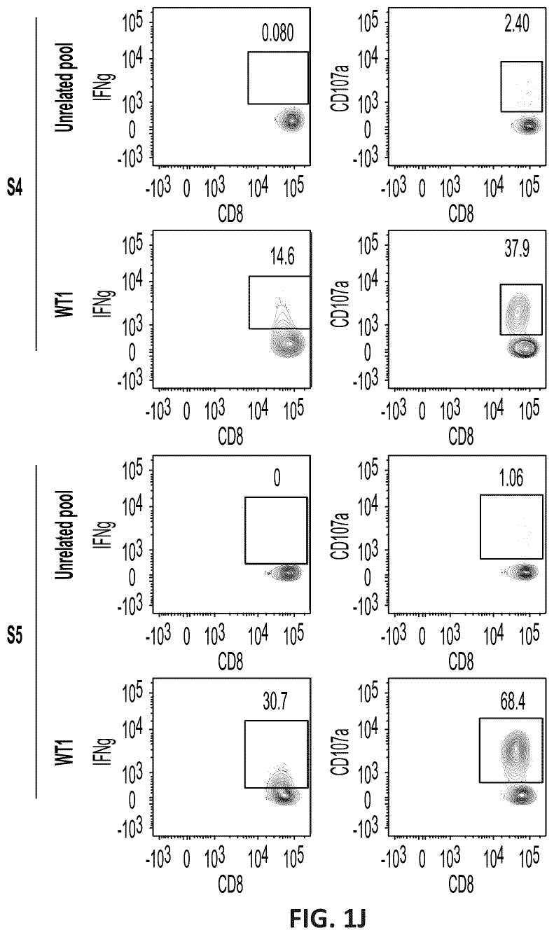

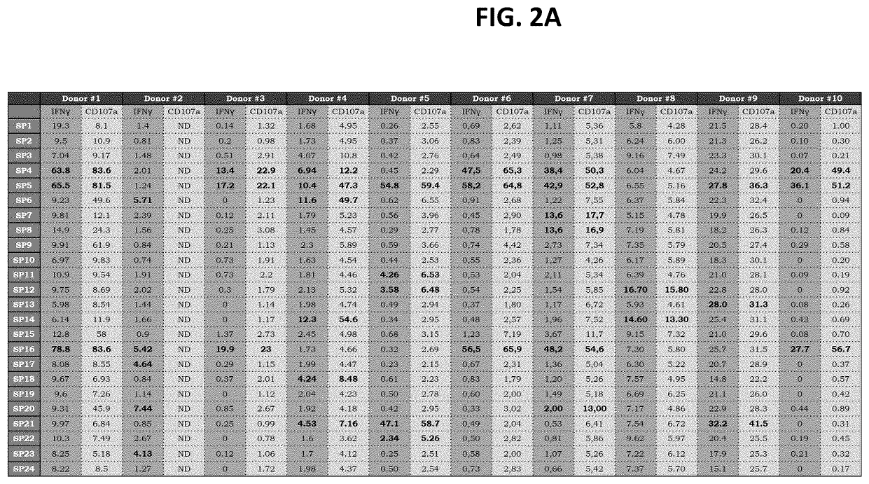

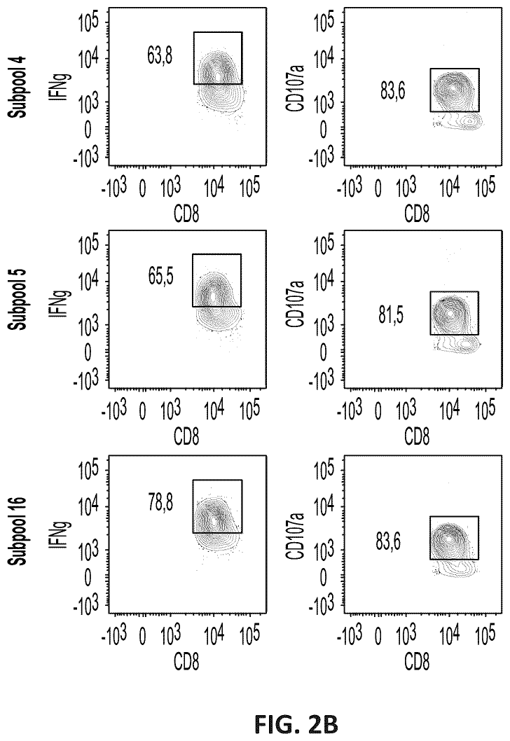

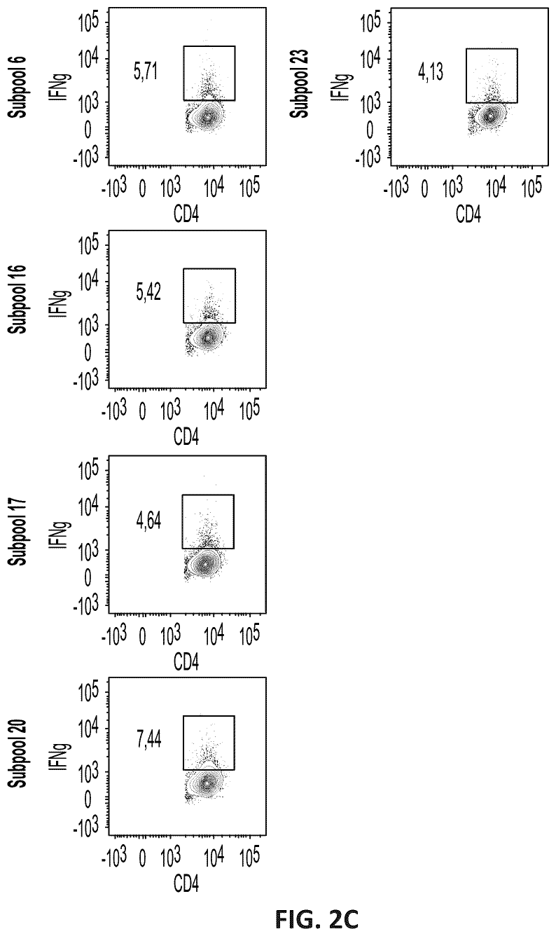

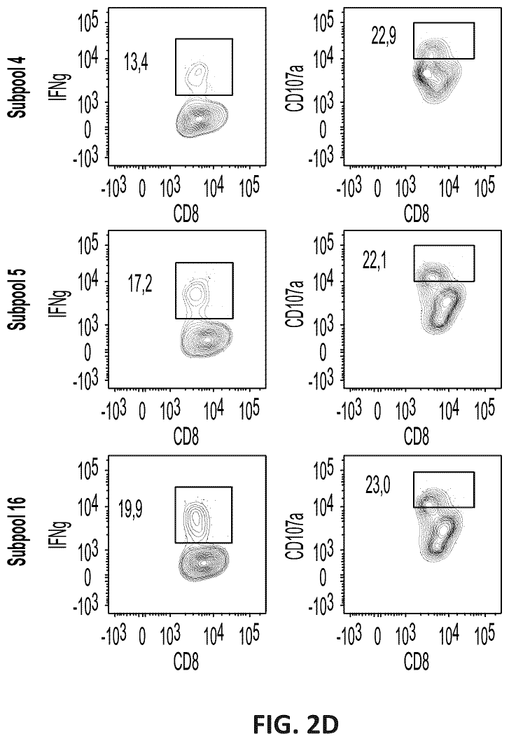

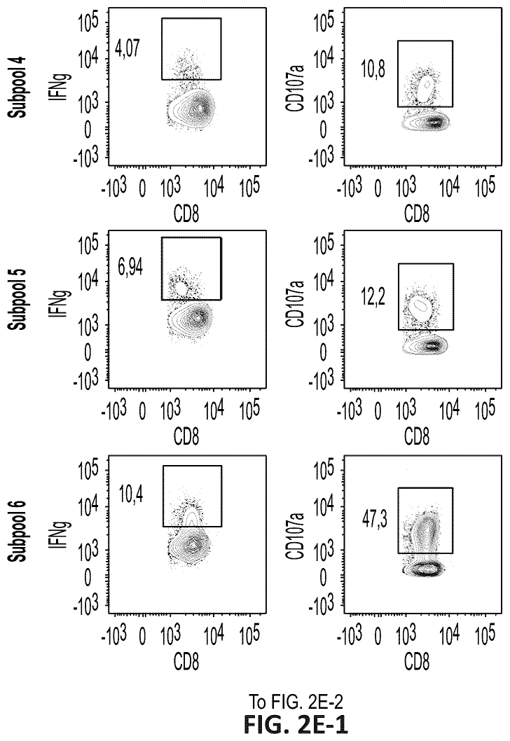

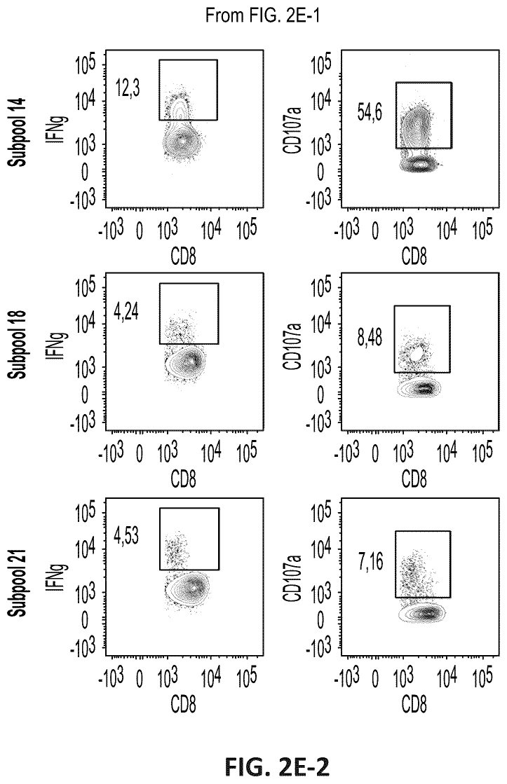

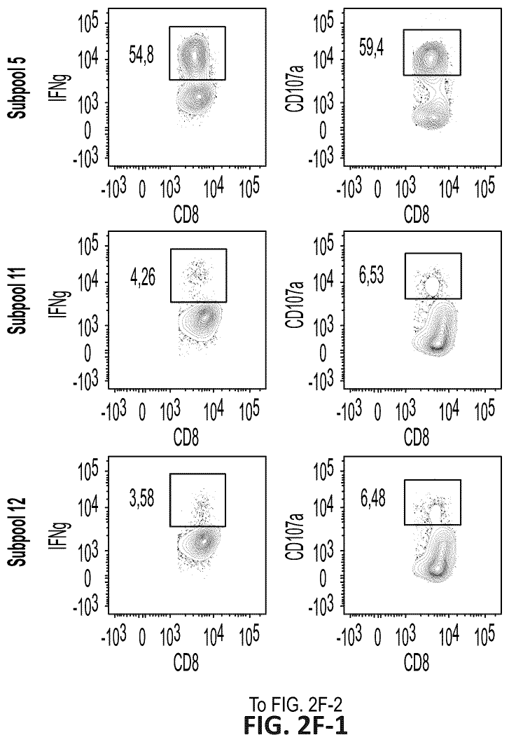

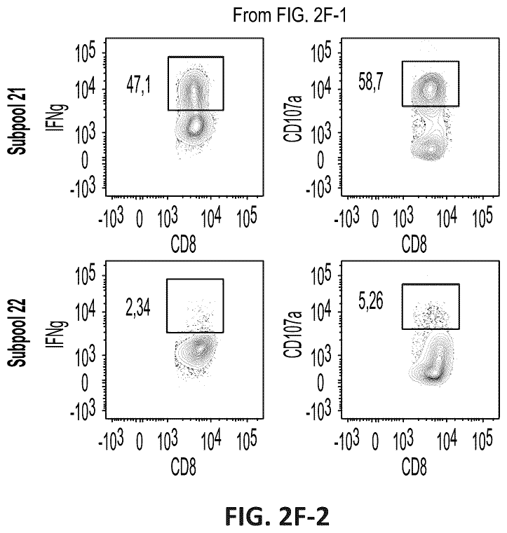

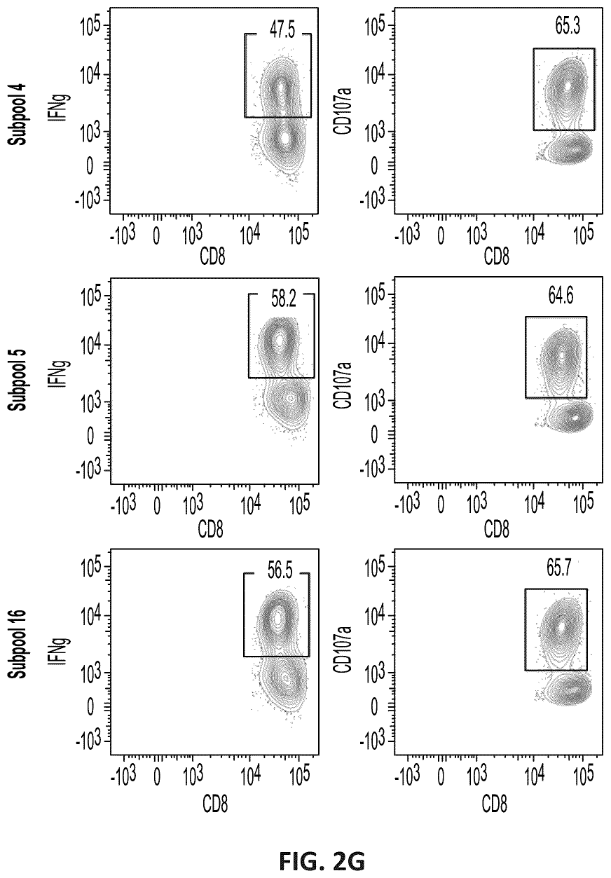

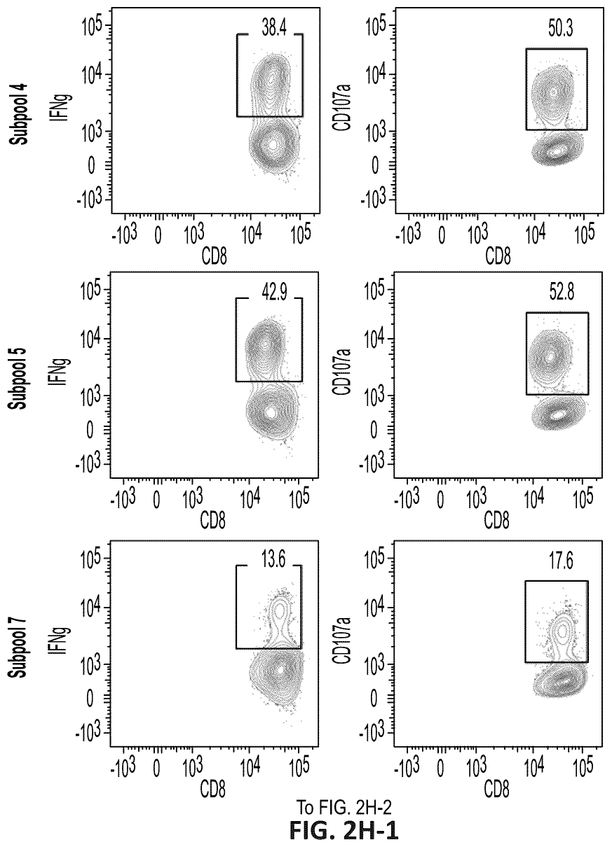

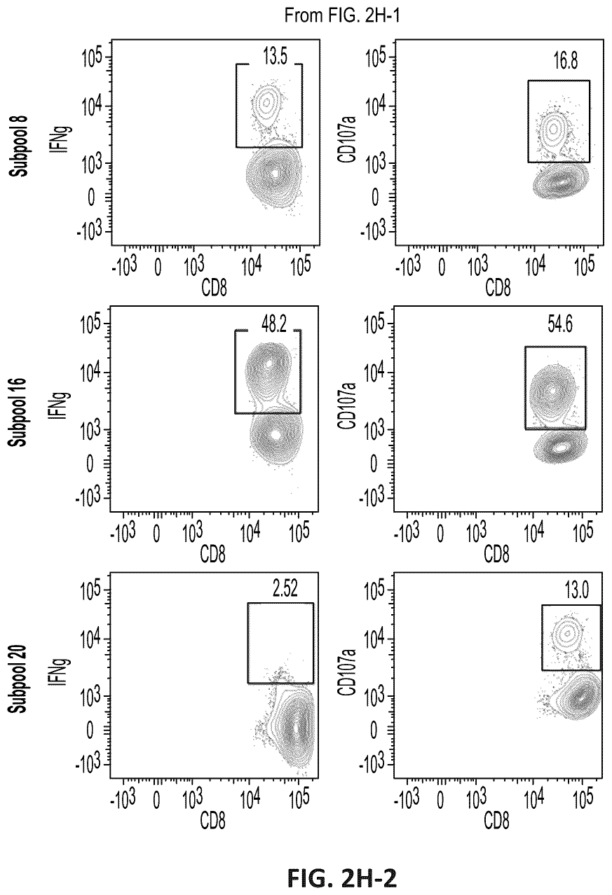

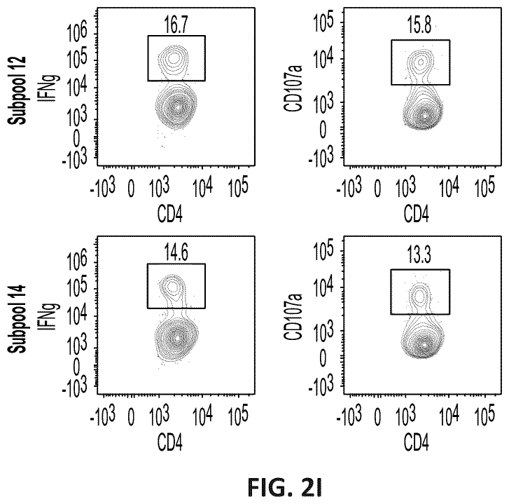

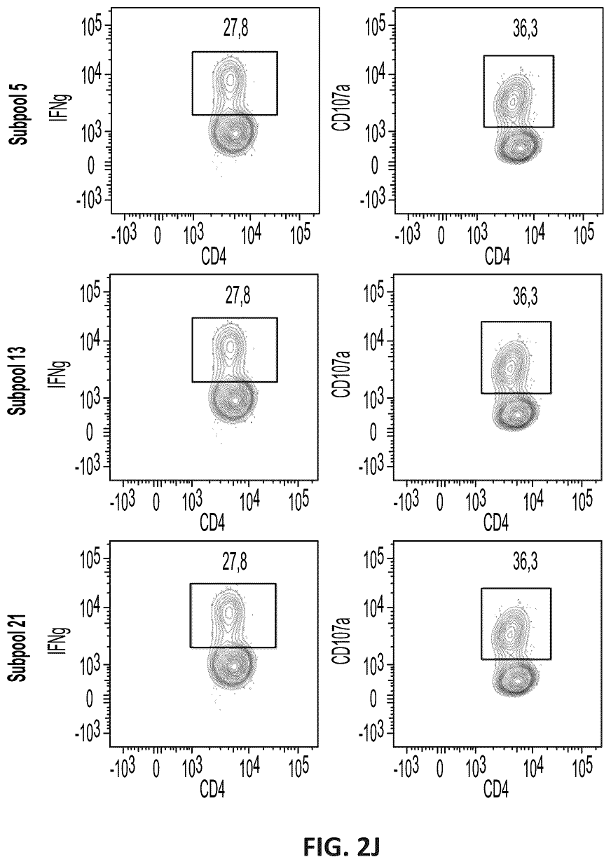

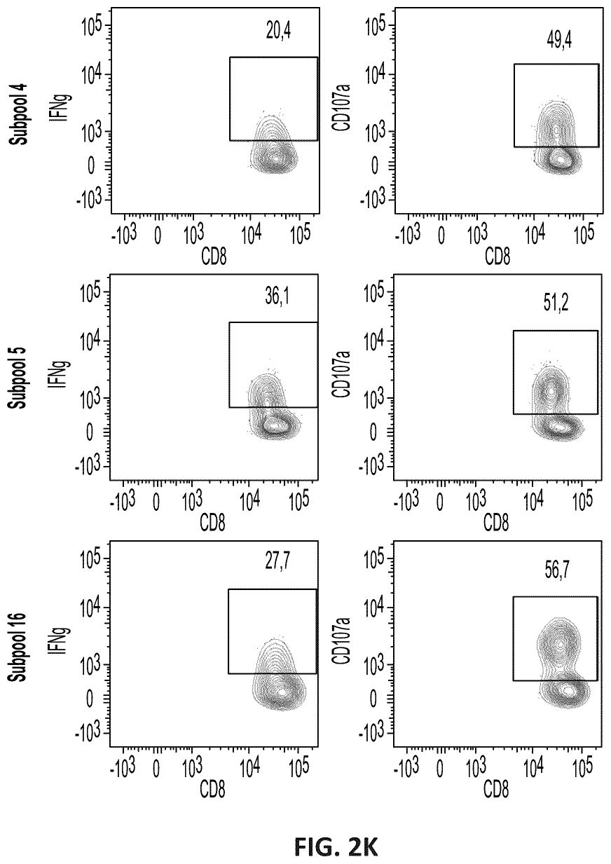

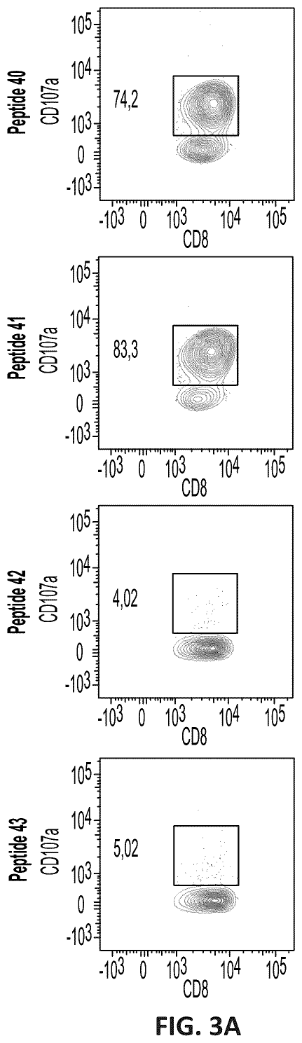

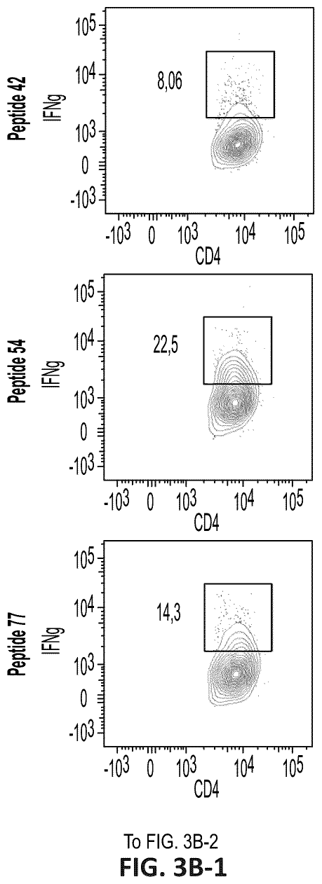

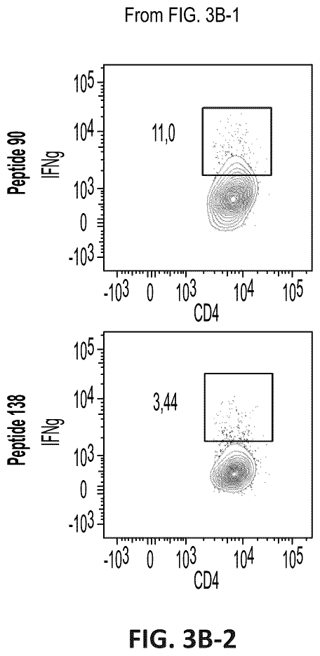

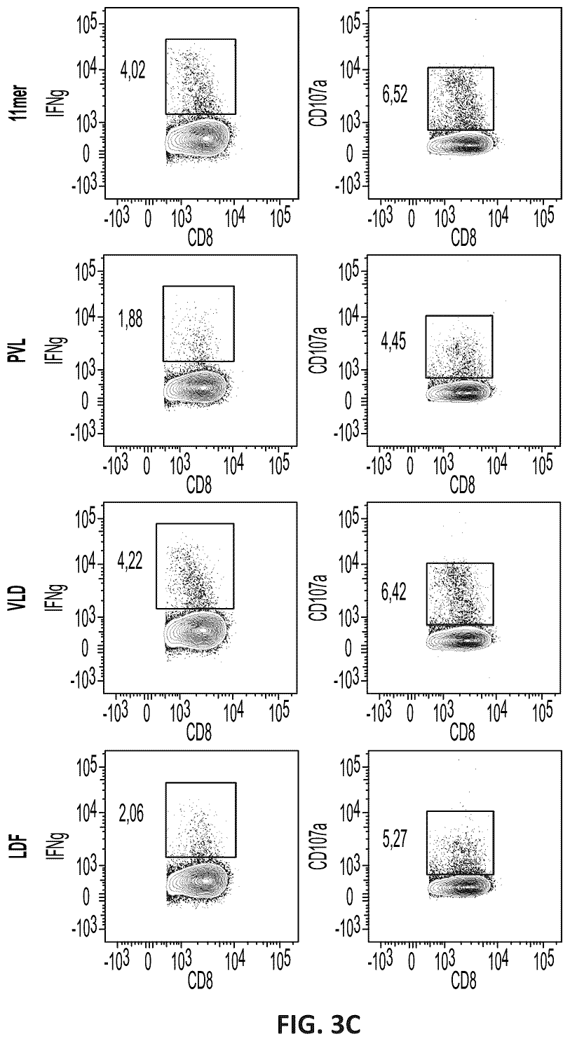

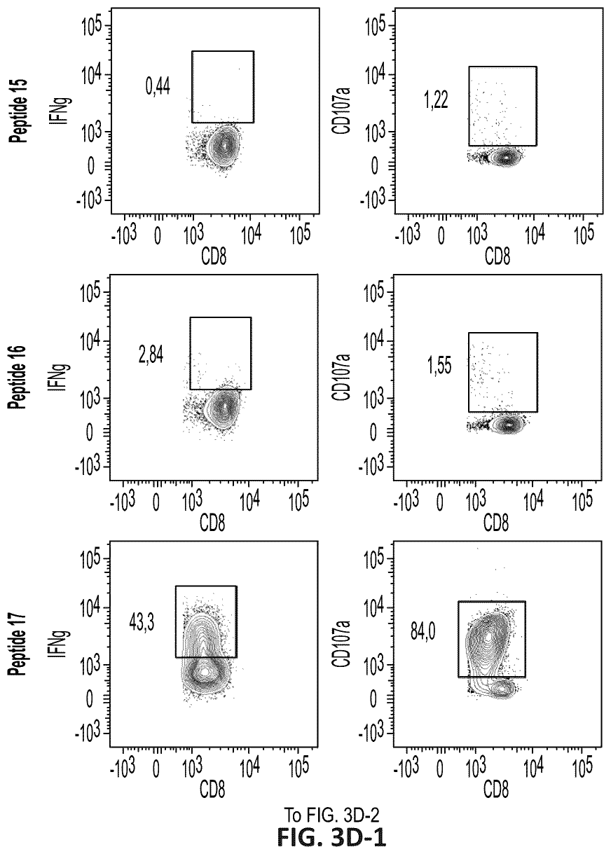

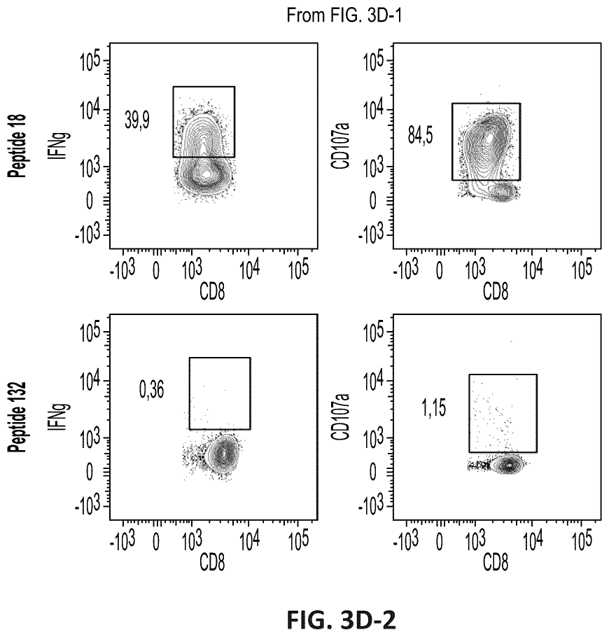

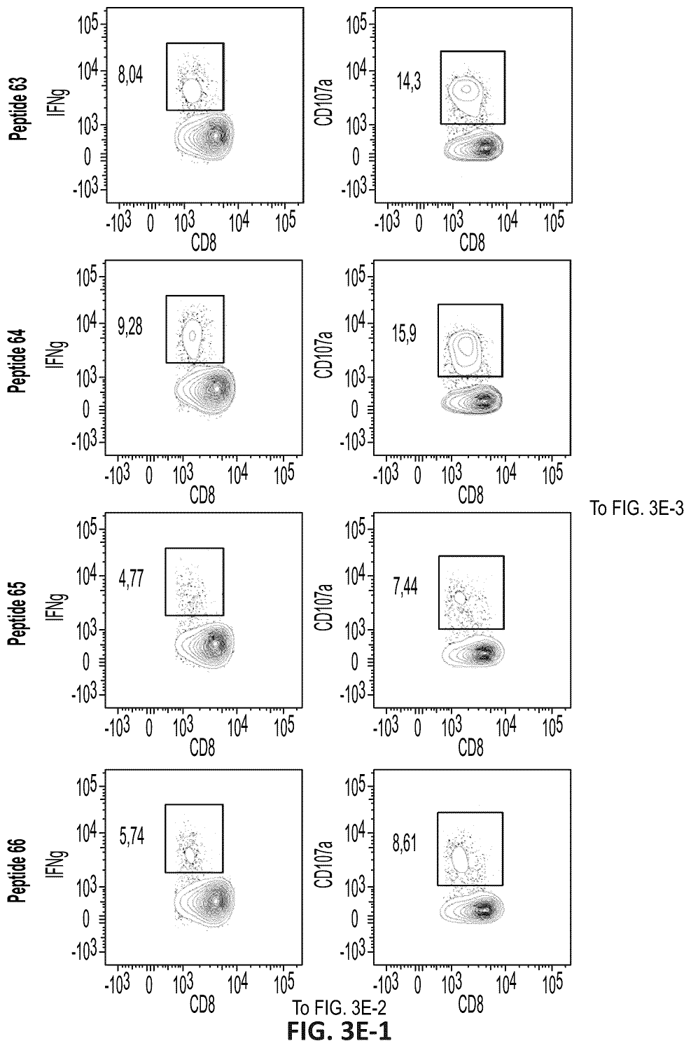

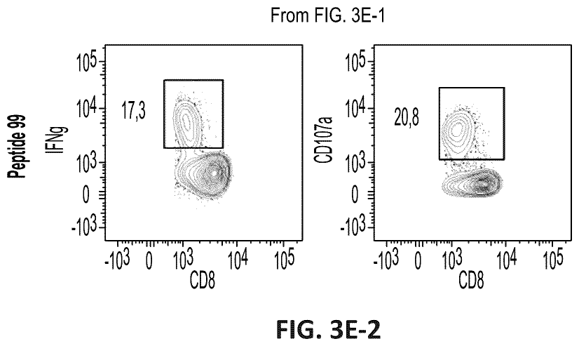

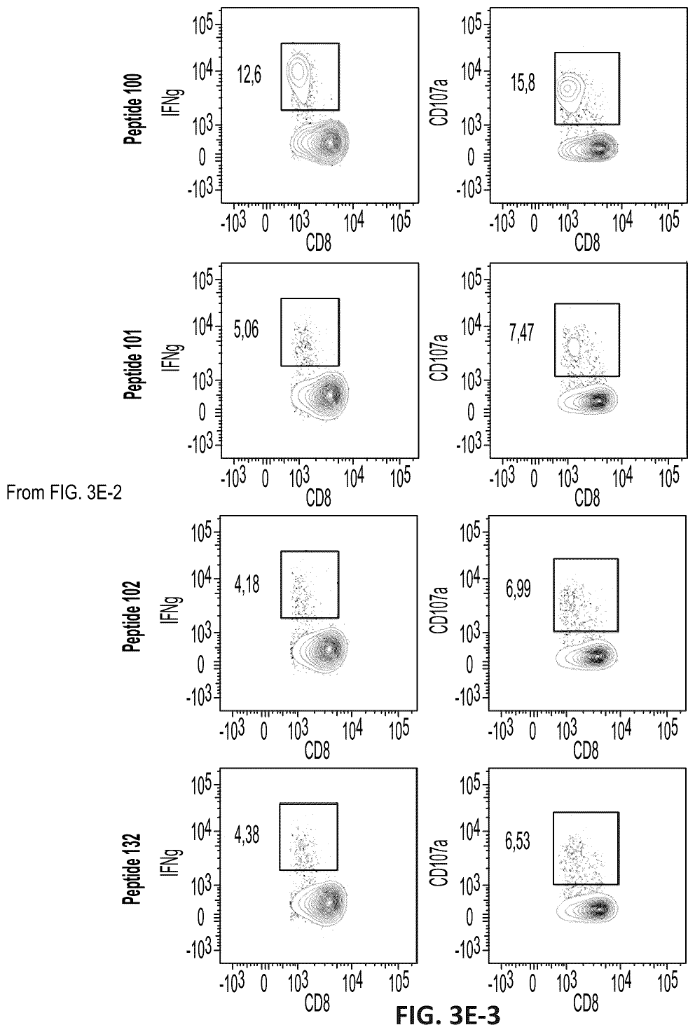

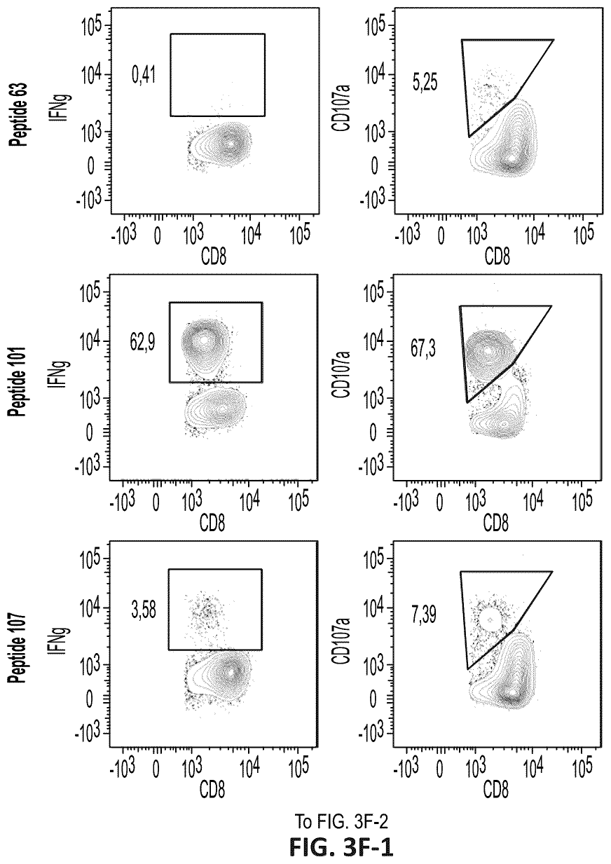

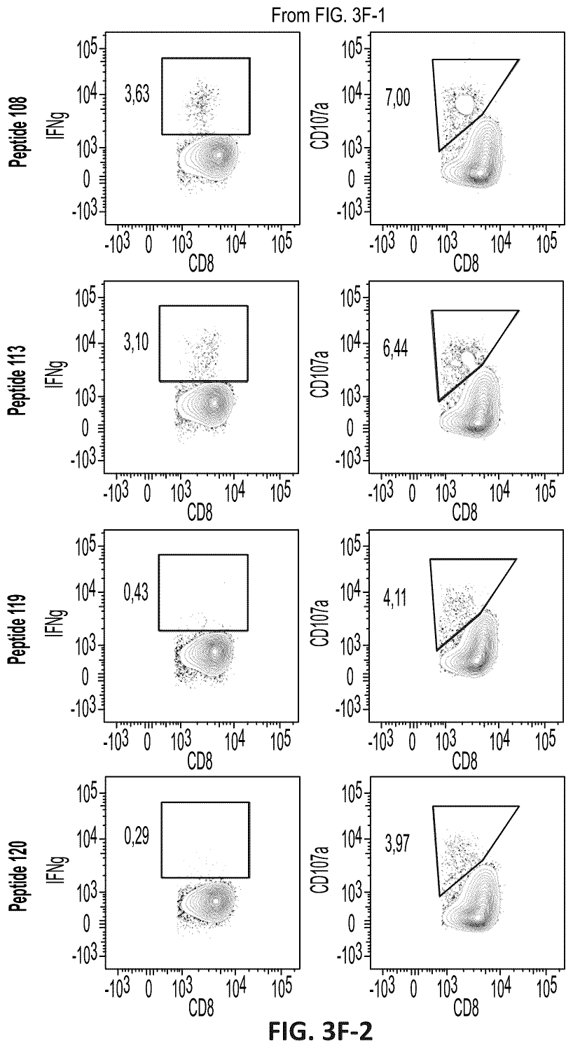

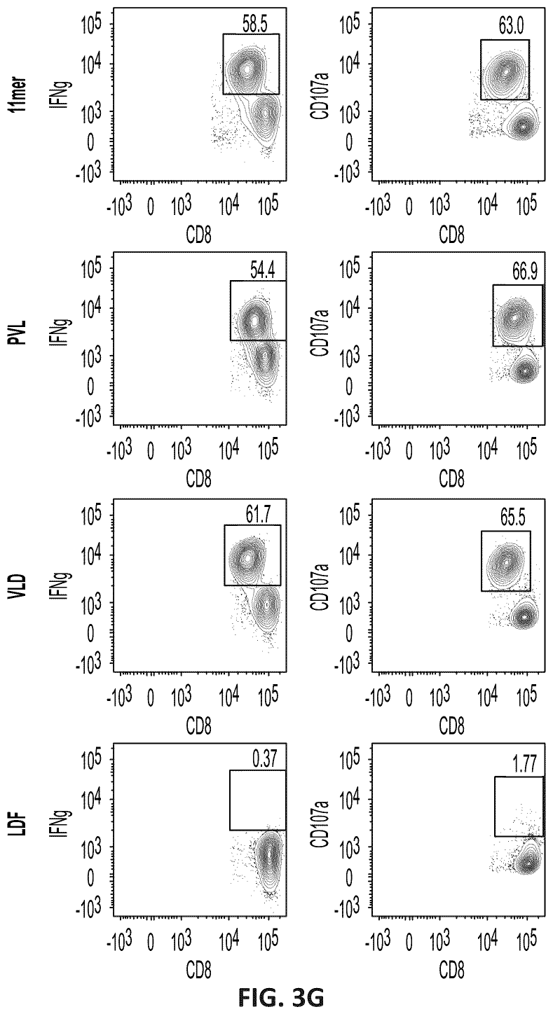

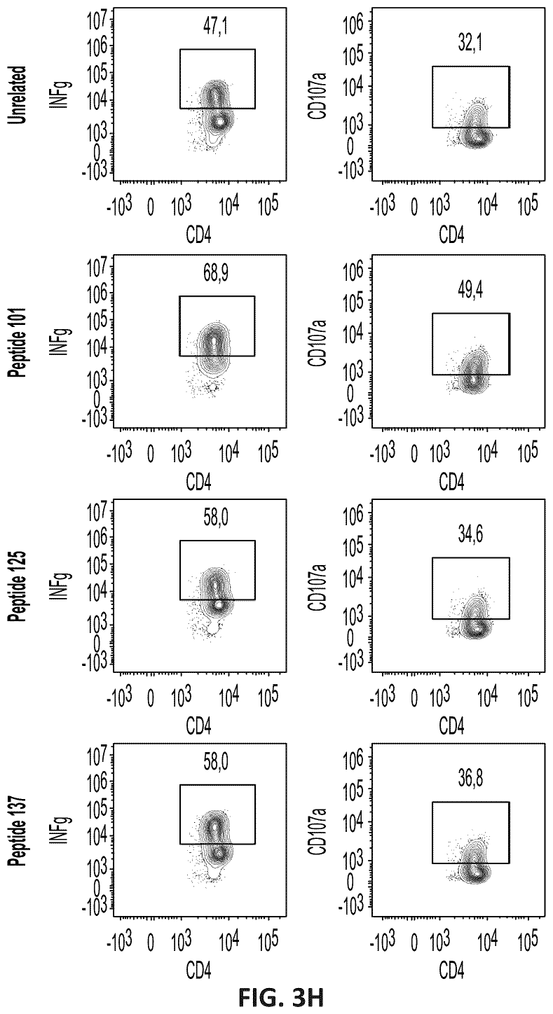

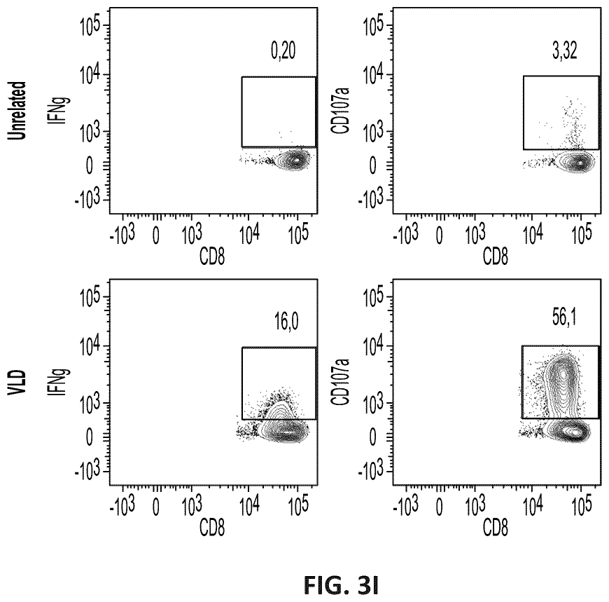

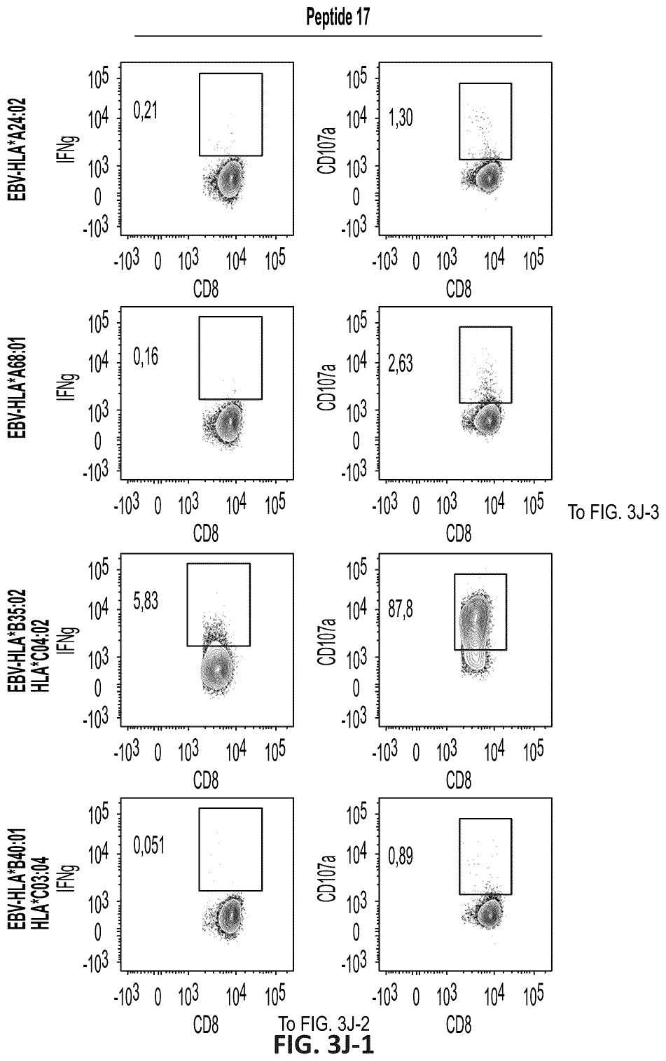

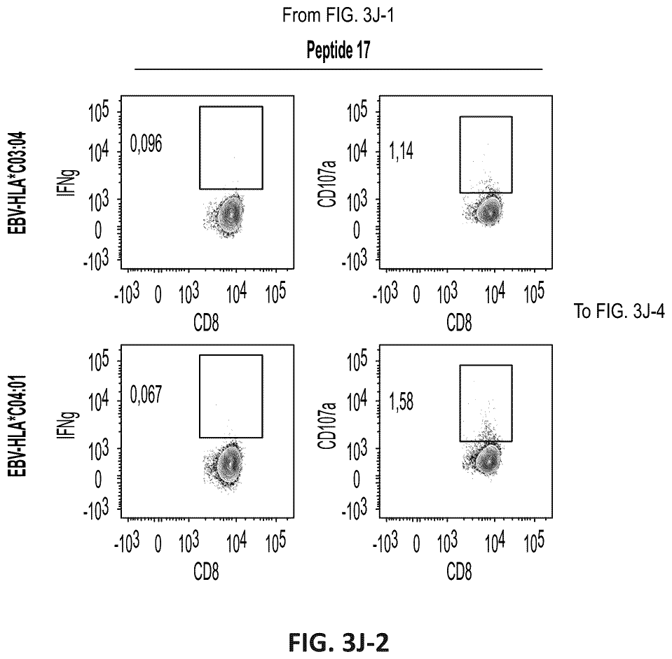

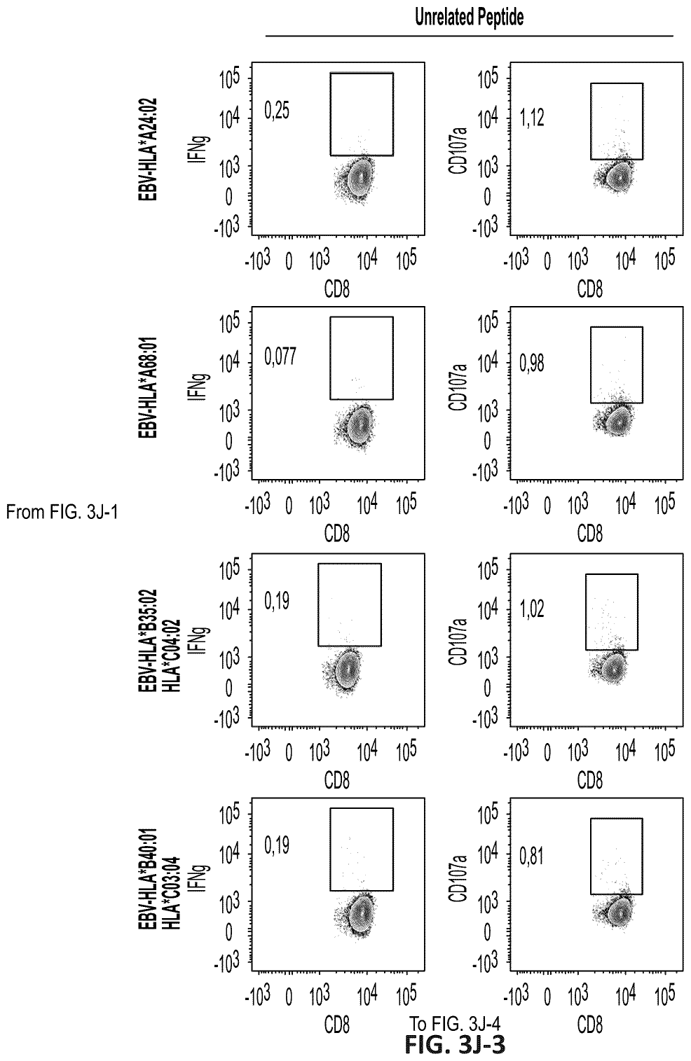

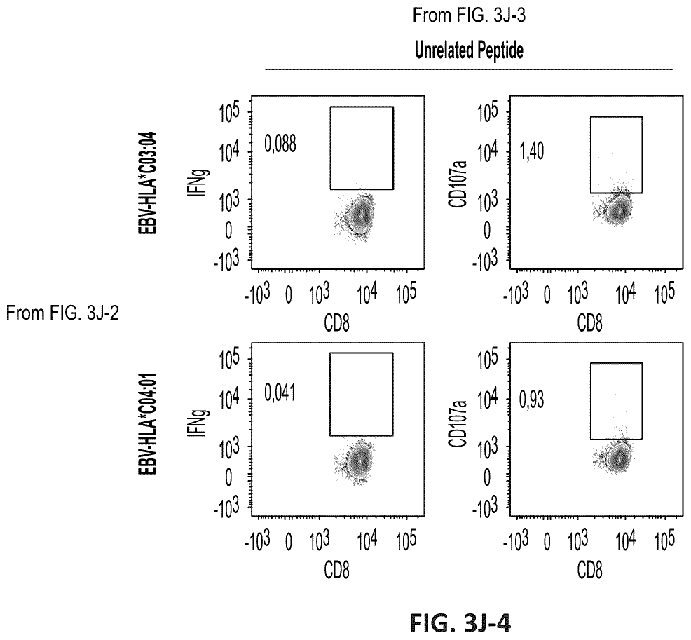

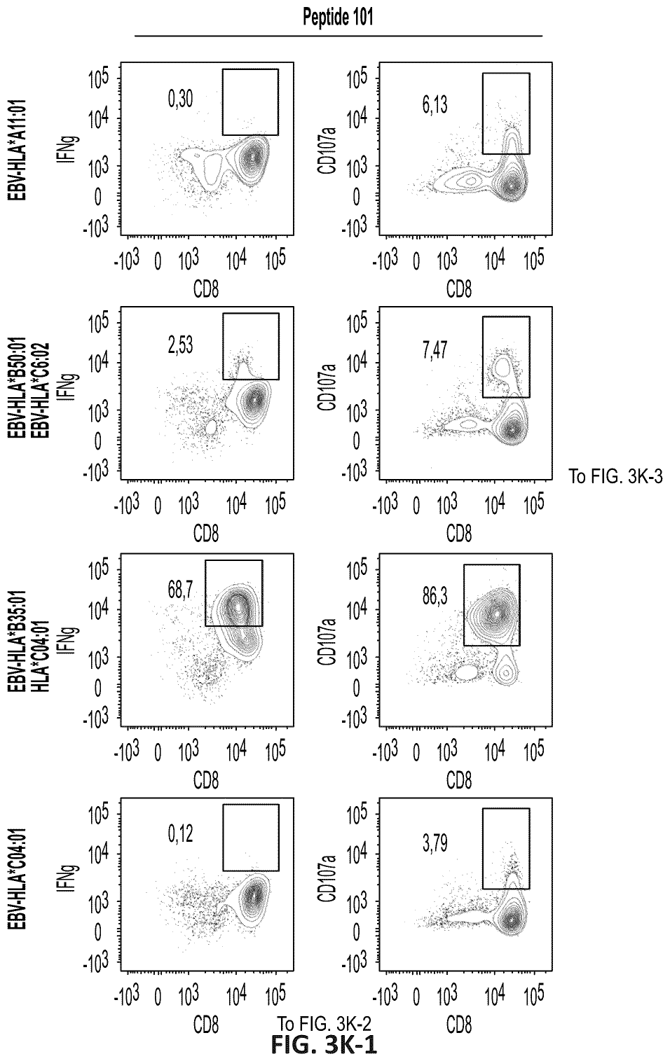

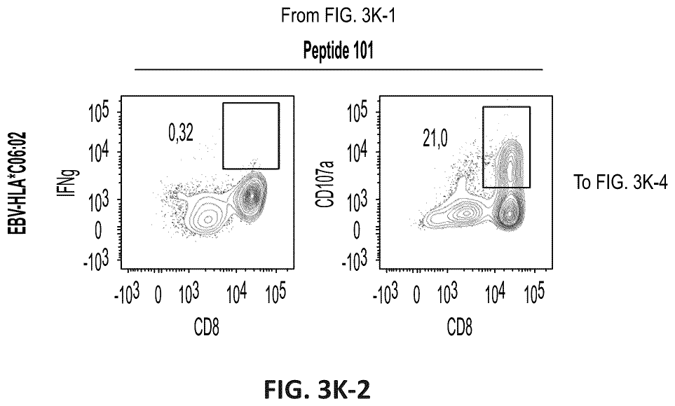

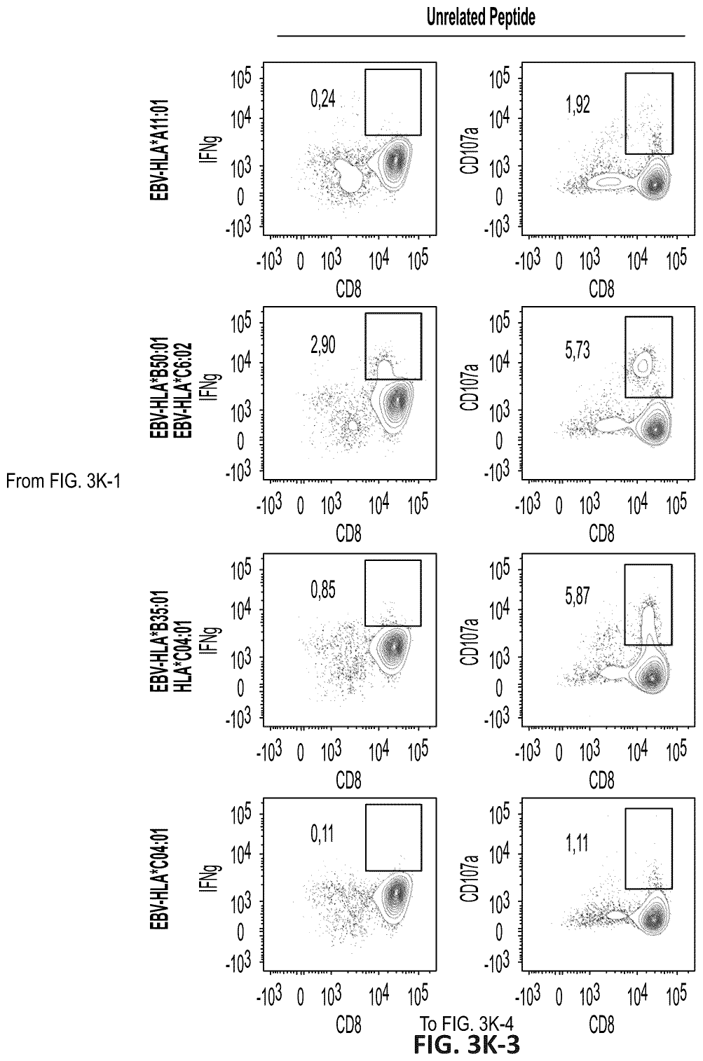

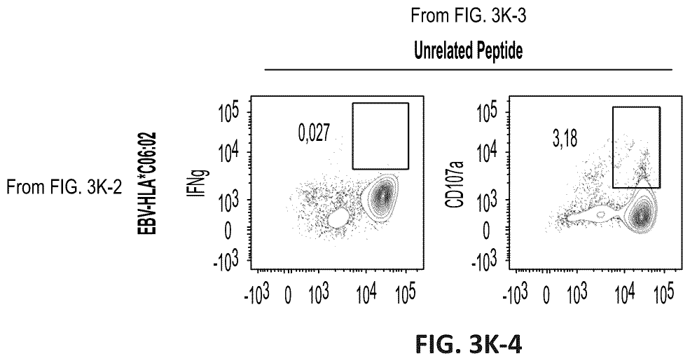

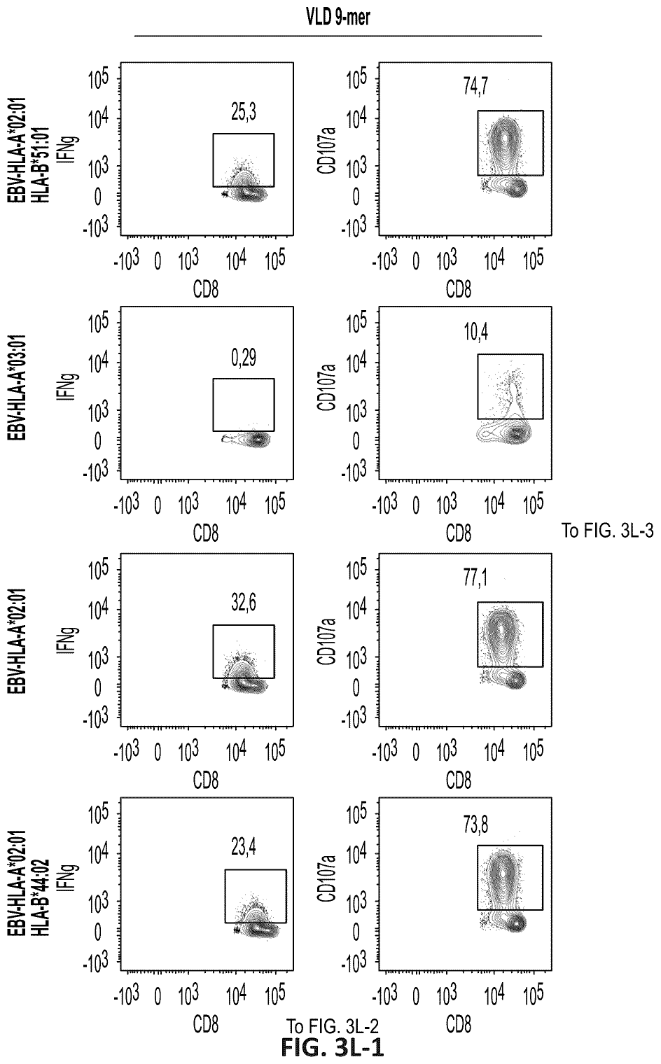

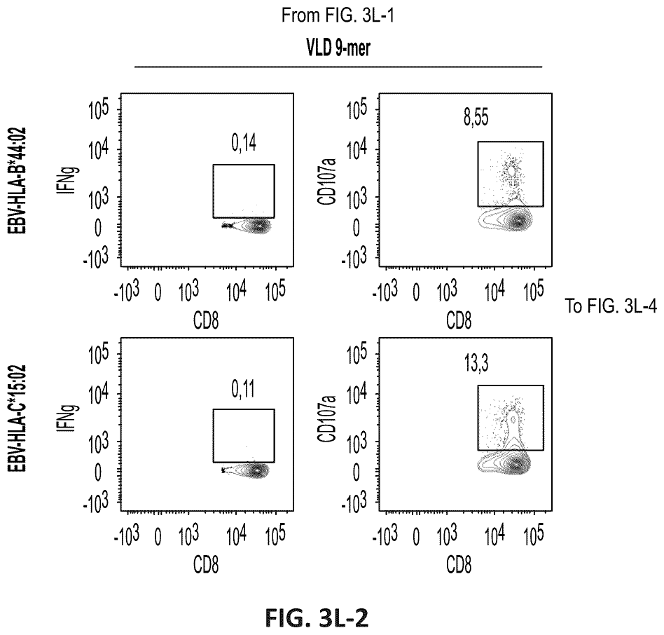

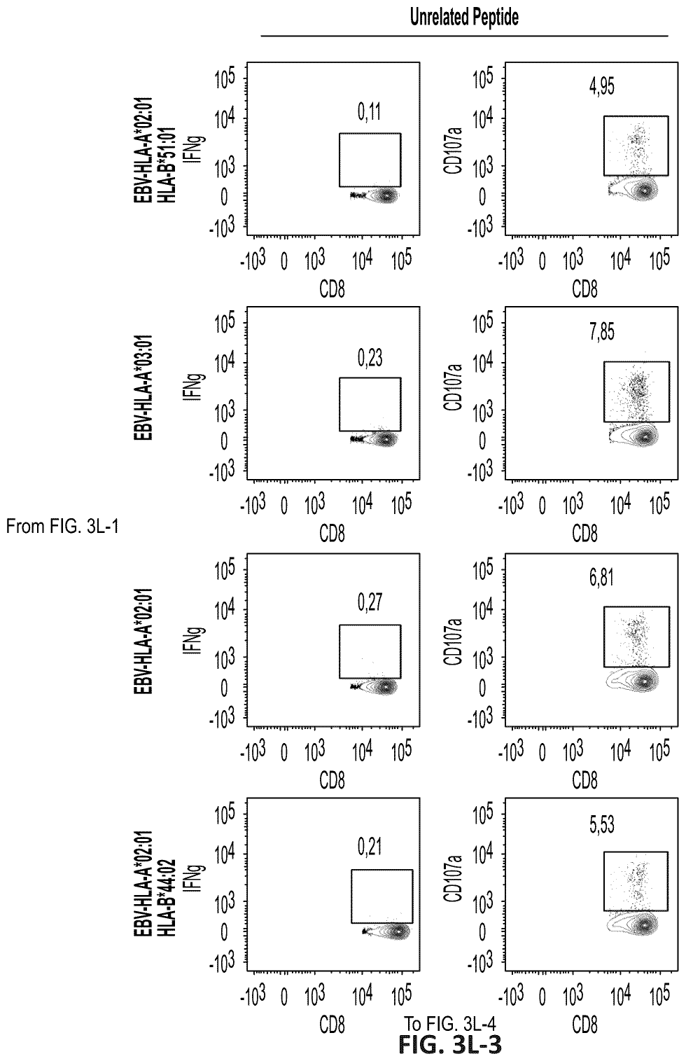

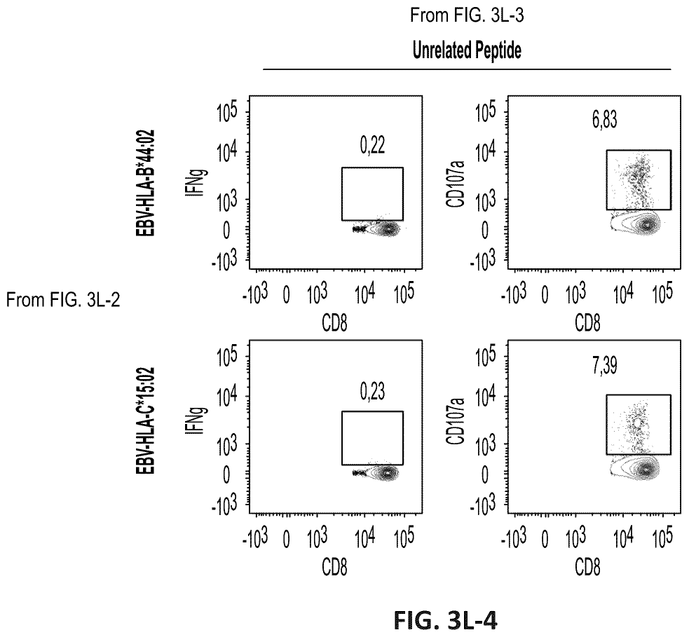

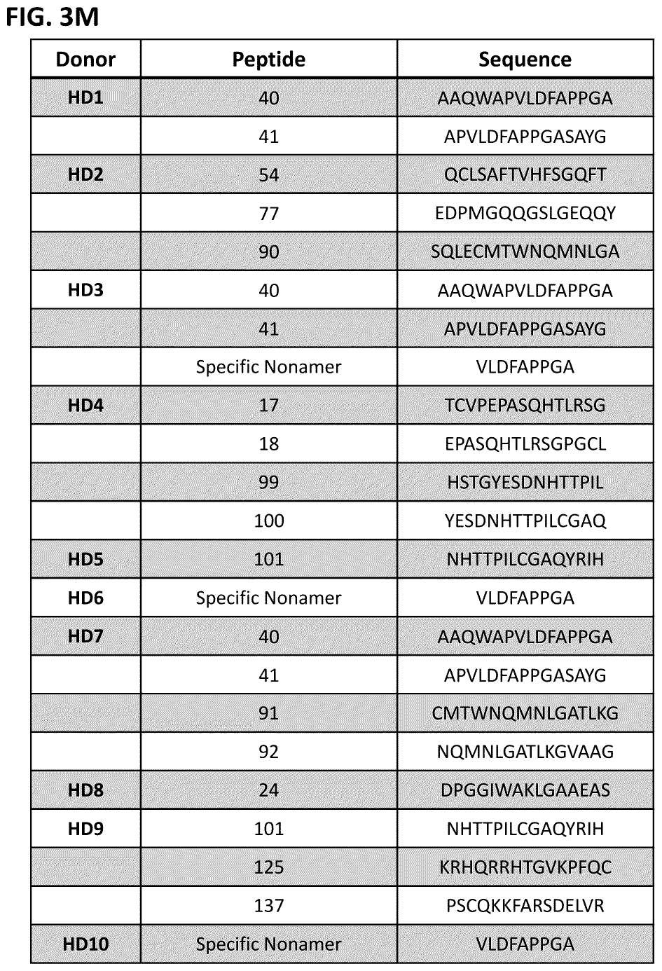

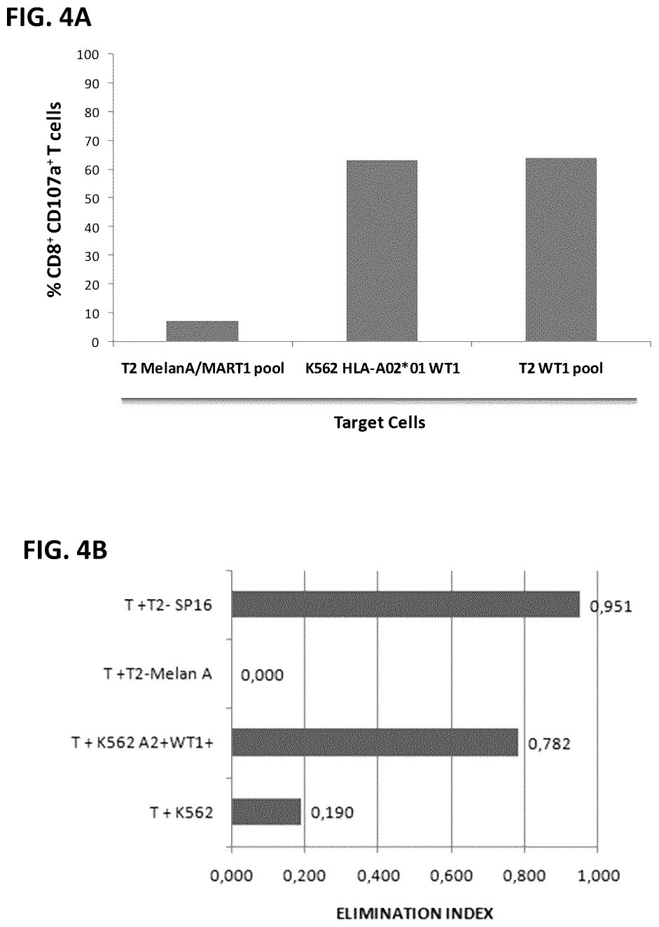

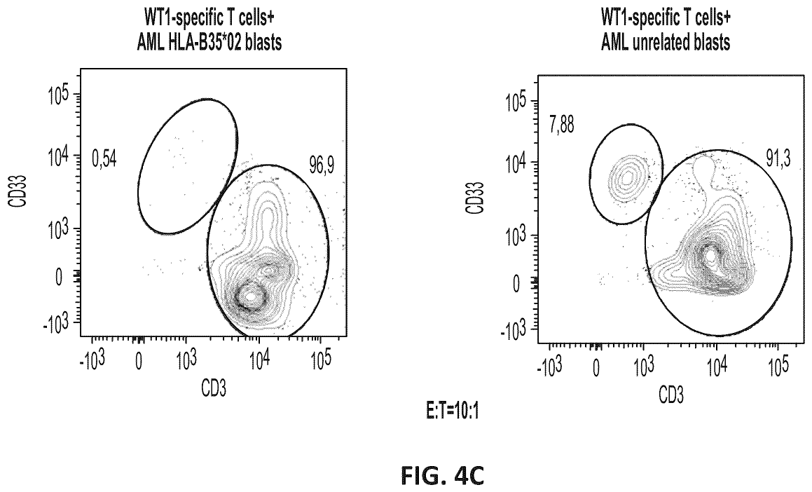

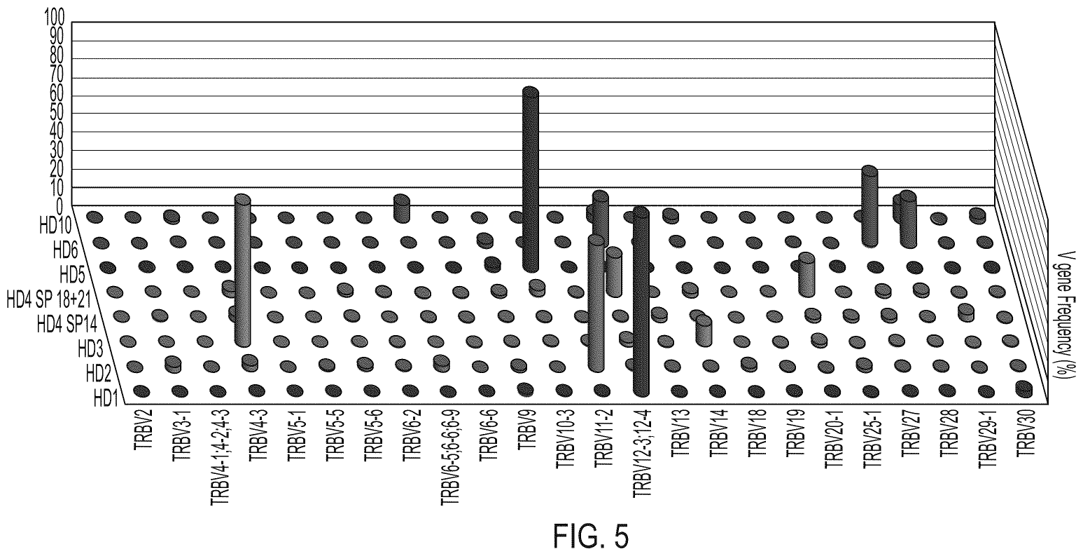

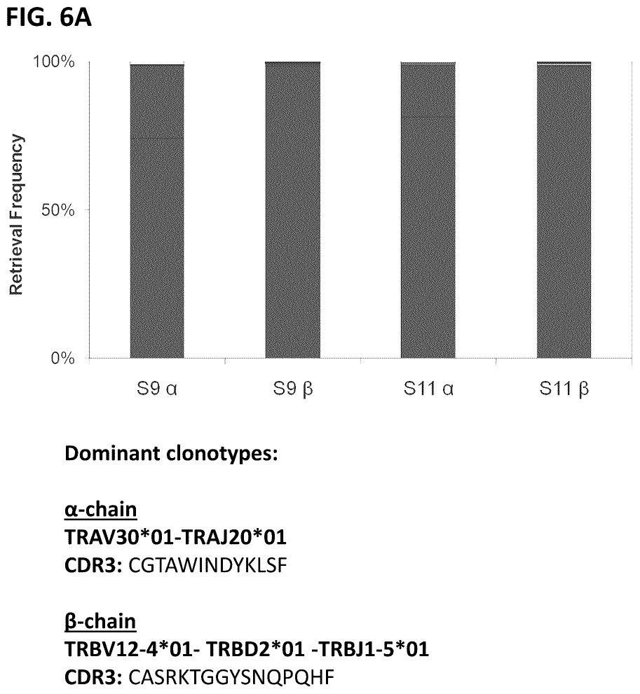

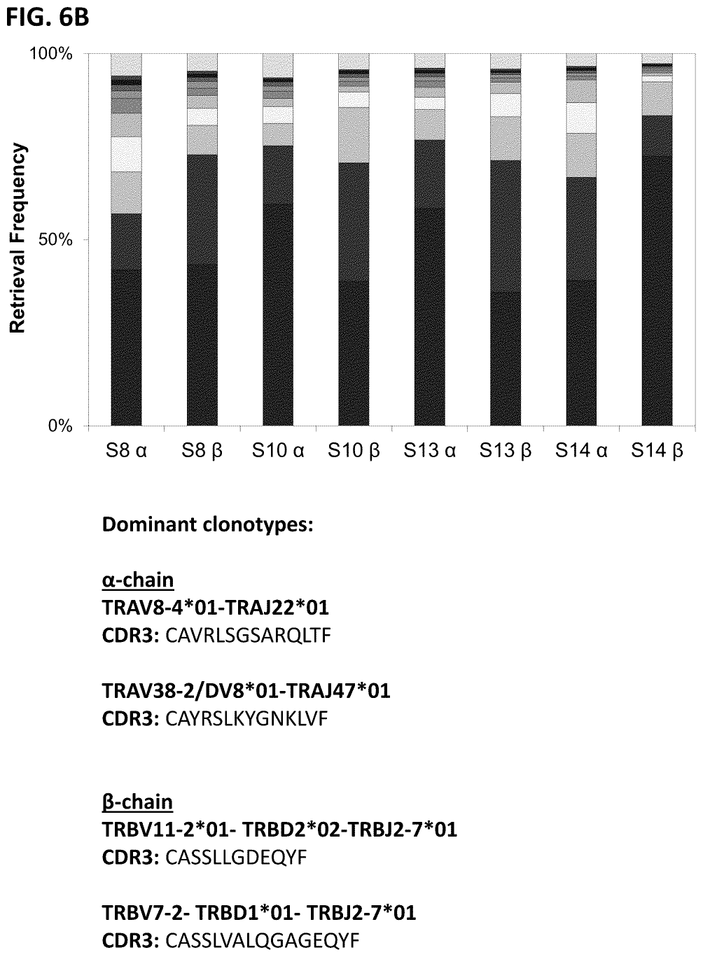

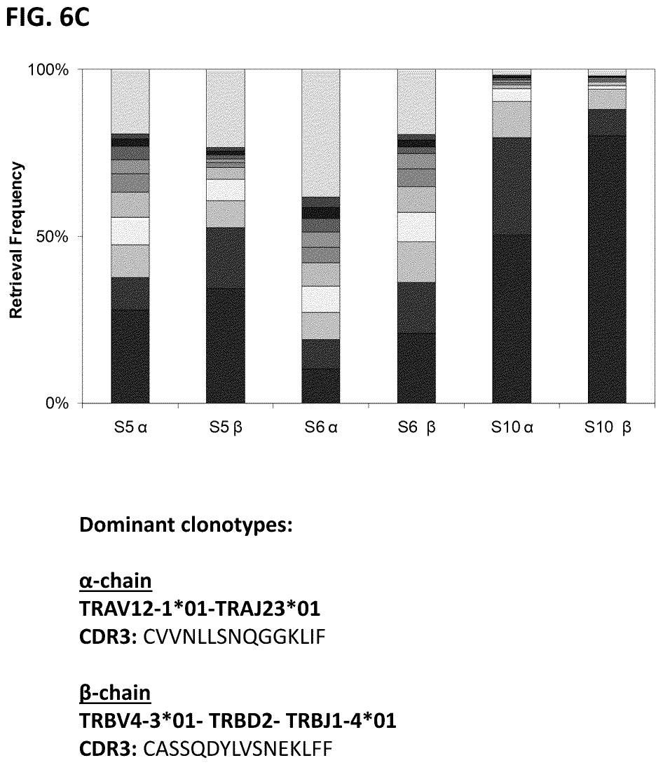

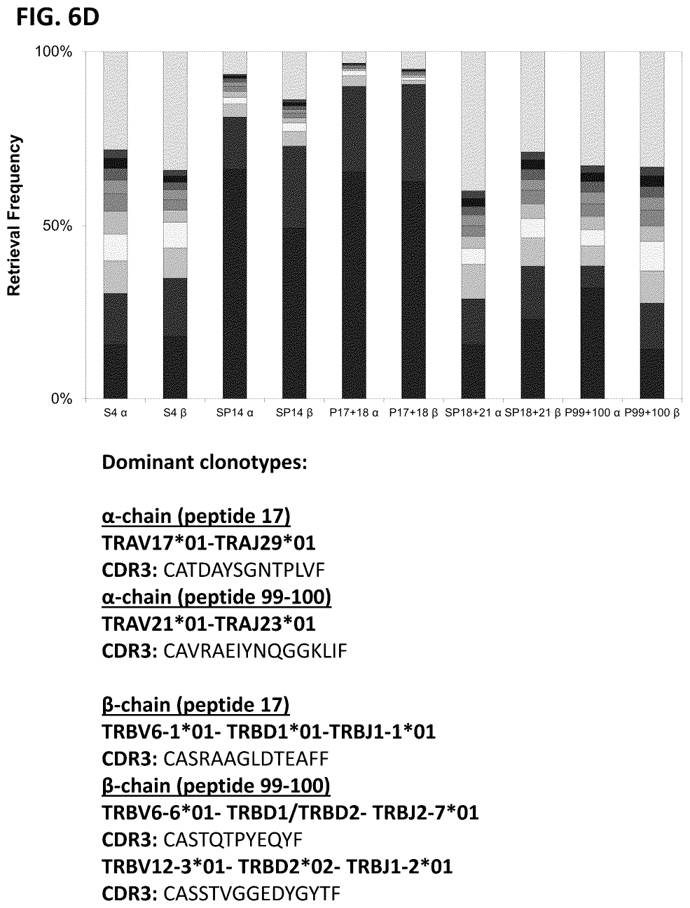

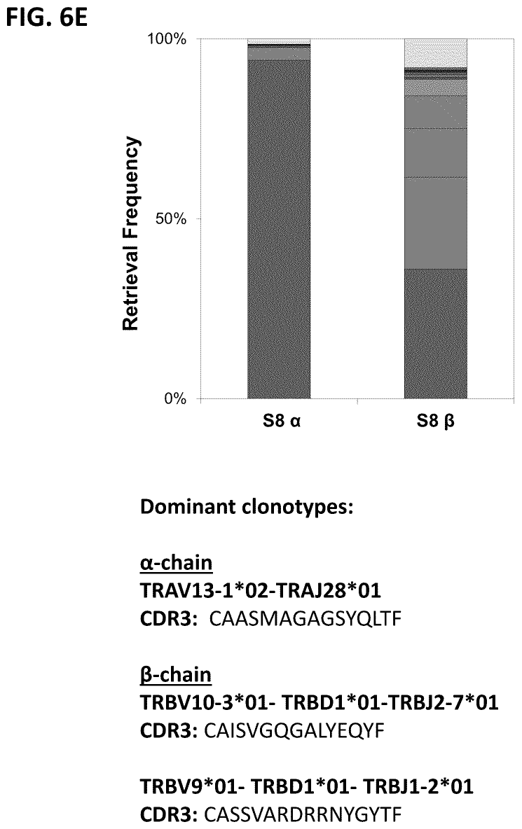

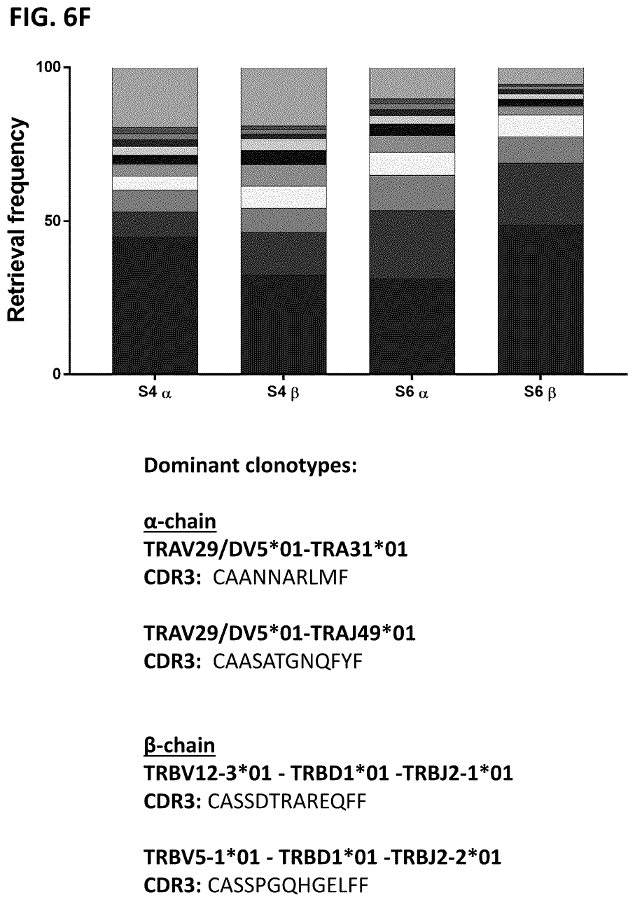

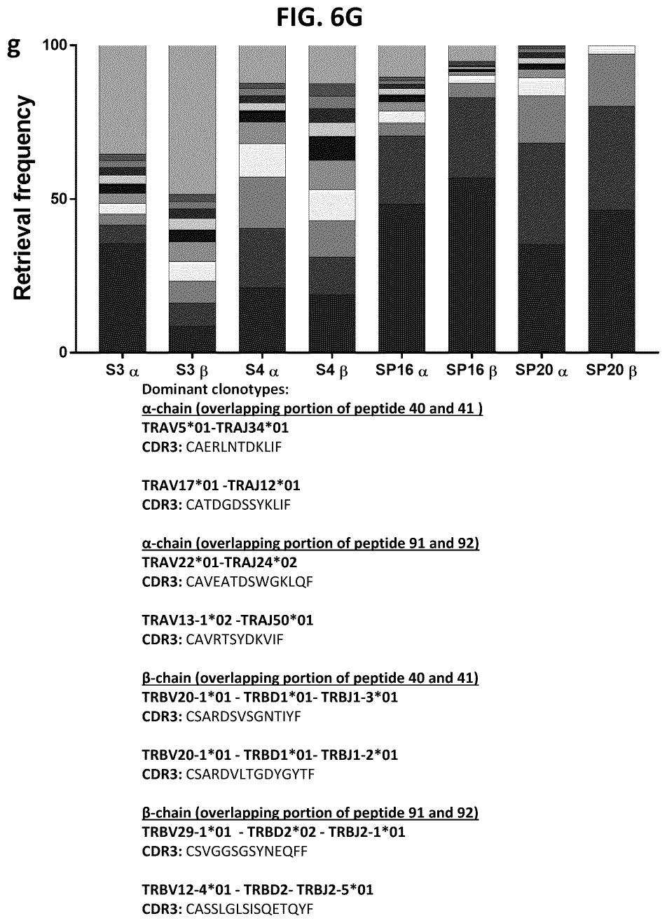

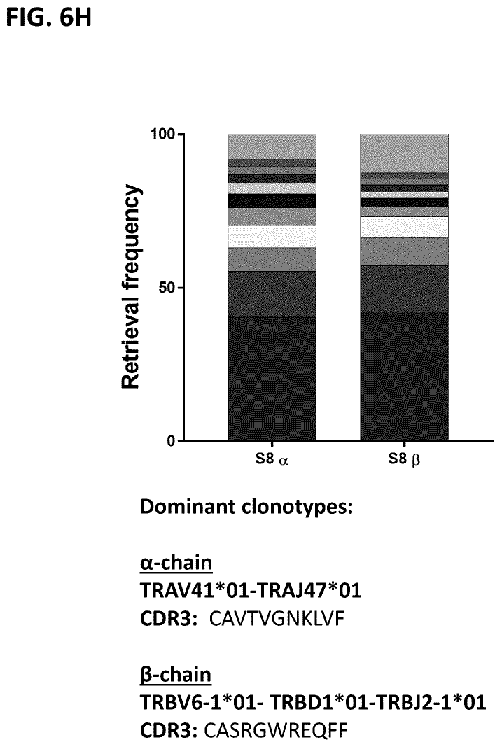

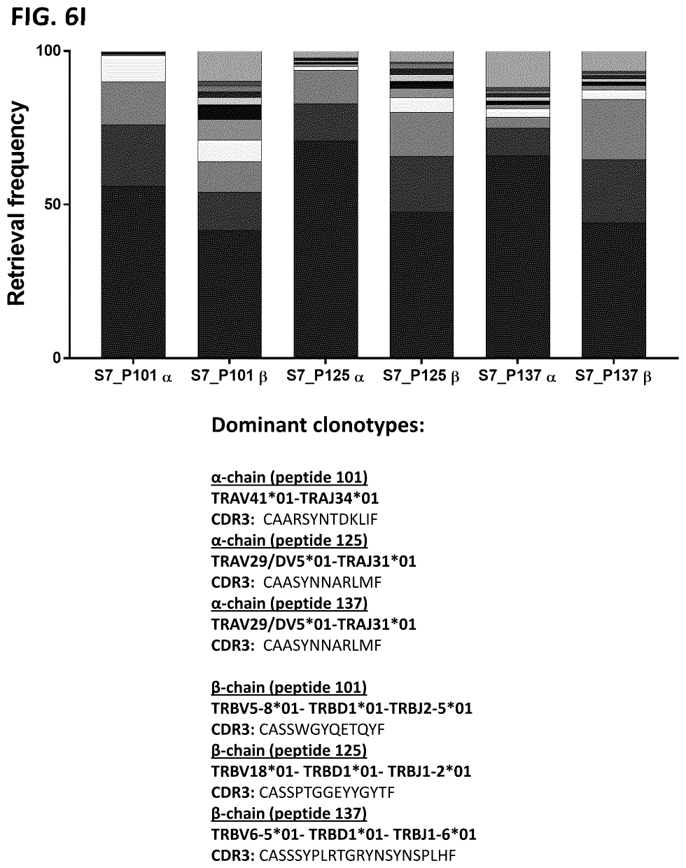

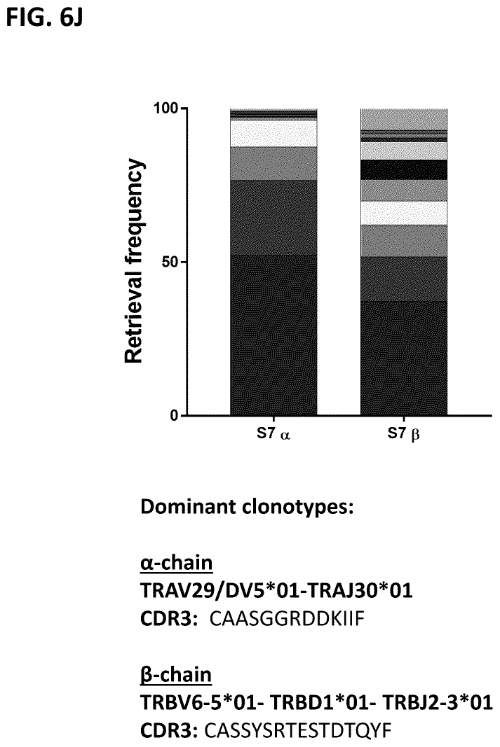

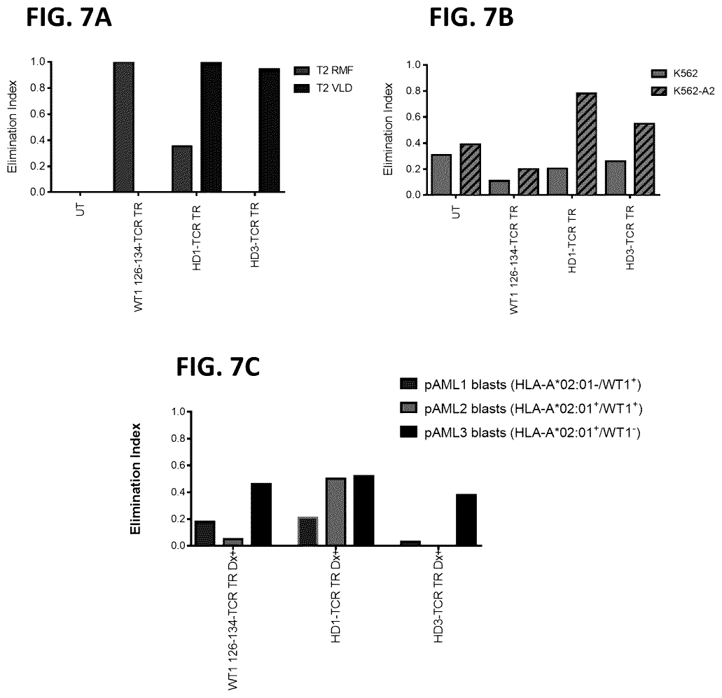

Peripheral blood mononuclear cells of ten healthy donors (HD) were stimulated with pooled, overlapping WT1 15-mer peptides for 26-30 hours, enriched for CD137⁺ cells, and expanded for 9-19 days. Expanded T-cells were re-stimulated for 6 hours with autologous antigen presenting cells (APCs) loaded with an unrelated peptide pool or WT1 peptide pool. Additionally, negative (T-cells unstimulated) and positive (T-cells cultured in the presence of PMA and lonomycin) controls were included in the experimental setting (not shown). Dot plots indicate the results of the intracellular staining for IFNγ production and CD107a exposure on cell surface. After several re-stimulations with autologous APCs loaded with WT1 peptide pool, T-cells specificity was tested by intracellular staining as previously described. Results showed an enrichment of WT1-specific T-cells in the CD8 T cell compartment for HD1 (FIG. 1A), HD3 (FIG. 1C), HD4 (FIG. 1D), HD5 (FIG. 1E), HD6 (FIG. 1F), HD7 (FIG. 1G), and HD10 (FIG. 1J) and in the CD4 T cell compartment for HD2 (FIG. 1B), HD8 (FIG. 1H) and HD9 (FIG. 1I). WT1, Wilms Tumor 1; PMA, Phorbol 12-myristate 13-acetate; IFNγ, interferon-γ; S, stimulation.

FIGS. 2A-2K. Grid and plots showing the identification of WT1-immunogenic peptides by a mapping grid strategy

Epitopes recognized by T-cells sensitized in vitro by repeated stimulations with the pool of overlapping WT1 peptides were identified by intracellular staining. In particular, the percentage of specific T-cells responding to the mapping grid of subpools of WT1 pentadecapeptides loaded on APCs was assessed. Additionally, negative (T-cells unstimulated and T-cells co-cultured with APCs loaded with an unrelated peptide pool) and positive (T-cells cultured in the presence of PMA and lonomycin) controls were included in the experimental setting (T-cells unstimulated and PMA/lono conditions are not shown).

(FIG. 2A) Deconvolution grid indicating the percentage of T-cells expressing IFNγ and CD107a after co-culture with APCs loaded with the different subpools (denoted SP1-24). IFNγ and CD107a values in bold text denote subpools that contain the WT1 epitope recognized by the T-cells. Representative dot plots relative to the co-culture of the T-cells with APCs loaded with the responsive subpools and indicating the expression of IFNγ and CD107a are reported. Dominant responses were observed for: subpools 4, 5, 16 in HD1 (FIG. 2B), HD3 (FIG. 2D), HD6 (FIG. 2G), HD7 (FIG. 2H), HD10 (FIG. 2K); subpools 6, 16, 17, 20, 23 in HD2 (FIG. 2C); subpools 4, 5, 6, 14, 18, 21 in HD4 (FIG. 2E); subpools 5, 11, 12, 21, 22 in HD5 (FIG. 2F); subpools 12, 14 for HD8 (FIG. 2I); subpools 5, 13, 21 for HD9 (FIG. 2J). For HD7, we also observed an increased IFNγ secretion and CD107a expression in response to subpools 7, 8, 20, even though at lower percentages compared to the response observed with subpools 4, 5, 16. SP, subpools; WT1, Wilms Tumor 1; APC, antigen-presenting cells; PMA, Phorbol 12-myristate 13-acetate; IFNγ, interferon-γ.

FIGS. 3A-3M. Epitope specificity of the WT1-specific T cells generated by sensitization with the pooled peptides.

In order to validate the WT1 immunogenic peptides, T-cells expanded from each HD were co-cultured for 6 hours in the presence of APCs loaded with the peptides identified after deconvolution of the mapping grid and with at least one unrelated peptide as negative control. Additionally, negative (T cells unstimulated) and positive (T cells cultured in the presence of PMA and lonomycin) controls were included in the experimental setting (not shown). Dot plots show for each HD the results of the intracellular staining for IFNγ and/or surface CD107a. Enrichment of CD107a and/or IFNγ positive cells was respectively observed for T-cells co-cultured with peptides 40 and 41 for HD1 (FIG. 3Aa) and not for peptide 42 and 43 (unrelated peptides); peptides 54, 77, 90 for HD2 (FIG. 3B) and not for peptide 42 and 138 (unrelated peptides); peptide VLDFAPPGA (SEQ ID NO: 157, VLD, which is a nonamer of the peptide represented by SEQ ID NO: 117 (referred to as “11 mer” in FIG. 3c)) for HD3 (FIG. 3C) and low response with peptides PVLDFAPPG (SEQ ID NO: 158, PVL, which is another nonamer of the peptide represented by SEQ ID NO: 117) and LDFAPPGAS (SEQ ID NO: 159, LDF, which is a nonamer of the peptide represented by SEQ ID NO: 116, previously described as an immunogenic peptide (Doubrovina, E. et al. (2012) Blood 120: 1633-1646)); peptides 17, 18, 99, 100 for HD4 (FIG. 3D, FIG. 3E) and not for the unrelated peptides (15, 16, 63-66, 101, 102 and 132); peptide 101 for HD5 (FIG. 3F) and not for the unrelated peptides (63, 107, 108, 113, 119 and 120); peptide VLDFAPPGA (SEQ ID NO: 157, VLD, which is a nonamer of the peptide represented by SEQ ID NO: 117 (referred to as “11 mer” in FIG. 3c)) for HD6 (FIG. 3G) and peptide PVLDFAPPG (SEQ ID NO: 158, PVL, which is another nonamer of the peptide represented by SEQ ID NO: 117) and not for peptide LDFAPPGAS (SEQ ID NO: 159, LDF, which is a nonamer of the peptide represented by SEQ ID NO: 116, previously described as an immunogenic peptide (Doubrovina, E. et al. (2012) Blood 120: 1633-1646)); peptides 101, 125, 137 for HD9 (FIG. 3H); peptide VLDFAPPGA (SEQ ID NO: 157, VLD, which is a nonamer of the peptide represented by SEQ ID NO: 117 (referred to as “11 mer” in FIG. 3c)) for HD10 (FIG. 3I) and not for the unrelated peptide. For HD7 and HD8, due to a reduced fitness of T cells, it was not possible to perform functional tests to verify the peptide predicted by the deconvolution of the mapping grid, i.e. peptides 40, 41, 91, 92 for HD7 and peptide 24 for HD8.

In order to determine the HLA restriction of the WT1 epitopes identified for HD4, HD5 and HD10 T-cells, donor DNA was sequenced to determine the HLA typing. Afterwards, WT1-specific T-cells were co-cultured with different antigen presenting EBV-BLCL cell lines, each one harboring a specific HLA allele of interest that was identified by sequencing of the HD4, HD5 or HD10 DNA. The EBV-BLCL cells were pulsed with peptide 17 for HD4, peptide 101 for HD5 and peptide VLDFAPPGA (SEQ ID NO: 157) or with an unrelated control peptide. After co-culture for 6 hours, we observed a substantial response to WT1 by the WT1-specific T-cells that had been co-cultured with EBV-BLCL cells expressing the HLA-B*3502 allele and pulsed with peptide 17 for HD4 (FIG. 3J), EBV-BLCL cells expressing the HLA-B*3501 allele and pulsed with peptide 101 for HD5 (FIG. 3K) and EBV-BLCL cells expressing the HLA-A*0201 allele and pulsed with peptide VLDFAPPGA (SEQ ID NO: 157) for HD10 (FIG. 3L). (FIG. 3M) Table showing the peptides recognized by T-cells expanded from HD1-HD10. For HD3, HD6 and HD10, the specific nonamer overlapping peptides 40 and 41 and eliciting an immune response is shown. Wilms' Tumor 1; APC, antigen-presenting cells; PMA, 2; Phorbol 12-myristate 13-acetate; IFNγ, interferon-γ; S, stimulation.

FIGS. 4A-4C. Graphs and plots showing that expanded T-cells of HD1, HD3 and HD4 recognize a naturally processed WT1 epitope

(FIG. 4A) Graph depicting CD107 expression by CD8⁺ T-cells expanded from HD1 following co-culture with T2 cells pulsed with WT1 pool, K562 cells genetically modified to express the HLA-A*0201 allele and to overexpress the WT1 protein, or T2 cells pulsed with the non-specific control MelanA/MART1 pool as a negative control.

(FIG. 4B) Graph depicting the results of experiments to determine the ability of HD3 expanded T-cells to target WT1-expressing cells. The results are represented as an elimination index, which is calculated as the total number of target cells still present after co-culture with the WT1-specific T-cells divided by the total number of target cells alone. HD3 T-cells were co-cultured with T2 cells pulsed with the subpool 16 (SP16) containing the immunogenic peptide eliciting the immune response; T2 cells pulsed with the MelanA/MART1 pool (Melan A) as negative control; K562 cells either wild type (K562) or genetically modified in order to express both the HLA-A*0201 allele and to overexpress the WT1 protein (K562 A2+WT1+).

(FIG. 4C) Plots depicting the results of experiments to determine the ability of WT1-specific T-cells from HD4 to eliminate target cells. HD4 T-cells were co-cultured with primary CD33+ blasts harvested from a HLA-B*3502 patient at a ratio of 10:1 or, as control, with leukemic cells from a patient not harboring the HLA-B*3502 allele. After 3 days of co-culture, results indicate a nearly complete clearance of the CD33+ HLA-B*3502 blasts when seeded with WT1-specific T-cells (CD3⁺ cells). E, effector; T, target.

FIG. 5. Graph showing results of Vβ profiling of WT1-specific T-cells

WT1-specific T-cells generated from the different HDs after several stimulations with the WT1 pool were stained with the Vβ Immunoprofiling kit in order to determine the clonality of the population. In particular, the expression of the variable (V) genes of the β-chain was determined by FACS analysis. Results indicate the expression of a highly dominant Vβ gene in HD1 (TRBV12-3; 12-4), HD2 (TRBV11-2), HD3 (TRBV4-3), HD5 (TRBV20-1) whereas for HD4, HD6, HD10 a clear enrichment of a defined Vβ was not detected. HD4 SP14 indicates T cells stimulated with subpool 14 which contains peptides 17-18 eliciting the highest immune response; HD4 SP18+21 indicates T cells stimulated with subpools 18 and 21 which contain peptides 63-64-65-66 and 99-100-101-102, respectively, eliciting a minimal immune response as shown in FIG. 3. For HD7, HD8 and HD9, it was not possible to perform the VR Immunoprofiling analysis due to a reduced cell fitness.

FIGS. 6A-6J. Graphs showing results of TCR sequencing of enriched WT1-specific T-cells over time

T-cells generated from each healthy donor included in the experimental setting were characterized by TCR αβ sequencing after several stimulations with the WT1 pool. Sequencing results indicated the presence of predominant clonotypes for HD1 (FIG. 6A), HD2 (FIG. 6B) and HD3 (FIG. 6C), HD4 (FIG. 6D), HD5 (FIG. 6E), HD6 (FIG. 6F), HD7 (FIG. 6G), HD8 (FIG. 6H), HD9 (FIG. 6I), HD10 (FIG. 6J). Bar charts depict the ten most predominant CDR3 amino acid sequences identified at each time point (e.g. S9 corresponds to the sequencing results obtained following the 9^{th} round of stimulation). For each bar, starting from the x-axis, the bottom segment represents the most predominant CDR sequence. The next nine most predominant sequences are stacked above the bottom segment and are ordered by decreasing frequency going upwards. The remaining sequences are grouped together in top segment. WT1, Wilms Tumor 1; CDR3, complementarity determining region 3; S, stimulation.

FIGS. 7A-7C. Functional activity of genetically-modified T lymphocytes.

T cells isolated from PBMCs of healthy individuals were transduced with a bidirectional lentiviral vector encoding for the α and the β chain of TCRs isolated from HD1 and HD3. As control we transduced T cells with a previously published TCR recognizing the WT1 126-134 (RMFPNAPYL; SEQ ID NO: 255) peptide when presented by the HLA-A*0201 allele. Transfer (TR) T lymphocytes were co-cultured for 3 days with (a) T2 cells either pulsed or not with the WT1 126-134 peptide or with the VLDFAPPGA (SEQ ID NO: 157) peptide (effector:target ratio=1:1); (b) K562 cells either wild type (K562) or genetically modified in order to express the HLA-A*0201 allele (effector:target ratio=1:1); (c) 3 different primary AML blasts selected according to the expression of the HLA-A*0201 allele and of the WT1 antigen (effector:target ratio=5:1). For the co-culture with T2 and K562 cell lines, we included untransduced T cells as control. Results indicated the ability of each TCR in recognizing the target peptide when presented by the HLA-A*0201 allele (FIG. 7a) and the greater potential of HD1 TCR-transduced T cells in mediating a specific and near complete elimination of K562 cells harbouring the HLA*A0201 allele compared to HD3-TR T cells. Of note, no substantial killing of target cells was observed in the co-culture of K562 HLA*A0201 cells with WT1 126-134 TR T cells (FIG. 7b). These results were further confirmed by the outcome of the co-culture experiment performed using as target cells primary AML blasts derived from 3 different AML patients (pAML1 blasts: WT1-/HLA-A*0201+; pAML2 and pAML3 blasts: WT1+/HLA-A*0201+). In this experimental setting, each individual T cell population was sorted with specific dextramers, before co-culture with targets, to enrich the purity of effector cells. We observed a greater elimination of both pAML blasts harbouring the HLA-A*0201 allele upon co-culture with HD1 TR T cells, whereas only blasts from pAML3 were recognized by HD3 T and WT1 126-134 T cells. UT, untransduced, pAML, primary acute myeloid leukemia; TR, transfer; Dx, dextramer.

### DETAILED DESCRIPTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

T-Cell Receptor

During antigen processing, antigens are degraded inside cells and then carried to the cell surface by major histocompatibility complex (MHC) molecules. T-cells are able to recognise this peptide:MHC complex at the surface of the antigen presenting cell. There are two different classes of MHC molecules: MHC I and MHC II, each class delivers peptides from different cellular compartments to the cell surface.

A T cell receptor (TCR) is a molecule which can be found on the surface of T-cells that is responsible for recognizing antigens bound to MHC molecules. The naturally-occurring TCR heterodimer consists of an alpha (a) and beta (13) chain in around 95% of T-cells, whereas around 5% of T-cells have TCRs consisting of gamma (γ) and delta (6) chains.

Engagement of a TCR with antigen and MHC results in activation of the T lymphocyte on which the TCR is expressed through a series of biochemical events mediated by associated enzymes, co-receptors, and specialized accessory molecules.

Each chain of a natural TCR is a member of the immunoglobulin superfamily and possesses one N-terminal immunoglobulin (Ig)-variable (V) domain, one Ig-constant (C) domain, a transmembrane/cell membrane-spanning region, and a short cytoplasmic tail at the C-terminal end.

The variable domain of both the TCR α chain and β chain have three hypervariable or complementarity determining regions (CDRs). A TCR α chain or β chain, for example, comprises a CDR1, a CDR2, and a CDR3 in amino to carboxy terminal order. In general, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule.

A constant domain of a TCR may consist of short connecting sequences in which a cysteine residue forms a disulfide bond, making a link between the two chains.

An α chain of a TCR of the present invention may have a constant domain encoded by a TRAC gene. An example amino acid sequence of an α chain constant domain encoded by a TRAC gene is a shown below:

(SEQ ID NO: 128)
IQNPDPAVYQLRDSKSSDKSVCLFTDFDSQTNVSQSKDSDVYITDKT
VLDMRSMDFKSNSAVAWSNKSDFACANAFNNSIIPEDTFFPSPESSC
DVKLVEKSFETDTNLNFQNLSVIGFRILLLKVAGFNLLMTLRLWSS

A TCR of the present invention may comprise an α chain comprising the amino acid sequence of SEQ ID NO: 128 or a variant thereof having at least 75%, at least 80%, at least 85%, at least 90%, at least 95%, at least 96%, at least 97%, at least 98%, at least 99% sequence identity thereto, preferably at least 75% sequence identity thereto.

A β chain of a TCR of the present invention may have a constant domain encoded by a TRBC1 or a TRBC2 gene. An example amino acid sequence of a β chain constant domain encoded by a TRBC1 gene is a shown below:

(SEQ ID NO: 129)
DLNKVFPPEVAVFEPSEAEISHTQKATLVCLATGFFPDHVELSWWVN
GKEVHSGVSTDPQPLKEQPALNDSRYCLSSRLRVSATFWQNPRNHFR
CQVQFYGLSENDEWTQDRAKPVTQIVSAEAWGRADCGFTSVSYQQGV
LSATILYEILLGKATLYAVLVSALVLMAMVKRKDF

An example amino acid sequence of a β chain constant domain encoded by a TRBC2 gene is a shown below:

(SEQ ID NO: 130)
DLKNVFPPEVAVFEPSEAEISHTQKATLVCLATGFYPDHVELSWWVN
GKEVHSGVSTDPQPLKEQPALNDSRYCLSSRLRVSATFWQNPRNHFR
CQVQFYGLSENDEWTQDRAKPVTQIVSAEAWGRADCGFTSESYQQGV
LSATILYEILLGKATLYAVLVSALVLMAMVKRKDSRG

A TCR of the present invention may comprise a β chain comprising the amino acid sequence of SEQ ID NO: 129, SEQ ID NO: 130, or variants of SEQ ID NOs: 129 and 130 having at least 75%, at least 80%, at least 85%, at least 90%, at least 95%, at least 96%, at least 97%, at least 98%, at least 99% sequence identity thereto, preferably at least 75% sequence identity thereto.

The TCR of the present invention may have one or more additional cysteine residues in each of the α and β chains such that the TCR may comprise two or more disulphide bonds in the constant domains.

The structure allows the TCR to associate with other molecules like CD3 which possess three distinct chains (γ, δ, and ε) in mammals and the ζ-chain. These accessory molecules have negatively charged transmembrane regions and are vital to propagating the signal from the TCR into the cell. The CD3− and ζ-chains, together with the TCR, form what is known as the T cell receptor complex.

The signal from the T cell complex is enhanced by simultaneous binding of the MHC molecules by a specific co-receptor. For helper T-cells, this co-receptor is CD4 (specific for class II MHC); whereas for cytotoxic T-cells, this co-receptor is CD8 (specific for class I MHC). The co-receptor allows prolonged engagement between the antigen presenting cell and the T cell and recruits essential molecules (e.g., LCK) inside the cell involved in the signalling of the activated T lymphocyte.

Accordingly, as used herein the term “T-cell receptor” (TCR) refers to molecule capable of recognising a peptide when presented by an MHC molecule. The molecule may be a heterodimer of two chains α and β (or optionally γ and δ) or it may be a single chain TCR construct. A TCR of the present invention may be a soluble TCR, e.g. omitting or altering one or more constant domains. A TCR of the present invention may comprise a constant domain.

The present invention also provides an α chain or a β chain from such a T cell receptor.

The TCR of the present invention may be a hybrid TCR comprising sequences derived from more than one species. For example, it has surprisingly been found that murine TCRs are more efficiently expressed in human T-cells than human TCRs. The TCR may therefore comprise a human variable region and murine sequences within a constant region.

A disadvantage of this approach is that the murine constant sequences may trigger an immune response, leading to rejection of the transferred T-cells. However, the conditioning regimens used to prepare patients for adoptive T-cell therapy may result in sufficient immunosuppression to allow the engraftment of T-cells expressing murine sequences.

Complementarity Determining (CDR) Regions

The portion of the TCR that establishes the majority of the contacts with the antigenic peptide bound to the major histocompatibility complex (MHC) is the complementarity determining region 3 (CDR3), which is unique for each T cell clone. The CDR3 region is generated upon somatic rearrangement events occurring in the thymus and involving non-contiguous genes belonging to the variable (V), diversity (D, for β and δ chains) and joining (J) genes. Furthermore, random nucleotides inserted/deleted at the rearranging loci of each TCR chain gene greatly increase diversity of the highly variable CDR3 sequence. Thus, the frequency of a specific CDR3 sequence in a biological sample indicates the abundance of a specific T cell population. The great diversity of the TCR repertoire in healthy human beings provides a wide range protection towards a variety of foreign antigens presented by MHC molecules on the surface of antigen presenting cells. In this regard, it is of note that theoretically up to 10¹⁵ different TCRs can be generated in the thymus.

T-cell receptor diversity is focused on CDR3 and this region is primarily responsible for antigen recognition.

The sequences of the CDR3 regions of the TCR of the present invention may be selected from those set out in Table 1 below. A TCR may comprise CDRs that comprise or consist of a CDR3α and a CDR3β pair described below.

The CDRs may, for example, comprise one, two, or three substitutions, additions or deletions from the given sequence, provided that the TCR retains the capacity to bind a WT1 peptide when presented by an MHC molecule.

As used herein, the term “protein” includes single-chain polypeptide molecules as well as multiple-polypeptide complexes where individual constituent polypeptides are linked by covalent or non-covalent means. As used herein, the term “polypeptide” refers to a polymer in which the monomers are amino acids and are joined together through peptide or disulphide bonds.

Variants, Derivatives, Analogues, Homologues and Fragments

In addition to the specific proteins and polynucleotides mentioned herein, the present invention also encompasses the use of variants, derivatives, analogues, homologues and fragments thereof.

In the context of the present invention, a variant of any given sequence is a sequence in which the specific sequence of residues (whether amino acid or nucleic acid residues) has been modified in such a manner that the polypeptide or polynucleotide in question substantially retains at least one of its endogenous functions. A variant sequence can be obtained by addition, deletion, substitution, modification, replacement and/or variation of at least one residue present in the naturally-occurring protein.

A variant amino acid sequence of the present invention referred to as having up to three amino acid substitutions, additions or deletions may have, for example, one, two or three amino acid substitutions, additions or deletions.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide substantially retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Proteins used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

A substitution may involve replacement of an amino acid for a similar amino acid (a conservative substitution). A similar amino acid is one which has a side chain moiety with related properties as grouped together, for example as shown below:
(i) basic side chains: lysine (K), arginine (R), histidine (H);
(ii) acidic side chains: aspartic acid (D) and glutamic acid (E);
(iii) uncharged polar side chains: asparagine (N), glutamine (Q), serine (S), threonine (T) and tyrosine (Y); or
(iv) non-polar side chains: glycine (G), alanine (A), valine (V), leucine (L), isoleucine (I), proline (P), phenylalanine (F), methionine (M), tryptophan (W) and cysteine (C).

Any amino acid changes should maintain the capacity of the TCR to bind WT1 peptide presented by MHC molecules.

Variant sequences may comprise amino acid substitutions, additions, deletions and/or insertions. The variation may be concentrated in one or more regions, such as the constant regions, the linker, or the framework regions of the α or β chains, or they may be spread throughout the TCR molecule.

Conservative substitutions, additions or deletions may be made, for example according to the Table below. Amino acids in the same block in the second column and preferably in the same line in the third column may be substituted for each other:

| | | |
|---|---|---|
| ALIPHATIC | Non-polar | GAP |
| | | ILV |
| | Polar—uncharged | CSTM |
| | | NQ |
| | Polar—charged | DE |
| | | KR |
| AROMATIC | | HFWY |

The present invention also encompasses homologous substitution (substitution and replacement are both used herein to mean the interchange of an existing amino acid residue, with an alternative residue), e.g. like-for-like substitution such as basic for basic, acidic for acidic, polar for polar etc. Non-homologous substitution may also occur e.g. from one class of residue to another or alternatively involving the inclusion of unnatural amino acids, such as ornithine.

The term “variant” as used herein may mean an entity having a certain homology with the wild type amino acid sequence or the wild type nucleotide sequence. The term “homology” can be equated with “identity”.

A variant sequence may include an amino acid sequence which may be at least 50%, 55%, 65%, 75%, 85% or 90% identical, preferably at least 95%, at least 97%, or at least 99% identical to the subject sequence. Typically, the variants will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

A variant sequence may include a nucleotide sequence which may be at least 40%, 45%, 50%, 55%, 65%, 75%, 85% or 90% identical, preferably at least 95%, at least 97%, or at least 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Identity comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percentage homology, preferably percentage sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Major Histocompatability Complex (MHC) Molecules

Typically, TCRs bind to peptides as part of peptide:MHC complex.

The MHC molecule may be an MHC class I or II molecule. The complex may be on the surface of an antigen presenting cell, such as a dendritic cell or a B cell, or any other cell, including cancer cells, or it may be immobilised by, for example, coating on to a bead or plate.

The human leukocyte antigen system (HLA) is the name of the gene complex which encodes major histocompatibility complex (MHC) in humans and includes HLA class I antigens (A, B & C) and HLA class II antigens (DP, DQ, & DR). HLA alleles A, B and C present peptides derived mainly from intracellular proteins, e.g. proteins expressed within the cell. This is of particular relevance since WT1 is an intracellular protein.

During T-cell development in vivo, T-cells undergo a positive selection step to ensure recognition of self MHCs followed by a negative step to remove T-cells that bind too strongly to MHC which present self-antigens. As a consequence, certain T-cells and the TCRs they express will only recognise peptides presented by certain types of MHC molecules—i.e. those encoded by particular HLA alleles. This is known as HLA restriction.

One HLA allele of interest is HLA-A*0201, which is expressed in the vast majority (>50%) of the Caucasian population. Accordingly, TCRs which bind WT1 peptides presented by MHC encoded by HLA-A*0201 (i.e. are HLA-A*0201 restricted) are advantageous since an immunotherapy making use of such TCRs will be suitable for treating a large proportion of the Caucasian population.

Other HLA-A alleles of interest are HLA-A*0101, HLA-A*2402, and HLA-A*0301.

Widely expressed HLA-B alleles of interest are HLA-B*3501, HLA-B*0702 and HLA-B*3502.

A TCR of the present invention may be HLA-A*0201-restricted.

In one aspect, where a TCR of the present invention comprises a CDR3α comprising the amino acid sequence of CGTAWINDYKLSF (SEQ ID NO: 3) or a variant thereof having up to three amino acid substitutions, additions or deletions, and a CDR3β comprising the amino acid sequence of CASRKTGGYSNQPQHF (SEQ ID NO: 8) or a variant thereof having up to three amino acid substitutions, additions or deletions, the TCR is HLA-A*0201 restricted.

In another aspect, where a TCR of the present invention comprises a CDR3α comprising the amino acid sequence of CVVNLLSNQGGKLIF (SEQ ID NO: 36) or a variant thereof having up to three amino acid substitutions, additions or deletions, and a CDR3β comprising the amino acid sequence of CASSQDYLVSNEKLFF (SEQ ID NO: 41) or a variant thereof having up to three amino acid substitutions, additions or deletions, the TCR is HLA-A*0201 restricted.

In another aspect, where a TCR of the present invention comprises a CDR3α comprising the amino acid sequence of CAANNARLMF (SEQ ID NO: 92) or a variant thereof having up to three amino acid substitutions, additions or deletions, and a CDR3β comprising the amino acid sequence of CASSDTRAREQFF (SEQ ID NO: 97) or a variant thereof having up to three amino acid substitutions, additions or deletions, the TCR is HLA-A*0201 restricted.

In another aspect, where a TCR of the present invention comprises a CDR3α comprising the amino acid sequence of CAERLNTDKLIF (SEQ ID NO: 103) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions, the TCR is HLA-A*0201 restricted.

In another aspect, where a TCR of the present invention comprises a CDR3α comprising the amino acid sequence of CAASGGRDDKIIF (SEQ ID NO: 214) or a variant thereof having up to three amino acid substitutions, additions or deletions, and a CDR3β comprising the amino acid sequence of CASSYSRTESTDTQYF (SEQ ID NO: 219) or a variant thereof having up to three amino acid substitutions, additions or deletions, the TCR is HLA-A*0201 restricted.

In another aspect, where a TCR of the present invention comprises a CDR3α comprising the amino acid sequence of CAANNARLMF (SEQ ID NO: 92) or a variant thereof having up to three amino acid substitutions, additions or deletions, and a CDR3β comprising the amino acid sequence of CASSPGQHGELFF (SEQ ID NO: 271) or a variant thereof having up to three amino acid substitutions, additions or deletions, the TCR is HLA-A*0201 restricted.

In another aspect, where a TCR of the present invention comprises a CDR3α comprising the amino acid sequence of CAASATGNQFYF (SEQ ID NO: 266) or a variant thereof having up to three amino acid substitutions, additions or deletions, and a CDR3β comprising the amino acid sequence of CASSDTRAREQFF (SEQ ID NO: 97) or a variant thereof having up to three amino acid substitutions, additions or deletions, the TCR is HLA-A*0201 restricted.

In another aspect, where a TCR of the present invention comprises a CDR3α comprising the amino acid sequence of CAASATGNQFYF (SEQ ID NO: 266) or a variant thereof having up to three amino acid substitutions, additions or deletions, and a CDR3β comprising the amino acid sequence of CASSPGQHGELFF (SEQ ID NO: 271) or a variant thereof having up to three amino acid substitutions, additions or deletions, the TCR is HLA-A*0201 restricted.

In another aspect, where a TCR of the present invention comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions, the TCR is HLA-A*0201 restricted.

In another aspect, where a TCR of the present invention comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions, the TCR is HLA-A*0201 restricted.

In another aspect, where a TCR of the present invention comprises a CDR3α comprising the amino acid sequence of CAERLNTDKLIF (SEQ ID NO: 103) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions, the TCR is HLA-A*0201 restricted.

In one embodiment, a TCR of the present invention that is HLA-A*0201 restricted binds to a WT1 peptide comprising amino acid sequence APVLDFAPPGA (SEQ ID NO: 117) or a variant thereof having up to three amino acid substitutions, additions or deletions.

In one aspect, the present invention provides a TCR which binds a Wilms tumour 1 protein (WT1) peptide when presented by a major histocompatibility complex (MHC), wherein the TCR comprises a CDR3α comprising the amino acid sequence of CGTAWINDYKLSF (SEQ ID NO: 3) or a variant thereof having up to three amino acid substitutions, additions or deletions, and a CDR3β comprising the amino acid sequence of CASRKTGGYSNQPQHF (SEQ ID NO: 8) or a variant thereof having up to three amino acid substitutions, additions or deletions, wherein the TCR is HLA-A*0201 restricted, and wherein the WT1 peptide comprises the amino acid sequence of APVLDFAPPGA (SEQ ID NO: 117) or a variant thereof having up to three amino acid substitutions, additions or deletions.

In another aspect, the present invention provides a TCR which binds a Wilms tumour 1 protein (WT1) peptide when presented by a major histocompatibility complex (MHC), wherein the TCR comprises a CDR3α comprising the amino acid sequence of CVVNLLSNQGGKLIF (SEQ ID NO: 36) or a variant thereof having up to three amino acid substitutions, additions or deletions, and a CDR3β comprising the amino acid sequence of CASSQDYLVSNEKLFF (SEQ ID NO: 41) or a variant thereof having up to three amino acid substitutions, additions or deletions, wherein the TCR is HLA-A*0201 restricted, and wherein the WT1 peptide comprises the amino acid sequence of APVLDFAPPGA (SEQ ID NO: 117) or a variant thereof having up to three amino acid substitutions, additions or deletions.

In another aspect, the present invention provides a TCR which binds a Wilms tumour 1 protein (WT1) peptide when presented by a major histocompatibility complex (MHC), wherein the TCR comprises a CDR3α comprising the amino acid sequence of CAANNARLMF (SEQ ID NO: 92) or a variant thereof having up to three amino acid substitutions, additions or deletions, and a CDR3β comprising the amino acid sequence of CASSDTRAREQFF (SEQ ID NO: 97) or a variant thereof having up to three amino acid substitutions, additions or deletions, wherein the TCR is HLA-A*0201 restricted, and wherein the WT1 peptide comprises the amino acid sequence of APVLDFAPPGA (SEQ ID NO: 117) or a variant thereof having up to three amino acid substitutions, additions or deletions.

In another aspect, the present invention provides a TCR which binds a Wilms tumour 1 protein (WT1) peptide when presented by a major histocompatibility complex (MHC), wherein the TCR comprises a CDR3α comprising the amino acid sequence of CAERLNTDKLIF (SEQ ID NO: 103) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions, wherein the TCR is HLA-A*0201 restricted, and wherein the WT1 peptide comprises the amino acid sequence of APVLDFAPPGA (SEQ ID NO: 117) or a variant thereof having up to three amino acid substitutions, additions or deletions.

In another aspect, the present invention provides a TCR which binds a Wilms tumour 1 protein (WT1) peptide when presented by a major histocompatibility complex (MHC), wherein the TCR comprises a CDR3α comprising the amino acid sequence of CAASGGRDDKIIF (SEQ ID NO: 214) or a variant thereof having up to three amino acid substitutions, additions or deletions, and a CDR3β comprising the amino acid sequence of CASSYSRTESTDTQYF (SEQ ID NO: 219) or a variant thereof having up to three amino acid substitutions, additions or deletions, wherein the TCR is HLA-A*0201 restricted, and wherein the WT1 peptide comprises the amino acid sequence of APVLDFAPPGA (SEQ ID NO: 117) or a variant thereof having up to three amino acid substitutions, additions or deletions.

In another aspect, the present invention provides a TCR which binds a Wilms tumour 1 protein (WT1) peptide when presented by a major histocompatibility complex (MHC), wherein the TCR comprises a CDR3α comprising the amino acid sequence of CAANNARLMF (SEQ ID NO: 92) or a variant thereof having up to three amino acid substitutions, additions or deletions, and a CDR3β comprising the amino acid sequence of CASSPGQHGELFF (SEQ ID NO: 271) or a variant thereof having up to three amino acid substitutions, additions or deletions, wherein the TCR is HLA-A*0201 restricted, and wherein the WT1 peptide comprises the amino acid sequence of APVLDFAPPGA (SEQ ID NO: 117) or a variant thereof having up to three amino acid substitutions, additions or deletions.

In another aspect, the present invention provides a TCR which binds a Wilms tumour 1 protein (WT1) peptide when presented by a major histocompatibility complex (MHC), wherein the TCR comprises a CDR3α comprising the amino acid sequence of CAASATGNQFYF (SEQ ID NO: 266) or a variant thereof having up to three amino acid substitutions, additions or deletions, and a CDR3β comprising the amino acid sequence of CASSDTRAREQFF (SEQ ID NO: 97) or a variant thereof having up to three amino acid substitutions, additions or deletions, wherein the TCR is HLA-A*0201 restricted, and wherein the WT1 peptide comprises the amino acid sequence of APVLDFAPPGA (SEQ ID NO: 117) or a variant thereof having up to three amino acid substitutions, additions or deletions.

In another aspect, the present invention provides a TCR which binds a Wilms tumour 1 protein (WT1) peptide when presented by a major histocompatibility complex (MHC), wherein the TCR comprises a CDR3α comprising the amino acid sequence of CAASATGNQFYF (SEQ ID NO: 266) or a variant thereof having up to three amino acid substitutions, additions or deletions, and a CDR3β comprising the amino acid sequence of CASSPGQHGELFF (SEQ ID NO: 271) or a variant thereof having up to three amino acid substitutions, additions or deletions, wherein the TCR is HLA-A*0201 restricted, and wherein the WT1 peptide comprises the amino acid sequence of APVLDFAPPGA (SEQ ID NO: 117) or a variant thereof having up to three amino acid substitutions, additions or deletions.

In another aspect, the present invention provides a TCR which binds a Wilms tumour 1 protein (WT1) peptide when presented by a major histocompatibility complex (MHC), wherein the TCR comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions, wherein the TCR is HLA-A*0201 restricted, and wherein the WT1 peptide comprises the amino acid sequence of APVLDFAPPGA (SEQ ID NO: 117) or a variant thereof having up to three amino acid substitutions, additions or deletions.

In another aspect, the present invention provides a TCR which binds a Wilms tumour 1 protein (WT1) peptide when presented by a major histocompatibility complex (MHC), wherein the TCR comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions, wherein the TCR is HLA-A*0201 restricted, and wherein the WT1 peptide comprises the amino acid sequence of APVLDFAPPGA (SEQ ID NO: 117) or a variant thereof having up to three amino acid substitutions, additions or deletions.

In another aspect, the present invention provides a TCR which binds a Wilms tumour 1 protein (WT1) peptide when presented by a major histocompatibility complex (MHC), wherein the TCR comprises a CDR3α comprising the amino acid sequence of CAERLNTDKLIF (SEQ ID NO: 103) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions, wherein the TCR is HLA-A*0201 restricted, and wherein the WT1 peptide comprises the amino acid sequence of APVLDFAPPGA (SEQ ID NO: 117) or a variant thereof having up to three amino acid substitutions, additions or deletions.

Another widely expressed HLA allele of interest is HLA-B*3501. A TCR of the present invention may be HLA-B*3501 restricted.

In one aspect, where a TCR of the present invention comprises a CDR3α comprising the amino acid sequence of CAASMAGAGSYQLTF (SEQ ID NO: 75) or a variant thereof having up to three amino acid substitutions, additions or deletions, and a CDR3β comprising the amino acid sequence of CAISVGQGALYEQYF (SEQ ID NO: 80) or a variant thereof having up to three amino acid substitutions, additions or deletions, the TCR is HLA-B*3501 restricted.

Thus, in another aspect, the present invention provides a TCR which binds a Wilms tumour 1 protein (WT1) peptide when presented by a major histocompatibility complex (MHC), wherein the TCR comprises a CDR3α comprising the amino acid sequence of CAASMAGAGSYQLTF (SEQ ID NO: 75) or a variant thereof having up to three amino acid substitutions, additions or deletions, and a CDR3β comprising the amino acid sequence of CAISVGQGALYEQYF (SEQ ID NO: 80) or a variant thereof having up to three amino acid substitutions, additions or deletions, wherein the TCR is HLA-B*3501 restricted, and wherein the WT1 peptide comprises the amino acid sequence of NHTTPILCGAQYRIH (SEQ ID NO: 127) or a variant thereof having up to three amino acid substitutions, additions or deletions.

In one aspect, where a TCR of the present invention comprises a CDR3α comprising the amino acid sequence of CAASMAGAGSYQLTF (SEQ ID NO: 75) or a variant thereof having up to three amino acid substitutions, additions or deletions, and a CDR3β comprising the amino acid sequence of CASSVARDRRNYGYTF (SEQ ID NO: 86) or a variant thereof having up to three amino acid substitutions, additions or deletions, the TCR is HLA-B*3501 restricted.

Thus, in another aspect, the present invention provides a TCR which binds a Wilms tumour 1 protein (WT1) peptide when presented by a major histocompatibility complex (MHC), wherein the TCR comprises a CDR3α comprising the amino acid sequence of CAASMAGAGSYQLTF (SEQ ID NO: 75) or a variant thereof having up to three amino acid substitutions, additions or deletions, and a CDR3β comprising the amino acid sequence of CASSVARDRRNYGYTF (SEQ ID NO: 86) or a variant thereof having up to three amino acid substitutions, additions or deletions, wherein the TCR is HLA-B*3501 restricted, and wherein the WT1 peptide comprises the amino acid sequence of NHTTPILCGAQYRIH (SEQ ID NO: 127) or a variant thereof having up to three amino acid substitutions, additions or deletions.

Another widely expressed HLA allele of interest is HLA-B*3502. A TCR of the present invention may be HLA-B*3502 restricted.

In one aspect, where a TCR of the present invention comprises a CDR3α comprising the amino acid sequence of CATDAYSGNTPLVF (SEQ ID NO: 47) or a variant thereof having up to three amino acid substitutions, additions or deletions, and a CDR3β comprising the amino acid sequence of CASRAAGLDTEAFF (SEQ ID NO: 57) or a variant thereof having up to three amino acid substitutions, additions or deletions, the TCR is HLA-B*3502 restricted.

Thus, in one aspect, the present invention provides a TCR which binds a Wilms tumour 1 protein (WT1) peptide when presented by a major histocompatibility complex (MHC), wherein the TCR comprises a CDR3α comprising the amino acid sequence of CATDAYSGNTPLVF (SEQ ID NO: 47) or a variant thereof having up to three amino acid substitutions, additions or deletions, and a CDR3β comprising the amino acid sequence of CASRAAGLDTEAFF (SEQ ID NO: 57) or a variant thereof having up to three amino acid substitutions, additions or deletions, wherein the TCR is HLA-B*3502 restricted, and wherein the WT1 peptide comprises the amino acid sequence of EPASQHTLRSG (SEQ ID NO: 123) or a variant thereof having up to three amino acid substitutions, additions or deletions.

We have demonstrated that T-cells expressing TCRs of the present invention which bind to WT1 peptides comprising an amino acid sequence of EPASQHTLRSG (SEQ ID NO: 123) are able to selectively eliminate cancer (AML) cells expressing the HLA-B*3502 allele—see Example 4 and FIG. 4c.

In one aspect, where a TCR of the present invention binds to a WT1 peptide comprising an amino acid sequence of EPASQHTLRSG (SEQ ID NO: 123), or a variant thereof having up to three amino acid substitutions, additions or deletions, the TCR is HLA-B*3502 restricted.

In one embodiment, where a TCR of the present invention binds to a WT1 peptide comprising an amino acid sequence of APVLDFAPPGA (SEQ ID NO: 117) or a variant thereof having up to three amino acid substitutions, additions or deletions, the TCR is HLA-A*0201 restricted.

In one embodiment, where a TCR of the present invention binds to a WT1 peptide comprising an amino acid sequence of NHTTPILCGAQYRIH (SEQ ID NO: 127) or a variant thereof having up to three amino acid substitutions, additions or deletions, the TCR is HLA-B*3501 restricted.

Wilms Tumor 1 (WT1) Protein

Wilms tumor 1 (WT1) is an intracellular protein encoding a zinc finger transcription factor that plays an important role in cell growth and differentiation (Yang, L. et al. Leukemia 21, 868-876 (2007)). It is widely expressed on a variety of hematological and solid tumors, while showing limited expression on other tissues (gonads, uterus, kidney, mesothelium, progenitor cells in different tissues). Recent evidence suggests that WT1 plays a role in leukemogenesis and tumorigenesis.

WT1 has several isoforms, some of which result from alternative splicing of mRNA transcripts encoding WT1. The complete amino acid sequence of a WT1 isoform was previously published (Gessler, M. et al. Nature; 343(6260):774-778; (1990)). This particular isoform consists of 575 amino acids and includes a first 126 amino acids at the N terminus which are lacking in the exon 5+ and the TS= isoforms of WT1.

An example WT1 protein has the amino acid sequence set out in UniProt entry J3KNN9. Another example WT1 protein has the amino acid sequence set out below:

(SEQ ID NO: 131)
SRQRPHPGALRNPTACPLPHFPPSLPPTHSPTHPPRAGTAAQAPGPR
RLLAAILDFLLLQDPASTCVPEPASQHTLRSGPGCLQQPEQQGVRDP
GGIWAKLGAAEASAERLQGRRSRGASGSEPQQMGSDVRDLNALLPAV
PSLGGGGGCALPVSGAAQWAPVLDFAPPGASAYGSLGGPAPPPAPPP
PPPPPPHSFIKQEPSWGGAEPHEEQCLSAFTVHFSGQFTGTAGACRY
GPFGPPPPSQASSGQARMFPNAPYLPSCLESQPAIRNQGYSTVTFDG
TPSYGHTPSHHAAQFPNHSFKHEDPMGQQGSLGEQQYSVPPPVYGCH
TPTDSCTGSQALLLRTPYSSDNLYQMTSQLECMTWNQMNLGATLKGV
AAGSSSSVKWTEGQSNHSTGYESDNHTTPILCGAQYRIHTHGVFRGI
QDVRRVPGVAPTLVRSASETSEKRPFMCAYPGCNKRYFKLSHLQMHS
RKHTGEKPYQCDFKDCERRFSRSDQLKRHQRRHTGVKPFQCKTCQRK
FSRSDHLKTHTRTHTGKTSEKPFSCRWPSCQKKFARSDELVRHHNMH
QRNMTKLQLAL

WT1 Peptides

As used herein the term peptide refers to a plurality of amino acid residues linked by peptide bonds. As defined herein a peptide may consist of less than about 30, less than about 25, less than about 20, less than 19, less than 18, less than 17, less than 16, less than 15, less than 14, less than 13, less than 12, less than 11, less than 10, less than 9, less than 8, less than 7, less than 6, or less than 5 amino acid residues in length. Preferably, a peptide is about 5 to 20 amino acids in length, more preferably, a peptide is about 8 to 15 amino acid residues in length.

The TCRs of the present invention bind to a WT1 peptide when presented by an MHC. As used herein, the term WT1 peptide is understood to mean a peptide comprising an amino acid sequence derived from a WT1 protein.

For example, a WT1 peptide may comprise at least 5, at least 6, at least 7, at least 8, at least 9, at least 10, at least 11, at least 12, at least 13, at least 14, at least 15, at least 16, at least 17, at least 18, at least 19, at least 20, or at least 25 contiguous amino acid residues of a WT1 protein amino acid sequence.

The WT1 peptide may comprise or consist of the amino acid sequence of APVLDFAPPGA (SEQ ID NO: 117) or a variant thereof having up to three amino acid substitutions, additions or deletions. Examples of WT1 peptides comprising the amino acid sequence are AAQWAPVLDFAPPGA (SEQ ID NO: 115) and APVLDFAPPGASAYG (SEQ ID NO: 116).

The WT1 peptide may comprise or consist of an amino acid sequence selected from the group consisting of QCLSAFTVHFSGQFT (SEQ ID NO: 118), EDPMGQQGSLGEQQY (SEQ ID NO: 119), SQLECMTWNQMNLGA (SEQ ID NO: 120), and variants of SEQ ID NOs: 118-120 each having up to three amino acid substitutions, additions or deletions.

The WT1 peptide may comprise or consist of an amino acid sequence selected from the group consisting of EPASQHTLRSG (SEQ ID NO: 123), YESDNHTTPIL (SEQ ID NO: 126), and variants of SEQ ID NOs: 123 and 126 each having up to three amino acid substitutions, additions or deletions. Example WT1 peptides may have an amino acid sequence selected from the group consisting of TCVPEPASQHTLRSG (SEQ ID NO: 121), EPASQHTLRSGPGCL (SEQ ID NO: 122), HSTGYESDNHTTPIL (SEQ ID NO: 124) and YESDNHTTPILCGAQ (SEQ ID NO: 125).

The WT1 peptide may comprise or consist of the amino acid sequence of NHTTPILCGAQYRIH (SEQ ID NO: 127) or a variant thereof having up to three amino acid substitutions, additions or deletions.

The WT1 peptide may comprise or consist of the amino acid sequence of NQMNLGATLKG (SEQ ID NO: 250) or a variant thereof having up to three amino acid substitutions, additions or deletions. Example WT1 peptides may have an amino acid sequence selected from the group consisting of CMTWNQMNLGATLKG (SEQ ID NO: 248) and NQMNLGATLKGVAAG (SEQ ID NO: 249).

The WT1 peptide may comprise or consist of the amino acid sequence of DPGGIWAKLGAAEAS (SEQ ID NO: 251) or a variant thereof having up to three amino acid substitutions, additions or deletions.

The WT1 peptide may comprise or consist of an amino acid sequence selected from the group consisting of NHTTPILCGAQYRIH (SEQ ID NO: 252), KRHQRRHTGVKPFQC (SEQ ID NO: 253), PSCQKKFARSDELVR (SEQ ID NO: 254), and variants of SEQ ID NOs: 252, 253 and 254 each having up to three amino acid substitutions, additions or deletions.

In some embodiments, for WT1 peptides which bind to MHC molecules encoded by HLA-A*0201 allele it may be preferred that the amino acids at position 2 of the peptide (i.e. the second amino acid from the N-terminus) are leucine or methionine, although isoleucine, valine, alanine and threonine may also be preferable. It may also be preferred that the amino acid at position 9 or 10 is valine, leucine or isoleucine, although alanine, methionine and threonine may also be preferable. The preferred MHC binding motifs of other HLA alleles are disclosed in Celis et al (Molecular Immunology, Vol. 31, 8, December 1994, pages 1423 to 1430).

Various uses of the WT1 peptides described herein are contemplated by the present invention. For example, the WT1 peptides described herein may be administered to a subject, e.g. a human subject. Administration of the WT1 peptides of the present invention may elicit an immune response against cells expressing or overexpressing WT1 protein, i.e. the WT1 peptides are immunogenic WT1 peptides.

Thus in another aspect, the present invention provides an isolated immunogenic WT1 peptide comprising an amino acid sequence selected from the group consisting of EPASQHTLRSG (SEQ ID NO: 123), YESDNHTTPIL (SEQ ID NO: 126), NHTTPILCGAQYRIH (SEQ ID NO: 127), QCLSAFTVHFSGQFT (SEQ ID NO: 118), EDPMGQQGSLGEQQY (SEQ ID NO: 119), SQLECMTWNQMNLGA (SEQ ID NO: 120), APVLDFAPPGA (SEQ ID NO: 117), NQMNLGATLKG (SEQ ID NO: 250), DPGGIWAKLGAAEAS (SEQ ID NO: 251), NHTTPILCGAQYRIH (SEQ ID NO: 252), KRHQRRHTGVKPFQC (SEQ ID NO: 253), PSCQKKFARSDELVR (SEQ ID NO: 254), and variants thereof each having up to three amino acid substitutions, additions or deletions.

The WT1 peptides described herein, e.g. WT1 peptides comprising an amino acid sequence selected from the group consisting of EPASQHTLRSG (SEQ ID NO: 123) and YESDNHTTPIL (SEQ ID NO: 126), NHTTPILCGAQYRIH (SEQ ID NO: 127), QCLSAFTVHFSGQFT (SEQ ID NO: 118), EDPMGQQGSLGEQQY (SEQ ID NO: 119), SQLECMTWNQMNLGA (SEQ ID NO: 120), APVLDFAPPGA (SEQ ID NO: 117), NQMNLGATLKG (SEQ ID NO: 250), DPGGIWAKLGAAEAS (SEQ ID NO: 251), NHTTPILCGAQYRIH (SEQ ID NO: 252), KRHQRRHTGVKPFQC (SEQ ID NO: 253) PSCQKKFARSDELVR (SEQ ID NO: 254), and variants thereof each having up to three amino acid substitutions, additions or deletions, may be used to screen for and/or identify new TCR sequences which bind to WT1 cells. For example, T2 cells may be pulsed with a WT1 peptide mentioned in the present invention and incubated with a T-cell population isolated from a donor. In this approach, expression of cytokines, e.g. CD107a and IFNγ, may be indicative of T-cells which recognise WT1 peptides.

Accordingly, in one aspect, the present invention provides a T-cell receptor (TCR), which binds to a Wilms tumour 1 protein (WT1) peptide when presented by a major histocompatibility complex (MHC), wherein the WT1 peptide comprises an amino acid sequence selected from the group consisting of EPASQHTLRSG (SEQ ID NO: 123), YESDNHTTPIL (SEQ ID NO: 126), NHTTPILCGAQYRIH (SEQ ID NO: 127), QCLSAFTVHFSGQFT (SEQ ID NO: 118), EDPMGQQGSLGEQQY (SEQ ID NO: 119), SQLECMTWNQMNLGA (SEQ ID NO: 120), APVLDFAPPGA (SEQ ID NO: 117), NQMNLGATLKG (SEQ ID NO: 250), DPGGIWAKLGAAEAS (SEQ ID NO: 251), NHTTPILCGAQYRIH (SEQ ID NO: 252), KRHQRRHTGVKPFQC (SEQ ID NO: 253), PSCQKKFARSDELVR (SEQ ID NO: 254), and variants thereof each having up to three amino acid substitutions, additions or deletions.

TCR Sequences

We have determined the amino acid sequences for TCRs that bind to WT1 peptides described herein. In particular, we have determined the amino acid sequences of the TCR CDRs, which are important for WT1 peptide recognition and binding.

Thus, in one embodiment, the present invention provides a TCR comprising a CDR3α comprising the amino acid sequence of CGTAWINDYKLSF (SEQ ID NO: 3) or a variant thereof having up to three amino acid substitutions, additions or deletions, and a CDR3β comprising the amino acid sequence of CASRKTGGYSNQPQHF (SEQ ID NO: 8) or a variant thereof having up to three amino acid substitutions, additions or deletions, which binds to a WT1 peptide comprising the amino acid sequence of APVLDFAPPGA (SEQ ID NO: 117) or a variant thereof having up to three amino acid substitutions, additions or deletions when presented by an MHC.

Thus, in one embodiment, the present invention provides, a TCR comprising a CDR3α comprising the amino acid sequence of CVVNLLSNQGGKLIF (SEQ ID NO: 36) or a variant thereof having up to three amino acid substitutions, additions or deletions, and a CDR3β comprising the amino acid sequence of CASSQDYLVSNEKLFF (SEQ ID NO: 41) or a variant thereof having up to three amino acid substitutions, additions or deletions, which binds to a WT1 peptide comprising the amino acid sequence of APVLDFAPPGA (SEQ ID NO: 117) or a variant thereof having up to three amino acid substitutions, additions or deletions when presented by an MHC.

Thus, in one embodiment, the present invention provides, a TCR comprising a CDR3α comprising the amino acid sequence of CAANNARLMF (SEQ ID NO: 92) or a variant thereof having up to three amino acid substitutions, additions or deletions, and a CDR3β comprising the amino acid sequence of CASSDTRAREQFF (SEQ ID NO: 97) or a variant thereof having up to three amino acid substitutions, additions or deletions, which binds to a WT1 peptide comprising the amino acid sequence of APVLDFAPPGA (SEQ ID NO: 117) or a variant thereof having up to three amino acid substitutions, additions or deletions when presented by an MHC.

Thus, in one embodiment, the present invention provides, a TCR comprising a CDR3α comprising the amino acid sequence of CAERLNTDKLIF (SEQ ID NO: 103) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions, which binds to a WT1 peptide comprising the amino acid sequence of APVLDFAPPGA (SEQ ID NO: 117) or a variant thereof having up to three amino acid substitutions, additions or deletions when presented by an MHC.

In one embodiment, the present invention provides, a TCR comprising a CDR3α comprising the amino acid sequence of CAVEATDSWGKLQF (SEQ ID NO: 108) or a variant thereof having up to three amino acid substitutions, additions or deletions, and a CDR3β comprising the amino acid sequence of CSVGGSGSYNEQFF (SEQ ID NO: 169) or a variant thereof having up to three amino acid substitutions, additions or deletions, which binds to a WT1 peptide comprising the amino acid sequence of NQMNLGATLKG (SEQ ID NO: 250) or a variant thereof having up to three amino acid substitutions, additions or deletions when presented by an MHC.

In one embodiment, the present invention provides, a TCR comprising a CDR3α comprising the amino acid sequence of CAVRTSYDKVIF (SEQ ID NO: 113) or a variant thereof having up to three amino acid substitutions, additions or deletions, and a CDR3β comprising the amino acid sequence of CSVGGSGSYNEQFF (SEQ ID NO: 169) or a variant thereof having up to three amino acid substitutions, additions or deletions, which binds to a WT1 peptide comprising the amino acid sequence of NQMNLGATLKG (SEQ ID NO: 250) or a variant thereof having up to three amino acid substitutions, additions or deletions when presented by an MHC.

Thus, in one embodiment, the present invention provides, a TCR comprising a CDR3α comprising the amino acid sequence of CAASGGRDDKIIF (SEQ ID NO: 214) or a variant thereof having up to three amino acid substitutions, additions or deletions, and a CDR3β comprising the amino acid sequence of CASSYSRTESTDTQYF (SEQ ID NO: 219) or a variant thereof having up to three amino acid substitutions, additions or deletions, which binds to a WT1 peptide comprising the amino acid sequence of APVLDFAPPGA (SEQ ID NO: 117) or a variant thereof having up to three amino acid substitutions, additions or deletions when presented by an MHC.

The present invention also provides a TCR comprising:
a CDR3α comprising the amino acid sequence of CAVRLSGSARQLTF (SEQ ID NO: 14) or a variant thereof having up to three amino acid substitutions, additions or deletions, and a CDR3β comprising the amino acid sequence of CASSLLGDEQYF (SEQ ID NO: 24) or a variant thereof having up to three amino acid substitutions, additions or deletions;
a CDR3α comprising the amino acid sequence of CAVRLSGSARQLTF (SEQ ID NO: 14) or a variant thereof having up to three amino acid substitutions, additions or deletions, and a CDR3β comprising the amino acid sequence of CASSLVALQGAGEQYF (SEQ ID NO: 30) or a variant thereof having up to three amino acid substitutions, additions or deletions;
a CDR3α comprising the amino acid sequence of CAYRSLKYGNKLVF (SEQ ID NO: 19) or a variant thereof having up to three amino acid substitutions, additions or deletions, and a CDR3β comprising the amino acid sequence of CASSLLGDEQYF (SEQ ID NO: 24) or a variant thereof having up to three amino acid substitutions, additions or deletions; or a CDR3α comprising the amino acid sequence of CAYRSLKYGNKLVF (SEQ ID NO: 19) or a variant thereof having up to three amino acid substitutions, additions or deletions, and a CDR3β comprising the amino acid sequence of CASSLVALQGAGEQYF (SEQ ID NO: 30) or a variant thereof having up to three amino acid substitutions, additions or deletions;
wherein the TCR binds to a WT1 peptide comprising or consisting of an amino acid sequence selected from the group consisting of QCLSAFTVHFSGQFT (SEQ ID NO: 118), EDPMGQQGSLGEQQY (SEQ ID NO: 119), and SQLECMTWNQMNLGA (SEQ ID NO: 120) or variants thereof each having up to three amino acid substitutions, additions or deletions when presented by an MHC.

Thus, in one embodiment, there is provided a TCR comprising a CDR3α comprising the amino acid sequence of CAVRLSGSARQLTF (SEQ ID NO: 14) or a variant thereof having up to three amino acid substitutions, additions or deletions, and a CDR3β comprising the amino acid sequence of CASSLLGDEQYF (SEQ ID NO: 24) or a variant thereof having up to three amino acid substitutions, additions or deletions, which binds to a WT1 peptide comprising or consisting of the amino acid sequence of QCLSAFTVHFSGQFT (SEQ ID NO: 118) or a variant thereof having up to three amino acid substitutions, additions or deletions when presented by an MHC.

In one embodiment, there is provided a TCR comprising a CDR3α comprising the amino acid sequence of CAVRLSGSARQLTF (SEQ ID NO: 14) or a variant thereof having up to three amino acid substitutions, additions or deletions, and a CDR3β comprising the amino acid sequence of CASSLLGDEQYF (SEQ ID NO: 24) or a variant thereof having up to three amino acid substitutions, additions or deletions, which binds to a WT1 peptide comprising or consisting of the amino acid sequence of EDPMGQQGSLGEQQY (SEQ ID NO: 119) or a variant thereof having up to three amino acid substitutions, additions or deletions when presented by an MHC.

In one embodiment, there is provided a TCR comprising a CDR3α comprising the amino acid sequence of CAVRLSGSARQLTF (SEQ ID NO: 14) or a variant thereof having up to three amino acid substitutions, additions or deletions, and a CDR3β comprising the amino acid sequence of CASSLLGDEQYF (SEQ ID NO: 24) or a variant thereof having up to three amino acid substitutions, additions or deletions, which binds to a WT1 peptide comprising or consisting of the amino acid sequence of SQLECMTWNQMNLGA (SEQ ID NO: 120) or a variant thereof having up to three amino acid substitutions, additions or deletions when presented by an MHC.

In one embodiment, there is provided a TCR comprising a CDR3α comprising the amino acid sequence of CAVRLSGSARQLTF (SEQ ID NO: 14) or a variant thereof having up to three amino acid substitutions, additions or deletions, and a CDR3β comprising the amino acid sequence of CASSLVALQGAGEQYF (SEQ ID NO: 30) or a variant thereof having up to three amino acid substitutions, additions or deletions, which binds to a WT1 peptide comprising or consisting of the amino acid sequence of QCLSAFTVHFSGQFT (SEQ ID NO: 118) or a variant thereof having up to three amino acid substitutions, additions or deletions when presented by an MHC.

In one embodiment, there is provided a TCR comprising a CDR3α comprising the amino acid sequence of CAVRLSGSARQLTF (SEQ ID NO: 14) or a variant thereof having up to three amino acid substitutions, additions or deletions, and a CDR3β comprising the amino acid sequence of CASSLVALQGAGEQYF (SEQ ID NO: 30) or a variant thereof having up to three amino acid substitutions, additions or deletions, which binds to a WT1 peptide comprising or consisting of the of EDPMGQQGSLGEQQY (SEQ ID NO: 119) or a variant thereof having up to three amino acid substitutions, additions or deletions when presented by an MHC.

In one embodiment, there is provided a TCR comprising a CDR3α comprising the amino acid sequence of CAVRLSGSARQLTF (SEQ ID NO: 14) or a variant thereof having up to three amino acid substitutions, additions or deletions, and a CDR3β comprising the amino acid sequence of CASSLVALQGAGEQYF (SEQ ID NO: 30) or a variant thereof having up to three amino acid substitutions, additions or deletions, which binds to a WT1 peptide comprising or consisting of the amino acid sequence of SQLECMTWNQMNLGA (SEQ ID NO: 120) or a variant thereof having up to three amino acid substitutions, additions or deletions when presented by an MHC.

In one embodiment, there is provided a TCR comprising a CDR3α comprising the amino acid sequence of CAYRSLKYGNKLVF (SEQ ID NO: 19) or a variant thereof having up to three amino acid substitutions, additions or deletions, and a CDR3β comprising the amino acid sequence of CASSLLGDEQYF (SEQ ID NO: 24) or a variant thereof having up to three amino acid substitutions, additions or deletions, which binds to a WT1 peptide comprising or consisting of the amino acid sequence of QCLSAFTVHFSGQFT (SEQ ID NO: 118) or a variant thereof having up to three amino acid substitutions, additions or deletions when presented by an MHC.

In one embodiment, there is provided a TCR comprising a CDR3α comprising the amino acid sequence of CAYRSLKYGNKLVF (SEQ ID NO: 19) or a variant thereof having up to three amino acid substitutions, additions or deletions, and a CDR3β comprising the amino acid sequence of CASSLLGDEQYF (SEQ ID NO: 24) or a variant thereof having up to three amino acid substitutions, additions or deletions, which binds to a WT1 peptide comprising or consisting of the amino acid sequence of EDPMGQQGSLGEQQY (SEQ ID NO: 119) or a variant thereof having up to three amino acid substitutions, additions or deletions when presented by an MHC.

In one embodiment, there is provided a TCR comprising a CDR3α comprising the amino acid sequence of CAYRSLKYGNKLVF (SEQ ID NO: 19) or a variant thereof having up to three amino acid substitutions, additions or deletions, and a CDR3β comprising the amino acid sequence of CASSLLGDEQYF (SEQ ID NO: 24) or a variant thereof having up to three amino acid substitutions, additions or deletions, which binds to a WT1 peptide comprising or consisting of the amino acid sequence of SQLECMTWNQMNLGA (SEQ ID NO: 120) or a variant thereof having up to three amino acid substitutions, additions or deletions when presented by an MHC.

In one embodiment, there is provided a TCR comprising a CDR3α comprising the amino acid sequence of CAYRSLKYGNKLVF (SEQ ID NO: 19) or a variant thereof having up to three amino acid substitutions, additions or deletions, and a CDR3β comprising the amino acid sequence of CASSLVALQGAGEQYF (SEQ ID NO: 30) or a variant thereof having up to three amino acid substitutions, additions or deletions, which binds to a WT1 peptide comprising or consisting of the amino acid sequence of QCLSAFTVHFSGQFT (SEQ ID NO: 118) or a variant thereof having up to three amino acid substitutions, additions or deletions when presented by an MHC.

In one embodiment, there is provided a TCR comprising a CDR3α comprising the amino acid sequence of CAYRSLKYGNKLVF (SEQ ID NO: 19) or a variant thereof having up to three amino acid substitutions, additions or deletions, and a CDR3β comprising the amino acid sequence of CASSLVALQGAGEQYF (SEQ ID NO: 30) or a variant thereof having up to three amino acid substitutions, additions or deletions, which binds to a WT1 peptide comprising or consisting of an amino acid sequence of EDPMGQQGSLGEQQY (SEQ ID NO: 119) or a variant thereof having up to three amino acid substitutions, additions or deletions when presented by an MHC.

In one embodiment, there is provided a TCR comprising a CDR3α comprising the amino acid sequence of CAYRSLKYGNKLVF (SEQ ID NO: 19) or a variant thereof having up to three amino acid substitutions, additions or deletions, and a CDR3β comprising the amino acid sequence of CASSLVALQGAGEQYF (SEQ ID NO: 30) or a variant thereof having up to three amino acid substitutions, additions or deletions, which binds to a WT1 peptide comprising or consisting of an amino acid sequence of SQLECMTWNQMNLGA (SEQ ID NO: 120) or a variant thereof having up to three amino acid substitutions, additions or deletions when presented by an MHC.

Further provided by the present invention is a TCR comprising a CDR3α comprising the amino acid sequence of CATDAYSGNTPLVF (SEQ ID NO: 47) or a variant thereof having up to three amino acid substitutions, additions or deletions, and a CDR3β comprising the amino acid sequence of CASRAAGLDTEAFF (SEQ ID NO: 57) or a variant thereof having up to three amino acid substitutions, additions or deletions, which binds to a WT1 peptide comprising comprising or consisting of an amino acid sequence of EPASQHTLRSG (SEQ ID NO: 123) or a variant thereof having up to three amino acid substitutions, additions or deletions when presented by an MHC.

Further provided by the present invention is a TCR comprising a CDR3α comprising the amino acid sequence of CAVRAEIYNQGGKLIF (SEQ ID NO: 52) or a variant thereof having up to three amino acid substitutions, additions or deletions, and a CDR3β comprising the amino acid sequence of CASTQTPYEQYF (SEQ ID NO: 63) or a variant thereof having up to three amino acid substitutions, additions or deletions, which binds to a WT1 peptide comprising or consisting of an amino acid sequence of YESDNHTTPIL (SEQ ID NO: 126) or a variant thereof having up to three amino acid substitutions, additions or deletions when presented by an MHC.

Further provided by the present invention is a TCR comprising a CDR3α comprising the amino acid sequence of CAVRAEIYNQGGKLIF (SEQ ID NO: 52) or a variant thereof having up to three amino acid substitutions, additions or deletions, and a CDR3β comprising the amino acid sequence of CASSTVGGEDYGYTF (SEQ ID NO: 69) or a variant thereof having up to three amino acid substitutions, additions or deletions, which binds to a WT1 peptide comprising comprising or consisting of an amino acid sequence of YESDNHTTPIL (SEQ ID NO: 126) or a variant thereof having up to three amino acid substitutions, additions or deletions when presented by an MHC.

Further provided by the present invention is a TCR comprising a CDR3α comprising the amino acid sequence of CAASMAGAGSYQLTF (SEQ ID NO: 75) or a variant thereof having up to three amino acid substitutions, additions or deletions, and a CDR3β comprising the amino acid sequence of CAISVGQGALYEQYF (SEQ ID NO: 80) or a variant thereof having up to three amino acid substitutions, additions or deletions, which binds to a WT1 peptide comprising the amino acid sequence of NHTTPILCGAQYRIH (SEQ ID NO: 127) or a variant thereof having up to three amino acid substitutions, additions or deletions when presented by an MHC.

Further provided by the present invention is a TCR comprising a CDR3α comprising the amino acid sequence of CAASMAGAGSYQLTF (SEQ ID NO: 75) or a variant thereof having up to three amino acid substitutions, additions or deletions, and a CDR3β comprising the amino acid sequence of CASSVARDRRNYGYTF (SEQ ID NO: 86) or a variant thereof having up to three amino acid substitutions, additions or deletions, which binds to a WT1 peptide comprising the amino acid sequence of NHTTPILCGAQYRIH (SEQ ID NO: 127) or a variant thereof having up to three amino acid substitutions, additions or deletions when presented by an MHC.

Further provided by the present invention is a TCR comprising a CDR3α comprising the amino acid sequence of CAVTVGNKLVF (SEQ ID NO: 175) or a variant thereof having up to three amino acid substitutions, additions or deletions, and a CDR3β comprising the amino acid sequence of CASRGWREQFF (SEQ ID NO: 180) or a variant thereof having up to three amino acid substitutions, additions or deletions, which binds to a WT1 peptide comprising the amino acid sequence of DPGGIWAKLGAAEAS (SEQ ID NO: 251) or a variant thereof having up to three amino acid substitutions, additions or deletions when presented by an MHC.

Further provided by the present invention is a TCR comprising a CDR3α comprising the amino acid sequence of CAARSYNTDKLIF (SEQ ID NO: 186) or a variant thereof having up to three amino acid substitutions, additions or deletions, and a CDR3β comprising the amino acid sequence of CASSWGYQETQYF (SEQ ID NO: 196) or a variant thereof having up to three amino acid substitutions, additions or deletions, which binds to a WT1 peptide comprising the amino acid sequence of NHTTPILCGAQYRIH (SEQ ID NO: 252) or a variant thereof having up to three amino acid substitutions, additions or deletions when presented by an MHC.

Further provided by the present invention is a TCR comprising a CDR3α comprising the amino acid sequence of CAASYNNARLMF (SEQ ID NO: 191) or a variant thereof having up to three amino acid substitutions, additions or deletions, and a CDR3β comprising the amino acid sequence of CASSPTGGEYYGYTF (SEQ ID NO: 202) or a variant thereof having up to three amino acid substitutions, additions or deletions, which binds to a WT1 peptide comprising the amino acid sequence of KRHQRRHTGVKPFQC (SEQ ID NO: 253) or a variant thereof having up to three amino acid substitutions, additions or deletions when presented by an MHC.

Further provided by the present invention is a TCR comprising a CDR3α comprising the amino acid sequence of CAASYNNARLMF (SEQ ID NO: 191) or a variant thereof having up to three amino acid substitutions, additions or deletions, and a CDR3β comprising the amino acid sequence of CASSSYPLRTGRYNSYNSPLHF (SEQ ID NO: 208) or a variant thereof having up to three amino acid substitutions, additions or deletions, which binds to a WT1 peptide comprising the amino acid sequence of PSCQKKFARSDELVR (SEQ ID NO: 254) or a variant thereof having up to three amino acid substitutions, additions or deletions when presented by an MHC.

Further provided by the present invention is a TCR comprising a CDR3α comprising the amino acid sequence of CAANNARLMF (SEQ ID NO: 92) or a variant thereof having up to three amino acid substitutions, additions or deletions, and a CDR3β comprising the amino acid sequence of CASSPGQHGELFF (SEQ ID NO: 271) or a variant thereof having up to three amino acid substitutions, additions or deletions, which binds to a WT1 peptide comprising the amino acid sequence of APVLDFAPPGA (SEQ ID NO: 117) or a variant thereof having up to three amino acid substitutions, additions or deletions when presented by an MHC.

Further provided by the present invention is a TCR comprising a CDR3α comprising the amino acid sequence of CAASATGNQFYF (SEQ ID NO: 266) or a variant thereof having up to three amino acid substitutions, additions or deletions, and a CDR3β comprising the amino acid sequence of CASSDTRAREQFF (SEQ ID NO: 97) or a variant thereof having up to three amino acid substitutions, additions or deletions, which binds to a WT1 peptide comprising the amino acid sequence of APVLDFAPPGA (SEQ ID NO: 117) or a variant thereof having up to three amino acid substitutions, additions or deletions when presented by an MHC.

Further provided by the present invention is a TCR comprising a CDR3α comprising the amino acid sequence of CAASATGNQFYF (SEQ ID NO: 266) or a variant thereof having up to three amino acid substitutions, additions or deletions, and a CDR3β comprising the amino acid sequence of CASSPGQHGELFF (SEQ ID NO: 271) or a variant thereof having up to three amino acid substitutions, additions or deletions, which binds to a WT1 peptide comprising the amino acid sequence of APVLDFAPPGA (SEQ ID NO: 117) or a variant thereof having up to three amino acid substitutions, additions or deletions when presented by an MHC.

Further provided by the present invention is a TCR comprising a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions, which binds to a WT1 peptide comprising the amino acid sequence of APVLDFAPPGA (SEQ ID NO: 117) or a variant thereof having up to three amino acid substitutions, additions or deletions when presented by an MHC.

Further provided by the present invention is a TCR comprising comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions, which binds to a WT1 peptide comprising the amino acid sequence of APVLDFAPPGA (SEQ ID NO: 117) or a variant thereof having up to three amino acid substitutions, additions or deletions when presented by an MHC.

Further provided by the present invention is a TCR comprising a CDR3α comprising the amino acid sequence of CAVEATDSWGKLQF (SEQ ID NO: 108) or a variant thereof having up to three amino acid substitutions, additions or deletions, and a CDR3β comprising the amino acid sequence of CASSLGLSISQETQYF (SEQ ID NO: 288) or a variant thereof having up to three amino acid substitutions, additions or deletions, which binds to a WT1 peptide comprising the amino acid sequence of NQMNLGATLKG (SEQ ID NO: 250) or a variant thereof having up to three amino acid substitutions, additions or deletions when presented by an MHC.

Further provided by the present invention is a TCR comprising a CDR3α comprising the amino acid sequence of CAVRTSYDKVIF (SEQ ID NO: 113) or a variant thereof having up to three amino acid substitutions, additions or deletions, and a CDR3β comprising the amino acid sequence of CASSLGLSISQETQYF (SEQ ID NO: 288) or a variant thereof having up to three amino acid substitutions, additions or deletions, which binds to a WT1 peptide comprising the amino acid sequence of NQMNLGATLKG (SEQ ID NO: 250) or a variant thereof having up to three amino acid substitutions, additions or deletions when presented by an MHC.

Further provided by the present invention is a TCR comprising a CDR3α comprising the amino acid sequence of CAERLNTDKLIF (SEQ ID NO: 103) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions, which binds to a WT1 peptide comprising the amino acid sequence of APVLDFAPPGA (SEQ ID NO: 117) or a variant thereof having up to three amino acid substitutions, additions or deletions when presented by an MHC.

Further provided by the present invention is a TCR comprising a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions, which binds to a WT1 peptide comprising the amino acid sequence of APVLDFAPPGA (SEQ ID NO: 117) or a variant thereof having up to three amino acid substitutions, additions or deletions when presented by an MHC.

Further provided by the present invention is a TCR comprising a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions, which binds to a WT1 peptide comprising the amino acid sequence of APVLDFAPPGA (SEQ ID NO: 117) or a variant thereof having up to three amino acid substitutions, additions or deletions when presented by an MHC.

Example TCR amino acid sequences of the present invention are provided in Table 1.

TABLE 1
Donor: HD1

| Chain | Region | Amino acid sequence | SEQ ID NO |
|---|---|---|---|
| Alpha (α) | CDR1α | KALYS | SEQ ID NO: 1 |
| | CDR2α | LLKGGEQ | SEQ ID NO: 2 |
| | CDR3α | CGTAWINDYKLSF | SEQ ID NO: 3 |
| | Variable | METLLKVLSGTLLWQLTWVRSQQPVQSPQAVILREGEDAVIN | SEQ ID NO: 4 |
| | | CSSSKALYSVHWYRQKHGEAPVFLMILLKGGEQKGHEKISAS | |
| | | FNEKKQQSSLYLTASQLSYSGTYFCGTAWINDYKLSFGAGTT | |
| | | VTVRAN | |
| | Full - with | METLLKVLSGTLLWQLTWVRSQQPVQSPQAVILREGEDAVIN | SEQ ID NO: 5 |
| | TRAC constant | CSSSKALYSVHWYRQKHGEAPVFLMILLKGGEQKGHEKISAS | |
| | domain | FNEKKQQSSLYLTASQLSYSGTYFCGTAWINDYKLSFGAGTT | |
| | | VTVRANIQNPDPAVYQLRDSKSSDKSVCLFTDFDSQTNVSQS | |
| | | KDSDVYITDKTVLDMRSMDFKSNSAVAWSNKSDFACANAFNN | |
| | | SIIPEDTFFPSPESSCDVKLVEKSFETDTNLNFQNLSVIGFR | |
| | | ILLLKVAGFNLLMTLRLWSS | |
| Beta (β) | CDR1β | SGHDY | SEQ ID NO: 6 |
| | CDR2β | FNNNVP | SEQ ID NO: 7 |
| | CDR3β | CASRKTGGYSNQPQHF | SEQ ID NO: 8 |
| | Variable | MGSWTLCCVSLCILVAKHTDAGVIQSPRHEVTEMGQEVTLRC | SEQ ID NO: 9 |
| | | KPISGHDYLFWYRQTMMRGLELLIYFNNNVPIDDSGMPEDRF | |
| | | SAKMPNASFSTLKIQPSEPRDSAVYFCASRKTGGYSNQPQHF | |
| | | GDGTRLSILE | |
| | Full - with | MGSWTLCCVSLCILVAKHTDAGVIQSPRHEVTEMGQEVTLRC | SEQ ID NO: 10 |
| | TRBC1 | KPISGHDYLFWYRQTMMRGLELLIYFNNNVPIDDSGMPEDRF | |
| | constant | SAKMPNASFSTLKIQPSEPRDSAVYFCASRKTGGYSNQPQHF | |
| | domain | GDGTRLSILEDLNKVFPPEVAVFEPSEAEISHTQKATLVCLA | |
| | | TGFFPDHVELSWWVNGKEVHSGVSTDPQPLKEQPALNDSRYC | |
| | | LSSRLRVSATFWQNPRNHFRCQVQFYGLSENDEWTQDRAKPV | |
| | | TQIVSAEAWGRADCGFTSVSYQQGVLSATILYEILLGKATLY | |
| | | AVLVSALVLMAMVKRKDF | |
| | Full - with | MGSWTLCCVSLCILVAKHTDAGVIQSPRHEVTEMGQEVTLRC | SEQ ID NO: 11 |
| | TRBC2 | KPISGHDYLFWYRQTMMRGLELLIYFNNNVPIDDSGMPEDRF | |
| | constant | SAKMPNASFSTLKIQPSEPRDSAVYFCASRKTGGYSNQPQHF | |
| | domain | GDGTRLSILEDLKNVFPPEVAVFEPSEAEISHTQKATLVCLA | |
| | | TGFYPDHVELSWWVNGKEVHSGVSTDPQPLKEQPALNDSRYC | |
| | | LSSRLRVSATFWQNPRNHFRCQVQFYGLSENDEWTQDRAKPV | |
| | | TQIVSAEAWGRADCGFTSESYQQGVLSATILYEILLGKATLY | |
| | | AVLVSALVLMAMVKRKDSRG | |

Donor: HD2

| Clonotype | Chain | Region | Sequence | SEQ ID NO |
|---|---|---|---|---|
| HD2-1 | Alpha (α) | CDR1α | SSVPPY | SEQ ID NO: 12 |
| | | CDR2α | YTSAATLV | SEQ ID NO: 13 |
| | | CDR3α | CAVRLSGSARQLTF | SEQ ID NO: 14 |
| | | Variable domain | MLLLLVPVLEVIFTLGGTRAQSVTQLGSHVSV | SEQ ID NO: 15 |
| | | | SEGALVLLRCNYSSSVPPYLFWYVQYPNQGLQ | |
| | | | LLLKYTSAATLVKGINGFEAEFKKSETSFHLT | |
| | | | KPSAHMSDAAEYFCAVRLSGSARQLTFGSGTQ | |
| | | | LTVLPD | |
| | | Full - with TRAC | MLLLLVPVLEVIFTLGGTRAQSVTQLGSHVSV | SEQ ID NO: 16 |
| | | constant domain | SEGALVLLRCNYSSSVPPYLFWYVQYPNQGLQ | |
| | | | LLLKYTSAATLVKGINGFEAEFKKSETSFHLT | |
| | | | KPSAHMSDAAEYFCAVRLSGSARQLTFGSGTQ | |
| | | | LTVLPDIQNPDPAVYQLRDSKSSDKSVCLFTD | |
| | | | FDSQTNVSQSKDSDVYITDKTVLDMRSMDFKS | |
| | | | NSAVAWSNKSDFACANAFNNSIIPEDTFFPSP | |
| | | | ESSCDVKLVEKSFETDTNLNFQNLSVIGFRIL | |
| | | | LLKVAGFNLLMTLRLWSS | |
| HD2-2 | Alpha (α) | CDR1α | TSESDYY | SEQ ID NO: 17 |
| | | CDR2α | QEAYKQQN | SEQ ID NO: 18 |
| | | CDR3α | CAYRSLKYGNKLVF | SEQ ID NO: 19 |
| | | Variable domain | MACPGFLWALVISTCLEFSMAQTVTQSQPEMS | SEQ ID NO: 20 |
| | | | VQEAETVTLSCTYDTSESDYYLFWYKQPPSRQ | |
| | | | MILVIRQEAYKQQNATENRFSVNFQKAAKSFS | |
| | | | LKISDSQLGDAAMYFCAYRSLKYGNKLVFGAG | |
| | | | TILRVKSY | |
| | | Full - with TRAC | MACPGFLWALVISTCLEFSMAQTVTQSQPEMS | SEQ ID NO: 21 |
| | | constant domain | VQEAETVTLSCTYDTSESDYYLFWYKQPPSRQ | |
| | | | MILVIRQEAYKQQNATENRFSVNFQKAAKSFS | |
| | | | LKISDSQLGDAAMYFCAYRSLKYGNKLVFGAG | |
| | | | TILRVKSYIQNPDPAVYQLRDSKSSDKSVCLF | |
| | | | TDFDSQTNVSQSKDSDVYITDKTVLDMRSMDF | |
| | | | KSNSAVAWSNKSDFACANAFNNSIIPEDTFFP | |
| | | | SPESSCDVKLVEKSFETDTNLNFQNLSVIGFR | |
| | | | ILLLKVAGFNLLMTLRLWSS | |
| HD2-1β | Beta (β) | CDR1β | SGHAT | SEQ ID NO: 22 |
| | | CDR2β | FQNNGV | SEQ ID NO: 23 |
| | | CDR3β | CASSLLGDEQYF | SEQ ID NO: 24 |
| | | Variable domain | MGTRLLCWAALCLLGAELTEAGVAQSPRYKII | SEQ ID NO: 25 |
| | | | EKRQSVAFWCNPISGHATLYWYQQILGQGPKL | |
| | | | LIQFQNNGVVDDSQLPKDRFSAERLKGVDSTL | |
| | | | KIQPAKLEDSAVYLCASSLLGDEQYFGPGTRL | |
| | | | TVTE | |
| | | Full - with TRBC1 | MGTRLLCWAALCLLGAELTEAGVAQSPRYKII | SEQ ID NO: 26 |
| | | constant domain | EKRQSVAFWCNPISGHATLYWYQQILGQGPKL | |
| | | | LIQFQNNGVVDDSQLPKDRFSAERLKGVDSTL | |
| | | | KIQPAKLEDSAVYLCASSLLGDEQYFGPGTRL | |
| | | | TVTEDLNKVFPPEVAVFEPSEAEISHTQKATL | |
| | | | VCLATGFFPDHVELSWWVNGKEVHSGVSTDPQ | |
| | | | PLKEQPALNDSRYCLSSRLRVSATFWQNPRNH | |
| | | | FRCQVQFYGLSENDEWTQDRAKPVTQIVSAEA | |
| | | | WGRADCGFTSVSYQQGVLSATILYEILLGKAT | |
| | | | LYAVLVSALVLMAMVKRKDF | |
| | | Full - with TRBC2 | MGTRLLCWAALCLLGAELTEAGVAQSPRYKII | SEQ ID NO: 27 |
| | | constant domain | EKRQSVAFWCNPISGHATLYWYQQILGQGPKL | |
| | | | LIQFQNNGVVDDSQLPKDRFSAERLKGVDSTL | |
| | | | KIQPAKLEDSAVYLCASSLLGDEQYFGPGTRL | |
| | | | TVTEDLKNVFPPEVAVFEPSEAEISHTQKATL | |
| | | | VCLATGFYPDHVELSWWVNGKEVHSGVSTDPQ | |
| | | | PLKEQPALNDSRYCLSSRLRVSATFWQNPRNH | |
| | | | FRCQVQFYGLSENDEWTQDRAKPVTQIVSAEA | |
| | | | WGRADCGFTSESYQQGVLSATILYEILLGKAT | |
| | | | LYAVLVSALVLMAMVKRKDSRG | |
| HD2-2β | Beta (β) | CDR1β | SGHTA | SEQ ID NO: 28 |
| | | CDR2β | FQGNSA | SEQ ID NO: 29 |
| | | CDR3β | CASSLVALQGAGEQYF | SEQ ID NO: 30 |
| | | Variable domain | MGTRLLFWVAFCLLGADHTGAGVSQSPSNKVT | SEQ ID NO: 31 |
| | | | EKGKDVELRCDPISGHTALYWYRQSLGQGLEF | |
| | | | LIYFQGNSAPDKSGLPSDRFSAERTGGSVSTL | |
| | | | TIQRTQQEDSAVYLCASSLVALQGAGEQYFGP | |
| | | | GTRLTVTE | |
| | | Full - with TRBC1 | MGTRLLFWVAFCLLGADHTGAGVSQSPSNKVT | SEQ ID NO: 32 |
| | | constant domain | EKGKDVELRCDPISGHTALYWYRQSLGQGLEF | |
| | | | LIYFQGNSAPDKSGLPSDRFSAERTGGSVSTL | |
| | | | TIQRTQQEDSAVYLCASSLVALQGAGEQYFGP | |
| | | | GTRLTVTEDLNKVFPPEVAVFEPSEAEISHTQ | |
| | | | KATLVCLATGFFPDHVELSWWVNGKEVHSGVS | |
| | | | TDPQPLKEQPALNDSRYCLSSRLRVSATFWQN | |
| | | | PRNHFRCQVQFYGLSENDEWTQDRAKPVTQIV | |
| | | | SAEAWGRADCGFTSVSYQQGVLSATILYEILL | |
| | | | GKATLYAVLVSALVLMAMVKRKDF | |
| | | Full - with TRBC2 | MGTRLLFWVAFCLLGADHTGAGVSQSPSNKVT | SEQ ID NO: 33 |
| | | constant domain | EKGKDVELRCDPISGHTALYWYRQSLGQGLEF | |
| | | | LIYFQGNSAPDKSGLPSDRFSAERTGGSVSTL | |
| | | | TIQRTQQEDSAVYLCASSLVALQGAGEQYFGP | |
| | | | GTRLTVTEDLKNVFPPEVAVFEPSEAEISHTQ | |
| | | | KATLVCLATGFYPDHVELSWWVNGKEVHSGVS | |
| | | | TDPQPLKEQPALNDSRYCLSSRLRVSATFWQN | |
| | | | PRNHFRCQVQFYGLSENDEWTQDRAKPVTQIV | |
| | | | SAEAWGRADCGFTSESYQQGVLSATILYEILL | |
| | | | GKATLYAVLVSALVLMAMVKRKDSRG | |

Donor: HD3

| Chain | Type | Sequence | SEQ ID NO |
|---|---|---|---|
| Alpha (α) | CDR1α | NSASQS | SEQ ID NO: 34 |
| | CDR2α | VYSSGN | SEQ ID NO: 35 |
| | CDR3α | CVVNLLSNQGGKLIF | SEQ ID NO: 36 |
| | Variable | MISLRVLLVILWLQLSWVWSQRKEVEQDPGPFNVPEGATVAFNCTYS | SEQ ID NO: 37 |
| | domain | NSASQSFFWYRQDCRKEPKLLMSVYSSGNEDGRFTAQLNRASQYISL | |
| | | LIRDSKLSDSATYLCVVNLLSNQGGKLIFGQGTELSVKPN | |
| | Full - with | MISLRVLLVILWLQLSWVWSQRKEVEQDPGPFNVPEGATVAFNCTYS | SEQ ID NO: 38 |
| | TRAC | NSASQSFFWYRQDCRKEPKLLMSVYSSGNEDGRFTAQLNRASQYISL | |
| | constant | LIRDSKLSDSATYLCVVNLLSNQGGKLIFGQGTELSVKPNIQNPDPA | |
| | domain | VYQLRDSKSSDKSVCLFTDFDSQTNVSQSKDSDVYITDKTVLDMRSM | |
| | | DFKSNSAVAWSNKSDFACANAFNNSIIPEDTFFPSPESSCDVKLVEK | |
| | | SFETDTNLNFQNLSVIGFRILLLKVAGFNLLMTLRLWSS | |
| Beta (β) | CDR1β | LGHNA | SEQ ID NO: 39 |
| | CDR2β | YSLEER | SEQ ID NO: 40 |
| | CDR3β | CASSQDYLVSNEKLFF | SEQ ID NO: 41 |
| | Variable | MGCRLLCCAVLCLLGAGELVPMETGVTQTPRHLVMGMTNKKSLKCEQ | SEQ ID NO: 42 |
| | domain | HLGHNAMYWYKQSAKKPLELMFVYSLEERVENNSVPSRFSPECPNSS | |
| | | HLFLHLHTLQPEDSALYLCASSQDYLVSNEKLFFGSGTQLSVLE | |
| | Full - with | MGCRLLCCAVLCLLGAGELVPMETGVTQTPRHLVMGMTNKKSLKCEQ | SEQ ID NO: 43 |
| | TRBC1 | HLGHNAMYWYKQSAKKPLELMFVYSLEERVENNSVPSRFSPECPNSS | |
| | constant | HLFLHLHTLQPEDSALYLCASSQDYLVSNEKLFFGSGTQLSVLEDLN | |
| | domain | KVFPPEVAVFEPSEAEISHTQKATLVCLATGFFPDHVELSWWVNGKE | |
| | | VHSGVSTDPQPLKEQPALNDSRYCLSSRLRVSATFWQNPRNHFRCQV | |
| | | QFYGLSENDEWTQDRAKPVTQIVSAEAWGRADCGFTSVSYQQGVLSA | |
| | | TILYEILLGKATLYAVLVSALVLMAMVKRKDF | |
| | Full - with | MGCRLLCCAVLCLLGAGELVPMETGVTQTPRHLVMGMTNKKSLKCEQ | SEQ ID NO: 44 |
| | TRBC2 | HLGHNAMYWYKQSAKKPLELMFVYSLEERVENNSVPSRFSPECPNSS | |
| | constant | HLFLHLHTLQPEDSALYLCASSQDYLVSNEKLFFGSGTQLSVLEDLK | |
| | domain | NVFPPEVAVFEPSEAEISHTQKATLVCLATGFYPDHVELSWWVNGKE | |
| | | VHSGVSTDPQPLKEQPALNDSRYCLSSRLRVSATFWQNPRNHFRCQV | |
| | | QFYGLSENDEWTQDRAKPVTQIVSAEAWGRADCGFTSESYQQGVLSA | |
| | | TILYEILLGKATLYAVLVSALVLMAMVKRKDSRG | |

Donor: HD4

| Clonotype | Chain | Region | Sequence | SEQ ID NO |
|---|---|---|---|---|
| HD4-1 | Alpha (α) | CDR1α | TSINN | SEQ ID NO: 45 |
| | | CDR2α | IRSNERE | SEQ ID NO: 46 |
| | | CDR3α | CATDAYSGNTPLVF | SEQ ID NO: 47 |
| | | Variable domain | METLLGVSLVILWLQLARVNSQQGEEDPQALS | SEQ ID NO: 48 |
| | | | IQEGENATMNCSYKTSINNLQWYRQNSGRGLV | |
| | | | HLILIRSNEREKHSGRLRVTLDTSKKSSSLLI | |
| | | | TASRAADTASYFCATDAYSGNTPLVFGKGTRL | |
| | | | SVIAN | |
| | | Full - with TRAC | METLLGVSLVILWLQLARVNSQQGEEDPQALS | SEQ ID NO: 49 |
| | | constant domain | IQEGENATMNCSYKTSINNLQWYRQNSGRGLV | |
| | | | HLILIRSNEREKHSGRLRVTLDTSKKSSSLLI | |
| | | | TASRAADTASYFCATDAYSGNTPLVFGKGTRL | |
| | | | SVIANIQNPDPAVYQLRDSKSSDKSVCLFTDF | |
| | | | DSQTNVSQSKDSDVYITDKTVLDMRSMDFKSN | |
| | | | SAVAWSNKSDFACANAFNNSIIPEDTFFPSPE | |
| | | | SSCDVKLVEKSFETDTNLNFQNLSVIGFRILL | |
| | | | LKVAGFNLLMTLRLWSS | |
| HD4-2 | Alpha (α) | CDR1α | DSAIYN | SEQ ID NO: 50 |
| | | CDR2α | IQSSQRE | SEQ ID NO: 51 |
| | | CDR3α | CAVRAEIYNQGGKLIF | SEQ ID NO: 52 |
| | | Variable domain | METLLGLLILWLQLQWVSSKQEVTQIPAALSV | SEQ ID NO: 53 |
| | | | PEGENLVLNCSFTDSAIYNLQWFRQDPGKGLT | |
| | | | SLLLIQSSQREQTSGRLNASLDKSSGRSTLYI | |
| | | | AASQPGDSATYLCAVRAEIYNQGGKLIFGQGT | |
| | | | ELSVKPN | |
| | | Full - with TRAC | METLLGLLILWLQLQWVSSKQEVTQIPAALSV | SEQ ID NO: 54 |
| | | constant domain | PEGENLVLNCSFTDSAIYNLQWFRQDPGKGLT | |
| | | | SLLLIQSSQREQTSGRLNASLDKSSGRSTLYI | |
| | | | AASQPGDSATYLCAVRAEIYNQGGKLIFGQGT | |
| | | | ELSVKPNIQNPDPAVYQLRDSKSSDKSVCLFT | |
| | | | DFDSQTNVSQSKDSDVYITDKTVLDMRSMDFK | |
| | | | SNSAVAWSNKSDFACANAFNNSIIPEDTFFPS | |
| | | | PESSCDVKLVEKSFETDTNLNFQNLSVIGFRI | |
| | | | LLLKVAGFNLLMTLRLWSS | |
| HD4-1 | Beta (β) | CDR1β | MNHNS | SEQ ID NO: 55 |
| | | CDR2β | SASEGT | SEQ ID NO: 56 |
| | | CDR3β | CASRAAGLDTEAFF | SEQ ID NO: 57 |
| | | Variable domain | MSIGLLCCVAFSLLWASPVNAGVTQTPKFQVL | SEQ ID NO: 58 |
| | | | KTGQSMTLQCAQDMNHNSMYWYRQDPGMGLRL | |
| | | | IYYSASEGTTDKGEVPNGYNVSRLNKREFSLR | |
| | | | LESAAPSQTSVYFCASRAAGLDTEAFFGQGTR | |
| | | | LTVVE | |
| | | Full - with TRBC1 | MSIGLLCCVAFSLLWASPVNAGVTQTPKFQVL | SEQ ID NO: 59 |
| | | constant domain | KTGQSMTLQCAQDMNHNSMYWYRQDPGMGLRL | |
| | | | IYYSASEGTTDKGEVPNGYNVSRLNKREFSLR | |
| | | | LESAAPSQTSVYFCASRAAGLDTEAFFGQGTR | |
| | | | LTVVEDLNKVFPPEVAVFEPSEAEISHTQKAT | |
| | | | LVCLATGFFPDHVELSWWVNGKEVHSGVSTDP | |
| | | | QPLKEQPALNDSRYCLSSRLRVSATFWQNPRN | |
| | | | HFRCQVQFYGLSENDEWTQDRAKPVTQIVSAE | |
| | | | AWGRADCGFTSVSYQQGVLSATILYEILLGKA | |
| | | | TLYAVLVSALVLMAMVKRKDF | |
| | | Full - with TRBC2 | MSIGLLCCVAFSLLWASPVNAGVTQTPKFQVL | SEQ ID NO: 60 |
| | | constant domain | KTGQSMTLQCAQDMNHNSMYWYRQDPGMGLRL | |
| | | | IYYSASEGTTDKGEVPNGYNVSRLNKREFSLR | |
| | | | LESAAPSQTSVYFCASRAAGLDTEAFFGQGTR | |
| | | | LTVVEDLKNVFPPEVAVFEPSEAEISHTQKAT | |
| | | | LVCLATGFYPDHVELSWWVNGKEVHSGVSTDP | |
| | | | QPLKEQPALNDSRYCLSSRLRVSATFWQNPRN | |
| | | | HFRCQVQFYGLSENDEWTQDRAKPVTQIVSAE | |
| | | | AWGRADCGFTSESYQQGVLSATILYEILLGKA | |
| | | | TLYAVLVSALVLMAMVKRKDSRG | |
| HD4-2 | Beta (β) | CDR1β | MNHNY | SEQ ID NO: 61 |
| | | CDR2β | SVGAGI | SEQ ID NO: 62 |
| | | CDR3β | CASTQTPYEQYF | SEQ ID NO: 63 |
| | | Variable domain | MSISLLCCAAFPLLWAGPVNAGVTQTPKFRIL | SEQ ID NO: 64 |
| | | | KIGQSMTLQCTQDMNHNYMYWYRQDPGMGLKL | |
| | | | IYYSVGAGITDKGEVPNGYNVSRSTTEDFPLR | |
| | | | LELAAPSQTSVYFCASTQTPYEQYFGPGTRLT | |
| | | | VTE | |
| | | Full - with TRBC1 | MSISLLCCAAFPLLWAGPVNAGVTQTPKFRIL | SEQ ID NO: 65 |
| | | constant domain | KIGQSMTLQCTQDMNHNYMYWYRQDPGMGLKL | |
| | | | IYYSVGAGITDKGEVPNGYNVSRSTTEDFPLR | |
| | | | LELAAPSQTSVYFCASTQTPYEQYFGPGTRLT | |
| | | | VTEDLNKVFPPEVAVFEPSEAEISHTQKATLV | |
| | | | CLATGFFPDHVELSWWVNGKEVHSGVSTDPQP | |
| | | | LKEQPALNDSRYCLSSRLRVSATFWQNPRNHF | |
| | | | RCQVQFYGLSENDEWTQDRAKPVTQIVSAEAW | |
| | | | GRADCGFTSVSYQQGVLSATILYEILLGKATL | |
| | | | YAVLVSALVLMAMVKRKDF | |
| | | Full - with TRBC2 | MSISLLCCAAFPLLWAGPVNAGVTQTPKFRIL | SEQ ID NO: 66 |
| | | constant domain | KIGQSMTLQCTQDMNHNYMYWYRQDPGMGLKL | |
| | | | IYYSVGAGITDKGEVPNGYNVSRSTTEDFPLR | |
| | | | LELAAPSQTSVYFCASTQTPYEQYFGPGTRLT | |
| | | | VTEDLKNVFPPEVAVFEPSEAEISHTQKATLV | |
| | | | CLATGFYPDHVELSWWVNGKEVHSGVSTDPQP | |
| | | | LKEQPALNDSRYCLSSRLRVSATFWQNPRNHF | |
| | | | RCQVQFYGLSENDEWTQDRAKPVTQIVSAEAW | |
| | | | GRADCGFTSESYQQGVLSATILYEILLGKATL | |
| | | | YAVLVSALVLMAMVKRKDSRG | |
| HD4-3 | Beta (β) | CDR1β | SGHNS | SEQ ID NO: 67 |
| | | CDR2β | FNNNVP | SEQ ID NO: 68 |
| | | CDR3β | CASSTVGGEDYGYTF | SEQ ID NO: 69 |
| | | Variable domain | MDSWTFCCVSLCILVAKHTDAGVIQSPRHEVT | SEQ ID NO: 70 |
| | | | EMGQEVTLRCKPISGHNSLFWYRQTMMRGLEL | |
| | | | LIYFNNNVPIDDSGMPEDRFSAKMPNASFSTL | |
| | | | KIQPSEPRDSAVYFCASSTVGGEDYGYTFGSG | |
| | | | TRLTVVE | |
| | | Full - with TRBC1 | MDSWTFCCVSLCILVAKHTDAGVIQSPRHEVT | SEQ ID NO: 71 |
| | | constant domain | EMGQEVTLRCKPISGHNSLFWYRQTMMRGLEL | |
| | | | LIYFNNNVPIDDSGMPEDRFSAKMPNASFSTL | |
| | | | KIQPSEPRDSAVYFCASSTVGGEDYGYTFGSG | |
| | | | TRLTVVEDLNKVFPPEVAVFEPSEAEISHTQK | |
| | | | ATLVCLATGFFPDHVELSWWVNGKEVHSGVST | |
| | | | DPQPLKEQPALNDSRYCLSSRLRVSATFWQNP | |
| | | | RNHFRCQVQFYGLSENDEWTQDRAKPVTQIVS | |
| | | | AEAWGRADCGFTSVSYQQGVLSATILYEILLG | |
| | | | KATLYAVLVSALVLMAMVKRKDF | |
| | | Full - with TRBC2 | MDSWTFCCVSLCILVAKHTDAGVIQSPRHEVT | SEQ ID NO: 72 |
| | | constant domain | EMGQEVTLRCKPISGHNSLFWYRQTMMRGLEL | |
| | | | LIYFNNNVPIDDSGMPEDRFSAKMPNASFSTL | |
| | | | KIQPSEPRDSAVYFCASSTVGGEDYGYTFGSG | |
| | | | TRLTVVEDLKNVFPPEVAVFEPSEAEISHTQK | |
| | | | ATLVCLATGFYPDHVELSWWVNGKEVHSGVST | |
| | | | DPQPLKEQPALNDSRYCLSSRLRVSATFWQNP | |
| | | | RNHFRCQVQFYGLSENDEWTQDRAKPVTQIVS | |
| | | | AEAWGRADCGFTSESYQQGVLSATILYEILLG | |
| | | | KATLYAVLVSALVLMAMVKRKDSRG | |

Donor: HD5

| Clonotype | Chain | Region | Sequence | SEQ ID NO |
|---|---|---|---|---|
| HD5-1 | Alpha (α) | CDR1α | DSASNY | SEQ ID NO: 73 |
| | | CDR2α | IRSNVGE | SEQ ID NO: 74 |
| | | CDR3α | CAASMAGAGSYQLTF | SEQ ID NO: 75 |
| | | Variable domain | MTSIRAVFIFLWLQLDLVNGENVEQHPSTLSV | SEQ ID NO: 76 |
| | | | QEGDSAVIKCTYSDSASNYFPWYKQELGKRPQ | |
| | | | LIIDIRSNVGEKKDQRIAVTLNKTAKHFSLHI | |
| | | | TETQPEDSAVYFCAASMAGAGSYQLTFGKGTK | |
| | | | LSVIPN | |
| | | Full - with TRAC | MTSIRAVFIFLWLQLDLVNGENVEQHPSTLSV | SEQ ID NO: 77 |
| | | constant domain | QEGDSAVIKCTYSDSASNYFPWYKQELGKRPQ | |
| | | | LIIDIRSNVGEKKDQRIAVTLNKTAKHFSLHI | |
| | | | TETQPEDSAVYFCAASMAGAGSYQLTFGKGTK | |
| | | | LSVIPNIQNPDPAVYQLRDSKSSDKSVCLFTD | |
| | | | FDSQTNVSQSKDSDVYITDKTVLDMRSMDFKS | |
| | | | NSAVAWSNKSDFACANAFNNSIIPEDTFFPSP | |
| | | | ESSCDVKLVEKSFETDTNLNFQNLSVIGFRIL | |
| | | | LLKVAGFNLLMTLRLWSS | |
| HD5-1 | Beta (β) | CDR1β | ENHRY | SEQ ID NO: 78 |
| | | CDR2β | SYGVKD | SEQ ID NO: 79 |
| | | CDR3β | CAISVGQGALYEQYF | SEQ ID NO: 80 |
| | | Variable domain | MGTRLFFYVALCLLWTGHMDAGITQSPRHKVT | SEQ ID NO: 81 |
| | | | ETGTPVTLRCHQTENHRYMYWYRQDPGHGLRL | |
| | | | IHYSYGVKDTDKGEVSDGYSVSRSKTEDFLLT | |
| | | | LESATSSQTSVYFCAISVGQGALYEQYFGPGT | |
| | | | RLTVTE | |
| | | Full - with TRBC1 | MGTRLFFYVALCLLWTGHMDAGITQSPRHKVT | SEQ ID NO: 82 |
| | | constant domain | ETGTPVTLRCHQTENHRYMYWYRQDPGHGLRL | |
| | | | IHYSYGVKDTDKGEVSDGYSVSRSKTEDFLLT | |
| | | | LESATSSQTSVYFCAISVGQGALYEQYFGPGT | |
| | | | RLTVTEDLNKVFPPEVAVFEPSEAEISHTQKA | |
| | | | TLVCLATGFFPDHVELSWWVNGKEVHSGVSTD | |
| | | | PQPLKEQPALNDSRYCLSSRLRVSATFWQNPR | |
| | | | NHFRCQVQFYGLSENDEWTQDRAKPVTQIVSA | |
| | | | EAWGRADCGFTSVSYQQGVLSATILYEILLGK | |
| | | | ATLYAVLVSALVLMAMVKRKDF | |
| | | Full - with TRBC2 | MGTRLFFYVALCLLWTGHMDAGITQSPRHKVT | SEQ ID NO: 83 |
| | | constant domain | ETGTPVTLRCHQTENHRYMYWYRQDPGHGLRL | |
| | | | IHYSYGVKDTDKGEVSDGYSVSRSKTEDFLLT | |
| | | | LESATSSQTSVYFCAISVGQGALYEQYFGPGT | |
| | | | RLTVTEDLKNVFPPEVAVFEPSEAEISHTQKA | |
| | | | TLVCLATGFYPDHVELSWWVNGKEVHSGVSTD | |
| | | | PQPLKEQPALNDSRYCLSSRLRVSATFWQNPR | |
| | | | NHFRCQVQFYGLSENDEWTQDRAKPVTQIVSA | |
| | | | EAWGRADCGFTSESYQQGVLSATILYEILLGK | |
| | | | ATLYAVLVSALVLMAMVKRKDSRG | |
| HD5-2 | Beta (β) | CDR1β | SGDLS | SEQ ID NO: 84 |
| | | CDR2β | YYNGEE | SEQ ID NO: 85 |
| | | CDR3β | CASSVARDRRNYGYTF | SEQ ID NO: 86 |
| | | Variable domain | MGFRLLCCVAFCLLGAGPVDSGVTQTPKHLIT | SEQ ID NO: 87 |
| | | | ATGQRVTLRCSPRSGDLSVYWYQQSLDQGLQF | |
| | | | LIQYYNGEERAKGNILERFSAQQFPDLHSELN | |
| | | | LSSLELGDSALYFCASSVARDRRNYGYTFGSG | |
| | | | TRLTVVE | |
| | | Full - with TRBC1 | MGFRLLCCVAFCLLGAGPVDSGVTQTPKHLIT | SEQ ID NO: 88 |
| | | constant domain | ATGQRVTLRCSPRSGDLSVYWYQQSLDQGLQF | |
| | | | LIQYYNGEERAKGNILERFSAQQFPDLHSELN | |
| | | | LSSLELGDSALYFCASSVARDRRNYGYTFGSG | |
| | | | TRLTVVEDLNKVFPPEVAVFEPSEAEISHTQK | |
| | | | ATLVCLATGFFPDHVELSWWVNGKEVHSGVST | |
| | | | DPQPLKEQPALNDSRYCLSSRLRVSATFWQNP | |
| | | | RNHFRCQVQFYGLSENDEWTQDRAKPVTQIVS | |
| | | | AEAWGRADCGFTSVSYQQGVLSATILYEILLG | |
| | | | KATLYAVLVSALVLMAMVKRKDF | |
| | | Full - with TRBC2 | MGFRLLCCVAFCLLGAGPVDSGVTQTPKHLIT | SEQ ID NO: 89 |
| | | constant domain | ATGQRVTLRCSPRSGDLSVYWYQQSLDQGLQF | |
| | | | LIQYYNGEERAKGNILERFSAQQFPDLHSELN | |
| | | | LSSLELGDSALYFCASSVARDRRNYGYTFGSG | |
| | | | TRLTVVEDLKNVFPPEVAVFEPSEAEISHTQK | |
| | | | ATLVCLATGFYPDHVELSWWVNGKEVHSGVST | |
| | | | DPQPLKEQPALNDSRYCLSSRLRVSATFWQNP | |
| | | | RNHFRCQVQFYGLSENDEWTQDRAKPVTQIVS | |
| | | | AEAWGRADCGFTSESYQQGVLSATILYEILLG | |
| | | | KATLYAVLVSALVLMAMVKRKDSRG | |

Donor: HD6

| Clonotype | Chain | Region | Amino acid sequence | SEQ ID NO |
|---|---|---|---|---|
| HD6-1 | Alpha (α) | CDR1α | NSMFDY | SEQ ID NO: 90 |
| | | CDR2α | ISSIKDK | SEQ ID NO: 91 |
| | | CDR3α | CAANNARLMF | SEQ ID NO: 92 |
| | | Variable domain | MAMLLGASVLILWLQPDWVNSQQKNDDQQVKQ | SEQ ID NO: 93 |
| | | | NSPSLSVQEGRISILNCDYTNSMFDYFLWYKK | |
| | | | YPAEGPTFLISISSIKDKNEDGRFTVFLNKSA | |
| | | | KHLSLHIVPSQPGDSAVYFCAANNARLMFGDG | |
| | | | TQLVVKPN | |
| | | Full - with TRAC | MAMLLGASVLILWLQPDWVNSQQKNDDQQVKQ | SEQ ID NO: 94 |
| | | constant domain | NSPSLSVQEGRISILNCDYTNSMFDYFLWYKK | |
| | | | YPAEGPTFLISISSIKDKNEDGRFTVFLNKSA | |
| | | | KHLSLHIVPSQPGDSAVYFCAANNARLMFGDG | |
| | | | TQLVVKPNIQNPDPAVYQLRDSKSSDKSVCLF | |
| | | | TDFDSQTNVSQSKDSDVYITDKTVLDMRSMDF | |
| | | | KSNSAVAWSNKSDFACANAFNNSIIPEDTFFP | |
| | | | SPESSCDVKLVEKSFETDTNLNFQNLSVIGFR | |
| | | | ILLLKVAGFNLLMTLRLWSS | |
| HD6-2 | Alpha (α) | CDR1α | NSMFDY | SEQ ID NO: 264 |
| | | CDR2α | ISSIKDK | SEQ ID NO: 265 |
| | | CDR3α | CAASATGNQFYF | SEQ ID NO: 266 |
| | | Variable domain | MAMLLGASVLILWLQPDWVNSQQKNDDQQVKQ | SEQ ID NO: 267 |
| | | | NSPSLSVQEGRISILNCDYTNSMFDYFLWYKK | |
| | | | YPAEGPTFLISISSIKDKNEDGRFTVFLNKSA | |
| | | | KHLSLHIVPSQPGDSAVYFCAASATGNQFYFG | |
| | | | TGTSLTVIPN | |
| | | Full - with TRAC | MAMLLGASVLILWLQPDWVNSQQKNDDQQVKQ | SEQ ID NO: 268 |
| | | constant domain | NSPSLSVQEGRISILNCDYTNSMFDYFLWYKK | |
| | | | YPAEGPTFLISISSIKDKNEDGRFTVFLNKSA | |
| | | | KHLSLHIVPSQPGDSAVYFCAASATGNQFYFG | |
| | | | TGTSLTVIPNIQNPDPAVYQLRDSKSSDKSVC | |
| | | | LFTDFDSQTNVSQSKDSDVYITDKTVLDMRSM | |
| | | | DFKSNSAVAWSNKSDFACANAFNNSIIPEDTF | |
| | | | FPSPESSCDVKLVEKSFETDTNLNFQNLSVIG | |
| | | | FRILLLKVAGFNLLMTLRLWSS | |
| HD6-1 | Beta (β) | CDR1β | SGHNS | SEQ ID NO: 95 |
| | | CDR2β | FNNNVP | SEQ ID NO: 96 |
| | | CDR3β | CASSDTRAREQFF | SEQ ID NO: 97 |
| | | Variable domain | MDSWTFCCVSLCILVAKHTDAGVIQSPRHEVT | SEQ ID NO: 98 |
| | | | EMGQEVTLRCKPISGHNSLFWYRQTMMRGLEL | |
| | | | LIYFNNNVPIDDSGMPEDRFSAKMPNASFSTL | |
| | | | KIQPSEPRDSAVYFCASSDTRAREQFFGPGTR | |
| | | | LTVLE | |
| | | Full - with TRBC1 | MDSWTFCCVSLCILVAKHTDAGVIQSPRHEVT | SEQ ID NO: 99 |
| | | constant domain | EMGQEVTLRCKPISGHNSLFWYRQTMMRGLEL | |
| | | | LIYFNNNVPIDDSGMPEDRFSAKMPNASFSTL | |
| | | | KIQPSEPRDSAVYFCASSDTRAREQFFGPGTR | |
| | | | LTVLEDLNKVFPPEVAVFEPSEAEISHTQKAT | |
| | | | LVCLATGFFPDHVELSWWVNGKEVHSGVSTDP | |
| | | | QPLKEQPALNDSRYCLSSRLRVSATFWQNPRN | |
| | | | HFRCQVQFYGLSENDEWTQDRAKPVTQIVSAE | |
| | | | AWGRADCGFTSVSYQQGVLSATILYEILLGKA | |
| | | | TLYAVLVSALVLMAMVKRKDF | |
| | | Full - with TRBC2 | MDSWTFCCVSLCILVAKHTDAGVIQSPRHEVT | SEQ ID NO: 100 |
| | | constant domain | EMGQEVTLRCKPISGHNSLFWYRQTMMRGLEL | |
| | | | LIYFNNNVPIDDSGMPEDRFSAKMPNASFSTL | |
| | | | KIQPSEPRDSAVYFCASSDTRAREQFFGPGTR | |
| | | | LTVLEDLKNVFPPEVAVFEPSEAEISHTQKAT | |
| | | | LVCLATGFYPDHVELSWWVNGKEVHSGVSTDP | |
| | | | QPLKEQPALNDSRYCLSSRLRVSATFWQNPRN | |
| | | | HFRCQVQFYGLSENDEWTQDRAKPVTQIVSAE | |
| | | | AWGRADCGFTSESYQQGVLSATILYEILLGKA | |
| | | | TLYAVLVSALVLMAMVKRKDSRG | |
| HD6-2 | Beta (β) | CDR1β | SGHRS | SEQ ID NO: 269 |
| | | CDR2β | YFSETQ | SEQ ID NO: 270 |
| | | CDR3β | CASSPGQHGELFF | SEQ ID NO: 271 |
| | | Variable domain | MGSRLLCWVLLCLLGAGPVKAGVTQTPRYLIK | SEQ ID NO: 272 |
| | | | TRGQQVTLSCSPISGHRSVSWYQQTPGQGLQF | |
| | | | LFEYFSETQRNKGNFPGRFSGRQFSNSRSEMN | |
| | | | VSTLELGDSALYLCASSPGQHGELFFGEGSRL | |
| | | | TVLE | |
| | | Full - with TRBC1 | MGSRLLCWVLLCLLGAGPVKAGVTQTPRYLIK | SEQ ID NO: 273 |
| | | constant domain | TRGQQVTLSCSPISGHRSVSWYQQTPGQGLQF | |
| | | | LFEYFSETQRNKGNFPGRFSGRQFSNSRSEMN | |
| | | | VSTLELGDSALYLCASSPGQHGELFFGEGSRL | |
| | | | TVLEDLNKVFPPEVAVFEPSEAEISHTQKATL | |
| | | | VCLATGFFPDHVELSWWVNGKEVHSGVSTDPQ | |
| | | | PLKEQPALNDSRYCLSSRLRVSATFWQNPRNH | |
| | | | FRCQVQFYGLSENDEWTQDRAKPVTQIVSAEA | |
| | | | WGRADCGFTSVSYQQGVLSATILYEILLGKAT | |
| | | | LYAVLVSALVLMAMVKRKDF | |
| | | Full - with TRBC2 | MGSRLLCWVLLCLLGAGPVKAGVTQTPRYLIK | SEQ ID NO: 274 |
| | | constant domain | TRGQQVTLSCSPISGHRSVSWYQQTPGQGLQF | |
| | | | LFEYFSETQRNKGNFPGRFSGRQFSNSRSEMN | |
| | | | VSTLELGDSALYLCASSPGQHGELFFGEGSRL | |
| | | | TVLEDLKNVFPPEVAVFEPSEAEISHTQKATL | |
| | | | VCLATGFYPDHVELSWWVNGKEVHSGVSTDPQ | |
| | | | PLKEQPALNDSRYCLSSRLRVSATFWQNPRNH | |
| | | | FRCQVQFYGLSENDEWTQDRAKPVTQIVSAEA | |
| | | | WGRADCGFTSESYQQGVLSATILYEILLGKAT | |
| | | | LYAVLVSALVLMAMVKRKDSRG | |

Donor: HD7

| Clonotype | Chain | Region | Sequence | SEQ ID NO |
|---|---|---|---|---|
| HD7-1 | Alpha (α) | CDR1α | DSSSTY | SEQ ID NO: 101 |
| | | CDR2α | IFSNMDM | SEQ ID NO: 102 |
| | | CDR3α | CAERLNTDKLIF | SEQ ID NO: 103 |
| | | Variable domain | MKTFAGFSFLFLWLQLDCMSRGEDVEQSLFLS | SEQ ID NO: 104 |
| | | | VREGDSSVINCTYTDSSSTYLYWYKQEPGAGL | |
| | | | QLLTYIFSNMDMKQDQRLTVLLNKKDKHLSLR | |
| | | | IADTQTGDSAIYFCAERLNTDKLIFGTGTRLQ | |
| | | | VFPN | |
| | | Full - with TRAC | MKTFAGFSFLFLWLQLDCMSRGEDVEQSLFLS | SEQ ID NO: 105 |
| | | constant domain | VREGDSSVINCTYTDSSSTYLYWYKQEPGAGL | |
| | | | QLLTYIFSNMDMKQDQRLTVLLNKKDKHLSLR | |
| | | | IADTQTGDSAIYFCAERLNTDKLIFGTGTRLQ | |
| | | | VFPNIQNPDPAVYQLRDSKSSDKSVCLFTDFD | |
| | | | SQTNVSQSKDSDVYITDKTVLDMRSMDFKSNS | |
| | | | AVAWSNKSDFACANAFNNSIIPEDTFFPSPES | |
| | | | SCDVKLVEKSFETDTNLNFQNLSVIGFRILLL | |
| | | | KVAGFNLLMTLRLWSS | |
| HD7-2 | Alpha (α) | CDR1α | DSVNN | SEQ ID NO: 106 |
| | | CDR2α | IPSGT | SEQ ID NO: 107 |
| | | CDR3α | CAVEATDSWGKLQF | SEQ ID NO: 108 |
| | | Variable domain | MKRILGALLGLLSAQVCCVRGIQVEQSPPDLI | SEQ ID NO: 109 |
| | | | LQEGANSTLRCNFSDSVNNLQWFHQNPWGQLI | |
| | | | NLFYIPSGTKQNGRLSATTVATERYSLLYISS | |
| | | | SQTTDSGVYFCAVEATDSWGKLQFGAGTQVVV | |
| | | | TPD | |
| | | Full - with TRAC | MKRILGALLGLLSAQVCCVRGIQVEQSPPDLI | SEQ ID NO: 110 |
| | | constant domain | LQEGANSTLRCNFSDSVNNLQWFHQNPWGQLI | |
| | | | NLFYIPSGTKQNGRLSATTVATERYSLLYISS | |
| | | | SQTTDSGVYFCAVEATDSWGKLQFGAGTQVVV | |
| | | | TPDIQNPDPAVYQLRDSKSSDKSVCLFTDFDS | |
| | | | QTNVSQSKDSDVYITDKTVLDMRSMDFKSNSA | |
| | | | VAWSNKSDFACANAFNNSIIPEDTFFPSPESS | |
| | | | CDVKLVEKSFETDTNLNFQNLSVIGFRILLLK | |
| | | | VAGFNLLMTLRLWSS | |
| HD7-3 | Alpha (α) | CDR1α | DSASNY | SEQ ID NO: 111 |
| | | CDR2α | IRSNVGE | SEQ ID NO: 112 |
| | | CDR3α | CAVRTSYDKVIF | SEQ ID NO: 113 |
| | | Variable domain | MTSIRAVFIFLWLQLDLVNGENVEQHPSTLSV | SEQ ID NO: 114 |
| | | | QEGDSAVIKCTYSDSASNYFPWYKQELGKRPQ | |
| | | | LIIDIRSNVGEKKDQRIAVTLNKTAKHFSLHI | |
| | | | TETQPEDSAVYFCAVRTSYDKVIFGPGTSLSV | |
| | | | IPN | |
| | | Full - with TRAC | MTSIRAVFIFLWLQLDLVNGENVEQHPSTLSV | SEQ ID NO: 160 |
| | | constant domain | QEGDSAVIKCTYSDSASNYFPWYKQELGKRPQ | |
| | | | LIIDIRSNVGEKKDQRIAVTLNKTAKHFSLHI | |
| | | | TETQPEDSAVYFCAVRTSYDKVIFGPGTSLSV | |
| | | | IPNIQNPDPAVYQLRDSKSSDKSVCLFTDFDS | |
| | | | QTNVSQSKDSDVYITDKTVLDMRSMDFKSNSA | |
| | | | VAWSNKSDFACANAFNNSIIPEDTFFPSPESS | |
| | | | CDVKLVEKSFETDTNLNFQNLSVIGFRILLLK | |
| | | | VAGFNLLMTLRLWSS | |
| HD7-4 | Alpha (α) | CDR1α | TSINN | SEQ ID NO: 275 |
| | | CDR2α | IRSNERE | SEQ ID NO: 276 |
| | | CDR3α | CATDGDSSYKLIF | SEQ ID NO: 277 |
| | | Variable domain | METLLGVSLVILWLQLARVNSQQGEEDPQALS | SEQ ID NO: 278 |
| | | | IQEGENATMNCSYKTSINNLQWYRQNSGRGLV | |
| | | | HLILIRSNEREKHSGRLRVTLDTSKKSSSLLI | |
| | | | TASRAADTASYFCATDGDSSYKLIFGSGTRLL | |
| | | | VRPD | |
| | | Full- with TRAC | METLLGVSLVILWLQLARVNSQQGEEDPQALS | SEQ ID NO: 279 |
| | | constant domain | IQEGENATMNCSYKTSINNLQWYRQNSGRGLV | |
| | | | HLILIRSNEREKHSGRLRVTLDTSKKSSSLLI | |
| | | | TASRAADTASYFCATDGDSSYKLIFGSGTRLL | |
| | | | VRPDIQNPDPAVYQLRDSKSSDKSVCLFTDFD | |
| | | | SQTNVSQSKDSDVYITDKTVLDMRSMDFKSNS | |
| | | | AVAWSNKSDFACANAFNNSIIPEDTFFPSPES | |
| | | | SCDVKLVEKSFETDTNLNFQNLSVIGFRILLL | |
| | | | KVAGFNLLMTLRLWSS | |
| HD7-1 | Beta (β) | CDR1β | DFQATT | SEQ ID NO: 161 |
| | | CDR2β | SNEGSKA | SEQ ID NO: 162 |
| | | CDR3β | CSARDSVSGNTIYF | SEQ ID NO: 163 |
| | | Variable domain | MLLLLLLLGPGISLLLPGSLAGSGLGAVVSQH | SEQ ID NO: 164 |
| | | | PSWVICKSGTSVKIECRSLDFQATTMFWYRQF | |
| | | | PKQSLMLMATSNEGSKATYEQGVEKDKFLINH | |
| | | | ASLTLSTLTVTSAHPEDSSFYICSARDSVSGN | |
| | | | TIYFGEGSWLTVVE | |
| | | Full - with TRBC1 | MLLLLLLLGPGISLLLPGSLAGSGLGAVVSQH | SEQ ID NO: 165 |
| | | constant domain | PSWVICKSGTSVKIECRSLDFQATTMFWYRQF | |
| | | | PKQSLMLMATSNEGSKATYEQGVEKDKFLINH | |
| | | | ASLTLSTLTVTSAHPEDSSFYICSARDSVSGN | |
| | | | TIYFGEGSWLTVVEDLNKVFPPEVAVFEPSEA | |
| | | | EISHTQKATLVCLATGFFPDHVELSWWVNGKE | |
| | | | VHSGVSTDPQPLKEQPALNDSRYCLSSRLRVS | |
| | | | ATFWQNPRNHFRCQVQFYGLSENDEWTQDRAK | |
| | | | PVTQIVSAEAWGRADCGFTSVSYQQGVLSATI | |
| | | | LYEILLGKATLYAVLVSALVLMAMVKRKDF | |
| | | Full - with TRBC2 | MLLLLLLLGPGISLLLPGSLAGSGLGAVVSQH | SEQ ID NO: 166 |
| | | constant domain | PSWVICKSGTSVKIECRSLDFQATTMFWYRQF | |
| | | | PKQSLMLMATSNEGSKATYEQGVEKDKFLINH | |
| | | | ASLTLSTLTVTSAHPEDSSFYICSARDSVSGN | |
| | | | TIYFGEGSWLTVVEDLKNVFPPEVAVFEPSEA | |
| | | | EISHTQKATLVCLATGFYPDHVELSWWVNGKE | |
| | | | VHSGVSTDPQPLKEQPALNDSRYCLSSRLRVS | |
| | | | ATFWQNPRNHFRCQVQFYGLSENDEWTQDRAK | |
| | | | PVTQIVSAEAWGRADCGFTSESYQQGVLSATI | |
| | | | LYEILLGKATLYAVLVSALVLMAMVKRKDSRG | |
| HD7-2 | Beta (β) | CDR1β | SQVTM | SEQ ID NO: 167 |
| | | CDR2β | ANQGSEA | SEQ ID NO: 168 |
| | | CDR3β | CSVGGSGSYNEQFF | SEQ ID NO: 169 |
| | | Variable domain | MLSLLLLLLGLGSVFSAVISQKPSRDICQRGT | SEQ ID NO: 170 |
| | | | SLTIQCQVDSQVTMMFWYRQQPGQSLTLTATA | |
| | | | NQGSEATYESGFVIDKFPISRPNLTFSTLTVS | |
| | | | NMSPEDSSIYLCSVGGSGSYNEQFFGPGTRLT | |
| | | | VLE | |
| | | Full - with TRBC1 | MLSLLLLLLGLGSVFSAVISQKPSRDICQRGT | SEQ ID NO: 171 |
| | | constant domain | SLTIQCQVDSQVTMMFWYRQQPGQSLTLTATA | |
| | | | NQGSEATYESGFVIDKFPISRPNLTFSTLTVS | |
| | | | NMSPEDSSIYLCSVGGSGSYNEQFFGPGTRLT | |
| | | | VLEDLNKVFPPEVAVFEPSEAEISHTQKATLV | |
| | | | CLATGFFPDHVELSWWVNGKEVHSGVSTDPQP | |
| | | | LKEQPALNDSRYCLSSRLRVSATFWQNPRNHF | |
| | | | RCQVQFYGLSENDEWTQDRAKPVTQIVSAEAW | |
| | | | GRADCGFTSVSYQQGVLSATILYEILLGKATL | |
| | | | YAVLVSALVLMAMVKRKDF | |
| | | Full - with TRBC2 | MLSLLLLLLGLGSVFSAVISQKPSRDICQRGT | SEQ ID NO: 172 |
| | | constant domain | SLTIQCQVDSQVTMMFWYRQQPGQSLTLTATA | |
| | | | NQGSEATYESGFVIDKFPISRPNLTFSTLTVS | |
| | | | NMSPEDSSIYLCSVGGSGSYNEQFFGPGTRLT | |
| | | | VLEDLKNVFPPEVAVFEPSEAEISHTQKATLV | |
| | | | CLATGFYPDHVELSWWVNGKEVHSGVSTDPQP | |
| | | | LKEQPALNDSRYCLSSRLRVSATFWQNPRNHF | |
| | | | RCQVQFYGLSENDEWTQDRAKPVTQIVSAEAW | |
| | | | GRADCGFTSESYQQGVLSATILYEILLGKATL | |
| | | | YAVLVSALVLMAMVKRKDSRG | |
| HD7-3 | Beta (β) | CDR1β | DFQATT | SEQ ID NO: 280 |
| | | CDR2β | SNEGSKA | SEQ ID NO: 281 |
| | | CDR3β | CSARDVLTGDYGYTF | SEQ ID NO: 282 |
| | | Variable domain | MLLLLLLLGPGISLLLPGSLAGSGLGAVVSQH | SEQ ID NO: 283 |
| | | | PSWVICKSGTSVKIECRSLDFQATTMFWYRQF | |
| | | | PKQSLMLMATSNEGSKATYEQGVEKDKFLINH | |
| | | | ASLTLSTLTVTSAHPEDSSFYICSARDVLTGD | |
| | | | YGYTFGSGTRLTVV | |
| | | Full - with TRBC1 | MLLLLLLLGPGISLLLPGSLAGSGLGAVVSQH | SEQ ID NO: 284 |
| | | constant domain | PSWVICKSGTSVKIECRSLDFQATTMFWYRQF | |
| | | | PKQSLMLMATSNEGSKATYEQGVEKDKFLINH | |
| | | | ASLTLSTLTVTSAHPEDSSFYICSARDVLTGD | |
| | | | YGYTFGSGTRLTVVEDLNKVFPPEVAVFEPSE | |
| | | | AEISHTQKATLVCLATGFFPDHVELSWWVNGK | |
| | | | EVHSGVSTDPQPLKEQPALNDSRYCLSSRLRV | |
| | | | SATFWQNPRNHFRCQVQFYGLSENDEWTQDRA | |
| | | | KPVTQIVSAEAWGRADCGFTSVSYQQGVLSAT | |
| | | | ILYEILLGKATLYAVLVSALVLMAMVKRKDF | |
| | | Full - with TRBC2 | MLLLLLLLGPGISLLLPGSLAGSGLGAVVSQH | SEQ ID NO: 285 |
| | | constant domain | PSWVICKSGTSVKIECRSLDFQATTMFWYRQF | |
| | | | PKQSLMLMATSNEGSKATYEQGVEKDKFLINH | |
| | | | ASLTLSTLTVTSAHPEDSSFYICSARDVLTGD | |
| | | | YGYTFGSGTRLTVVEDLKNVFPPEVAVFEPSE | |
| | | | AEISHTQKATLVCLATGFYPDHVELSWWVNGK | |
| | | | EVHSGVSTDPQPLKEQPALNDSRYCLSSRLRV | |
| | | | SATFWQNPRNHFRCQVQFYGLSENDEWTQDRA | |
| | | | KPVTQIVSAEAWGRADCGFTSESYQQGVLSAT | |
| | | | ILYEILLGKATLYAVLVSALVLMAMVKRKDSR | |
| | | | G | |
| HD7-4 | Beta (β) | CDR1β | SGHDY | SEQ ID NO: 286 |
| | | CDR2β | FNNNVP | SEQ ID NO: 287 |
| | | CDR3β | CASSLGLSISQETQYF | SEQ ID NO: 288 |
| | | Variable domain | MGSWTLCCVSLCILVAKHTDAGVIQSPRHEVT | SEQ ID NO: 289 |
| | | | EMGQEVTLRCKPISGHDYLFWYRQTMMRGLEL | |
| | | | LIYFNNNVPIDDSGMPEDRFSAKMPNASFSTL | |
| | | | KIQPSEPRDSAVYFCASSLGLSISQETQYFGP | |
| | | | GTRLLVLE | |
| | | Full - with TRBC1 | MGSWTLCCVSLCILVAKHTDAGVIQSPRHEVT | SEQ ID NO: 290 |
| | | constant domain | EMGQEVTLRCKPISGHDYLFWYRQTMMRGLEL | |
| | | | LIYFNNNVPIDDSGMPEDRFSAKMPNASFSTL | |
| | | | KIQPSEPRDSAVYFCASSLGLSISQETQYFGP | |
| | | | GTRLLVLEDLNKVFPPEVAVFEPSEAEISHTQ | |
| | | | KATLVCLATGFFPDHVELSWWVNGKEVHSGVS | |
| | | | TDPQPLKEQPALNDSRYCLSSRLRVSATFWQN | |
| | | | PRNHFRCQVQFYGLSENDEWTQDRAKPVTQIV | |
| | | | SAEAWGRADCGFTSVSYQQGVLSATILYEILL | |
| | | | GKATLYAVLVSALVLMAMVKRKDF | |
| | | Full - with TRBC2 | MGSWTLCCVSLCILVAKHTDAGVIQSPRHEVT | SEQ ID NO: 291 |
| | | constant domain | EMGQEVTLRCKPISGHDYLFWYRQTMMRGLEL | |
| | | | LIYFNNNVPIDDSGMPEDRFSAKMPNASFSTL | |
| | | | KIQPSEPRDSAVYFCASSLGLSISQETQYFGP | |
| | | | GTRLLVLEDLKNVFPPEVAVFEPSEAEISHTQ | |
| | | | KATLVCLATGFYPDHVELSWWVNGKEVHSGVS | |
| | | | TDPQPLKEQPALNDSRYCLSSRLRVSATFWQN | |
| | | | PRNHFRCQVQFYGLSENDEWTQDRAKPVTQIV | |
| | | | SAEAWGRADCGFTSESYQQGVLSATILYEILL | |
| | | | GKATLYAVLVSALVLMAMVKRKDSRG | |

Donor: HD8

| Chain | Region | Amino acid sequence | SEQ ID NO |
|---|---|---|---|
| Alpha (α) | CDR1α | VGISA | SEQ ID NO: 173 |
| | CDR2α | LSSGK | SEQ ID NO: 174 |
| | CDR3α | CAVTVGNKLVF | SEQ ID NO: 175 |
| | Variable | MVKIRQFLLAILWLQLSCVSAAKNEVEQSPQNLTAQEGEFIT | SEQ ID NO: 176 |
| | domain | INCSYSVGISALHWLQQHPGGGIVSLFMLSSGKKKHGRLIAT | |
| | | INIQEKHSSLHITASHPRDSAVYICAVTVGNKLVFGAGTILR | |
| | | VKSY | |
| | Full - with | MVKIRQFLLAILWLQLSCVSAAKNEVEQSPQNLTAQEGEFIT | SEQ ID NO: 177 |
| | TRAC constant | INCSYSVGISALHWLQQHPGGGIVSLFMLSSGKKKHGRLIAT | |
| | domain | INIQEKHSSLHITASHPRDSAVYICAVTVGNKLVFGAGTILR | |
| | | VKSYIQNPDPAVYQLRDSKSSDKSVCLFTDFDSQTNVSQSKD | |
| | | SDVYITDKTVLDMRSMDFKSNSAVAWSNKSDFACANAFNNSI | |
| | | IPEDTFFPSPESSCDVKLVEKSFETDTNLNFQNLSVIGFRIL | |
| | | LLKVAGFNLLMTLRLWSS | |
| Beta (β) | CDR1β | MNHNS | SEQ ID NO: 178 |
| | CDR2β | SASEGT | SEQ ID NO: 179 |
| | CDR3β | CASRGWREQFF | SEQ ID NO: 180 |
| | Variable | MSIGLLCCVAFSLLWASPVNAGVTQTPKFQVLKTGQSMTLQC | SEQ ID NO: 181 |
| | domain | AQDMNHNSMYWYRQDPGMGLRLIYYSASEGTTDKGEVPNGYN | |
| | | VSRLNKREFSLRLESAAPSQTSVYFCASRGWREQFFGPGTRL | |
| | | TVLE | |
| | Full - with | MSIGLLCCVAFSLLWASPVNAGVTQTPKFQVLKTGQSMTLQC | SEQ ID NO: 182 |
| | TRBC1 | AQDMNHNSMYWYRQDPGMGLRLIYYSASEGTTDKGEVPNGYN | |
| | constant | VSRLNKREFSLRLESAAPSQTSVYFCASRGWREQFFGPGTRL | |
| | domain | TVLEDLNKVFPPEVAVFEPSEAEISHTQKATLVCLATGFFPD | |
| | | HVELSWWVNGKEVHSGVSTDPQPLKEQPALNDSRYCLSSRLR | |
| | | VSATFWQNPRNHFRCQVQFYGLSENDEWTQDRAKPVTQIVSA | |
| | | EAWGRADCGFTSVSYQQGVLSATILYEILLGKATLYAVLVSA | |
| | | LVLMAMVKRKDF | |
| | Full - with | MSIGLLCCVAFSLLWASPVNAGVTQTPKFQVLKTGQSMTLQC | SEQ ID NO: 183 |
| | TRBC2 | AQDMNHNSMYWYRQDPGMGLRLIYYSASEGTTDKGEVPNGYN | |
| | constant | VSRLNKREFSLRLESAAPSQTSVYFCASRGWREQFFGPGTRL | |
| | domain | TVLEDLKNVFPPEVAVFEPSEAEISHTQKATLVCLATGFYPD | |
| | | HVELSWWVNGKEVHSGVSTDPQPLKEQPALNDSRYCLSSRLR | |
| | | VSATFWQNPRNHFRCQVQFYGLSENDEWTQDRAKPVTQIVSA | |
| | | EAWGRADCGFTSESYQQGVLSATILYEILLGKATLYAVLVSA | |
| | | LVLMAMVKRKDSRG | |

Donor: HD9

| Clonotype | Chain | Region | Sequence | SEQ ID NO |
|---|---|---|---|---|
| HD9-1 | Alpha (α) | CDR1α | VGISA | SEQ ID NO: 184 |
| | | CDR2α | LSSGK | SEQ ID NO: 185 |
| | | CDR3α | CAARSYNTDKLIF | SEQ ID NO: 186 |
| | | Variable domain | MVKIRQFLLAILWLQLSCVSAAKNEVEQSPQN | SEQ ID NO: 187 |
| | | | LTAQEGEFITINCSYSVGISALHWLQQHPGGG | |
| | | | IVSLFMLSSGKKKHGRLIATINIQEKHSSLHI | |
| | | | TASHPRDSAVYICAARSYNTDKLIFGTGTRLQ | |
| | | | VFPN | |
| | | Full - with TRAC | MVKIRQFLLAILWLQLSCVSAAKNEVEQSPQN | SEQ ID NO: 188 |
| | | constant domain | LTAQEGEFITINCSYSVGISALHWLQQHPGGG | |
| | | | IVSLFMLSSGKKKHGRLIATINIQEKHSSLHI | |
| | | | TASHPRDSAVYICAARSYNTDKLIFGTGTRLQ | |
| | | | VFPNIQNPDPAVYQLRDSKSSDKSVCLFTDFD | |
| | | | SQTNVSQSKDSDVYITDKTVLDMRSMDFKSNS | |
| | | | AVAWSNKSDFACANAFNNSIIPEDTFFPSPES | |
| | | | SCDVKLVEKSFETDTNLNFQNLSVIGFRILLL | |
| | | | KVAGFNLLMTLRLWSS | |
| HD9-2 | Alpha (α) | CDR1α | NSMFDY | SEQ ID NO: 189 |
| | | CDR2α | ISSIKDK | SEQ ID NO: 190 |
| | | CDR3α | CAASYNNARLMF | SEQ ID NO: 191 |
| | | Variable domain | MAMLLGASVLILWLQPDWVNSQQKNDDQQVKQ | SEQ ID NO: 192 |
| | | | NSPSLSVQEGRISILNCDYTNSMFDYFLWYKK | |
| | | | YPAEGPTFLISISSIKDKNEDGRFTVFLNKSA | |
| | | | KHLSLHIVPSQPGDSAVYFCAASYNNARLMFG | |
| | | | DGTQLVVKPN | |
| | | Full - with TRAC | MAMLLGASVLILWLQPDWVNSQQKNDDQQVKQ | SEQ ID NO: 193 |
| | | constant domain | NSPSLSVQEGRISILNCDYTNSMFDYFLWYKK | |
| | | | YPAEGPTFLISISSIKDKNEDGRFTVFLNKSA | |
| | | | KHLSLHIVPSQPGDSAVYFCAASYNNARLMFG | |
| | | | DGTQLVVKPNIQNPDPAVYQLRDSKSSDKSVC | |
| | | | LFTDFDSQTNVSQSKDSDVYITDKTVLDMRSM | |
| | | | DFKSNSAVAWSNKSDFACANAFNNSIIPEDTF | |
| | | | FPSPESSCDVKLVEKSFETDTNLNFQNLSVIG | |
| | | | FRILLLKVAGFNLLMTLRLWSS | |
| HD9-1 | Beta (β) | CDR1β | SGHTS | SEQ ID NO: 194 |
| | | CDR2β | YDEGEE | SEQ ID NO: 195 |
| | | CDR3β | CASSWGYQETQYF | SEQ ID NO: 196 |
| | | Variable domain | MGPRLLFWALLCLLGTGPVEAGVTQSPTHLIK | SEQ ID NO: 197 |
| | | | TRGQQATLRCSPISGHTSVYWYQQALGLGLQF | |
| | | | LLWYDEGEERNRGNFPPRFSGRQFPNYSSELN | |
| | | | VNALELEDSALYLCASSWGYQETQYFGPGTRL | |
| | | | LVLE | |
| | | Full - with TRBC1 | MGPRLLFWALLCLLGTGPVEAGVTQSPTHLIK | SEQ ID NO: 198 |
| | | constant domain | TRGQQATLRCSPISGHTSVYWYQQALGLGLQF | |
| | | | LLWYDEGEERNRGNFPPRFSGRQFPNYSSELN | |
| | | | VNALELEDSALYLCASSWGYQETQYFGPGTRL | |
| | | | LVLEDLNKVFPPEVAVFEPSEAEISHTQKATL | |
| | | | VCLATGFFPDHVELSWWVNGKEVHSGVSTDPQ | |
| | | | PLKEQPALNDSRYCLSSRLRVSATFWQNPRNH | |
| | | | FRCQVQFYGLSENDEWTQDRAKPVTQIVSAEA | |
| | | | WGRADCGFTSVSYQQGVLSATILYEILLGKAT | |
| | | | LYAVLVSALVLMAMVKRKDF | |
| | | Full - with TRBC2 | MGPRLLFWALLCLLGTGPVEAGVTQSPTHLIK | SEQ ID NO: 199 |
| | | constant domain | TRGQQATLRCSPISGHTSVYWYQQALGLGLQF | |
| | | | LLWYDEGEERNRGNFPPRFSGRQFPNYSSELN | |
| | | | VNALELEDSALYLCASSWGYQETQYFGPGTRL | |
| | | | LVLEDLKNVFPPEVAVFEPSEAEISHTQKATL | |
| | | | VCLATGFYPDHVELSWWVNGKEVHSGVSTDPQ | |
| | | | PLKEQPALNDSRYCLSSRLRVSATFWQNPRNH | |
| | | | FRCQVQFYGLSENDEWTQDRAKPVTQIVSAEA | |
| | | | WGRADCGFTSESYQQGVLSATILYEILLGKAT | |
| | | | LYAVLVSALVLMAMVKRKDSRG | |
| HD9-2 | Beta (β) | CDR1β | KGHSH | SEQ ID NO: 200 |
| | | CDR2β | LQKENI | SEQ ID NO: 201 |
| | | CDR3β | CASSPTGGEYYGYTF | SEQ ID NO: 202 |
| | | Variable domain | MDTRVLCCAVICLLGAGLSNAGVMQNPRHLVR | SEQ ID NO: 203 |
| | | | RRGQEARLRCSPMKGHSHVYWYRQLPEEGLKF | |
| | | | MVYLQKENIIDESGMPKERFSAEFPKEGPSIL | |
| | | | RIQQVVRGDSAAYFCASSPTGGEYYGYTFGSG | |
| | | | TRLTVVE | |
| | | Full - with TRBC1 | MDTRVLCCAVICLLGAGLSNAGVMQNPRHLVR | SEQ ID NO: 204 |
| | | constant domain | RRGQEARLRCSPMKGHSHVYWYRQLPEEGLKF | |
| | | | MVYLQKENIIDESGMPKERFSAEFPKEGPSIL | |
| | | | RIQQVVRGDSAAYFCASSPTGGEYYGYTFGSG | |
| | | | TRLTVVEDLNKVFPPEVAVFEPSEAEISHTQK | |
| | | | ATLVCLATGFFPDHVELSWWVNGKEVHSGVST | |
| | | | DPQPLKEQPALNDSRYCLSSRLRVSATFWQNP | |
| | | | RNHFRCQVQFYGLSENDEWTQDRAKPVTQIVS | |
| | | | AEAWGRADCGFTSVSYQQGVLSATILYEILLG | |
| | | | KATLYAVLVSALVLMAMVKRKDF | |
| | | Full - with TRBC2 | MDTRVLCCAVICLLGAGLSNAGVMQNPRHLVR | SEQ ID NO: 205 |
| | | constant domain | RRGQEARLRCSPMKGHSHVYWYRQLPEEGLKF | |
| | | | MVYLQKENIIDESGMPKERFSAEFPKEGPSIL | |
| | | | RIQQVVRGDSAAYFCASSPTGGEYYGYTFGSG | |
| | | | TRLTVVEDLKNVFPPEVAVFEPSEAEISHTQK | |
| | | | ATLVCLATGFYPDHVELSWWVNGKEVHSGVST | |
| | | | DPQPLKEQPALNDSRYCLSSRLRVSATFWQNP | |
| | | | RNHFRCQVQFYGLSENDEWTQDRAKPVTQIVS | |
| | | | AEAWGRADCGFTSESYQQGVLSATILYEILLG | |
| | | | KATLYAVLVSALVLMAMVKRKDSRG | |
| HD9-3 | Beta (β) | CDR1β | MNHEY | SEQ ID NO: 206 |
| | | CDR2β | SVGAGI | SEQ ID NO: 207 |
| | | CDR3β | CASSSYPLRTGRYNSYNSPLHF | SEQ ID NO: 208 |
| | | Variable domain | MSIGLLCCAALSLLWAGPVNAGVTQTPKFQVL | SEQ ID NO: 209 |
| | | | KTGQSMTLQCAQDMNHEYMSWYRQDPGMGLRL | |
| | | | IHYSVGAGITDQGEVPNGYNVSRSTTEDFPLR | |
| | | | LLSAAPSQTSVYFCASSSYPLRTGRYNSYNSP | |
| | | | LHFGNGTRLTVTE | |
| | | Full - with TRBC1 | MSIGLLCCAALSLLWAGPVNAGVTQTPKFQVL | SEQ ID NO: 210 |
| | | constant domain | KTGQSMTLQCAQDMNHEYMSWYRQDPGMGLRL | |
| | | | IHYSVGAGITDQGEVPNGYNVSRSTTEDFPLR | |
| | | | LLSAAPSQTSVYFCASSSYPLRTGRYNSYNSP | |
| | | | LHFGNGTRLTVTEDLNKVFPPEVAVFEPSEAE | |
| | | | ISHTQKATLVCLATGFFPDHVELSWWVNGKEV | |
| | | | HSGVSTDPQPLKEQPALNDSRYCLSSRLRVSA | |
| | | | TFWQNPRNHFRCQVQFYGLSENDEWTQDRAKP | |
| | | | VTQIVSAEAWGRADCGFTSVSYQQGVLSATIL | |
| | | | YEILLGKATLYAVLVSALVLMAMVKRKDF | |
| | | Full - with TRBC2 | MSIGLLCCAALSLLWAGPVNAGVTQTPKFQVL | SEQ ID NO: 211 |
| | | constant domain | KTGQSMTLQCAQDMNHEYMSWYRQDPGMGLRL | |
| | | | IHYSVGAGITDQGEVPNGYNVSRSTTEDFPLR | |
| | | | LLSAAPSQTSVYFCASSSYPLRTGRYNSYNSP | |
| | | | LHFGNGTRLTVTEDLKNVFPPEVAVFEPSEAE | |
| | | | ISHTQKATLVCLATGFYPDHVELSWWVNGKEV | |
| | | | HSGVSTDPQPLKEQPALNDSRYCLSSRLRVSA | |
| | | | TFWQNPRNHFRCQVQFYGLSENDEWTQDRAKP | |
| | | | VTQIVSAEAWGRADCGFTSESYQQGVLSATIL | |
| | | | YEILLGKATLYAVLVSALVLMAMVKRKDSRG | |

Donor: HD10

| Chain | Region | Amino acid sequence | SEQ ID NO |
|---|---|---|---|
| Alpha (α) | CDR1α | NSMFDY | SEQ ID NO: 212 |
| | CDR2α | ISSIKDK | SEQ ID NO: 213 |
| | CDR3α | CAASGGRDDKIIF | SEQ ID NO: 214 |
| | Variable | MAMLLGASVLILWLQPDWVNSQQKNDDQQVKQNSPSLSVQEG | SEQ ID NO: 215 |
| | domain | RISILNCDYTNSMFDYFLWYKKYPAEGPTFLISISSIKDKNE | |
| | | DGRFTVFLNKSAKHLSLHIVPSQPGDSAVYFCAASGGRDDKI | |
| | | IFGKGTRLHILPN | |
| | Full - with | MAMLLGASVLILWLQPDWVNSQQKNDDQQVKQNSPSLSVQEG | SEQ ID NO: 216 |
| | TRAC constant | RISILNCDYTNSMFDYFLWYKKYPAEGPTFLISISSIKDKNE | |
| | domain | DGRFTVFLNKSAKHLSLHIVPSQPGDSAVYFCAASGGRDDKI | |
| | | IFGKGTRLHILPNIQNPDPAVYQLRDSKSSDKSVCLFTDFDS | |
| | | QTNVSQSKDSDVYITDKTVLDMRSMDFKSNSAVAWSNKSDFA | |
| | | CANAFNNSIIPEDTFFPSPESSCDVKLVEKSFETDTNLNFQN | |
| | | LSVIGFRILLLKVAGFNLLMTLRLWSS | |
| Beta (β) | CDR1β | MNHEY | SEQ ID NO: 217 |
| | CDR2β | SVGAGI | SEQ ID NO: 218 |
| | CDR3β | CASSYSRTESTDTQYF | SEQ ID NO: 219 |
| | Variable | MSIGLLCCAALSLLWAGPVNAGVTQTPKFQVLKTGQSMTLQC | SEQ ID NO: 220 |
| | domain | AQDMNHEYMSWYRQDPGMGLRLIHYSVGAGITDQGEVPNGYN | |
| | | VSRSTTEDFPLRLLSAAPSQTSVYFCASSYSRTESTDTQYFG | |
| | | PGTRLTVLE | |
| | Full - with | MSIGLLCCAALSLLWAGPVNAGVTQTPKFQVLKTGQSMTLQC | SEQ ID NO: 221 |
| | TRBC1 | AQDMNHEYMSWYRQDPGMGLRLIHYSVGAGITDQGEVPNGYN | |
| | constant | VSRSTTEDFPLRLLSAAPSQTSVYFCASSYSRTESTDTQYFG | |
| | domain | PGTRLTVLEDLNKVFPPEVAVFEPSEAEISHTQKATLVCLAT | |
| | | GFFPDHVELSWWVNGKEVHSGVSTDPQPLKEQPALNDSRYCL | |
| | | SSRLRVSATFWQNPRNHFRCQVQFYGLSENDEWTQDRAKPVT | |
| | | QIVSAEAWGRADCGFTSVSYQQGVLSATILYEILLGKATLYA | |
| | | VLVSALVLMAMVKRKDF | |
| | Full - with | MSIGLLCCAALSLLWAGPVNAGVTQTPKFQVLKTGQSMTLQC | SEQ ID NO: 222 |
| | TRBC2 | AQDMNHEYMSWYRQDPGMGLRLIHYSVGAGITDQGEVPNGYN | |
| | constant | VSRSTTEDFPLRLLSAAPSQTSVYFCASSYSRTESTDTQYFG | |
| | domain | PGTRLTVLEDLKNVFPPEVAVFEPSEAEISHTQKATLVCLAT | |
| | | GFYPDHVELSWWVNGKEVHSGVSTDPQPLKEQPALNDSRYCL | |
| | | SSRLRVSATFWQNPRNHFRCQVQFYGLSENDEWTQDRAKPVT | |
| | | QIVSAEAWGRADCGFTSESYQQGVLSATILYEILLGKATLYA | |
| | | VLVSALVLMAMVKRKDSRG | |

Accordingly, the present invention provides isolated polypeptides comprising one or more amino acid sequences selected from the group consisting of SEQ ID NOs: 1-114 and 160-222, fragments, variants and homologues thereof.

In one aspect, the invention provides a TCR comprising a TCR alpha chain sequence selected from the group consisting of the HD1-HD10 alpha chain sequences of Table 1, and a TCR beta chain sequence independently selected from the group consisting of the HD1-HD10 beta chain sequences of Table 1.

Reduced Mispairing and Improved TCR Expression

The TCR of the invention may be expressed in a T-cell to alter the antigen specificity of the T-cell. TCR-transduced T-cells may express at least two TCR alpha and two TCR beta chains. While the endogenous TCR alpha/beta chains form a receptor that is self-tolerant, the introduced TCR alpha/beta chains form a receptor with defined specificity for the given target antigen.

However, TCR gene therapy requires sufficient expression of transferred TCRs. Transferred TCR might be diluted by the presence of the endogeneous TCR, resulting in suboptimal expression of the tumor specific TCR. Furthermore, mispairing between endogenous and introduced chains may occur to form novel receptors, which might display unexpected specificities for self-antigens and cause autoimmune damage when transferred into patients.

Hence, several strategies have been explored to reduce the risk of mispairing between endogenous and introduced TCR chains. Mutations of the TCR alpha/beta interface is one strategy currently employed to reduce unwanted mispairing. For example, the introduction of a cysteine in the constant domains of the alpha and beta chain allows the formation of a disulfide bond and enhances the pairing of the introduced chains while reducing mispairing with wild type chains.

Accordingly, the TCRs of the present invention may comprise one or more mutations at the α chain/β chain interface, such that when the α chain and the β chain are expressed in a T-cell, the frequency of mispairing between said chains and endogenous TCR α and β chains is reduced. In one embodiment, the one or more mutations introduce a cysteine residue into the constant region domain of each of the α chain and the β chain, wherein the cysteine residues are capable of forming a disulphide bond between the α chain and the β chain.

Another strategy to reduce mispairing relies on the introduction of polynucleotide sequences encoding siRNA, added to the genes encoding for the tumor specific TCR α and or β chains, and designed to limit the expression of the endogenous TCR genes (Okamoto S. Cancer research 69, 9003-9011, 2009).

Accordingly, the vector or polynucleotide encoding the TCRs of the present invention may comprise one or more siRNA or other agents aimed at limiting or abrogating the expression of the endogenous TCR genes.

It is also possible to combine artificial nucleases, such as zinc finger nucleases (ZFN), transcription activator-like effector nucleases (TALEN) or CRISPR/Cas systems, designed to target the constant regions of the endogenous genes, e.g. TCR genes (TRAC and, or TRBC), to obtain the permanent disruption of the endogenous TCR alpha and/or beta chain genes, thus allowing full expression of the tumor specific TCR and thus reducing or abrogating the risk of TCR mispairing. This process, known as the TCR gene editing proved superior to TCR gene transfer in vitro and in vivo (Provasi E., Genovese P., Nature Medicine May; 18(5):807-15; 2012).

Accordingly, the TCRs of the present invention may be used to edit T cell specificity by TCR disruption and genetic addition of the tumor specific TCR.

In addition, the genome editing technology allows targeted integration of a expression cassette, comprising a polynucleotide encoding a TCR of the present invention, and optionally one or more promoter regions and/or other expression control sequences, into an endogenous gene disrupted by the artificial nucleases (Lombardo A., Nature biotechnology 25, 1298-1306; 2007).

Accordingly, the TCRs of the present invention may be used to edit T-cell specificity by targeted integration of a polynucleotide encoding a TCR of the present invention at a genomic region. The integration may be targeted by an artificial nuclease.

Another strategy developed to increase expression of the transferred TCR and to reduce TCR mispairing consists in “murinization,” which replaces the human TCR α and TCR β constant regions (e.g. the TRAC, TRBC1 and TRBC2 regions) by their murine counterparts. Murizination of TCR constant regions is described in, for example, Sommermeyer and Uckert J Immunol; 2010 (184:6223-6231). Accordingly, the TCRs of the present invention may be murinized.

Isolated Polynucleotide

The present invention relates to an isolated polynucleotide encoding a TCR receptor of the invention or a part thereof, such as the α chain and/or the β chain, a variable domain or a portion thereof.

The isolated polynucleotide may be double or single stranded, and may be RNA or DNA.

It will be understood by a skilled person that numerous different polynucleotides can encode the same polypeptide as a result of the degeneracy of the genetic code. In addition, it is to be understood that the skilled person may, using routine techniques, make nucleotide substitutions, additions or deletions that do not affect the polypeptide sequence encoded by the polynucleotides of the invention to reflect the codon usage of any particular host organism in which the polypeptides of the invention are to be expressed.

The polynucleotides described herein may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with Mrna or Cdna obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

Examples of nucleotide sequences encoding TCRs according to the present invention are provided in the Table 2.

TABLE 2

| Donor | Chain | Nucleotide sequence | SEQ ID NO |
|---|---|---|---|
| HD1 | α (with | ATGGAGACTCTCCTGAAAGTGCTTTCAGGCACCTTGTTGTGGcAGTTGACCTGGG | SEQ ID NO: 132 |
| | TRAC) | TGAGAAGCCAACAACCAGTGCAGAGTCCTCAAGCCGTGATCCTCCGAGAAGGGGA | |
| | | AGATGCTGTCATCAACTGCAGTTCCTCCAAGGCTTTATATTCTGTACACTGGTAC | |
| | | AGGCAGAAGCATGGTGAAGCACCCGTCTTCCTGATGATATTACTGAAGGGTGGAG | |
| | | AACAGAAGGGTCATGAAAAAATATCTGCTTCATTTAATGAAAAAAAGCAGCAAAG | |
| | | CTCCCTGTACCTTACGGCCTCCCAGCTCAGTTACTCAGGAACCTACTTCTGCGGC | |
| | | ACAGCTTGGATTAACGACTACAAGCTCAGCTTTGGAGCCGGAACCACAGTAACTG | |
| | | TAAGAGCAAATATCCAGAACCCTGACCCTGCCGTGTACCAGCTGAGAGACTCTAA | |
| | | ATCCAGTGACAAGTCTGTCTGCCTATTCACCGATTTTGATTCTCAAACAAATGTG | |
| | | TCACAAAGTAAGGATTCTGATGTGTATATCACAGACAAAACTGTGCTAGACATGA | |
| | | GGTCTATGGACTTCAAGAGCAACAGTGCTGTGGCCTGGAGCAACAAATCTGACTT | |
| | | TGCATGTGCAAACGCCTTCAACAACAGCATTATTCCAGAAGACACCTTCTTCCCC | |
| | | AGCCCAGAAAGTTCCTGTGATGTCAAGCTGGTCGAGAAAAGCTTTGAAACAGATA | |
| | | CGAACCTAAACTTTCAAAACCTGTCAGTGATTGGGTTCCGAATCCTCCTCCTGAA | |
| | | AGTGGCCGGGTTTAATCTGCTCATGACGCTGCGGCTGTGGTCCAGC | |
| | β (with | ATGGGCTCCTGGACCCTCTGCTGTGTGTCCCTTTGCATCCTGGTAGCAAAGCACA | SEQ ID NO: 133 |
| | TRBC1) | CAGATGCTGGAGTTATCCAGTCACCCCGGCACGAGGTGACAGAGATGGGACAAGA | |
| | | AGTGACTCTGAGATGTAAACCAATTTCAGGACACGACTACCTTTTCTGGTACAGA | |
| | | CAGACCATGATGCGGGGACTGGAGTTGCTCATTTACTTTAACAACAACGTTCCGA | |
| | | TAGATGATTCAGGGATGCCCGAGGATCGATTCTCAGCTAAGATGCCTAATGCATC | |
| | | ATTCTCCACTCTGAAGATCCAGCCCTCAGAACCCAGGGACTCAGCTGTGTACTTC | |
| | | TGTGCCAGCAGAAAAACCGGGGGATATAGCAATCAGCCCCAGCATTTTGGTGATG | |
| | | GGACTCGACTCTCCATCCTAGAGGACCTGAACAAGGTGTTCCCACCCGAGGTCGC | |
| | | TGTGTTTGAGCCATCAGAAGCAGAGATCTCCCACACCCAAAAGGCCACACTGGTG | |
| | | TGCCTGGCCACAGGCTTCTTCCCCGACCACGTGGAGCTGAGCTGGTGGGTGAATG | |
| | | GGAAGGAGGTGCACAGTGGGGTCAGCACGGACCCGCAGCCCCTCAAGGAGCAGCC | |
| | | CGCCCTCAATGACTCCAGATACTGCCTGAGCAGCCGCCTGAGGGTCTCGGCCACC | |
| | | TTCTGGCAGAACCCCCGCAACCACTTCCGCTGTCAAGTCCAGTTCTACGGGCTCT | |
| | | CGGAGAATGACGAGTGGACCCAGGATAGGGCCAAACCCGTCACCCAGATCGTCAG | |
| | | CGCCGAGGCCTGGGGTAGAGCAGACTGTGGCTTTACCTCGGTGTCCTACCAGCAA | |
| | | GGGGTCCTGTCTGCCACCATCCTCTATGAGATCCTGCTAGGGAAGGCCACCCTGT | |
| | | ATGCTGTGCTGGTCAGCGCCCTTGTGTTGATGGCCATGGTCAAGAGAAAGGATTT | |
| | | C | |
| | β (with | ATGGGCTCCTGGACCCTCTGCTGTGTGTCCCTTTGCATCCTGGTAGCAAAGCACA | SEQ ID NO: 134 |
| | TRBC2) | CAGATGCTGGAGTTATCCAGTCACCCCGGCACGAGGTGACAGAGATGGGACAAGA | |
| | | AGTGACTCTGAGATGTAAACCAATTTCAGGACACGACTACCTTTTCTGGTACAGA | |
| | | CAGACCATGATGCGGGGACTGGAGTTGCTCATTTACTTTAACAACAACGTTCCGA | |
| | | TAGATGATTCAGGGATGCCCGAGGATCGATTCTCAGCTAAGATGCCTAATGCATC | |
| | | ATTCTCCACTCTGAAGATCCAGCCCTCAGAACCCAGGGACTCAGCTGTGTACTTC | |
| | | TGTGCCAGCAGAAAAACCGGGGGATATAGCAATCAGCCCCAGCATTTTGGTGATG | |
| | | GGACTCGACTCTCCATCCTAGAGGACCTGAAAAACGTGTTCCCACCCGAGGTCGC | |
| | | TGTGTTTGAGCCATCAGAAGCAGAGATCTCCCACACCCAAAAGGCCACACTGGTG | |
| | | TGCCTGGCCACAGGCTTCTACCCCGACCACGTGGAGCTGAGCTGGTGGGTGAATG | |
| | | GGAAGGAGGTGCACAGTGGGGTCAGCACAGACCCGCAGCCCCTCAAGGAGCAGCC | |
| | | CGCCCTCAATGACTCCAGATACTGCCTGAGCAGCCGCCTGAGGGTCTCGGCCACC | |
| | | TTCTGGCAGAACCCCCGCAACCACTTCCGCTGTCAAGTCCAGTTCTACGGGCTCT | |
| | | CGGAGAATGACGAGTGGACCCAGGATAGGGCCAAACCTGTCACCCAGATCGTCAG | |
| | | CGCCGAGGCCTGGGGTAGAGCAGACTGTGGCTTCACCTCCGAGTCTTACCAGCAA | |
| | | GGGGTCCTGTCTGCCACCATCCTCTATGAGATCTTGCTAGGGAAGGCCACCTTGT | |
| | | ATGCCGTGCTGGTCAGTGCCCTCGTGCTGATGGCCATGGTCAAGAGAAAGGATTC | |
| | | CAGAGGC | |
| HD2-1 | α (with | ATGCTCCTGCTGCTCGTCCCAGTGCTCGAGGTGATTTTTACCCTGGGAGGAACCA | SEQ ID NO: 135 |
| | TRAC) | GAGCCCAGTCGGTGACCCAGCTTGGCAGCCACGTCTCTGTCTCTGAAGGAGCCCT | |
| | | GGTTCTGCTGAGGTGCAACTACTCATCGTCTGTTCCACCATATCTCTTCTGGTAT | |
| | | GTGCAATACCCCAACCAAGGACTCCAGCTTCTCCTGAAGTACACATCAGCGGCCA | |
| | | CCCTGGTTAAAGGCATCAACGGTTTTGAGGCTGAATTTAAGAAGAGTGAAACCTC | |
| | | CTTCCACCTGACGAAACCCTCAGCCCATATGAGCGACGCGGCTGAGTACTTCTGT | |
| | | GCTGTGAGATTATCTGGTTCTGCAAGGCAACTGACCTTTGGATCTGGGACACAAT | |
| | | TGACTGTTTTACCTGATATCCAGAACCCTGACCCTGCCGTGTACCAGCTGAGAGA | |
| | | CTCTAAATCCAGTGACAAGTCTGTCTGCCTATTCACCGATTTTGATTCTCAAACA | |
| | | AATGTGTCACAAAGTAAGGATTCTGATGTGTATATCACAGACAAAACTGTGCTAG | |
| | | ACATGAGGTCTATGGACTTCAAGAGCAACAGTGCTGTGGCCTGGAGCAACAAATC | |
| | | TGACTTTGCATGTGCAAACGCCTTCAACAACAGCATTATTCCAGAAGACACCTTC | |
| | | TTCCCCAGCCCAGAAAGTTCCTGTGATGTCAAGCTGGTCGAGAAAAGCTTTGAAA | |
| | | CAGATACGAACCTAAACTTTCAAAACCTGTCAGTGATTGGGTTCCGAATCCTCCT | |
| | | CCTGAAAGTGGCCGGGTTTAATCTGCTCATGACGCTGCGGCTGTGGTCCAGC | |
| | β (with | ATGGGCACCAGGCTCCTCTGCTGGGCGGCCCTCTGTCTCCTGGGAGCAGAACTCA | SEQ ID NO: 136 |
| | TRBC1) | CAGAAGCTGGAGTTGCCCAGTCTCCCAGATATAAGATTATAGAGAAAAGGCAGAG | |
| | | TGTGGCTTTTIGGTGCAATCCTATATCTGGCCATGCTACCCTTTACTGGTACCAG | |
| | | CAGATCCTGGGACAGGGCCCAAAGCTTCTGATTCAGTTTCAGAATAACGGTGTAG | |
| | | TGGATGATTCACAGTTGCCTAAGGATCGATTTTCTGCAGAGAGGCTCAAAGGAGT | |
| | | AGACTCCACTCTCAAGATCCAGCCTGCAAAGCTTGAGGACTCGGCCGTGTATCTC | |
| | | TGTGCCAGCAGCTTACTGGGAGACGAGCAGTACTTCGGGCCGGGCACCAGGCTCA | |
| | | CGGTCACAGAGGACCTGAACAAGGTGTTCCCACCCGAGGTCGCTGTGTTTGAGCC | |
| | | ATCAGAAGCAGAGATCTCCCACACCCAAAAGGCCACACTGGTGTGCCTGGCCACA | |
| | | GGCTTCTTCCCCGACCACGTGGAGCTGAGCTGGTGGGTGAATGGGAAGGAGGTGC | |
| | | ACAGTGGGGTCAGCACGGACCCGCAGCCCCTCAAGGAGCAGCCCGCCCTCAATGA | |
| | | CTCCAGATACTGCCTGAGCAGCCGCCTGAGGGTCTCGGCCACCTTCTGGCAGAAC | |
| | | CCCCGCAACCACTTCCGCTGTCAAGTCCAGTTCTACGGGCTCTCGGAGAATGACG | |
| | | AGTGGACCCAGGATAGGGCCAAACCCGTCACCCAGATCGTCAGCGCCGAGGCCTG | |
| | | GGGTAGAGCAGACTGTGGCTTTACCTCGGTGTCCTACCAGCAAGGGGTCCTGTCT | |
| | | GCCACCATCCTCTATGAGATCCTGCTAGGGAAGGCCACCCTGTATGCTGTGCTGG | |
| | | TCAGCGCCCTIGTGTTGATGGCCATGGTCAAGAGAAAGGATTTC | |
| | β (with | ATGGGCACCAGGCTCCTCTGCTGGGCGGCCCTCTGTCTCCTGGGAGCAGAACTCA | SEQ ID NO: 137 |
| | TRBC2) | CAGAAGCTGGAGTTGCCCAGTCTCCCAGATATAAGATTATAGAGAAAAGGCAGAG | |
| | | TGTGGCTTTTIGGTGCAATCCTATATCTGGCCATGCTACCCTTTACTGGTACCAG | |
| | | CAGATCCTGGGACAGGGCCCAAAGCTTCTGATTCAGTTTCAGAATAACGGTGTAG | |
| | | TGGATGATTCACAGTTGCCTAAGGATCGATTTTCTGCAGAGAGGCTCAAAGGAGT | |
| | | AGACTCCACTCTCAAGATCCAGCCTGCAAAGCTTGAGGACTCGGCCGTGTATCTC | |
| | | TGTGCCAGCAGCTTACTGGGAGACGAGCAGTACTTCGGGCCGGGCACCAGGCTCA | |
| | | CGGTCACAGAGGACCTGAAAAACGTGTTCCCACCCGAGGTCGCTGTGTTTGAGCC | |
| | | ATCAGAAGCAGAGATCTCCCACACCCAAAAGGCCACACTGGTGTGCCTGGCCACA | |
| | | GGCTTCTACCCCGACCACGTGGAGCTGAGCTGGTGGGTGAATGGGAAGGAGGTGC | |
| | | ACAGTGGGGTCAGCACAGACCCGCAGCCCCTCAAGGAGCAGCCCGCCCTCAATGA | |
| | | CTCCAGATACTGCCTGAGCAGCCGCCTGAGGGTCTCGGCCACCTTCTGGCAGAAC | |
| | | CCCCGCAACCACTTCCGCTGTCAAGTCCAGTTCTACGGGCTCTCGGAGAATGACG | |
| | | AGTGGACCCAGGATAGGGCCAAACCTGTCACCCAGATCGTCAGCGCCGAGGCCTG | |
| | | GGGTAGAGCAGACTGTGGCTTCACCTCCGAGTCTTACCAGCAAGGGGTCCTGTCT | |
| | | GCCACCATCCTCTATGAGATCTTGCTAGGGAAGGCCACCTTGTATGCCGTGCTGG | |
| | | TCAGTGCCCTCGTGCTGATGGCCATGGTCAAGAGAAAGGATTCCAGAGGC | |
| HD2-2 | α (with | ATGGCATGCCCTGGCTTCCTGTGGGCACTIGTGATCTCCACCTGTCTTGAATTTA | SEQ ID NO: 138 |
| | TRAC) | GCATGGCTCAGACAGTCACTCAGTCTCAACCAGAGATGTCTGTGCAGGAGGCAGA | |
| | | GACCGTGACCCTGAGCTGCACATATGACACCAGTGAGAGTGATTATTATTTATTC | |
| | | TGGTACAAGCAGCCTCCCAGCAGGCAGATGATTCTCGTTATTCGCCAAGAAGCTT | |
| | | ATAAGCAACAGAATGCAACAGAGAATCGTTTCTCTGTGAACTTCCAGAAAGCAGC | |
| | | CAAATCCTTCAGTCTCAAGATCTCAGACTCACAGCTGGGGGATGCCGCGATGTAT | |
| | | TTCTGTGCTTATAGGAGTCTAAAATATGGAAACAAACTGGTCTTTGGCGCAGGAA | |
| | | CCATTCTGAGAGTCAAGTCCTATATCCAGAACCCTGACCCTGCCGTGTACCAGCT | |
| | | GAGAGACTCTAAATCCAGTGACAAGTCTGTCTGCCTATTCACCGATTTTGATTCT | |
| | | CAAACAAATGTGTCACAAAGTAAGGATTCTGATGTGTATATCACAGACAAAACTG | |
| | | TGCTAGACATGAGGTCTATGGACTTCAAGAGCAACAGTGCTGTGGCCTGGAGCAA | |
| | | CAAATCTGACTTTGCATGTGCAAACGCCTTCAACAACAGCATTATTCCAGAAGAC | |
| | | ACCTTCTTCCCCAGCCCAGAAAGTTCCTGTGATGTCAAGCTGGTCGAGAAAAGCT | |
| | | TTGAAACAGATACGAACCTAAACTTTCAAAACCTGTCAGTGATTGGGTTCCGAAT | |
| | | CCTCCTCCTGAAAGTGGCCGGGTTTAATCTGCTCATGACGCTGCGGCTGTGGTCC | |
| | | AGC | |
| | β (with | ATGGGCACCAGGCTCCTCTTCTGGGTGGCCTTCTGTCTCCTGGGGGCAGATCACA | SEQ ID NO: 139 |
| | TRBC1) | CAGGAGCTGGAGTCTCCCAGTCCCCCAGTAACAAGGTCACAGAGAAGGGAAAGGA | |
| | | TGTAGAGCTCAGGTGTGATCCAATTTCAGGTCATACTGCCCTTTACTGGTACCGA | |
| | | CAGAGCCTGGGGCAGGGCCTGGAGTTTTTAATTTACTTCCAAGGCAACAGTGCAC | |
| | | CAGACAAATCAGGGCTGCCCAGTGATCGCTTCTCTGCAGAGAGGACTGGGGGATC | |
| | | CGTCTCCACTCTGACGATCCAGCGCACACAGCAGGAGGACTCGGCCGTGTATCTC | |
| | | TGTGCCAGCAGCTTGGTAGCTTTACAGGGTGCGGGCGAGCAGTACTTCGGGCCGG | |
| | | GCACCAGGCTCACGGTCACAGAGGACCTGAACAAGGTGTTCCCACCCGAGGTCGC | |
| | | TGTGTTTGAGCCATCAGAAGCAGAGATCTCCCACACCCAAAAGGCCACACTGGTG | |
| | | TGCCTGGCCACAGGCTTCTTCCCCGACCACGTGGAGCTGAGCTGGTGGGTGAATG | |
| | | GGAAGGAGGTGCACAGTGGGGTCAGCACGGACCCGCAGCCCCTCAAGGAGCAGCC | |
| | | CGCCCTCAATGACTCCAGATACTGCCTGAGCAGCCGCCTGAGGGTCTCGGCCACC | |
| | | TTCTGGCAGAACCCCCGCAACCACTTCCGCTGTCAAGTCCAGTTCTACGGGCTCT | |
| | | CGGAGAATGACGAGTGGACCCAGGATAGGGCCAAACCCGTCACCCAGATCGTCAG | |
| | | CGCCGAGGCCTGGGGTAGAGCAGACTGTGGCTTTACCTCGGTGTCCTACCAGCAA | |
| | | GGGGTCCTGTCTGCCACCATCCTCTATGAGATCCTGCTAGGGAAGGCCACCCTGT | |
| | | ATGCTGTGCTGGTCAGCGCCCTIGTGTTGATGGCCATGGTCAAGAGAAAGGATTT | |
| | | C | |
| | β (with | ATGGGCACCAGGCTCCTCTTCTGGGTGGCCTTCTGTCTCCTGGGGGCAGATCACA | SEQ ID NO: 140 |
| | TRBC2) | cAGGAGCTGGAGTCTCCCAGTCCCCCAGTAACAAGGTCACAGAGAAGGGAAAGGA | |
| | | TGTAGAGCTCAGGTGTGATCCAATTTCAGGTCATACTGCCCTTTACTGGTACCGA | |
| | | CAGAGCCTGGGGCAGGGCCTGGAGTTTTTAATTTACTTCCAAGGCAACAGTGCAC | |
| | | CAGACAAATCAGGGCTGCCCAGTGATCGCTTCTCTGCAGAGAGGACTGGGGGATC | |
| | | CGTCTCCACTCTGACGATCCAGCGCACACAGCAGGAGGACTCGGCCGTGTATCTC | |
| | | TGTGCCAGCAGCTTGGTAGCTTTACAGGGTGCGGGCGAGCAGTACTTCGGGCCGG | |
| | | GCACCAGGCTCACGGTCACAGAGGACCTGAAAAACGTGTTCCCACCCGAGGTCGC | |
| | | TGTGTTTGAGCCATCAGAAGCAGAGATCTCCCACACCCAAAAGGCCACACTGGTG | |
| | | TGCCTGGCCACAGGCTTCTACCCCGACCACGTGGAGCTGAGCTGGTGGGTGAATG | |
| | | GGAAGGAGGTGCACAGTGGGGTCAGCACAGACCCGCAGCCCCTCAAGGAGCAGCC | |
| | | CGCCCTCAATGACTCCAGATACTGCCTGAGCAGCCGCCTGAGGGTCTCGGCCACC | |
| | | TTCTGGCAGAACCCCCGCAACCACTTCCGCTGTCAAGTCCAGTTCTACGGGCTCT | |
| | | CGGAGAATGACGAGTGGACCCAGGATAGGGCCAAACCTGTCACCCAGATCGTCAG | |
| | | CGCCGAGGCCTGGGGTAGAGCAGACTGTGGCTTCACCTCCGAGTCTTACCAGCAA | |
| | | GGGGTCCTGTCTGCCACCATCCTCTATGAGATCTTGCTAGGGAAGGCCACCTTGT | |
| | | ATGCCGTGCTGGTCAGTGCCCTCGTGCTGATGGCCATGGTCAAGAGAAAGGATTC | |
| | | CAGAGGC | |
| HD3 | α (with | ATGATATCCTTGAGAGTTTTACTGGTGATCCTGTGGCTTCAGTTAAGCTGGGTTT | SEQ ID NO: 141 |
| | TRAC) | GGAGCCAACGGAAGGAGGTGGAGCAGGATCCTGGACCCTTCAATGTTCCAGAGGG | |
| | | AGCCACTGTCGCTTTCAACTGTACTTACAGCAACAGTGCTTCTCAGTCTTTCTTC | |
| | | TGGTACAGACAGGATTGCAGGAAAGAACCTAAGTTGCTGATGTCCGTATACTCCA | |
| | | GTGGTAATGAAGATGGAAGGTTTACAGCACAGCTCAATAGAGCCAGCCAGTATAT | |
| | | TTCCCTGCTCATCAGAGACTCCAAGCTCAGTGATTCAGCCACCTACCTCTGTGTG | |
| | | GTGAACCTCCTGTCTAACCAGGGAGGAAAGCTTATCTTCGGACAGGGAACGGAGT | |
| | | TATCTGTGAAACCCAATATCCAGAACCCTGACCCTGCCGTGTACCAGCTGAGAGA | |
| | | CTCTAAATCCAGTGACAAGTCTGTCTGCCTATTCACCGATTTTGATTCTCAAACA | |
| | | AATGTGTCACAAAGTAAGGATTCTGATGTGTATATCACAGACAAAACTGTGCTAG | |
| | | ACATGAGGTCTATGGACTTCAAGAGCAACAGTGCTGTGGCCTGGAGCAACAAATC | |
| | | TGACTTTGCATGTGCAAACGCCTTCAACAACAGCATTATTCCAGAAGACACCTTC | |
| | | TTCCCCAGCCCAGAAAGTTCCTGTGATGTCAAGCTGGTCGAGAAAAGCTTTGAAA | |
| | | CAGATACGAACCTAAACTTTCAAAACCTGTCAGTGATTGGGTTCCGAATCCTCCT | |
| | | CCTGAAAGTGGCCGGGTTTAATCTGCTCATGACGCTGCGGCTGTGGTCCAGC | |
| | β (with | ATGGGCTGCAGGCTGCTCTGCTGTGCGGTTCTCTGTCTCCTGGGAGCGGGTGAGT | SEQ ID NO: 142 |
| | TRBC1) | TGGTCCCCATGGAAACGGGAGTTACGCAGACACCAAGACACCTGGTCATGGGAAT | |
| | | GACAAATAAGAAGTCTTTGAAATGTGAACAACATCTGGGTCATAACGCTATGTAT | |
| | | TGGTACAAGCAAAGTGCTAAGAAGCCACTGGAGCTCATGTTTGTCTACAGTCTIG | |
| | | AAGAACGGGTTGAAAACAACAGTGTGCCAAGTCGCTTCTCACCTGAATGCCCCAA | |
| | | CAGCTCTCACTTATTCCTTCACCTACACACCCTGCAGCCAGAAGACTCGGCCCTG | |
| | | TATCTCTGCGCCAGCAGCCAAGATTACTTGGTTTCTAATGAAAAACTGTTTTTTG | |
| | | GCAGTGGAACCCAGCTCTCTGTCTIGGAGGACCTGAACAAGGTGTTCCCACCCGA | |
| | | GGTCGCTGTGTTTGAGCCATCAGAAGCAGAGATCTCCCACACCCAAAAGGCCACA | |
| | | CTGGTGTGCCTGGCCACAGGCTTCTTCCCCGACCACGTGGAGCTGAGCTGGTGGG | |
| | | TGAATGGGAAGGAGGTGCACAGTGGGGTCAGCACGGACCCGCAGCCCCTCAAGGA | |
| | | GCAGCCCGCCCTCAATGACTCCAGATACTGCCTGAGCAGCCGCCTGAGGGTCTCG | |
| | | GCCACCTTCTGGCAGAACCCCCGCAACCACTTCCGCTGTCAAGTCCAGTTCTACG | |
| | | GGCTCTCGGAGAATGACGAGTGGACCCAGGATAGGGCCAAACCCGTCACCCAGAT | |
| | | CGTCAGCGCCGAGGCCTGGGGTAGAGCAGACTGTGGCTTTACCTCGGTGTCCTAC | |
| | | CAGCAAGGGGTCCTGTCTGCCACCATCCTCTATGAGATCCTGCTAGGGAAGGCCA | |
| | | CCCTGTATGCTGTGCTGGTCAGCGCCCTTGTGTTGATGGCCATGGTCAAGAGAAA | |
| | | GGATTTC | |
| | β (with | ATGGGCTGCAGGCTGCTCTGCTGTGCGGTTCTCTGTCTCCTGGGAGCGGGTGAGT | SEQ ID NO: 143 |
| | TRBC2) | TGGTCCCCATGGAAACGGGAGTTACGCAGACACCAAGACACCTGGTCATGGGAAT | |
| | | GACAAATAAGAAGTCTTTGAAATGTGAACAACATCTGGGTCATAACGCTATGTAT | |
| | | TGGTACAAGCAAAGTGCTAAGAAGCCACTGGAGCTCATGTTTGTCTACAGTCTTG | |
| | | AAGAACGGGTTGAAAACAACAGTGTGCCAAGTCGCTTCTCACCTGAATGCCCCAA | |
| | | CAGCTCTCACTTATTCCTTCACCTACACACCCTGCAGCCAGAAGACTCGGCCCTG | |
| | | TATCTCTGCGCCAGCAGCCAAGATTACTTGGTTTCTAATGAAAAACTGTTTTTTG | |
| | | GCAGTGGAACCCAGCTCTCTGTCTTGGAGGACCTGAAAAACGTGTTCCCACCCGA | |
| | | GGTCGCTGTGTTTGAGCCATCAGAAGCAGAGATCTCCCACACCCAAAAGGCCACA | |
| | | CTGGTGTGCCTGGCCACAGGCTTCTACCCCGACCACGTGGAGCTGAGCTGGTGGG | |
| | | TGAATGGGAAGGAGGTGCACAGTGGGGTCAGCACAGACCCGCAGCCCCTCAAGGA | |
| | | GCAGCCCGCCCTCAATGACTCCAGATACTGCCTGAGCAGCCGCCTGAGGGTCTCG | |
| | | GCCACCTTCTGGCAGAACCCCCGCAACCACTTCCGCTGTCAAGTCCAGTTCTACG | |
| | | GGCTCTCGGAGAATGACGAGTGGACCCAGGATAGGGCCAAACCTGTCACCCAGAT | |
| | | CGTCAGCGCCGAGGCCTGGGGTAGAGCAGACTGTGGCTTCACCTCCGAGTCTTAC | |
| | | CAGCAAGGGGTCCTGTCTGCCACCATCCTCTATGAGATCTTGCTAGGGAAGGCCA | |
| | | CCTTGTATGCCGTGCTGGTCAGTGCCCTCGTGCTGATGGCCATGGTCAAGAGAAA | |
| | | GGATTCCAGAGGC | |
| HD4-1 | α1 (with | ATGGAAACTCTCCTGGGAGTGTCTTTGGTGATTCTATGGCTTCAACTGGCTAGGG | SEQ ID NO: 144 |
| | TRAC) | TGAACAGTCAACAGGGAGAAGAGGATCCTCAGGCCTTGAGCATCCAGGAGGGTGA | |
| | | AAATGCCACCATGAACTGCAGTTACAAAACTAGTATAAACAATTTACAGTGGTAT | |
| | | AGACAAAATTCAGGTAGAGGCCTTGTCCACCTAATTTTAATACGTTCAAATGAAA | |
| | | GAGAGAAACACAGTGGAAGATTAAGAGTCACGCTTGACACTTCCAAGAAAAGCAG | |
| | | TTCCTTGTTGATCACGGCTTCCCGGGCAGCAGACACTGCTTCTTACTTCTGTGCT | |
| | | ACGGACGCGTATTCAGGAAACACACCTCTTGTCTTTGGAAAGGGCACAAGACTTT | |
| | | CTGTGATTGCAAATATCCAGAACCCTGACCCTGCCGTGTACCAGCTGAGAGACTC | |
| | | TAAATCCAGTGACAAGTCTGTCTGCCTATTCACCGATTTTGATTCTCAAACAAAT | |
| | | GTGTCACAAAGTAAGGATTCTGATGTGTATATCACAGACAAAACTGTGCTAGACA | |
| | | TGAGGTCTATGGACTTCAAGAGCAACAGTGCTGTGGCCTGGAGCAACAAATCTGA | |
| | | CTTTGCATGTGCAAACGCCTTCAACAACAGCATTATTCCAGAAGACACCTTCTTC | |
| | | CCCAGCCCAGAAAGTTCCTGTGATGTCAAGCTGGTCGAGAAAAGCTTTGAAACAG | |
| | | ATACGAACCTAAACTTTCAAAACCTGTCAGTGATTGGGTTCCGAATCCTCCTCCT | |
| | | GAAAGTGGCCGGGTTTAATCTGCTCATGACGCTGCGGCTGTGGTCCAGC | |
| | β1 (with | ATGAGCATCGGGCTCCTGTGCTGTGTGGCCTTTTCTCTCCTGTGGGCAAGTCCAG | SEQ ID NO: 145 |
| | TRBC1) | TGAATGCTGGTGTCACTCAGACCCCAAAATTCCAGGTCCTGAAGACAGGACAGAG | |
| | | CATGACACTGCAGTGTGCCCAGGATATGAACCATAACTCCATGTACTGGTATCGA | |
| | | CAAGACCCAGGCATGGGACTGAGGCTGATTTATTACTCAGCTTCTGAGGGTACCA | |
| | | CTGACAAAGGAGAAGTCCCCAATGGCTACAATGTCTCCAGATTAAACAAACGGGA | |
| | | GTTCTCGCTCAGGCTGGAGTCGGCTGCTCCCTCCCAGACATCTGTGTACTTCTGT | |
| | | GCCAGCAGGGCAGCAGGGTTGGACACTGAAGCTTTCTTTGGACAAGGCACCAGAC | |
| | | TCACAGTIGTAGAGGACCTGAACAAGGTGTTCCCACCCGAGGTCGCTGTGTTTGA | |
| | | GCCATCAGAAGCAGAGATCTCCCACACCCAAAAGGCCACACTGGTGTGCCTGGCC | |
| | | ACAGGCTTCTTCCCCGACCACGTGGAGCTGAGCTGGTGGGTGAATGGGAAGGAGG | |
| | | TGCACAGTGGGGTCAGCACGGACCCGCAGCCCCTCAAGGAGCAGCCCGCCCTCAA | |
| | | TGACTCCAGATACTGCCTGAGCAGCCGCCTGAGGGTCTCGGCCACCTTCTGGCAG | |
| | | AACCCCCGCAACCACTTCCGCTGTCAAGTCCAGTTCTACGGGCTCTCGGAGAATG | |
| | | ACGAGTGGACCCAGGATAGGGCCAAACCCGTCACCCAGATCGTCAGCGCCGAGGC | |
| | | CTGGGGTAGAGCAGACTGTGGCTTTACCTCGGTGTCCTACCAGCAAGGGGTCCTG | |
| | | TCTGCCACCATCCTCTATGAGATCCTGCTAGGGAAGGCCACCCTGTATGCTGTGC | |
| | | TGGTCAGCGCCCTIGTGTTGATGGCCATGGTCAAGAGAAAGGATTTC | |
| | β1 (with | ATGAGCATCGGGCTCCTGTGCTGTGTGGCCTTTTCTCTCCTGTGGGCAAGTCCAG | SEQ ID NO: 146 |
| | TRBC2) | TGAATGCTGGTGTCACTCAGACCCCAAAATTCCAGGTCCTGAAGACAGGACAGAG | |
| | | CATGACACTGCAGTGTGCCCAGGATATGAACCATAACTCCATGTACTGGTATCGA | |
| | | CAAGACCCAGGCATGGGACTGAGGCTGATTTATTACTCAGCTTCTGAGGGTACCA | |
| | | CTGACAAAGGAGAAGTCCCCAATGGCTACAATGTCTCCAGATTAAACAAACGGGA | |
| | | GTTCTCGCTCAGGCTGGAGTCGGCTGCTCCCTCCCAGACATCTGTGTACTTCTGT | |
| | | GCCAGCAGGGCAGCAGGGTTGGACACTGAAGCTTTCTTTGGACAAGGCACCAGAC | |
| | | TCACAGTIGTAGAGGACCTGAAAAACGTGTTCCCACCCGAGGTCGCTGTGTTTGA | |
| | | GCCATCAGAAGCAGAGATCTCCCACACCCAAAAGGCCACACTGGTGTGCCTGGCC | |
| | | ACAGGCTTCTACCCCGACCACGTGGAGCTGAGCTGGTGGGTGAATGGGAAGGAGG | |
| | | TGCACAGTGGGGTCAGCACAGACCCGCAGCCCCTCAAGGAGCAGCCCGCCCTCAA | |
| | | TGACTCCAGATACTGCCTGAGCAGCCGCCTGAGGGTCTCGGCCACCTTCTGGCAG | |
| | | AACCCCCGCAACCACTTCCGCTGTCAAGTCCAGTTCTACGGGCTCTCGGAGAATG | |
| | | ACGAGTGGACCCAGGATAGGGCCAAACCTGTCACCCAGATCGTCAGCGCCGAGGC | |
| | | CTGGGGTAGAGCAGACTGTGGCTTCACCTCCGAGTCTTACCAGCAAGGGGTCCTG | |
| | | TCTGCCACCATCCTCTATGAGATCTTGCTAGGGAAGGCCACCTTGTATGCCGTGC | |
| | | TGGTCAGTGCCCTCGTGCTGATGGCCATGGTCAAGAGAAAGGATTCCAGAGGC | |
| HD4-2 | α2 (with | ATGGAGACCCTCTTGGGCCTGCTTATCCTTTGGCTGCAGCTGCAATGGGTGAGCA | SEQ ID NO: 147 |
| | TRAC) | GCAAACAGGAGGTGACGCAGATTCCTGCAGCTCTGAGTGTCCCAGAAGGAGAAAA | |
| | | CTTGGTTCTCAACTGCAGTTTCACTGATAGCGCTATTTACAACCTCCAGTGGTTT | |
| | | AGGCAGGACCCTGGGAAAGGTCTCACATCTCTGTTGCTTATTCAGTCAAGTCAGA | |
| | | GAGAGCAAACAAGTGGAAGACTTAATGCCTCGCTGGATAAATCATCAGGACGTAG | |
| | | TACTTTATACATTGCAGCTTCTCAGCCTGGTGACTCAGCCACCTACCTCTGTGCT | |
| | | GTCCGGGCAGAGATTTATAACCAGGGAGGAAAGCTTATCTTCGGACAGGGAACGG | |
| | | AGTTATCTGTGAAACCCAATATCCAGAACCCTGACCCTGCCGTGTACCAGCTGAG | |
| | | AGACTCTAAATCCAGTGACAAGTCTGTCTGCCTATTCACCGATTTTGATTCTCAA | |
| | | ACAAATGTGTCACAAAGTAAGGATTCTGATGTGTATATCACAGACAAAACTGTGC | |
| | | TAGACATGAGGTCTATGGACTTCAAGAGCAACAGTGCTGTGGCCTGGAGCAACAA | |
| | | ATCTGACTTTGCATGTGCAAACGCCTTCAACAACAGCATTATTCCAGAAGACACC | |
| | | TTCTTCCCCAGCCCAGAAAGTTCCTGTGATGTCAAGCTGGTCGAGAAAAGCTTTG | |
| | | AAACAGATACGAACCTAAACTTTCAAAACCTGTCAGTGATTGGGTTCCGAATCCT | |
| | | CCTCCTGAAAGTGGCCGGGTTTAATCTGCTCATGACGCTGCGGCTGTGGTCCAGC | |
| | β2 (with | ATGAGCATCAGCCTCCTGTGCTGTGCAGCCTTTCCTCTCCTGTGGGCAGGTCCAG | SEQ ID NO: 148 |
| | TRBC1) | TGAATGCTGGTGTCACTCAGACCCCAAAATTCCGCATCCTGAAGATAGGACAGAG | |
| | | CATGACACTGCAGTGTACCCAGGATATGAACCATAACTACATGTACTGGTATCGA | |
| | | CAAGACCCAGGCATGGGGCTGAAGCTGATTTATTATTCAGTTGGTGCTGGTATCA | |
| | | CTGATAAAGGAGAAGTCCCGAATGGCTACAACGTCTCCAGATCAACCACAGAGGA | |
| | | TTTCCCGCTCAGGCTGGAGTTGGCTGCTCCCTCCCAGACATCTGTGTACTTCTGT | |
| | | GCCAGTACCCAAACTCCCTACGAGCAGTACTTCGGGCCGGGCACCAGGCTCACGG | |
| | | TCACAGAGGACCTGAACAAGGTGTTCCCACCCGAGGTCGCTGTGTTTGAGCCATC | |
| | | AGAAGCAGAGATCTCCCACACCCAAAAGGCCACACTGGTGTGCCTGGCCACAGGC | |
| | | TTCTTCCCCGACCACGTGGAGCTGAGCTGGTGGGTGAATGGGAAGGAGGTGCACA | |
| | | GTGGGGTCAGCACGGACCCGCAGCCCCTCAAGGAGCAGCCCGCCCTCAATGACTC | |
| | | CAGATACTGCCTGAGCAGCCGCCTGAGGGTCTCGGCCACCTTCTGGCAGAACCCC | |
| | | CGCAACCACTTCCGCTGTCAAGTCCAGTTCTACGGGCTCTCGGAGAATGACGAGT | |
| | | GGACCCAGGATAGGGCCAAACCCGTCACCCAGATCGTCAGCGCCGAGGCCTGGGG | |
| | | TAGAGCAGACTGTGGCTTTACCTCGGTGTCCTACCAGCAAGGGGTCCTGTCTGCC | |
| | | ACCATCCTCTATGAGATCCTGCTAGGGAAGGCCACCCTGTATGCTGTGCTGGTCA | |
| | | GCGCCCTIGTGTTGATGGCCATGGTCAAGAGAAAGGATTTC | |
| | β2 (with | ATGAGCATCAGCCTCCTGTGCTGTGCAGCCTTTCCTCTCCTGTGGGCAGGTCCAG | SEQ ID NO: 149 |
| | TRBC2) | TGAATGCTGGTGTCACTCAGACCCCAAAATTCCGCATCCTGAAGATAGGACAGAG | |
| | | CATGACACTGCAGTGTACCCAGGATATGAACCATAACTACATGTACTGGTATCGA | |
| | | CAAGACCCAGGCATGGGGCTGAAGCTGATTTATTATTCAGTTGGTGCTGGTATCA | |
| | | CTGATAAAGGAGAAGTCCCGAATGGCTACAACGTCTCCAGATCAACCACAGAGGA | |
| | | TTTCCCGCTCAGGCTGGAGTTGGCTGCTCCCTCCCAGACATCTGTGTACTTCTGT | |
| | | GCCAGTACCCAAACTCCCTACGAGCAGTACTTCGGGCCGGGCACCAGGCTCACGG | |
| | | TCACAGAGGACCTGAAAAACGTGTTCCCACCCGAGGTCGCTGTGTTTGAGCCATC | |
| | | AGAAGCAGAGATCTCCCACACCCAAAAGGCCACACTGGTGTGCCTGGCCACAGGC | |
| | | TTCTACCCCGACCACGTGGAGCTGAGCTGGTGGGTGAATGGGAAGGAGGTGCACA | |
| | | GTGGGGTCAGCACAGACCCGCAGCCCCTCAAGGAGCAGCCCGCCCTCAATGACTC | |
| | | CAGATACTGCCTGAGCAGCCGCCTGAGGGTCTCGGCCACCTTCTGGCAGAACCCC | |
| | | CGCAACCACTTCCGCTGTCAAGTCCAGTTCTACGGGCTCTCGGAGAATGACGAGT | |
| | | GGACCCAGGATAGGGCCAAACCTGTCACCCAGATCGTCAGCGCCGAGGCCTGGGG | |
| | | TAGAGCAGACTGTGGCTTCACCTCCGAGTCTTACCAGCAAGGGGTCCTGTCTGCC | |
| | | ACCATCCTCTATGAGATCTTGCTAGGGAAGGCCACCTIGTATGCCGTGCTGGTCA | |
| | | GTGCCCTCGTGCTGATGGCCATGGTCAAGAGAAAGGATTCCAGAGGC | |
| | β3 (with | ATGGACTCCTGGACCTTCTGCTGTGTGTCCCTTTGCATCCTGGTAGCGAAGCATA | SEQ ID NO: 150 |
| | TRBC1) | CAGATGCTGGAGTTATCCAGTCACCCCGCCATGAGGTGACAGAGATGGGACAAGA | |
| | | AGTGACTCTGAGATGTAAACCAATTTCAGGCCACAACTCCCTTTTCTGGTACAGA | |
| | | CAGACCATGATGCGGGGACTGGAGTTGCTCATTTACTTTAACAACAACGTTCCGA | |
| | | TAGATGATTCAGGGATGCCCGAGGATCGATTCTCAGCTAAGATGCCTAATGCATC | |
| | | ATTCTCCACTCTGAAGATCCAGCCCTCAGAACCCAGGGACTCAGCTGTGTACTTC | |
| | | TGTGCCAGCAGCACAGTGGGAGGGGAGGATTATGGCTACACCTTCGGTTCGGGGA | |
| | | CCAGGTTAACCGTTGTAGAGGACCTGAACAAGGTGTTCCCACCCGAGGTCGCTGT | |
| | | GTTTGAGCCATCAGAAGCAGAGATCTCCCACACCCAAAAGGCCACACTGGTGTGC | |
| | | CTGGCCACAGGCTTCTTCCCCGACCACGTGGAGCTGAGCTGGTGGGTGAATGGGA | |
| | | AGGAGGTGCACAGTGGGGTCAGCACGGACCCGCAGCCCCTCAAGGAGCAGCCCGC | |
| | | CCTCAATGACTCCAGATACTGCCTGAGCAGCCGCCTGAGGGTCTCGGCCACCTTC | |
| | | TGGCAGAACCCCCGCAACCACTTCCGCTGTCAAGTCCAGTTCTACGGGCTCTCGG | |
| | | AGAATGACGAGTGGACCCAGGATAGGGCCAAACCCGTCACCCAGATCGTCAGCGC | |
| | | CGAGGCCTGGGGTAGAGCAGACTGTGGCTTTACCTCGGTGTCCTACCAGCAAGGG | |
| | | GTCCTGTCTGCCACCATCCTCTATGAGATCCTGCTAGGGAAGGCCACCCTGTATG | |
| | | CTGTGCTGGTCAGCGCCCTIGTGTTGATGGCCATGGTCAAGAGAAAGGATTTC | |
| | β3 (with | ATGGACTCCTGGACCTTCTGCTGTGTGTCCCTTTGCATCCTGGTAGCGAAGCATA | SEQ ID NO: 151 |
| | TRBC2) | CAGATGCTGGAGTTATCCAGTCACCCCGCCATGAGGTGACAGAGATGGGACAAGA | |
| | | AGTGACTCTGAGATGTAAACCAATTTCAGGCCACAACTCCCTTTTCTGGTACAGA | |
| | | CAGACCATGATGCGGGGACTGGAGTTGCTCATTTACTTTAACAACAACGTTCCGA | |
| | | TAGATGATTCAGGGATGCCCGAGGATCGATTCTCAGCTAAGATGCCTAATGCATC | |
| | | ATTCTCCACTCTGAAGATCCAGCCCTCAGAACCCAGGGACTCAGCTGTGTACTTC | |
| | | TGTGCCAGCAGCACAGTGGGAGGGGAGGATTATGGCTACACCTTCGGTTCGGGGA | |
| | | CCAGGTTAACCGTTGTAGAGGACCTGAAAAACGTGTTCCCACCCGAGGTCGCTGT | |
| | | GTTTGAGCCATCAGAAGCAGAGATCTCCCACACCCAAAAGGCCACACTGGTGTGC | |
| | | CTGGCCACAGGCTTCTACCCCGACCACGTGGAGCTGAGCTGGTGGGTGAATGGGA | |
| | | AGGAGGTGCACAGTGGGGTCAGCACAGACCCGCAGCCCCTCAAGGAGCAGCCCGC | |
| | | CCTCAATGACTCCAGATACTGCCTGAGCAGCCGCCTGAGGGTCTCGGCCACCTTC | |
| | | TGGCAGAACCCCCGCAACCACTTCCGCTGTCAAGTCCAGTTCTACGGGCTCTCGG | |
| | | AGAATGACGAGTGGACCCAGGATAGGGCCAAACCTGTCACCCAGATCGTCAGCGC | |
| | | CGAGGCCTGGGGTAGAGCAGACTGTGGCTTCACCTCCGAGTCTTACCAGCAAGGG | |
| | | GTCCTGTCTGCCACCATCCTCTATGAGATCTTGCTAGGGAAGGCCACCTTGTATG | |
| | | CCGTGCTGGTCAGTGCCCTCGTGCTGATGGCCATGGTCAAGAGAAAGGATTCCAG | |
| | | AGGC | |
| HD5 | α (with | ATGACATCCATTCGAGCTGTATTTATATTCCTGTGGCTGCAGCTGGACTTGGTGA | SEQ ID NO: 152 |
| | TRAC) | ATGGAGAGAATGTGGAGCAGCATCCTTCAACCCTGAGTGTCCAGGAGGGAGACAG | |
| | | CGCTGTTATCAAGTGTACTTATTCAGACAGTGCCTCAAACTACTTCCCTTGGTAT | |
| | | AAGCAAGAACTTGGAAAAAGACCTCAGCTTATTATAGACATTCGTTCAAATGTGG | |
| | | GCGAAAAGAAAGACCAACGAATTGCTGTTACATTGAACAAGACAGCCAAACATTT | |
| | | CTCCCTGCACATCACAGAGACCCAACCTGAAGACTCGGCTGTCTACTTCTGTGCA | |
| | | GCAAGTATGGCTGGGGCTGGGAGTTACCAACTCACTTTCGGGAAGGGGACCAAAC | |
| | | TCTCGGTCATACCAAATATCCAGAACCCTGACCCTGCCGTGTACCAGCTGAGAGA | |
| | | CTCTAAATCCAGTGACAAGTCTGTCTGCCTATTCACCGATTTTGATTCTCAAACA | |
| | | AATGTGTCACAAAGTAAGGATTCTGATGTGTATATCACAGACAAAACTGTGCTAG | |
| | | ACATGAGGTCTATGGACTTCAAGAGCAACAGTGCTGTGGCCTGGAGCAACAAATC | |
| | | TGACTTTGCATGTGCAAACGCCTTCAACAACAGCATTATTCCAGAAGACACCTTC | |
| | | TTCCCCAGCCCAGAAAGTTCCTGTGATGTCAAGCTGGTCGAGAAAAGCTTTGAAA | |
| | | CAGATACGAACCTAAACTTTCAAAACCTGTCAGTGATTGGGTTCCGAATCCTCCT | |
| | | CCTGAAAGTGGCCGGGTTTAATCTGCTCATGACGCTGCGGCTGTGGTCCAGC | |
| | β1 (with | ATGGGCACAAGGITGTTCTTCTATGTGGCCCTTTGTCTCCTGTGGACAGGACACA | SEQ ID NO: 153 |
| | TRBC1) | TGGATGCTGGAATCACCCAGAGCCCAAGACACAAGGTCACAGAGACAGGAACACC | |
| | | AGTGACTCTGAGATGTCACCAGACTGAGAACCACCGCTATATGTACTGGTATCGA | |
| | | CAAGACCCGGGGCATGGGCTGAGGCTGATCCATTACTCATATGGTGTTAAAGATA | |
| | | CTGACAAAGGAGAAGTCTCAGATGGCTATAGTGTCTCTAGATCAAAGACAGAGGA | |
| | | TTTCCTCCTCACTCTGGAGTCCGCTACCAGCTCCCAGACATCTGTGTACTTCTGT | |
| | | GCCATCTCGGTGGGACAGGGGGCCCTCTACGAGCAGTACTTCGGGCCGGGCACCA | |
| | | GGCTCACGGTCACAGAGGACCTGAACAAGGTGTTCCCACCCGAGGTCGCTGTGTT | |
| | | TGAGCCATCAGAAGCAGAGATCTCCCACACCCAAAAGGCCACACTGGTGTGCCTG | |
| | | GCCACAGGCTTCTTCCCCGACCACGTGGAGCTGAGCTGGTGGGTGAATGGGAAGG | |
| | | AGGTGCACAGTGGGGTCAGCACGGACCCGCAGCCCCTCAAGGAGCAGCCCGCCCT | |
| | | CAATGACTCCAGATACTGCCTGAGCAGCCGCCTGAGGGTCTCGGCCACCTTCTGG | |
| | | CAGAACCCCCGCAACCACTTCCGCTGTCAAGTCCAGTTCTACGGGCTCTCGGAGA | |
| | | ATGACGAGTGGACCCAGGATAGGGCCAAACCCGTCACCCAGATCGTCAGCGCCGA | |
| | | GGCCTGGGGTAGAGCAGACTGTGGCTTTACCTCGGTGTCCTACCAGCAAGGGGTC | |
| | | CTGTCTGCCACCATCCTCTATGAGATCCTGCTAGGGAAGGCCACCCTGTATGCTG | |
| | | TGCTGGTCAGCGCCCTIGTGTTGATGGCCATGGTCAAGAGAAAGGATTTC | |
| | β1 (with | ATGGGCACAAGGITGTTCTTCTATGTGGCCCTTTGTCTCCTGTGGACAGGACACA | SEQ ID NO: 154 |
| | TRBC2) | TGGATGCTGGAATCACCCAGAGCCCAAGACACAAGGTCACAGAGACAGGAACACC | |
| | | AGTGACTCTGAGATGTCACCAGACTGAGAACCACCGCTATATGTACTGGTATCGA | |
| | | CAAGACCCGGGGCATGGGCTGAGGCTGATCCATTACTCATATGGTGTTAAAGATA | |
| | | CTGACAAAGGAGAAGTCTCAGATGGCTATAGTGTCTCTAGATCAAAGACAGAGGA | |
| | | TTTCCTCCTCACTCTGGAGTCCGCTACCAGCTCCCAGACATCTGTGTACTTCTGT | |
| | | GCCATCTCGGTGGGACAGGGGGCCCTCTACGAGCAGTACTTCGGGCCGGGCACCA | |
| | | GGCTCACGGTCACAGAGGACCTGAAAAACGTGTTCCCACCCGAGGTCGCTGTGTT | |
| | | TGAGCCATCAGAAGCAGAGATCTCCCACACCCAAAAGGCCACACTGGTGTGCCTG | |
| | | GCCACAGGCTTCTACCCCGACCACGTGGAGCTGAGCTGGTGGGTGAATGGGAAGG | |
| | | AGGTGCACAGTGGGGTCAGCACAGACCCGCAGCCCCTCAAGGAGCAGCCCGCCCT | |
| | | CAATGACTCCAGATACTGCCTGAGCAGCCGCCTGAGGGTCTCGGCCACCTTCTGG | |
| | | CAGAACCCCCGCAACCACTTCCGCTGTCAAGTCCAGTTCTACGGGCTCTCGGAGA | |
| | | ATGACGAGTGGACCCAGGATAGGGCCAAACCTGTCACCCAGATCGTCAGCGCCGA | |
| | | GGCCTGGGGTAGAGCAGACTGTGGCTTCACCTCCGAGTCTTACCAGCAAGGGGTC | |
| | | CTGTCTGCCACCATCCTCTATGAGATCTTGCTAGGGAAGGCCACCTTGTATGCCG | |
| | | TGCTGGTCAGTGCCCTCGTGCTGATGGCCATGGTCAAGAGAAAGGATTCCAGAGG | |
| | | C | |
| | β2 (with | ATGGGCTTCAGGCTCCTCTGCTGTGTGGCCTTTTGTCTCCTGGGAGCAGGCCCAG | SEQ ID NO: 155 |
| | TRBC1) | TGGATTCTGGAGTCACACAAACCCCAAAGCACCTGATCACAGCAACTGGACAGCG | |
| | | AGTGACGCTGAGATGCTCCCCTAGGTCTGGAGACCTCTCTGTGTACTGGTACCAA | |
| | | CAGAGCCTGGACCAGGGCCTCCAGTTCCTCATTCAGTATTATAATGGAGAAGAGA | |
| | | GAGCAAAAGGAAACATTCTTGAACGATTCTCCGCACAACAGTTCCCTGACTTGCA | |
| | | CTCTGAACTAAACCTGAGCTCTCTGGAGCTGGGGGACTCAGCTTTGTATTTCTGT | |
| | | GCCAGCAGCGTAGCTCGGGACAGGCGGAACTATGGCTACACCTTCGGTTCGGGGA | |
| | | CCAGGTTAACCGTTGTAGAGGACCTGAACAAGGTGTTCCCACCCGAGGTCGCTGT | |
| | | GTTTGAGCCATCAGAAGCAGAGATCTCCCACACCCAAAAGGCCACACTGGTGTGC | |
| | | CTGGCCACAGGCTTCTTCCCCGACCACGTGGAGCTGAGCTGGTGGGTGAATGGGA | |
| | | AGGAGGTGCACAGTGGGGTCAGCACGGACCCGCAGCCCCTCAAGGAGCAGCCCGC | |
| | | CCTCAATGACTCCAGATACTGCCTGAGCAGCCGCCTGAGGGTCTCGGCCACCTTC | |
| | | TGGCAGAACCCCCGCAACCACTTCCGCTGTCAAGTCCAGTTCTACGGGCTCTCGG | |
| | | AGAATGACGAGTGGACCCAGGATAGGGCCAAACCCGTCACCCAGATCGTCAGCGC | |
| | | CGAGGCCTGGGGTAGAGCAGACTGTGGCTTTACCTCGGTGTCCTACCAGCAAGGG | |
| | | GTCCTGTCTGCCACCATCCTCTATGAGATCCTGCTAGGGAAGGCCACCCTGTATG | |
| | | CTGTGCTGGTCAGCGCCCTIGTGTTGATGGCCATGGTCAAGAGAAAGGATTTC | |
| | β2 (with | ATGGGCTTCAGGCTCCTCTGCTGTGTGGCCTTTTGTCTCCTGGGAGCAGGCCCAG | SEQ ID NO: 156 |
| | TRBC2) | TGGATTCTGGAGTCACACAAACCCCAAAGCACCTGATCACAGCAACTGGACAGCG | |
| | | AGTGACGCTGAGATGCTCCCCTAGGTCTGGAGACCTCTCTGTGTACTGGTACCAA | |
| | | CAGAGCCTGGACCAGGGCCTCCAGTTCCTCATTCAGTATTATAATGGAGAAGAGA | |
| | | GAGCAAAAGGAAACATTCTTGAACGATTCTCCGCACAACAGTTCCCTGACTTGCA | |
| | | CTCTGAACTAAACCTGAGCTCTCTGGAGCTGGGGGACTCAGCTTTGTATTTCTGT | |
| | | GCCAGCAGCGTAGCTCGGGACAGGCGGAACTATGGCTACACCTTCGGTTCGGGGA | |
| | | CCAGGTTAACCGTTGTAGAGGACCTGAAAAACGTGTTCCCACCCGAGGTCGCTGT | |
| | | GTTTGAGCCATCAGAAGCAGAGATCTCCCACACCCAAAAGGCCACACTGGTGTGC | |
| | | CTGGCCACAGGCTTCTACCCCGACCACGTGGAGCTGAGCTGGTGGGTGAATGGGA | |
| | | AGGAGGTGCACAGTGGGGTCAGCACAGACCCGCAGCCCCTCAAGGAGCAGCCCGC | |
| | | CCTCAATGACTCCAGATACTGCCTGAGCAGCCGCCTGAGGGTCTCGGCCACCTTC | |
| | | TGGCAGAACCCCCGCAACCACTTCCGCTGTCAAGTCCAGTTCTACGGGCTCTCGG | |
| | | AGAATGACGAGTGGACCCAGGATAGGGCCAAACCTGTCACCCAGATCGTCAGCGC | |
| | | CGAGGCCTGGGGTAGAGCAGACTGTGGCTTCACCTCCGAGTCTTACCAGCAAGGG | |
| | | GTCCTGTCTGCCACCATCCTCTATGAGATCTTGCTAGGGAAGGCCACCTTGTATG | |
| | | CCGTGCTGGTCAGTGCCCTCGTGCTGATGGCCATGGTCAAGAGAAAGGATTCCAG | |
| | | AGGC | |
| HD6 | α1 (with | atggccatgctcctgggggcatcagtgctgattctgtggcttcagccagactggg | SEQ ID NO: 223 |
| | TRAC) | taaacagtcaacagaagaatgatgaccagcaagttaagcaaaattcaccatccct | |
| | | gagcgtccaggaaggaagaatttctattctgaactgtgactatactaacagcatg | |
| | | tttgattatttcctatggtacaaaaaataccctgctgaaggtcctacattcctga | |
| | | tatctataagttccattaaggataaaaatgaagatggaagattcactgtcttctt | |
| | | aaacaaaagtgccaagcacctctctctgcacattgtgccctcccagcctggagac | |
| | | tctgcagtgtacttctgtgcagcaaacaatgccagactcatgtttggagatggaa | |
| | | ctcagctggtggtgaagcccaatatccagaaccctgaccctgccgtgtaccagct | |
| | | gagagactctaaatccagtgacaagtctgtctgcctattcaccgattttgattct | |
| | | caaacaaatgtgtcacaaagtaaggattctgatgtgtatatcacagacaaaactg | |
| | | tgctagacatgaggtctatggacttcaagagcaacagtgctgtggcctggagcaa | |
| | | caaatctgactttgcatgtgcaaacgccttcaacaacagcattattccagaagac | |
| | | accttcttccccagcccagaaagttcctgtgatgtcaagctggtcgagaaaagct | |
| | | ttgaaacagatacgaacctaaactttcaaaacctgtcagtgattgggttccgaat | |
| | | cctcctcctgaaagtggccgggtttaatctgctcatgacgctgcggctgtggtcc | |
| | | agc | |
| | α2 (with | ATGGCCATGCTCCTGGGGGCATCAGTGCTGATTCTGTGGCTTCAGCCAGACTGGG | SEQ ID NO: 292 |
| | TRAC) | TAAACAGTCAACAGAAGAATGATGACCAGCAAGTTAAGCAAAATTCACCATCCCT | |
| | | GAGCGTCCAGGAAGGAAGAATTTCTATTCTGAACTGTGACTATACTAACAGCATG | |
| | | TTTGATTATTTCCTATGGTACAAAAAATACCCTGCTGAAGGTCCTACATTCCTGA | |
| | | TATCTATAAGTTCCATTAAGGATAAAAATGAAGATGGAAGATTCACTGTCTTCTT | |
| | | AAACAAAAGTGCCAAGCACCTCTCTCTGCACATTGTGCCCTCCCAGCCTGGAGAC | |
| | | TCTGCAGTGTACTTCTGTGCAGCAAGCGCTACCGGTAACCAGTTCTATTTTGGGA | |
| | | CAGGGACAAGTTTGACGGTCATTCCAAATATCCAGAACCCTGACCCTGCCGTGTA | |
| | | CCAGCTGAGAGACTCTAAATCCAGTGACAAGTCTGTCTGCCTATTCACCGATTTT | |
| | | GATTCTCAAACAAATGTGTCACAAAGTAAGGATTCTGATGTGTATATCACAGACA | |
| | | AAACTGTGCTAGACATGAGGTCTATGGACTTCAAGAGCAACAGTGCTGTGGCCTG | |
| | | GAGCAACAAATCTGACTTTGCATGTGCAAACGCCTTCAACAACAGCATTATTCCA | |
| | | GAAGACACCTTCTTCCCCAGCCCAGAAAGTTCCTGTGATGTCAAGCTGGTCGAGA | |
| | | AAAGCTTTGAAACAGATACGAACCTAAACTTTCAAAACCTGTCAGTGATTGGGTT | |
| | | CCGAATCCTCCTCCTGAAAGTGGCCGGGTTTAATCTGCTCATGACGCTGCGGCTG | |
| | | TGGTCCAGCTGA | |
| | β1 (with | atggactcctggaccttctgctgtgtgtccctttgcatcctggtagcgaagcata | SEQ ID NO: 224 |
| | TRBC1) | cagatgctggagttatccagtcaccccgccatgaggtgacagagatgggacaaga | |
| | | agtgactctgagatgtaaaccaatttcaggccacaactcccttttctggtacaga | |
| | | cagaccatgatgcggggactggagttgctcatttactttaacaacaacgttccga | |
| | | tagatgattcagggatgcccgaggatcgattctcagctaagatgcctaatgcatc | |
| | | attctccactctgaagatccagccctcagaacccagggactcagctgtgtacttc | |
| | | tgtgccagcagtgataccagggcccgggagcagttcttcgggccagggacacggc | |
| | | tcaccgtgctagaggacctgaacaaggtgttcccacccgaggtcgctgtgtttga | |
| | | gccatcagaagcagagatctcccacacccaaaaggccacactggtgtgcctggcc | |
| | | acaggcttcttccccgaccacgtggagctgagctggtgggtgaatgggaaggagg | |
| | | tgcacagtggggtcagcacggacccgcagcccctcaaggagcagcccgccctcaa | |
| | | tgactccagatactgcctgagcagccgcctgagggtctcggccaccttctggcag | |
| | | aacccccgcaaccacttccgctgtcaagtccagttctacgggctctcggagaatg | |
| | | acgagtggacccaggatagggccaaacccgtcacccagatcgtcagcgccgaggc | |
| | | ctggggtagagcagactgtggctttacctcggtgtcctaccagcaaggggtcctg | |
| | | tctgccaccatcctctatgagatcctgctagggaaggccaccctgtatgctgtgc | |
| | | tggtcagcgcccttgtgttgatggccatggtcaagagaaaggatttc | |
| | β1 (with | atggactcctggaccttctgctgtgtgtccctttgcatcctggtagcgaagcata | SEQ ID NO: 225 |
| | TRBC2) | cagatgctggagttatccagtcaccccgccatgaggtgacagagatgggacaaga | |
| | | agtgactctgagatgtaaaccaatttcaggccacaactcccttttctggtacaga | |
| | | cagaccatgatgcggggactggagttgctcatttactttaacaacaacgttccga | |
| | | tagatgattcagggatgcccgaggatcgattctcagctaagatgcctaatgcatc | |
| | | attctccactctgaagatccagccctcagaacccagggactcagctgtgtacttc | |
| | | tgtgccagcagtgataccagggcccgggagcagttcttcgggccagggacacggc | |
| | | tcaccgtgctagaggacctgaaaaacgtgttcccacccgaggtcgctgtgtttga | |
| | | gccatcagaagcagagatctcccacacccaaaaggccacactggtgtgcctggcc | |
| | | acaggcttctaccccgaccacgtggagctgagctggtgggtgaatgggaaggagg | |
| | | tgcacagtggggtcagcacagacccgcagcccctcaaggagcagcccgccctcaa | |
| | | tgactccagatactgcctgagcagccgcctgagggtctcggccaccttctggcag | |
| | | aacccccgcaaccacttccgctgtcaagtccagttctacgggctctcggagaatg | |
| | | acgagtggacccaggatagggccaaacctgtcacccagatcgtcagcgccgaggc | |
| | | ctggggtagagcagactgtggcttcacctccgagtcttaccagcaaggggtcctg | |
| | | tctgccaccatcctctatgagatcttgctagggaaggccaccttgtatgccgtgc | |
| | | tggtcagtgccctcgtgctgatggccatggtcaagagaaaggattccagaggc | |
| | β2 (with | ATGGGCTCCAGGCTGCTCTGTIGGGTGCTGCTTTGTCTCCTGGGAGCAGGCCCAG | SEQ ID NO: 293 |
| | TRBC1) | TAAAGGCTGGAGTCACTCAAACTCCAAGATATCTGATCAAAACGAGAGGACAGCA | |
| | | AGTGACACTGAGCTGCTCCCCTATCTCTGGGCATAGGAGTGTATCCTGGTACCAA | |
| | | CAGACCCCAGGACAGGGCCTTCAGTTCCTCTTTGAATACTTCAGTGAGACACAGA | |
| | | GAAACAAAGGAAACTTCCCTGGTCGATTCTCAGGGCGCCAGTTCTCTAACTCTCG | |
| | | CTCTGAGATGAATGTGAGCACCTTGGAGCTGGGGGACTCGGCCCTTTATCTTTGC | |
| | | GCCAGCAGCCCTGGACAGCACGGGGAGCTGITTTTTGGAGAAGGCTCTAGGCTGA | |
| | | CCGTACTGGAGGACCTGAACAAGGTGTTCCCACCCGAGGTCGCTGTGTTTGAGCC | |
| | | ATCAGAAGCAGAGATCTCCCACACCCAAAAGGCCACACTGGTGTGCCTGGCCACA | |
| | | GGCTTCTTCCCCGACCACGTGGAGCTGAGCTGGTGGGTGAATGGGAAGGAGGTGC | |
| | | ACAGTGGGGTCAGCACGGACCCGCAGCCCCTCAAGGAGCAGCCCGCCCTCAATGA | |
| | | CTCCAGATACTGCCTGAGCAGCCGCCTGAGGGTCTCGGCCACCTTCTGGCAGAAC | |
| | | CCCCGCAACCACTTCCGCTGTCAAGTCCAGTTCTACGGGCTCTCGGAGAATGACG | |
| | | AGTGGACCCAGGATAGGGCCAAACCCGTCACCCAGATCGTCAGCGCCGAGGCCTG | |
| | | GGGTAGAGCAGACTGTGGCTTTACCTCGGTGTCCTACCAGCAAGGGGTCCTGTCT | |
| | | GCCACCATCCTCTATGAGATCCTGCTAGGGAAGGCCACCCTGTATGCTGTGCTGG | |
| | | TCAGCGCCCTIGTGTTGATGGCCATGGTCAAGAGAAAGGATTTC | |
| | β2 (with | ATGGGCTCCAGGCTGCTCTGTTGGGTGCTGCTTTGTCTCCTGGGAGCAGGCCCAG | SEQ ID NO: 294 |
| | TRBC2) | TAAAGGCTGGAGTCACTCAAACTCCAAGATATCTGATCAAAACGAGAGGACAGCA | |
| | | AGTGACACTGAGCTGCTCCCCTATCTCTGGGCATAGGAGTGTATCCTGGTACCAA | |
| | | CAGACCCCAGGACAGGGCCTTCAGTTCCTCTTTGAATACTTCAGTGAGACACAGA | |
| | | GAAACAAAGGAAACTTCCCTGGTCGATTCTCAGGGCGCCAGTTCTCTAACTCTCG | |
| | | CTCTGAGATGAATGTGAGCACCTTGGAGCTGGGGGACTCGGCCCTTTATCTTTGC | |
| | | GCCAGCAGCCCTGGACAGCACGGGGAGCTGITTTTTGGAGAAGGCTCTAGGCTGA | |
| | | CCGTACTGGAGGACCTGAAAAACGTGTTCCCACCCGAGGTCGCTGTGTTTGAGCC | |
| | | ATCAGAAGCAGAGATCTCCCACACCCAAAAGGCCACACTGGTGTGCCTGGCCACA | |
| | | GGCTTCTACCCCGACCACGTGGAGCTGAGCTGGTGGGTGAATGGGAAGGAGGTGC | |
| | | ACAGTGGGGTCAGCACAGACCCGCAGCCCCTCAAGGAGCAGCCCGCCCTCAATGA | |
| | | CTCCAGATACTGCCTGAGCAGCCGCCTGAGGGTCTCGGCCACCTTCTGGCAGAAC | |
| | | CCCCGCAACCACTTCCGCTGTCAAGTCCAGTTCTACGGGCTCTCGGAGAATGACG | |
| | | AGTGGACCCAGGATAGGGCCAAACCTGTCACCCAGATCGTCAGCGCCGAGGCCTG | |
| | | GGGTAGAGCAGACTGTGGCTTCACCTCCGAGTCTTACCAGCAAGGGGTCCTGTCT | |
| | | GCCACCATCCTCTATGAGATCTTGCTAGGGAAGGCCACCTTGTATGCCGTGCTGG | |
| | | TCAGTGCCCTCGTGCTGATGGCCATGGTCAAGAGAAAGGATTCCAGAGGC | |
| HD7 | α1 (with | atgaagacatttgctggattttcgttcctgtttttgtggctgcagctggactgta | SEQ ID NO: 226 |
| | TRAC) | tgagtagaggagaggatgtggagcagagtcttttcctgagtgtccgagagggaga | |
| | | cagctccgttataaactgcacttacacagacagctcctccacctacttatactgg | |
| | | tataagcaagaacctggagcaggtctccagttgctgacgtatattttttcaaata | |
| | | tggacatgaaacaagaccaaagactcactgttctattgaataaaaaggataaaca | |
| | | tctgtctctgcgcattgcagacacccagactggggactcagctatctacttctgt | |
| | | gcagagaggcttaacaccgacaagctcatctttgggactgggaccagattacaag | |
| | | tctttccaaatatccagaaccctgaccctgccgtgtaccagctgagagactctaa | |
| | | atccagtgacaagtctgtctgcctattcaccgattttgattctcaaacaaatgtg | |
| | | tcacaaagtaaggattctgatgtgtatatcacagacaaaactgtgctagacatga | |
| | | ggtctatggacttcaagagcaacagtgctgtggcctggagcaacaaatctgactt | |
| | | tgcatgtgcaaacgccttcaacaacagcattattccagaagacaccttcttcccc | |
| | | agcccagaaagttcctgtgatgtcaagctggtcgagaaaagctttgaaacagata | |
| | | cgaacctaaactttcaaaacctgtcagtgattgggttccgaatcctcctcctgaa | |
| | | agtggccgggtttaatctgctcatgacgctgcggctgtggtccagc | |
| | α2 (with | atgaagaggatattgggagctctgctggggctcttgagtgcccaggtttgctgtg | SEQ ID NO: 227 |
| | TRAC) | tgagaggaatacaagtggagcagagtcctccagacctgattctccaggagggagc | |
| | | caattccacgctgcggtgcaatttttctgactctgtgaacaatttgcagtggttt | |
| | | catcaaaacccttggggacagctcatcaacctgttttacattccctcagggacaa | |
| | | aacagaatggaagattaagcgccacgactgtcgctacggaacgctacagcttatt | |
| | | gtacatttcctcttcccagaccacagactcaggcgtttatttctgtgctgtggag | |
| | | gcaactgacagctgggggaaattgcagtttggagcagggacccaggttgtggtca | |
| | | ccccagatatccagaaccctgaccctgccgtgtaccagctgagagactctaaatc | |
| | | cagtgacaagtctgtctgcctattcaccgattttgattctcaaacaaatgtgtca | |
| | | caaagtaaggattctgatgtgtatatcacagacaaaactgtgctagacatgaggt | |
| | | ctatggacttcaagagcaacagtgctgtggcctggagcaacaaatctgactttgc | |
| | | atgtgcaaacgccttcaacaacagcattattccagaagacaccttcttccccagc | |
| | | ccagaaagttcctgtgatgtcaagctggtcgagaaaagctttgaaacagatacga | |
| | | acctaaactttcaaaacctgtcagtgattgggttccgaatcctcctcctgaaagt | |
| | | ggccgggtttaatctgctcatgacgctgcggctgtggtccagc | |
| | α3 (with | atgacatccattcgagctgtatttatattcctgtggctgcagctggacttggtga | SEQ ID NO: 228 |
| | TRAC) | atggagagaatgtggagcagcatccttcaaccctgagtgtccaggagggagacag | |
| | | cgctgttatcaagtgtacttattcagacagtgcctcaaactacttcccttggtat | |
| | | aagcaagaacttggaaaaagacctcagcttattatagacattcgttcaaatgtgg | |
| | | gcgaaaagaaagaccaacgaattgctgttacattgaacaagacagccaaacattt | |
| | | ctccctgcacatcacagagacccaacctgaagactcggctgtctacttctgtgca | |
| | | gtacgaacctcctacgacaaggtgatatttgggccagggacaagcttatcagtca | |
| | | ttccaaatatccagaaccctgaccctgccgtgtaccagctgagagactctaaatc | |
| | | cagtgacaagtctgtctgcctattcaccgattttgattctcaaacaaatgtgtca | |
| | | caaagtaaggattctgatgtgtatatcacagacaaaactgtgctagacatgaggt | |
| | | ctatggacttcaagagcaacagtgctgtggcctggagcaacaaatctgactttgc | |
| | | atgtgcaaacgccttcaacaacagcattattccagaagacaccttcttccccagc | |
| | | ccagaaagttcctgtgatgtcaagctggtcgagaaaagctttgaaacagatacga | |
| | | acctaaactttcaaaacctgtcagtgattgggttccgaatcctcctcctgaaagt | |
| | | ggccgggtttaatctgctcatgacgctgcggctgtggtccagc | |
| | α-4 (with | ATGGAAACTCTCCTGGGAGTGTCTTTGGTGATTCTATGGCTTCAACTGGCTAGGG | SEQ ID NO: 295 |
| | TRAC) | TGAACAGTCAACAGGGAGAAGAGGATCCTCAGGCCTTGAGCATCCAGGAGGGTGA | |
| | | AAATGCCACCATGAACTGCAGTTACAAAACTAGTATAAACAATTTACAGTGGTAT | |
| | | AGACAAAATTCAGGTAGAGGCCTTGTCCACCTAATTTTAATACGTTCAAATGAAA | |
| | | GAGAGAAACACAGTGGAAGATTAAGAGTCACGCTTGACACTTCCAAGAAAAGCAG | |
| | | TTCCTTGTTGATCACGGCTTCCCGGGCAGCAGACACTGCTTCTTACTTCTGTGCT | |
| | | ACGGACGGGGATAGCAGCTATAAATTGATCTTCGGGAGTGGGACCAGACTGCTGG | |
| | | TCAGGCCTGATATCCAGAACCCTGACCCTGCCGTGTACCAGCTGAGAGACTCTAA | |
| | | ATCCAGTGACAAGTCTGTCTGCCTATTCACCGATTTTGATTCTCAAACAAATGTG | |
| | | TCACAAAGTAAGGATTCTGATGTGTATATCACAGACAAAACTGTGCTAGACATGA | |
| | | GGTCTATGGACTTCAAGAGCAACAGTGCTGTGGCCTGGAGCAACAAATCTGACTT | |
| | | TGCATGTGCAAACGCCTTCAACAACAGCATTATTCCAGAAGACACCTTCTTCCCC | |
| | | AGCCCAGAAAGTTCCTGTGATGTCAAGCTGGTCGAGAAAAGCTTTGAAACAGATA | |
| | | CGAACCTAAACTTTCAAAACCTGTCAGTGATTGGGTTCCGAATCCTCCTCCTGAA | |
| | | AGTGGCCGGGITTAATCTGCTCATGACGCTGCGGCTGTGGTCCAGC | |
| | β1 (with | atgctgctgcttctgctgcttctggggccaggtataagcctccttctacctggga | SEQ ID NO: 229 |
| | TRBC1) | gcttggcaggctccgggcttggtgctgtcgtctctcaacatccgagctgggttat | |
| | | ctgtaagagtggaacctctgtgaagatcgagtgccgttccctggactttcaggcc | |
| | | acaactatgttttggtatcgtcagttcccgaaacagagtctcatgctgatggcaa | |
| | | cttccaatgagggctccaaggccacatacgagcaaggcgtcgagaaggacaagtt | |
| | | tctcatcaaccatgcaagcctgaccttgtccactctgacagtgaccagtgcccat | |
| | | cctgaagacagcagcttctacatctgcagtgctagggacagtgtgtctggaaaca | |
| | | ccatatattttggagagggaagttggctcactgttgtagaggacctgaacaaggt | |
| | | gttcccacccgaggtcgctgtgtttgagccatcagaagcagagatctcccacacc | |
| | | caaaaggccacactggtgtgcctggccacaggcttcttccccgaccacgtggagc | |
| | | tgagctggtgggtgaatgggaaggaggtgcacagtggggtcagcacggacccgca | |
| | | gcccctcaaggagcagcccgccctcaatgactccagatactgcctgagcagccgc | |
| | | ctgagggtctcggccaccttctggcagaacccccgcaaccacttccgctgtcaag | |
| | | tccagttctacgggctctcggagaatgacgagtggacccaggatagggccaaacc | |
| | | cgtcacccagatcgtcagcgccgaggcctggggtagagcagactgtggctttacc | |
| | | tcggtgtcctaccagcaaggggtcctgtctgccaccatcctctatgagatcctgc | |
| | | tagggaaggccaccctgtatgctgtgctggtcagcgcccttgtgttgatggccat | |
| | | ggtcaagagaaaggatttc | |
| | β1 (with | atgctgctgcttctgctgcttctggggccaggtataagcctccttctacctggga | SEQ ID NO: 230 |
| | TRBC2) | gcttggcaggctccgggcttggtgctgtcgtctctcaacatccgagctgggttat | |
| | | ctgtaagagtggaacctctgtgaagatcgagtgccgttccctggactttcaggcc | |
| | | acaactatgttttggtatcgtcagttcccgaaacagagtctcatgctgatggcaa | |
| | | cttccaatgagggctccaaggccacatacgagcaaggcgtcgagaaggacaagtt | |
| | | tctcatcaaccatgcaagcctgaccttgtccactctgacagtgaccagtgcccat | |
| | | cctgaagacagcagcttctacatctgcagtgctagggacagtgtgtctggaaaca | |
| | | ccatatattttggagagggaagttggctcactgttgtagaggacctgaaaaacgt | |
| | | gttcccacccgaggtcgctgtgtttgagccatcagaagcagagatctcccacacc | |
| | | caaaaggccacactggtgtgcctggccacaggcttctaccccgaccacgtggagc | |
| | | tgagctggtgggtgaatgggaaggaggtgcacagtggggtcagcacagacccgca | |
| | | gcccctcaaggagcagcccgccctcaatgactccagatactgcctgagcagccgc | |
| | | ctgagggtctcggccaccttctggcagaacccccgcaaccacttccgctgtcaag | |
| | | tccagttctacgggctctcggagaatgacgagtggacccaggatagggccaaacc | |
| | | tgtcacccagatcgtcagcgccgaggcctggggtagagcagactgtggcttcacc | |
| | | tccgagtcttaccagcaaggggtcctgtctgccaccatcctctatgagatcttgc | |
| | | tagggaaggccaccttgtatgccgtgctggtcagtgccctcgtgctgatggccat | |
| | | ggtcaagagaaaggattccagaggc | |
| | β2 (with | atgctgagtcttctgctccttctcctgggactaggctctgtgttcagtgctgtca | SEQ ID NO: 231 |
| | TRBC1) | tctctcaaaagccaagcagggatatctgtcaacgtggaacctccctgacgatcca | |
| | | gtgtcaagtcgatagccaagtcaccatgatgttctggtaccgtcagcaacctgga | |
| | | cagagcctgacactgatcgcaactgcaaatcagggctctgaggccacatatgaga | |
| | | gtggatttgtcattgacaagtttcccatcagccgcccaaacctaacattctcaac | |
| | | tctgactgtgagcaacatgagccctgaagacagcagcatatatctctgcagcgtt | |
| | | gggggtagcgggagttacaatgagcagttcttcgggccagggacacggctcaccg | |
| | | tgctagaggacctgaacaaggtgttcccacccgaggtcgctgtgtttgagccatc | |
| | | agaagcagagatctcccacacccaaaaggccacactggtgtgcctggccacaggc | |
| | | ttcttccccgaccacgtggagctgagctggtgggtgaatgggaaggaggtgcaca | |
| | | gtggggtcagcacggacccgcagcccctcaaggagcagcccgccctcaatgactc | |
| | | cagatactgcctgagcagccgcctgagggtctcggccaccttctggcagaacccc | |
| | | cgcaaccacttccgctgtcaagtccagttctacgggctctcggagaatgacgagt | |
| | | ggacccaggatagggccaaacccgtcacccagatcgtcagcgccgaggcctgggg | |
| | | tagagcagactgtggctttacctcggtgtcctaccagcaaggggtcctgtctgcc | |
| | | accatcctctatgagatcctgctagggaaggccaccctgtatgctgtgctggtca | |
| | | gcgcccttgtgttgatggccatggtcaagagaaaggatttc | |
| | β2 (with | atgctgagtcttctgctccttctcctgggactaggctctgtgttcagtgctgtca | SEQ ID NO: 232 |
| | TRBC2) | tctctcaaaagccaagcagggatatctgtcaacgtggaacctccctgacgatcca | |
| | | gtgtcaagtcgatagccaagtcaccatgatgttctggtaccgtcagcaacctgga | |
| | | cagagcctgacactgatcgcaactgcaaatcagggctctgaggccacatatgaga | |
| | | gtggatttgtcattgacaagtttcccatcagccgcccaaacctaacattctcaac | |
| | | tctgactgtgagcaacatgagccctgaagacagcagcatatatctctgcagcgtt | |
| | | gggggtagcgggagttacaatgagcagttcttcgggccagggacacggctcaccg | |
| | | tgctagaggacctgaaaaacgtgttcccacccgaggtcgctgtgtttgagccatc | |
| | | agaagcagagatctcccacacccaaaaggccacactggtgtgcctggccacaggc | |
| | | ttctaccccgaccacgtggagctgagctggtgggtgaatgggaaggaggtgcaca | |
| | | gtggggtcagcacagacccgcagcccctcaaggagcagcccgccctcaatgactc | |
| | | cagatactgcctgagcagccgcctgagggtctcggccaccttctggcagaacccc | |
| | | cgcaaccacttccgctgtcaagtccagttctacgggctctcggagaatgacgagt | |
| | | ggacccaggatagggccaaacctgtcacccagatcgtcagcgccgaggcctgggg | |
| | | tagagcagactgtggcttcacctccgagtcttaccagcaaggggtcctgtctgcc | |
| | | accatcctctatgagatcttgctagggaaggccaccttgtatgccgtgctggtca | |
| | | gtgccctcgtgctgatggccatggtcaagagaaaggattccagaggc | |
| | β-3 (with | ATGCTGCTGCTTCTGCTGCTTCTGGGGCCAGGTATAAGCCTCCTTCTACCTGGGA | SEQ ID NO: 296 |
| | TRBC1) | GCTTGGCAGGCTCCGGGCTTGGTGCTGTCGTCTCTCAACATCCGAGCTGGGTTAT | |
| | | CTGTAAGAGTGGAACCTCTGTGAAGATCGAGTGCCGTTCCCTGGACTTTCAGGCC | |
| | | ACAACTATGTTTTGGTATCGTCAGTTCCCGAAACAGAGTCTCATGCTGATGGCAA | |
| | | CTTCCAATGAGGGCTCCAAGGCCACATACGAGCAAGGCGTCGAGAAGGACAAGTT | |
| | | TCTCATCAACCATGCAAGCCTGACCTTGTCCACTCTGACAGTGACCAGTGCCCAT | |
| | | CCTGAAGACAGCAGCTTCTACATCTGCAGTGCTAGAGACGTACTGACAGGGGACT | |
| | | ATGGCTACACCTTCGGTTCGGGGACCAGGTTAACCGTTGTAGAGGACCTGAACAA | |
| | | GGTGTTCCCACCCGAGGTCGCTGTGTTTGAGCCATCAGAAGCAGAGATCTCCCAC | |
| | | ACCCAAAAGGCCACACTGGTGTGCCTGGCCACAGGCTTCTTCCCCGACCACGTGG | |
| | | AGCTGAGCTGGTGGGTGAATGGGAAGGAGGTGCACAGTGGGGTCAGCACGGACCC | |
| | | GCAGCCCCTCAAGGAGCAGCCCGCCCTCAATGACTCCAGATACTGCCTGAGCAGC | |
| | | CGCCTGAGGGTCTCGGCCACCTTCTGGCAGAACCCCCGCAACCACTTCCGCTGTC | |
| | | AAGTCCAGTTCTACGGGCTCTCGGAGAATGACGAGTGGACCCAGGATAGGGCCAA | |
| | | ACCCGTCACCCAGATCGTCAGCGCCGAGGCCTGGGGTAGAGCAGACTGTGGCTTT | |
| | | ACCTCGGTGTCCTACCAGCAAGGGGTCCTGTCTGCCACCATCCTCTATGAGATCC | |
| | | TGCTAGGGAAGGCCACCCTGTATGCTGTGCTGGTCAGCGCCCTIGTGTTGATGGC | |
| | | CATGGTCAAGAGAAAGGATTTC | |
| | β-3 (with | ATGCTGCTGCTTCTGCTGCTTCTGGGGCCAGGTATAAGCCTCCTTCTACCTGGGA | SEQ ID NO: 297 |
| | TRBC2) | GCTTGGCAGGCTCCGGGCTTGGTGCTGTCGTCTCTCAACATCCGAGCTGGGTTAT | |
| | | CTGTAAGAGTGGAACCTCTGTGAAGATCGAGTGCCGTTCCCTGGACTTTCAGGCC | |
| | | ACAACTATGTTTTGGTATCGTCAGTTCCCGAAACAGAGTCTCATGCTGATGGCAA | |
| | | CTTCCAATGAGGGCTCCAAGGCCACATACGAGCAAGGCGTCGAGAAGGACAAGTT | |
| | | TCTCATCAACCATGCAAGCCTGACCTTGTCCACTCTGACAGTGACCAGTGCCCAT | |
| | | CCTGAAGACAGCAGCTTCTACATCTGCAGTGCTAGAGACGTACTGACAGGGGACT | |
| | | ATGGCTACACCTTCGGTTCGGGGACCAGGTTAACCGTTGTAGAGGACCTGAAAAA | |
| | | CGTGTTCCCACCCGAGGTCGCTGTGTTTGAGCCATCAGAAGCAGAGATCTCCCAC | |
| | | ACCCAAAAGGCCACACTGGTGTGCCTGGCCACAGGCTTCTACCCCGACCACGTGG | |
| | | AGCTGAGCTGGTGGGTGAATGGGAAGGAGGTGCACAGTGGGGTCAGCACAGACCC | |
| | | GCAGCCCCTCAAGGAGCAGCCCGCCCTCAATGACTCCAGATACTGCCTGAGCAGC | |
| | | CGCCTGAGGGTCTCGGCCACCTTCTGGCAGAACCCCCGCAACCACTTCCGCTGTC | |
| | | AAGTCCAGTTCTACGGGCTCTCGGAGAATGACGAGTGGACCCAGGATAGGGCCAA | |
| | | ACCTGTCACCCAGATCGTCAGCGCCGAGGCCTGGGGTAGAGCAGACTGTGGCTTC | |
| | | ACCTCCGAGTCTTACCAGCAAGGGGTCCTGTCTGCCACCATCCTCTATGAGATCT | |
| | | TGCTAGGGAAGGCCACCTTGTATGCCGTGCTGGTCAGTGCCCTCGTGCTGATGGC | |
| | | CATGGTCAAGAGAAAGGATTCCAGAGGC | |
| | β-4 (with | ATGGGCTCCTGGACCCTCTGCTGTGTGTCCCTTTGCATCCTGGTAGCAAAGCACA | SEQ ID NO: 298 |
| | TRBC1) | CAGATGCTGGAGTTATCCAGTCACCCCGGCACGAGGTGACAGAGATGGGACAAGA | |
| | | AGTGACTCTGAGATGTAAACCAATTTCAGGACACGACTACCTTTTCTGGTACAGA | |
| | | CAGACCATGATGCGGGGACTGGAGTTGCTCATTTACTTTAACAACAACGTTCCGA | |
| | | TAGATGATTCAGGGATGCCCGAGGATCGATTCTCAGCTAAGATGCCTAATGCATC | |
| | | ATTCTCCACTCTGAAGATCCAGCCCTCAGAACCCAGGGACTCAGCTGTGTACTTC | |
| | | TGTGCCAGCAGTTTAGGACTGAGCATTTCCCAAGAGACCCAGTACTTCGGGCCAG | |
| | | GCACGCGGCTCCTGGTGCTCGAGGACCTGAACAAGGTGTTCCCACCCGAGGTCGC | |
| | | TGTGTTTGAGCCATCAGAAGCAGAGATCTCCCACACCCAAAAGGCCACACTGGTG | |
| | | TGCCTGGCCACAGGCTTCTTCCCCGACCACGTGGAGCTGAGCTGGTGGGTGAATG | |
| | | GGAAGGAGGTGCACAGTGGGGTCAGCACGGACCCGCAGCCCCTCAAGGAGCAGCC | |
| | | CGCCCTCAATGACTCCAGATACTGCCTGAGCAGCCGCCTGAGGGTCTCGGCCACC | |
| | | TTCTGGCAGAACCCCCGCAACCACTTCCGCTGTCAAGTCCAGTTCTACGGGCTCT | |
| | | CGGAGAATGACGAGTGGACCCAGGATAGGGCCAAACCCGTCACCCAGATCGTCAG | |
| | | CGCCGAGGCCTGGGGTAGAGCAGACTGTGGCTTTACCTCGGTGTCCTACCAGCAA | |
| | | GGGGTCCTGTCTGCCACCATCCTCTATGAGATCCTGCTAGGGAAGGCCACCCTGT | |
| | | ATGCTGTGCTGGTCAGCGCCCTIGTGTTGATGGCCATGGTCAAGAGAAAGGATTT | |
| | | C | |
| | β-4 (with | ATGGGCTCCTGGACCCTCTGCTGTGTGTCCCTTTGCATCCTGGTAGCAAAGCACA | SEQ ID NO: 299 |
| | TRBC2) | CAGATGCTGGAGTTATCCAGTCACCCCGGCACGAGGTGACAGAGATGGGACAAGA | |
| | | AGTGACTCTGAGATGTAAACCAATTTCAGGACACGACTACCTTTTCTGGTACAGA | |
| | | CAGACCATGATGCGGGGACTGGAGTTGCTCATTTACTTTAACAACAACGTTCCGA | |
| | | TAGATGATTCAGGGATGCCCGAGGATCGATTCTCAGCTAAGATGCCTAATGCATC | |
| | | ATTCTCCACTCTGAAGATCCAGCCCTCAGAACCCAGGGACTCAGCTGTGTACTTC | |
| | | TGTGCCAGCAGTTTAGGACTGAGCATTTCCCAAGAGACCCAGTACTTCGGGCCAG | |
| | | GCACGCGGCTCCTGGTGCTCGAGGACCTGAAAAACGTGTTCCCACCCGAGGTCGC | |
| | | TGTGTTTGAGCCATCAGAAGCAGAGATCTCCCACACCCAAAAGGCCACACTGGTG | |
| | | TGCCTGGCCACAGGCTTCTACCCCGACCACGTGGAGCTGAGCTGGTGGGTGAATG | |
| | | GGAAGGAGGTGCACAGTGGGGTCAGCACAGACCCGCAGCCCCTCAAGGAGCAGCC | |
| | | CGCCCTCAATGACTCCAGATACTGCCTGAGCAGCCGCCTGAGGGTCTCGGCCACC | |
| | | TTCTGGCAGAACCCCCGCAACCACTTCCGCTGTCAAGTCCAGTTCTACGGGCTCT | |
| | | CGGAGAATGACGAGTGGACCCAGGATAGGGCCAAACCTGTCACCCAGATCGTCAG | |
| | | CGCCGAGGCCTGGGGTAGAGCAGACTGTGGCTTCACCTCCGAGTCTTACCAGCAA | |
| | | GGGGTCCTGTCTGCCACCATCCTCTATGAGATCTTGCTAGGGAAGGCCACCTTGT | |
| | | ATGCCGTGCTGGTCAGTGCCCTCGTGCTGATGGCCATGGTCAAGAGAAAGGATTC | |
| | | CAGAGGC | |
| HD8 | α (with | atggtgaagatccggcaatttttgttggctattttgtggcttcagctaagctgtg | SEQ ID NO: 233 |
| | TRAC) | taagtgccgccaaaaatgaagtggagcagagtcctcagaacctgactgcccagga | |
| | | aggagaatttatcacaatcaactgcagttactcggtaggaataagtgccttacac | |
| | | tggctgcaacagcatccaggaggaggcattgtttccttgtttatgctgagctcag | |
| | | ggaagaagaagcatggaagattaattgccacaataaacatacaggaaaagcacag | |
| | | ctccctgcacatcacagcctcccatcccagagactctgccgtctacatctgtgct | |
| | | gtcacagtcggaaacaaactggtctttggcgcaggaaccattctgagagtcaagt | |
| | | cctatatccagaaccctgaccctgccgtgtaccagctgagagactctaaatccag | |
| | | tgacaagtctgtctgcctattcaccgattttgattctcaaacaaatgtgtcacaa | |
| | | agtaaggattctgatgtgtatatcacagacaaaactgtgctagacatgaggtcta | |
| | | tggacttcaagagcaacagtgctgtggcctggagcaacaaatctgactttgcatg | |
| | | tgcaaacgccttcaacaacagcattattccagaagacaccttcttccccagccca | |
| | | gaaagttcctgtgatgtcaagctggtcgagaaaagctttgaaacagatacgaacc | |
| | | taaactttcaaaacctgtcagtgattgggttccgaatcctcctcctgaaagtggc | |
| | | cgggtttaatctgctcatgacgctgcggctgtggtccagc | |
| | β (with | atgagcatcgggctcctgtgctgtgtggccttttctctcctgtgggcaagtccag | SEQ ID NO: 234 |
| | TRBC1) | tgaatgctggtgtcactcagaccccaaaattccaggtcctgaagacaggacagag | |
| | | catgacactgcagtgtgcccaggatatgaaccataactccatgtactggtatcga | |
| | | caagacccaggcatgggactgaggctgatttattactcagcttctgagggtacca | |
| | | ctgacaaaggagaagtccccaatggctacaatgtctccagattaaacaaacggga | |
| | | gttctcgctcaggctggagtcggctgctccctcccagacatctgtgtacttctgt | |
| | | gccagcagggggtggcgtgagcagttcttcgggccagggacacggctcaccgtgc | |
| | | tagaggacctgaacaaggtgttcccacccgaggtcgctgtgtttgagccatcaga | |
| | | agcagagatctcccacacccaaaaggccacactggtgtgcctggccacaggcttc | |
| | | ttccccgaccacgtggagctgagctggtgggtgaatgggaaggaggtgcacagtg | |
| | | gggtcagcacggacccgcagcccctcaaggagcagcccgccctcaatgactccag | |
| | | atactgcctgagcagccgcctgagggtctcggccaccttctggcagaacccccgc | |
| | | aaccacttccgctgtcaagtccagttctacgggctctcggagaatgacgagtgga | |
| | | cccaggatagggccaaacccgtcacccagatcgtcagcgccgaggcctggggtag | |
| | | agcagactgtggctttacctcggtgtcctaccagcaaggggtcctgtctgccacc | |
| | | atcctctatgagatcctgctagggaaggccaccctgtatgctgtgctggtcagcg | |
| | | cccttgtgttgatggccatggtcaagagaaaggatttc | |
| | β (with | atgagcatcgggctcctgtgctgtgtggccttttctctcctgtgggcaagtccag | SEQ ID NO: 235 |
| | TRBC2) | tgaatgctggtgtcactcagaccccaaaattccaggtcctgaagacaggacagag | |
| | | catgacactgcagtgtgcccaggatatgaaccataactccatgtactggtatcga | |
| | | caagacccaggcatgggactgaggctgatttattactcagcttctgagggtacca | |
| | | ctgacaaaggagaagtccccaatggctacaatgtctccagattaaacaaacggga | |
| | | gttctcgctcaggctggagtcggctgctccctcccagacatctgtgtacttctgt | |
| | | gccagcagggggtggcgtgagcagttcttcgggccagggacacggctcaccgtgc | |
| | | tagaggacctgaaaaacgtgttcccacccgaggtcgctgtgtttgagccatcaga | |
| | | agcagagatctcccacacccaaaaggccacactggtgtgcctggccacaggcttc | |
| | | taccccgaccacgtggagctgagctggtgggtgaatgggaaggaggtgcacagtg | |
| | | gggtcagcacagacccgcagcccctcaaggagcagcccgccctcaatgactccag | |
| | | atactgcctgagcagccgcctgagggtctcggccaccttctggcagaacccccgc | |
| | | aaccacttccgctgtcaagtccagttctacgggctctcggagaatgacgagtgga | |
| | | cccaggatagggccaaacctgtcacccagatcgtcagcgccgaggcctggggtag | |
| | | agcagactgtggcttcacctccgagtcttaccagcaaggggtcctgtctgccacc | |
| | | atcctctatgagatcttgctagggaaggccaccttgtatgccgtgctggtcagtg | |
| | | ccctcgtgctgatggccatggtcaagagaaaggattccagaggc | |
| HD9 | α1 (with | atggtgaagatccggcaatttttgttggctattttgtggcttcagctaagctgtg | SEQ ID NO: 236 |
| | TRAC) | taagtgccgccaaaaatgaagtggagcagagtcctcagaacctgactgcccagga | |
| | | aggagaatttatcacaatcaactgcagttactcggtaggaataagtgccttacac | |
| | | tggctgcaacagcatccaggaggaggcattgtttccttgtttatgctgagctcag | |
| | | ggaagaagaagcatggaagattaattgccacaataaacatacaggaaaagcacag | |
| | | ctccctgcacatcacagcctcccatcccagagactctgccgtctacatctgtgct | |
| | | gcccgatcttataacaccgacaagctcatctttgggactgggaccagattacaag | |
| | | tctttccaaatatccagaaccctgaccctgccgtgtaccagctgagagactctaa | |
| | | atccagtgacaagtctgtctgcctattcaccgattttgattctcaaacaaatgtg | |
| | | tcacaaagtaaggattctgatgtgtatatcacagacaaaactgtgctagacatga | |
| | | ggtctatggacttcaagagcaacagtgctgtggcctggagcaacaaatctgactt | |
| | | tgcatgtgcaaacgccttcaacaacagcattattccagaagacaccttcttcccc | |
| | | agcccagaaagttcctgtgatgtcaagctggtcgagaaaagctttgaaacagata | |
| | | cgaacctaaactttcaaaacctgtcagtgattgggttccgaatcctcctcctgaa | |
| | | agtggccgggtttaatctgctcatgacgctgcggctgtggtccagc | |
| | α2 (with | atggccatgctcctgggggcatcagtgctgattctgtggcttcagccagactggg | SEQ ID NO: 237 |
| | TRAC) | taaacagtcaacagaagaatgatgaccagcaagttaagcaaaattcaccatccct | |
| | | gagcgtccaggaaggaagaatttctattctgaactgtgactatactaacagcatg | |
| | | tttattatttcctatggtacaaaaaataccctgctgaaggtcctacattcctga | |
| | | tatctataagttccattaaggataaaaatgaagatggaagattcactgtcttctt | |
| | | aaacaaaagtgccaagcacctctctctgcacattgtgccctcccagcctggagac | |
| | | tctgcagtgtacttctgtgcagcaagttacaacaatgccagactcatgtttggag | |
| | | atggaactcagctggtggtgaagcccaatatccagaaccctgaccctgccgtgta | |
| | | ccagctgagagactctaaatccagtgacaagtctgtctgcctattcaccgatttt | |
| | | gattctcaaacaaatgtgtcacaaagtaaggattctgatgtgtatatcacagaca | |
| | | aaactgtgctagacatgaggtctatggacttcaagagcaacagtgctgtggcctg | |
| | | gagcaacaaatctgactttgcatgtgcaaacgccttcaacaacagcattattcca | |
| | | gaagacaccttcttccccagcccagaaagttcctgtgatgtcaagctggtcgaga | |
| | | aaagctttgaaacagatacgaacctaaactttcaaaacctgtcagtgattgggtt | |
| | | ccgaatcctcctcctgaaagtggccgggtttaatctgctcatgacgctgcggctg | |
| | | tggtccagc | |
| | α3 (with | atggccatgctcctgggggcatcagtgctgattctgtggcttcagccagactggg | SEQ ID NO: 238 |
| | TRAC) | taaacagtcaacagaagaatgatgaccagcaagttaagcaaaattcaccatccct | |
| | | gagcgtccaggaaggaagaatttctattctgaactgtgactatactaacagcatg | |
| | | tttgattatttcctatggtacaaaaaataccctgctgaaggtcctacattcctga | |
| | | tatctataagttccattaaggataaaaatgaagatggaagattcactgtcttctt | |
| | | aaacaaaagtgccaagcacctctctctgcacattgtgccctcccagcctggagac | |
| | | tctgcagtgtacttctgtgcagcaagttacaacaatgccagactcatgtttggag | |
| | | atggaactcagctggtggtgaagcccaatatccagaaccctgaccctgccgtgta | |
| | | ccagctgagagactctaaatccagtgacaagtctgtctgcctattcaccgatttt | |
| | | gattctcaaacaaatgtgtcacaaagtaaggattctgatgtgtatatcacagaca | |
| | | aaactgtgctagacatgaggtctatggacttcaagagcaacagtgctgtggcctg | |
| | | gagcaacaaatctgactttgcatgtgcaaacgccttcaacaacagcattattcca | |
| | | gaagacaccttcttccccagcccagaaagttcctgtgatgtcaagctggtcgaga | |
| | | aaagctttgaaacagatacgaacctaaactttcaaaacctgtcagtgattgggtt | |
| | | ccgaatcctcctcctgaaagtggccgggtttaatctgctcatgacgctgcggctg | |
| | | tggtccagc | |
| | β1 (with | atgggacccaggctcctcttctgggcactgctttgtctcctcggaacaggcccag | SEQ ID NO: 239 |
| | TRBC1) | tggaggctggagtcacacaaagtcccacacacctgatcaaaacgagaggacagca | |
| | | agcgactctgagatgctctcctatctctgggcacaccagtgtgtactggtaccaa | |
| | | caggccctgggtctgggcctccagttcctcctttggtatgacgagggtgaagaga | |
| | | gaaacagaggaaacttccctcctagattttcaggtcgccagttccctaattatag | |
| | | ctctgagctgaatgtgaacgccttggagctggaggactcggccctgtatctctgt | |
| | | gccagcagctgggggtaccaagagacccagtacttcgggccaggcacgcggctcc | |
| | | tggtgctcgaggacctgaacaaggtgttcccacccgaggtcgctgtgtttgagcc | |
| | | atcagaagcagagatctcccacacccaaaaggccacactggtgtgcctggccaca | |
| | | ggcttcttccccgaccacgtggagctgagctggtgggtgaatgggaaggaggtgc | |
| | | acagtggggtcagcacggacccgcagcccctcaaggagcagcccgccctcaatga | |
| | | ctccagatactgcctgagcagccgcctgagggtctcggccaccttctggcagaac | |
| | | ccccgcaaccacttccgctgtcaagtccagttctacgggctctcggagaatgacg | |
| | | agtggacccaggatagggccaaacccgtcacccagatcgtcagcgccgaggcctg | |
| | | gggtagagcagactgtggctttacctcggtgtcctaccagcaaggggtcctgtct | |
| | | gccaccatcctctatgagatcctgctagggaaggccaccctgtatgctgtgctgg | |
| | | tcagcgcccttgtgttgatggccatggtcaagagaaaggatttc | |
| | β1 (with | atgggacccaggctcctcttctgggcactgctttgtctcctcggaacaggcccag | SEQ ID NO: 240 |
| | TRBC2) | tggaggctggagtcacacaaagtcccacacacctgatcaaaacgagaggacagca | |
| | | agcgactctgagatgctctcctatctctgggcacaccagtgtgtactggtaccaa | |
| | | caggccctgggtctgggcctccagttcctcctttggtatgacgagggtgaagaga | |
| | | gaaacagaggaaacttccctcctagattttcaggtcgccagttccctaattatag | |
| | | ctctgagctgaatgtgaacgccttggagctggaggactcggccctgtatctctgt | |
| | | gccagcagctgggggtaccaagagacccagtacttcgggccaggcacgcggctcc | |
| | | tggtgctcgaggacctgaaaaacgtgttcccacccgaggtcgctgtgtttgagcc | |
| | | atcagaagcagagatctcccacacccaaaaggccacactggtgtgcctggccaca | |
| | | ggcttctaccccgaccacgtggagctgagctggtgggtgaatgggaaggaggtgc | |
| | | acagtggggtcagcacagacccgcagcccctcaaggagcagcccgccctcaatga | |
| | | ctccagatactgcctgagcagccgcctgagggtctcggccaccttctggcagaac | |
| | | ccccgcaaccacttccgctgtcaagtccagttctacgggctctcggagaatgacg | |
| | | agtggacccaggatagggccaaacctgtcacccagatcgtcagcgccgaggcctg | |
| | | gggtagagcagactgtggcttcacctccgagtcttaccagcaaggggtcctgtct | |
| | | gccaccatcctctatgagatcttgctagggaaggccaccttgtatgccgtgctgg | |
| | | tcagtgccctcgtgctgatggccatggtcaagagaaaggattccagaggc | |
| | β2 (with | atggacaccagagtactctgctgtgcggtcatctgtcttctgggggcaggtctct | SEQ ID NO: 241 |
| | TRBC1) | caaatgccggcgtcatgcagaacccaagacacctggtcaggaggaggggacagga | |
| | | ggcaagactgagatgcagcccaatgaaaggacacagtcatgtttactggtatcgg | |
| | | cagctcccagaggaaggtctgaaattcatggtttatctccagaaagaaaatatca | |
| | | tagatgagtcaggaatgccaaaggaacgattttctgctgaatttcccaaagaggg | |
| | | ccccagcatcctgaggatccagcaggtagtgcgaggagattcggcagcttatttc | |
| | | tgtgccagctcaccgacaggtggcgagtactatggctacaccttcggttcgggga | |
| | | ccaggttaaccgttgtagaggacctgaacaaggtgttcccacccgaggtcgctgt | |
| | | gtttgagccatcagaagcagagatctcccacacccaaaaggccacactggtgtgc | |
| | | ctggccacaggcttcttccccgaccacgtggagctgagctggtgggtgaatggga | |
| | | aggaggtgcacagtggggtcagcacggacccgcagcccctcaaggagcagcccgc | |
| | | cctcaatgactccagatactgcctgagcagccgcctgagggtctcggccaccttc | |
| | | tggcagaacccccgcaaccacttccgctgtcaagtccagttctacgggctctcgg | |
| | | agaatgacgagtggacccaggatagggccaaacccgtcacccagatcgtcagcgc | |
| | | cgaggcctggggtagagcagactgtggctttacctcggtgtcctaccagcaaggg | |
| | | gtcctgtctgccaccatcctctatgagatcctgctagggaaggccaccctgtatg | |
| | | ctgtgctggtcagcgcccttgtgttgatggccatggtcaagagaaaggatttc | |
| | β2 (with | atggacaccagagtactctgctgtgcggtcatctgtcttctgggggcaggtctct | SEQ ID NO: 242 |
| | TRBC2) | caaatgccggcgtcatgcagaacccaagacacctggtcaggaggaggggacagga | |
| | | ggcaagactgagatgcagcccaatgaaaggacacagtcatgtttactggtatcgg | |
| | | cagctcccagaggaaggtctgaaattcatggtttatctccagaaagaaaatatca | |
| | | tagatgagtcaggaatgccaaaggaacgattttctgctgaatttcccaaagaggg | |
| | | ccccagcatcctgaggatccagcaggtagtgcgaggagattcggcagcttatttc | |
| | | tgtgccagctcaccgacaggtggcgagtactatggctacaccttcggttcgggga | |
| | | ccaggttaaccgttgtagaggacctgaaaaacgtgttcccacccgaggtcgctgt | |
| | | gtttgagccatcagaagcagagatctcccacacccaaaaggccacactggtgtgc | |
| | | ctggccacaggcttctaccccgaccacgtggagctgagctggtgggtgaatggga | |
| | | aggaggtgcacagtggggtcagcacagacccgcagcccctcaaggagcagcccgc | |
| | | cctcaatgactccagatactgcctgagcagccgcctgagggtctcggccaccttc | |
| | | tggcagaacccccgcaaccacttccgctgtcaagtccagttctacgggctctcgg | |
| | | agaatgacgagtggacccaggatagggccaaacctgtcacccagatcgtcagcgc | |
| | | cgaggcctggggtagagcagactgtggcttcacctccgagtcttaccagcaaggg | |
| | | gtcctgtctgccaccatcctctatgagatcttgctagggaaggccaccttgtatg | |
| | | ccgtgctggtcagtgccctcgtgctgatggccatggtcaagagaaaggattccag | |
| | | aggc | |
| | β3 (with | atgagcatcggcctcctgtgctgtgcagccttgtctctcctgtgggcaggtccag | SEQ ID NO: 243 |
| | TRBC1) | tgaatgctggtgtcactcagaccccaaaattccaggtcctgaagacaggacagag | |
| | | catgacactgcagtgtgcccaggatatgaaccatgaatacatgtcctggtatcga | |
| | | caagacccaggcatggggctgaggctgattcattactcagttggtgctggtatca | |
| | | ctgaccaaggagaagtccccaatggctacaatgtctccagatcaaccacagagga | |
| | | tttcccgctcaggctgctgtcggctgctccctcccagacatctgtgtacttctgt | |
| | | gccagcagttcatacccccttcggacagggcgatacaactcctataattcacccc | |
| | | tccactttgggaacgggaccaggctcactgtgacagaggacctgaacaaggtgtt | |
| | | cccacccgaggtcgctgtgtttgagccatcagaagcagagatctcccacacccaa | |
| | | aaggccacactggtgtgcctggccacaggcttcttccccgaccacgtggagctga | |
| | | gctggtgggtgaatgggaaggaggtgcacagtggggtcagcacggacccgcagcc | |
| | | cctcaaggagcagcccgccctcaatgactccagatactgcctgagcagccgcctg | |
| | | agggtctcggccaccttctggcagaacccccgcaaccacttccgctgtcaagtcc | |
| | | agttctacgggctctcggagaatgacgagtggacccaggatagggccaaacccgt | |
| | | cacccagatcgtcagcgccgaggcctggggtagagcagactgtggctttacctcg | |
| | | gtgtcctaccagcaaggggtcctgtctgccaccatcctctatgagatcctgctag | |
| | | ggaaggccaccctgtatgctgtgctggtcagcgcccttgtgttgatggccatggt | |
| | | caagagaaaggatttctga | |
| | β3 (with | atgagcatcggcctcctgtgctgtgcagccttgtctctcctgtgggcaggtccag | SEQ ID NO: 244 |
| | TRBC2) | tgaatgctggtgtcactcagaccccaaaattccaggtcctgaagacaggacagag | |
| | | catgacactgcagtgtgcccaggatatgaaccatgaatacatgtcctggtatcga | |
| | | caagacccaggcatggggctgaggctgattcattactcagttggtgctggtatca | |
| | | ctgaccaaggagaagtccccaatggctacaatgtctccagatcaaccacagagga | |
| | | tttcccgctcaggctgctgtcggctgctccctcccagacatctgtgtacttctgt | |
| | | gccagcagttcatacccccttcggacagggcgatacaactcctataattcacccc | |
| | | tccactttgggaacgggaccaggctcactgtgacagaggacctgaaaaacgtgtt | |
| | | cccacccgaggtcgctgtgtttgagccatcagaagcagagatctcccacacccaa | |
| | | aaggccacactggtgtgcctggccacaggcttctaccccgaccacgtggagctga | |
| | | gctggtgggtgaatgggaaggaggtgcacagtggggtcagcacagacccgcagcc | |
| | | cctcaaggagcagcccgccctcaatgactccagatactgcctgagcagccgcctg | |
| | | agggtctcggccaccttctggcagaacccccgcaaccacttccgctgtcaagtcc | |
| | | agttctacgggctctcggagaatgacgagtggacccaggatagggccaaacctgt | |
| | | cacccagatcgtcagcgccgaggcctggggtagagcagactgtggcttcacctcc | |
| | | gagtcttaccagcaaggggtcctgtctgccaccatcctctatgagatcttgctag | |
| | | ggaaggccaccttgtatgccgtgctggtcagtgccctcgtgctgatggccatggt | |
| | | caagagaaaggattccagaggc | |
| HD10 | α (with | atggccatgctcctgggggcatcagtgctgattctgtggcttcagccagactggg | SEQ ID NO: 245 |
| | TRAC) | taaacagtcaacagaagaatgatgaccagcaagttaagcaaaattcaccatccct | |
| | | gagcgtccaggaaggaagaatttctattctgaactgtgactatactaacagcatg | |
| | | tttgattatttcctatggtacaaaaaataccctgctgaaggtcctacattcctga | |
| | | tatctataagttccattaaggataaaaatgaagatggaagattcactgtcttctt | |
| | | aaacaaaagtgccaagcacctctctctgcacattgtgccctcccagcctggagac | |
| | | tctgcagtgtacttctgtgcagcaagcggaggaagagatgacaagatcatctttg | |
| | | gaaaagggacacgacttcatattctccccaatatccagaaccctgaccctgccgt | |
| | | gtaccagctgagagactctaaatccagtgacaagtctgtctgcctattcaccgat | |
| | | tttgattctcaaacaaatgtgtcacaaagtaaggattctgatgtgtatatcacag | |
| | | acaaaactgtgctagacatgaggtctatggacttcaagagcaacagtgctgtggc | |
| | | ctggagcaacaaatctgactttgcatgtgcaaacgccttcaacaacagcattatt | |
| | | ccagaagacaccttcttccccagcccagaaagttcctgtgatgtcaagctggtcg | |
| | | agaaaagctttgaaacagatacgaacctaaactttcaaaacctgtcagtgattgg | |
| | | gttccgaatcctcctcctgaaagtggccgggtttaatctgctcatgacgctgcgg | |
| | | ctgtggtccagc | |
| | β (with | atgagcatcggcctcctgtgctgtgcagccttgtctctcctgtgggcaggtccag | SEQ ID NO: 246 |
| | TRBC1) | tgaatgctggtgtcactcagaccccaaaattccaggtcctgaagacaggacagag | |
| | | catgacactgcagtgtgcccaggatatgaaccatgaatacatgtcctggtatcga | |
| | | caagacccaggcatggggctgaggctgattcattactcagttggtgctggtatca | |
| | | ctgaccaaggagaagtccccaatggctacaatgtctccagatcaaccacagagga | |
| | | tttcccgctcaggctgctgtcggctgctccctcccagacatctgtgtacttctgt | |
| | | gccagcagctactcccggacagagagcacagatacgcagtattttggcccaggca | |
| | | cccggctgacagtgctcgaggacctgaacaaggtgttcccacccgaggtcgctgt | |
| | | gtttgagccatcagaagcagagatctcccacacccaaaaggccacactggtgtgc | |
| | | ctggccacaggcttcttccccgaccacgtggagctgagctggtgggtgaatggga | |
| | | aggaggtgcacagtggggtcagcacggacccgcagcccctcaaggagcagcccgc | |
| | | cctcaatgactccagatactgcctgagcagccgcctgagggtctcggccaccttc | |
| | | tggcagaacccccgcaaccacttccgctgtcaagtccagttctacgggctctcgg | |
| | | agaatgacgagtggacccaggatagggccaaacccgtcacccagatcgtcagcgc | |
| | | cgaggcctggggtagagcagactgtggctttacctcggtgtcctaccagcaaggg | |
| | | gtcctgtctgccaccatcctctatgagatcctgctagggaaggccaccctgtatg | |
| | | ctgtgctggtcagcgcccttgtgttgatggccatggtcaagagaaaggatttc | |
| | β (with | atgagcatcggcctcctgtgctgtgcagccttgtctctcctgtgggcaggtccag | SEQ ID NO: 247 |
| | TRBC2) | tgaatgctggtgtcactcagaccccaaaattccaggtcctgaagacaggacagag | |
| | | catgacactgcagtgtgcccaggatatgaaccatgaatacatgtcctggtatcga | |
| | | caagacccaggcatggggctgaggctgattcattactcagttggtgctggtatca | |
| | | ctgaccaaggagaagtccccaatggctacaatgtctccagatcaaccacagagga | |
| | | tttcccgctcaggctgctgtcggctgctccctcccagacatctgtgtacttctgt | |
| | | gccagcagctactcccggacagagagcacagatacgcagtattttggcccaggca | |
| | | cccggctgacagtgctcgaggacctgaaaaacgtgttcccacccgaggtcgctgt | |
| | | gtttgagccatcagaagcagagatctcccacacccaaaaggccacactggtgtgc | |
| | | ctggccacaggcttctaccccgaccacgtggagctgagctggtgggtgaatggga | |
| | | aggaggtgcacagtggggtcagcacagacccgcagcccctcaaggagcagcccgc | |
| | | cctcaatgactccagatactgcctgagcagccgcctgagggtctcggccaccttc | |
| | | tggcagaacccccgcaaccacttccgctgtcaagtccagttctacgggctctcgg | |
| | | agaatgacgagtggacccaggatagggccaaacctgtcacccagatcgtcagcgc | |
| | | cgaggcctggggtagagcagactgtggcttcacctccgagtcttaccagcaaggg | |
| | | gtcctgtctgccaccatcctctatgagatcttgctagggaaggccaccttgtatg | |
| | | ccgtgctggtcagtgccctcgtgctgatggccatggtcaagagaaaggattccag | |
| | | aggc | |

Accordingly, the present invention provides an isolated polynucleotide comprising one or more nucleotide sequences selected from the group consisting of SEQ ID NOs: 132-156, 223-247 and 292-299, or variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

The present invention also provides a TCR comprising an α chain encoded by a nucleotide sequence selected from the group consisting of SEQ ID NOs: 132, 135, 138, 141, 144, 147, 152, 223, 226, 227, 228, 233, 236, 237, 238, 245, 292, 295, and variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

The present invention also provides a TCR comprising a β chain encoded by a nucleotide sequence selected from the group consisting of SEQ ID Nos: 133, 134, 136, 137, 139, 140, 142, 143, 145, 146, 148, 149, 150, 151, 153, 154, 155, 156, 224, 225, 229, 230, 231, 232, 234, 235, 239, 240, 241, 242, 243, 244, 246, 247, 293, 294, 296-299, and variants thereof having at least 40%, at least 50%, at least 60%, at least 70%, at least 75%, at least 80%, at least 85%, at least 90%, at least 95%, at least 96%, at least 97%, at least 98%, or at least 99% sequence identity thereto.

Further provided by the present invention are isolated polynucleotide sequences derived from the sequences present in Table 2. For example, the present invention provides an isolated polynucleotide encoding a variable region of a TCR according to the present invention, wherein the isolated polynucleotide comprises a stretch of nucleotides of any one of SEQ ID Nos: 132-156, 223-247 and 292-299.

The variant sequences may have additions, deletions or substitutions, of one or more bases. If the variation involves addition(s) or deletion(s) they may either occur in threes or be balanced (i.e. an addition for each deletion) so that the variation does not cause a frame-shift for translation of the remainder of the sequence.

Some or all of the variations may be “silent” in the sense that they do not affect the sequence of the encoded protein due to the degeneracy of the genetic code.

Some or all of the variations may produce conservative amino acid substitutions, additions or deletions as explained above. The variation may be concentrated in one or more regions, such as the regions encoding the constant regions, the linker, or the framework regions of the α or β chains, or they may be spread throughout the molecule.

The variant sequence should retain the capacity to encode all or part of a TCR amino acid sequence which binds to a WT1 peptide.

Codon Optimisation

The polynucleotides used in the present invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Codon optimisation may also involve the removal of mRNA instability motifs and cryptic splice sites.

Vector

The present invention provides a vector comprising a polynucleotide described herein.

A vector is a tool that allows or facilitates the transfer of an entity from one environment to another. In accordance with the present invention, and by way of example, some vectors used in recombinant nucleic acid techniques allow entities, such as a segment of nucleic acid (e.g. a heterologous DNA segment, such as a heterologous Cdna segment), to be transferred into a target cell. The vector may serve the purpose of maintaining the heterologous nucleic acid (DNA or RNA) within the cell, facilitating the replication of the vector comprising a segment of nucleic acid, or facilitating the expression of the protein encoded by a segment of nucleic acid.

Vectors may be non-viral or viral. Examples of vectors used in recombinant nucleic acid techniques include, but are not limited to, plasmids, chromosomes, artificial chromosomes and viruses. The vector may be single stranded or double stranded. It may be linear and optionally the vector comprises one or more homology arms. The vector may also be, for example, a naked nucleic acid (e.g. DNA). In its simplest form, the vector may itself be a nucleotide of interest.

The vectors used in the invention may be, for example, plasmid or virus vectors and may include a promoter for the expression of a polynucleotide and optionally a regulator of the promoter.

Vectors comprising polynucleotides used in the invention may be introduced into cells using a variety of techniques known in the art, such as transformation, transfection and transduction. Several techniques are known in the art, for example transduction with recombinant viral vectors, such as retroviral, lentiviral, adenoviral, adeno-associated viral, baculoviral and herpes simplex viral vectors, Sleeping Beauty vectors; direct injection of nucleic acids and biolistic transformation.

Non-viral delivery systems include but are not limited to DNA transfection methods. Here, transfection includes a process using a non-viral vector to deliver a gene to a target cell. Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nature Biotechnology 1996 14; 556) and combinations thereof.

The term “transfection” is to be understood as encompassing the delivery of polynucleotides to cells by both viral and non-viral delivery.

In addition, the invention may employ gene targeting protocols, for example the delivery of DNA-modifying agents.

The term “vector” includes an expression vector i.e. a construct capable of in vivo or in vitro/ex vivo expression. Expression may be controlled by a vector sequence, or, for example in the case of insertion at a target site, expression may be controlled by a target sequence. A vector may be integrated or tethered to the cell's DNA.

Viral delivery systems include but are not limited to adenovirus vector, an adeno-associated viral (AAV) vector, a herpes viral vector, a retroviral vector, a lentiviral vector, and a baculoviral vector.

Retroviruses are RNA viruses with a life cycle different to that of lytic viruses. In this regard, a retrovirus is an infectious entity that replicates through a DNA intermediate. When a retrovirus infects a cell, its genome is converted to a DNA form by a reverse transcriptase enzyme. The DNA copy serves as a template for the production of new RNA genomes and virally encoded proteins necessary for the assembly of infectious viral particles.

There are many retroviruses, for example murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses.

A detailed list of retroviruses may be found in Coffin et al (“Retroviruses” 1997 Cold Spring Harbour Laboratory Press Eds: J M Coffin, SM Hughes, HE Varmus pp 758-763).

Lentiviruses also belong to the retrovirus family, but they can infect both dividing and non-dividing cells (Lewis et al (1992) EMBO J. 3053-3058).

The vector may be capable of transferring a nucleotide sequence encoding a WT1-specific TCR described herein to a cell, such as a T-cell, such that the cell expresses the WT1-specific TCR. Preferably the vector will be capable of sustained high-level expression in T-cells, so that the introduced TCR may compete successfully with the endogenous TCR for a limited pool of CD3 molecules.

Increasing the supply of CD3 molecules may increase TCR expression, for example, in a cell that has been modified to express the TCRs of the present invention. Accordingly, the vector of the present invention may further comprise one or more genes encoding CD3-gamma, CD3-delta, CD3-epsilon and/or CD3-zeta. In one embodiment, the vector of the present invention comprises a gene encoding CD3-zeta. The vector may comprise a gene encoding CD8. The vector may encode a selectable marker or a suicide gene, to increase the safety profile of the genetically engineered cell, e.g. a cell of the present invention, or a cell that has been modified to express the TCRs of the present invention (Bonini, Science 1997, Ciceri, Bonini Lancet Oncol. 2009, Oliveira et al., STM 2015). The genes comprised in the vector of the present invention may be linked by self-cleaving sequences, such as the 2A self-cleaving sequence.

Alternatively one or more separate vectors encoding a CD3 gene may be provided for co-transfer to a cell simultaneously, sequentially or separately with one or more vectors of the present invention, e.g. one or more vectors encoding TCRs of the present invention.

Cell

The present invention relates to a cell comprising a polynucleotide or a vector according to the present invention.

The cell may be a T-cell, a lymphocyte, or a stem cell. The T-cell, the lymphocyte, or the stem cell may be selected from the group consisting of CD4 cells, CD8 cells, naive T-cells, memory stem T-cells, central memory T-cells, double negative T-cells, effector memory T-cells, effector T-cells, Th0 cells, Tc0 cells, Th1 cells, Tc1 cells, Th2 cells, Tc2 cells, Th17 cells, Th22 cells, gamma/delta T-cells, natural killer (NK) cells, natural killer T (NKT) cells, hematopoietic stem cells and pluripotent stem cells.

The type of cell may be selected in order to provide desirable and advantageous in vivo persistence and to provide desirable and advantageous functions and characteristics to the cells of present invention.

The cell may have been isolated from a subject.

The cell of the present invention may be provided for use in adoptive cell transfer. As used herein the term “adoptive cell transfer” refers to the administration of a cell population to a patient. Typically, the cells are T-cells isolated from a subject and then genetically modified and cultured in vitro in order to express a TCR of the present invention before being administered to the patient.

Adoptive cell transfer may be allogenic or autologous.

By “autologous cell transfer” it is to be understood that the starting population of cells (which are then transduced according to a method of the invention, or are transduced with a vector according to the present invention) is obtained from the same subject as that to which the transduced T-cell population is administered. Autologous transfer is advantageous as it avoids problems associated with immunological incompatibility and are available to subjects irrespective of the availability of a genetically matched donor.

By “allogeneic cell transfer” is to be understood that the starting population of cells (which are then transduced according to a method of the invention, or are transduced with a vector according to the present invention) is obtained from a different subject as that to which the transduced cell population is administered. Preferably, the donor will be genetically matched to the subject to which the cells are administered to minimise the risk of immunological incompatibility. Alternatively, the donor may be mismatched and unrelated to the patient.

Suitable doses of transduced cell populations are such as to be therapeutically and/or prophylactically effective. The dose to be administered may depend on the subject and condition to be treated, and may be readily determined by a skilled person.

The cell may be derived from a T-cell isolated from a subject. The T-cell may be part of a mixed cell population isolated from the subject, such as a population of peripheral blood lymphocytes (PBL). T-cells within the PBL population may be activated by methods known in the art, such as using anti-CD3 and/or anti-CD28 antibodies or cell sized beads conjugated with anti-CD3 and/or anti-CD28 antibodies.

The T-cell may be a CD4⁺ helper T cell or a CD8⁺ cytotoxic T cell. The cell may be in a mixed population of CD4⁺ helper T cell/CD8⁺ cytotoxic T-cells. Polyclonal activation, for example using anti-CD3 antibodies optionally in combination with anti-CD28 antibodies will trigger the proliferation of CD4⁺ and CD8⁺ T-cells.

The cell may be isolated from the subject to which the genetically modified cell is to be adoptively transferred. In this respect, the cell may be made by isolating a T-cell from a subject, optionally activating the T-cell, transferring the TCR gene to the cell ex vivo. Subsequent immunotherapy of the subject may then be carried out by adoptive transfer of the TCR-transduced cells. As used herein this process refers to autologous T-cell transfer—i.e. the TCR-transduced cells are administered to the same subject from which the T-cells were originally derived.

Alternatively the T-cell may be isolated from a different subject, such that it is allogeneic. The T-cell may be isolated from a donor subject. For example, if the subject is undergoing allogeneic haematopoietic stem cell transplantation (Allo-HSCT) or solid organ transplantation or cell transplantation or stem cell therapy, the cell may be derived from the donor, from which the organs, tissues or cells are derived. The donor and the subject undergoing treatment may be siblings.

Alternatively the cell may be, or may be derived from, a stem cell, such as a haemopoietic stem cell (HSC). Gene transfer into HSCs does not lead to TCR expression at the cell surface as stem cells do not express CD3 molecules. However, when stem cells differentiate into lymphoid precursors that migrate to the thymus, the initiation of CD3 expression leads to the surface expression of the introduced TCR in thymocytes.

An advantage of this approach is that the mature T-cells, once produced, express only the introduced TCR and little or no endogenous TCR chains, because the expression of the introduced TCR chains suppresses rearrangement of endogenous TCR gene segments to form functional TCR alpha and beta genes. A further benefit is that the gene-modified stem cells are a continuous source of mature T-cells with the desired antigen specificity. The cell may therefore be a gene-modified stem cell, preferably a gene-modified hematopoeitic stem cell, which, upon differentiation, produces a T-cell expressing a TCR of the invention.

Other approaches known in the art may be used to reduce, limit, prevent, silence, or abrogate expression of endogenous genes in the cells of the present invention or cells prepared by the methods of the present invention.

As used herein the term “disrupting” refers to reducing, limiting, preventing, silencing, or abrogating expression of a gene. The person skilled in the art is able to use any method known in the art to disrupt an endogenous gene, e.g., any suitable method for genome editing, gene silencing, gene knock-down or gene knock-out.

For example, an endogenous gene may be disrupted with an artificial nuclease. An artificial nuclease is, e.g., an artificial restriction enzyme engineered to selectively target a specific polynucleotide sequence (e.g. encoding a gene of interest) and induce a double strand break in said polynucleotide sequence. Typically, the double strand break (DSB) will be repaired by error-prone non-homologous end joining (NHEJ) thereby resulting in the formation of a non-functional polynucleotide sequence, which may be unable to express an endogenous gene.

In some embodiments, the artificial nuclease is selected from the group consisting of zinc finger nucleases (ZFN), transcription activator-like effector nucleases (TALEN) and CRISPR/Cas (e.g. CRISPR/Cas9).

The methods of preparing a cell (e.g. a T-cell) of the present invention may comprise the step of targeted integration of a expression cassette into an endogenous gene (e.g. an endogenous TCR α chain gene and/or an endogenous TCR β chain gene). As used herein the term expression cassette refers to a polynucleotide sequence (e.g. a DNA polynucleotide sequence) comprising one or more polynucleotide sequences encoding one or more genes of interest such that said genes of interest are capable of expression. Endogenous sequences may facilitate expression from the expression cassette, and/or transcription control sequences within the expression cassette may facilitate expression. For example, the expression cassette may comprise a polynucleotide sequence of the present invention, or a polynucleotide sequence encoding a TCR of the present invention, operably linked to an expression control sequence, e.g. a promoter or an enhancer sequence. The one or more genes of interest may be located between one or more sets of restriction sites. Suitably, the restriction sites may facilitate the integration of the expression cassette into, e.g., a vector, a plasmid, or genomic DNA (e.g. host cell genomic DNA).

For example, an expression cassette of the present invention may be transferred from a first polynucleotide sequence, e.g. on a vector, to another by ‘cutting’, e.g. excising, the expression cassette using one or more suitable restriction enzymes and ‘pasting’, e.g. integrating, the expression cassette into a second polynucleotide sequence.

The expression cassette may comprise a polynucleotide of the present invention. The expression cassette may comprise a polynucleotide encoding one or more TCRs of the present invention. The expression cassette may further comprise an antibiotic resistance gene or other selectable marker gene that allows cells that have successfully integrated the expression cassette into their DNA to be identified. The polynucleotide sequences comprised in the expression cassette may be operably linked to expression control sequences, e.g. a suitable promoter or enhancer sequence. The person skilled in the art will be able to select suitable expression control sequences.

The present invention also contemplates a cell expressing a TCR of the present invention, which has been engineered to disrupt one or more endogenous MHC genes. Disruption of an endogenous MHC gene can reduce or prevent expression of MHC on the engineered cell surface. Accordingly, such an engineered cell with reduced or no MHC expression will have limited or no capacity to present antigens on its cell surface. Such a cell is particulary advantageous for adoptive cell transfer since the cell will be non-alloreactive, e.g., the cell will not present antigens which could be recognized by the immune system of a subject receiving the adoptively transferred cell. As a result, the transferred cell will not be recognized as ‘non-self’ and an adverse immune reaction to the cell can be avoided. Such a cell is termed a ‘universal cell’ since it is suitable for adoptive transfer to a variety of different hosts regardless of HLA type.

Accordingly, the present invention provides a method of preparing a non-alloreactive universal T-cell, which expresses a TCR of the present invention. Further provided by the present invention is a non-alloreactive universal T-cell, which expresses a TCR of the present invention.

The present invention further contemplates cells which have been engineered to disrupt one more endogenous genes to modify the cell to enhance advantageous properties, characteristics or functions of the cell and/or reduce undesirable properties, characteristics or functions. For example, by disrupting an endogenous cell the persistence, expansion, activity, resistance to exhaustion/senescence/inhibitory signals, homing capacity, or other cell functions may be modified. As used in this context, the term ‘modify’ refers to a change in one or more characteristics relative to an equivalent unmodified cell, e.g. a cell in which an endogenous gene has not been disrupted. For example, the change may be an increase, an enhancement or an introduction of a characteristic or function of the cell relative to an equivalent unmodified cell. Alternatively, the change may be a decrease, suppression or abrogation of a characteristic or function of the cell relative to an equivalent unmodified cell.

The polynucleotides and vectors of the present invention may be transferred into specific T-cell subsets, including CD4 and or CD8, naive, memory stem T cells, central memory, effector memory or effector cells, or in other cellular subsets such as to promote different in vivo length of persistence and function in the cells of the present invention.

The polynucleotides and vectors of the present invention may also be transferred into T-cell subsets such as naïve, memory stem T cells, central memory cells, effector memory cells, effectors.

The polynucleotides and vectors of the present invention may also be transferred into T-cell subsets with different polarizations, such as Th0/Tc0, Th1/Tc1, Th2/Tc2, Th17, Th22 or others, depending on the cytokine background most appropriate to target a particular tumor type.

Furthermore, the polynucleotides and vectors of the present invention encoding the antigen-specific regions of the TCRs of the present invention may be transferred in other cellular subsets, including gamma/delta T-cells, NK cells, NKT cells, hematopoietic stem cells or other cells, in order to obtain the therapeutic effect.

Further provided by the present invention is a method of preparing a cell, which comprises the step of transducing a cell in vitro or ex vivo with a vector of the present invention. Various methods for transduction of a cell with a vector are known in the art (see e.g. Sambrook et al).

The present invention also provides a method of producing a T-cell expressing a TCR of the invention by inducing the differentiation of a stem cell which comprises a polynucleotide or a vector of the present invention.

A population of cells may be purified selectively for cells that exhibit a specific phenotype or characteristic, and from other cells which do not exhibit that phenotype or characteristic, or exhibit it to a lesser degree. For example, a population of cells that expresses a specific marker (e.g. CD3, CD4, CD8, CD25, CD127, CD152, CXCR3, or CCR4) may be purified from a starting population of cells. Alternatively, or in addition, a population of cells that does not express another marker may be purified.

By “enriching” a population of cells for a certain type of cells it is to be understood that the concentration of that type of cells is increased within the population. The concentration of other types of cells may be concomitantly reduced.

Purification or enrichment may result in the population of cells being substantially pure of other types of cell.

Purifying or enriching for a population of cells expressing a specific marker (e.g. CD3, CD4, CD8, CD25, CD127, CD152, CXCR3, or CCR4) may be achieved by using an agent that binds to that marker, preferably substantially specifically to that marker. An agent that binds to a cellular marker may be an antibody, for example antibody which binds to CD3, CD4, CD8, CD25, CD127, CD152, CXCR3, or CCR4.

The term “antibody” refers to complete antibodies or antibody fragments capable of binding to a selected target, and including Fv, ScFv, F(ab′) and F(ab′) 2, monoclonal and polyclonal antibodies, engineered antibodies including chimeric, CDR-grafted and humanised antibodies, and artificially selected antibodies produced using phage display or alternative techniques.

In addition, alternatives to classical antibodies may also be used in the invention, for example “avibodies”, “avimers”, “anticalins”, “nanobodies” and “DARPins”.

The agents that bind to specific markers may be labelled so as to be identifiable using any of a number of techniques known in the art. The agent may be inherently labelled, or may be modified by conjugating a label thereto. By “conjugating” it is to be understood that the agent and label are operably linked. This means that the agent and label are linked together in a manner which enables both to carry out their function (e.g. binding to a marker, allowing fluorescent identification, or allowing separation when placed in a magnetic field) substantially unhindered. Suitable methods of conjugation are well known in the art and would be readily identifiable by the skilled person.

A label may allow, for example, the labelled agent and any cell to which it is bound to be purified from its environment (e.g. the agent may be labelled with a magnetic bead or an affinity tag, such as avidin), detected or both. Detectable markers suitable for use as a label include fluorophores (e.g. green, cherry, cyan and orange fluorescent proteins) and peptide tags (e.g. His tags, Myc tags, FLAG tags and HA tags).

A number of techniques for separating a population of cells expressing a specific marker are known in the art. These include magnetic bead-based separation technologies (e.g. closed-circuit magnetic bead-based separation), flow cytometry, fluorescence-activated cell sorting (FACS), affinity tag purification (e.g. using affinity columns or beads, such as biotin columns to separate avidin-labelled agents) and microscopy-based techniques.

It may also be possible to perform the separation using a combination of different techniques, such as a magnetic bead-based separation step followed by sorting of the resulting population of cells for one or more additional (positive or negative) markers by flow cytometry.

Clinical grade separation may be performed, for example, using the CliniMACS® system (Miltenyi). This is an example of a closed-circuit magnetic bead-based separation technology.

It is also envisaged that dye exclusion properties (e.g. side population or rhodamine labelling) or enzymatic activity (e.g. ALDH activity) may be used to enrich for HSCs.

Chimeric Molecules

In another aspect, the present invention provides a chimeric molecule comprising a TCR of the present invention, a TCR encoded by a polynucleotide of the present invention, or a portion thereof, conjugated to a non-cellular substrate. The conjugation may be covalent or non-covalent.

The non-cellular substrate may be a nanoparticle, an exosome, or any non-cellular substrate known in the art.

The chimeric molecule of the present invention may be soluble.

In another aspect the present invention provides a chimeric molecule comprising a TCR of the present invention, a TCR encoded by a polynucleotide of the present invention, or a portion thereof, conjugated to a toxin or an antibody.

The toxin or antibody may be cytotoxic. The toxin may be a cytotoxic molecule or compound, e.g. a radioactive molecule or compound. The TCR portion of the chimeric molecule may confer the ability to recognize cells expressing WT1 protein or peptides. Thus, the chimeric molecule may specifically recognize and/or bind to WT1-expressing tumor cells. Accordingly, the chimeric molecules of the present invention may provide WT1-targeted delivery of cytotoxic toxins, antibodies and/or compounds.

WT1-Related Diseases

WT1 is widely expressed on a variety of hematological and solid tumors, while showing limited expression on various healthy tissues (e.g. gonads, uterus, kidney, mesothelium, progenitor cells in different tissues). The present inventors have identified and determined the amino acid sequences of TCRs that recognise WT1 peptides. Furthermore, they have demonstrated that T-cells expressing TCRs according to the present invention target and kill cells which present WT1 peptide or overexpress WT1 protein.

Accordingly, the present invention provides a method for treating and/or preventing a disease associated with expression of WT1, which comprises the step of administering a TCR, an isolated polynucleotide, a vector, or a cell of the present invention to a subject in need thereof. The present invention also provides a method for treating and/or preventing a disease associated with expression of WT1, comprises the step of administering a cell prepared by the method of the present invention to a subject in need thereof.

Further provided by the present invention is a TCR of the present invention, an isolated polynucleotide of the present invention, a vector of the present invention, a cell according of the present invention, or a cell prepared by the method of the present invention for use in treating and/or preventing a disease associated with expression of WT1.

The term ‘preventing’ is intended to refer to averting, delaying, impeding or hindering the contraction of the disease. The treatment may, for example, prevent or reduce the likelihood of developing or contracting a disease associated with expression of WT1.

‘Treating’ as used herein refers to caring for a diseased subject, in order to ameliorate, cure or reduce the symptoms of the disease, or in order to reduce, halt or delay the progression of the disease.

The subject may be a human subject. The human subject may be a child. For example, the child may be less than 10 years in age, less than 9 years in age, less than 8 years in age, less than 7 years in age, less than 6 years in age, less than 5 years in age, less than 4 years in age, less than 3 years in age, or less than 2 years in age. The human subject may be an infant.

The subject may have been previously determined to be in need of a TCR, an isolated polynucleotide, a vector, or a cell of the present invention, or a cell prepared by the method of the present invention on the basis of expression of WT1. For example, the subject may have a cell population that exhibits increased expression of WT1 relative to a healthy control cell population. A variety of techniques known in the art may be used to determine WT1 expression—e.g. quantitative RT-PCR can be used to determine the amount of WT1 RNA transcript, which is indicative of WT1 protein expression. The person skilled in the art will also appreciate that WT1 protein expression may be determined by performing western blots using commercially available antibodies specific for WT1.

The subject may also have been previously identified as having an alteration (e.g. mutation or deletion) in a WT1 gene. Such an alteration may be hereditary. Thus, the disease associated with expression of WT1 may be a hereditary disease. Examples of hereditary diseases associated with expression of WT1 include but are not limited to WAGR (Wilms tumor-Aniridia-Genitourinary malformation-Retardation) syndrome, Denys-Drash syndrome (DDS), Frasier syndrome (FS), genitourinary anomalies (abnormalities of the reproductive and urinary systems) syndrome.

Subjects with hereditary diseases associated with expression of WT1 may be at higher risk of developing a proliferative disorder (e.g. a cancer).

The disease associated with expression of WT1 may be a proliferative disorder.

The proliferative disorder may be a hematological malignancy or a solid tumor. The hematological malignancy may be selected from the group consisting of acute myeloid leukemia (AML), chronic myeloid leukemia (CML), lymphoblastic leukemia, myelodisplastic syndromes, lymphoma, multiple myeloma, non Hodgkin lymphoma, and Hodgkin lymphoma.

The solid tumor may be selected from the group consisting of lung cancer, breast cancer, oesophageal cancer, gastric cancer, colon cancer, cholangiocarcinoma, pancreatic cancer, ovarian cancer, head and neck cancers, synovial sarcoma, angiosarcoma, osteosarcoma, thyroid cancer, endometrial cancer, neuroblastoma, rabdomyosarcoma, liver cancer, melanoma, prostate cancer, renal cancer, soft tissue sarcoma, urothelial cancer, biliary cancer, glioblastoma, mesothelioma, cervical cancer, and colorectal cancer.

The disease associated with expression of WT1 may be selected from a group consisting of acute myeloid leukemia (AML), chronic myeloid leukemia (CML), lymphoblastic leukemia, myelodisplastic syndromes, lymphoma, multiple myeloma, non Hodgkin lymphoma, and Hodgkin lymphoma, lung cancer, breast cancer, oesophageal cancer, gastric cancer, colon cancer, cholangiocarcinoma, pancreatic cancer, ovarian cancer, head and neck cancers, synovial sarcoma, angiosarcoma, osteosarcoma, thyroid cancer, endometrial cancer, neuroblastoma, rabdomyosarcoma, liver cancer, melanoma, prostate cancer, renal cancer, soft tissue sarcoma, urothelial cancer, biliary cancer, glioblastoma, mesothelioma, cervical cancer, and colorectal cancer.

Pharmaceutical Composition

The TCRs of the present invention, the polynucleotides of the present invention, the vectors of the present invention, the cells of the present invention, the cells prepared by the methods of the present invention, the chimeric molecules of the present invention, and the mixed cell population of the present invention may be formulated for administration to subjects with a pharmaceutically acceptable carrier, diluent or excipient. Suitable carriers and diluents include isotonic saline solutions, for example phosphate-buffered saline, and potentially contain human serum albumin.

Handling of the cell therapy products is preferably performed in compliance with FACT-JACIE International Standards for cellular therapy.

Method of Treatment

In another aspect, the present invention provides a method for treating and/or preventing a disease associated with expression of WT1, which comprises the step of administering a TCR of the present invention, an isolated polynucleotide of the present invention, a vector of the present invention, a cell of the present invention, a cell prepared by a method of the present invention, a chimeric molecule of the present invention, or a mixed cell population of the present invention to a subject in need thereof.

The subject may be a human subject. The subject may be a non-human animal subject.

The subject may have a disease associated with expression of WT1. The subject may be at risk of developing a diseases associated with expression of WT1. The subject may have been previously determined to be at risk of developing a disease associated with expression of WT1. The subject may have an increased risk of developing a disease associated with WT1.

The increased risk may have been determined by genetic screening and/or by reviewing the subject's family history. The subject may express genetic markers indicative of increased risk of developing a disease associated with expression of WT1.

Suitably, a person skilled in the art will be aware of genetic risk factors (e.g. genetic markers) associated with increased risk of developing a disease associated with WT1. The skilled person may be able to use any suitable method or technique known in the art to determine whether the subject has an increased risk of developing a disease associated with expression of WT1.

The subject may have previously received treatment for a disease associated with expression of WT1. The subject may be in remission. The subject may be resistant to chemotherapy. The subject may be resistant to an anti-WT1 therapy.

In one embodiment, the method for treating and/or preventing a disease associated with expression of WT1 comprises the step of administering a chemotherapy to the subject. The chemotherapy may be administered to the subject simultaneously, sequentially or separately with the TCR of the present invention, the isolated polynucleotide of the present invention, the vector of the present invention, the cell according of the present invention, the cell prepared by the method of the present invention, or the chimeric molecule of the present invention.

In another aspect, the present invention provides a method of treating and/or preventing a disease associated with expression of WT1, which comprises the step of administering a mixed cell population, wherein the mixed cell population comprises a plurality of cell populations each expressing a different TCR of the present invention.

In another aspect, the present invention provides a mixed cell population comprising a plurality of cell populations each expressing a different TCR of the present invention.

In another aspect, the present invention provides a method for preparing a mixed cell population comprising a plurality of cell populations each expressing a different TCR of the present invention, wherein the method comprises the step of transducing a cell in vitro or ex vivo with a vector of the present invention.

In another aspect, the present invention provides a mixed cell population for use in treating and/or preventing a disease associated with expression of WT1, wherein the mixed cell population comprises a plurality of cell populations each expressing a different TCR of the present invention.

For example, the mixed cell population may comprise a first cell population expressing a first TCR of the present invention and a second cell population expressing a second TCR of the present invention. For example, the mixed cell population may comprise a first cell population expressing a first TCR of the present invention, a second cell population expressing a second TCR of the present invention, and a third cell population expressing a third TCR of the present invention, and so on.

Each cell population of the mixed cell population may, for example, express a single TCR of the present invention only. The endogenous TCR genes of the cell populations in the mixed cell population may be disrupted or deleted. Expression of endogenous TCR genes of the cells in the mixed cell population may be disrupted, e.g. by gene editing with an artificial nuclease.

In another aspect, the present invention provides use of TCR of the present invention, an isolated polynucleotide of the present invention, a vector of the present invention, a cell of the present invention, a cell prepared by a method of the present invention, a chimeric molecule of the present invention, or a mixed cell population of the present invention, for the manufacture of a medicament for the treatment of a disease associated with expression of WT1.

Both human and veterinary treatments are within the scope of the present invention.

The practice of the present invention will employ, unless otherwise indicated, conventional techniques of cell biology, molecular biology, histology, immunology, oncology, which are within the capabilities of a person of ordinary skill in the art. Such techniques are explained in the literature.

See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

Various preferred features and embodiments of the present invention will now be described by way of non-limiting examples.

### EXAMPLES

### Example 1—Generation of Functional WT1-Specific Cytotoxic T-Lymphocytes (CTLs) from Healthy Donors (HDs)

In order to identify novel TCRs specific for WT1 epitopes restricted to different HLA alleles, we stimulated peripheral blood mononuclear cells (PBMCs) from ten different HDs with a pool of pentadecapeptides (15mer) with an 11 amino acid overlap spanning the complete sequence of the WT1 protein (see materials and methods). This peptide design ensures the optimal stimulation of both CD4⁺ and CD8⁺ T-cells.

After 26-30 hours of stimulation, we enriched T-cells expressing CD137. CD137 is molecule upregulated upon T cell receptor engagement and has been previously shown to be a reliable marker for the rapid identification, isolation and expansion in vitro of antigen-specific memory and naive CD4⁺ and CD8⁺ T-cells. The CD137-negative fraction was further depleted of the CD3 fraction, then irradiated at 30 Gy and used as antigen presenting cells (APCs) for the CD137⁺ fraction. Sorted CD137⁺ cells were expanded in vitro for ˜9 days and restimulated with autologous APCs represented by CD3-depleted cells or by immortalized autologous B cells loaded with the peptide pool every 7-14 days. This procedure led to the enrichment of the WT1-specific T lymphocytes as shown by the cytofluorimetric results presented in FIGS. 1A-1J. Functional characterization of T cells was performed at different time points. More in detail, T cells were co-cultured with autologous APCs loaded with the peptide pool and, after 6 hours of co-culture, expression of CD107a and IFNγ in the T-cell population was identified by intracellular staining. The expression of CD107a after antigen encounter indicates antigen-induced degranulation and lytic potential.

As a negative control, cells were stimulated with an unrelated peptide pool. Control stimulation resulted in minimal secretion of IFNγ and CD107a by T-cells derived from each healthy donor.

### Example 2—Mapping of WT1 Epitopes Eliciting a T Cell Response

To identify the WT1 epitope recognized by T-cells, IFNγ secretion was quantified after 6 hours of in vitro co-culture of the WT1-stimulated/enriched T-cells with autologous APCs loaded with peptide subpools each containing up to 12 peptides according to a mapping grid. The mapping grid consists of 24 subpools with each peptide being uniquely contained within two intersecting subpools (Doubrovina, E. et al. Blood 120, 1633-1646 (2012)). Results are summarized in FIG. 2a.

FACS analysis showed substantial expression of IFNγ and CD107a by the HD1-, HD3-, HD6-, HD7-, HD10-derived T-cells after stimulation with subpools 4, 5 and 16 (FIGS. 2b, 2d, 2g, 2h, 2k). Substantial expression of IFNγ was observed for HD2-derived T-cells stimulated by subpools 6, 16, 17, and 20 (FIG. 2c), whereas subpools 4, 5, 6 14, 18, 21 stimulated expression of IFNγ and CD107a by HD4-derived T-cells (FIG. 2e) and subpools 5, 11, 12, 21, 22 stimulated expression of IFNγ in HD5 (FIG. 2f). For HD7, we additionally observed an increased expression of IFNγ and CD107a, even though at a lower level compared to the one observed with subpools 4, 5 and 16, after stimulation with subpools 7, 8, 20 (FIG. 2h). Furthermore, we observed an increased expression of IFNγ and CD107a after stimulation of HD8-derived T cells with subpools 12 and 14 (FIG. 2i) and of HD9-derived T cells with subpools 5, 13, 21 (FIG. 2j).

Afterwards, the HD-derived T-cells were stimulated for 6 hours with APCs pulsed with the single pentadecapeptides shared by the subpools eliciting the highest immune response and with at least one unrelated 15mer. FACS analysis indicated an increased expression of CD107a and/or IFNγ for peptides 40 and 41 in HD1 T-cells (FIG. 3a), peptides 54, 77, 90 for HD2 T-cells (FIG. 3b), peptide VLDFAPPGA (SEQ ID NO: 157; which is a nonamer of the peptide represented by SEQ ID NO: 117) for HD3 T-cells (FIG. 3c), peptides 17, 18, 99, 100 for HD4 (FIG. 3d, e), peptide 101 for HD5 (FIG. 3f), peptide VLDFAPPGA (SEQ ID NO: 157; which is a nonamer of the peptide represented by SEQ ID NO: 117) for HD6 T-cells (FIG. 3g), peptides 101, 125 and 137 for HD9 T-cells (FIG. 3h), peptide VLDFAPPGA (SEQ ID NO: 157; which is a nonamer of the peptide represented by SEQ ID NO: 117) for HD10 T-cells (FIG. 3i). In this way, the dominant immunogenic sequences were identified. No relevant immune responses (i.e. increased expression of CD107a and/or IFNγ) were observed after co-culture with unrelated control peptides. For HD7 and HD8, due to the reduced cell fitness, it was not possible to perform culture experiments to identify the immunogenic peptides. Still, we could predict the recognized peptide by the deconvolution of the mapping grid. We identified the overlapping sequence of peptides 41 and 42 (originated from SP4, 5, 16) and the overlapping sequence of peptides 91 and 92 (originated from SP7, 8, 20) for HD7 and peptide 24 for HD8.

To determine the HLA-restriction of the WT1 immunogenic peptide recognized by T-cells expanded from HD4, HD5 and HD10, the T-cells from these were co-cultured for 6 hours with a panel of different target EBV-BLCL cells each expressing a different HLA-A or HLA-B allele that had been pulsed with a relevant peptide (peptide 17 for HD4; peptide 101 for HD5; peptide VLDFAPPGA (SEQ ID NO: 157) for HD10) or an unrelated control peptide. Results of this experiment showed that peptide 17 is presented by the HLA-B*3502 allele and is recognized by T-cells derived from HD4 (FIG. 3j), peptide 101 is presented by the HLA-B*3501 and is recognized by T-cells derived from HD5 (FIG. 3k); peptide VLDFAPPGA (SEQ ID NO: 157) is presented by the HLA-A*0201 and is recognized by T-cells derived from HD10 (FIG. 3I).

Sequences of the WT1 peptides recognized by WT1-specific T-cells expanded from HD1-HD10 are shown in Table 3 below.

TABLE 3

| Binding | | | |
|---|---|---|---|
| TCR | | | |
| donor | Peptide | Sequence | SEQ ID NO |
| HD1/HD3/ | 40 | AAQWAPVLDFAPPGA | SEQ ID NO: 115 |
| HD6/ | 41 | APVLDFAPPGASAYG | SEQ ID NO: 116 |
| HD7/HD10 | Overlapping | APVLDFAPPGA | SEQ ID NO: 117 |
| | sequence | | |
| | (“11 mer” | | |
| | in FIG. 3c) | | |
| HD2 | 54 | QCLSAFTVHFSGQFT | SEQ ID NO: 118 |
| | 77 | EDPMGQQGSLGEQQY | SEQ ID NO: 119 |
| | 90 | SQLECMTWNQMNLGA | SEQ ID NO: 120 |
| HD4 | 17 | TCVPEPASQHTLRSG | SEQ ID NO: 121 |
| | 18 | EPASQHTLRSGPGCL | SEQ ID NO: 122 |
| | Overlapping | EPASQHTLRSG | SEQ ID NO: 123 |
| | sequence | | |
| | 99 | HSTGYESDNHTTPIL | SEQ ID NO: 124 |
| | 100 | YESDNHTTPILCGAQ | SEQ ID NO: 125 |
| | Overlapping | YESDNHTTPIL | SEQ ID NO: 126 |
| | sequence | | |
| HD5 | 101 | NHTTPILCGAQYRIH | SEQ ID NO: 127 |
| HD7 | 91 | CMTWNQMNLGATLKG | SEQ ID NO: 248 |
| | 92 | NQMNLGATLKGVAAG | SEQ ID NO: 249 |
| | Overlapping | NQMNLGATLKG | SEQ ID NO: 250 |
| | sequence | | |
| HD8 | 24 | DPGGIWAKLGAAEAS | SEQ ID NO: 251 |
| HD9 | 101 | NHTTPILCGAQYRIH | SEQ ID NO: 252 |
| | 125 | KRHQRRHTGVKPFQC | SEQ ID NO: 253 |
| | 137 | PSCQKKFARSDELVR | SEQ ID NO: 254 |

### Example 3—WT1-Specific T-Cells Selectively Eliminate WT1 Expressing Cells

To determine the HLA restriction of the WT1 specific T-cells and their ability to eliminate WT1-expressing cells, T cells were co-cultured with different target cells.

HD1-Derived T-Cells

Being aware that HD1 harbors the HLA-A*0201 allele, we co-cultured enriched WT1-specific T-cells with different target cells: T2 cells pulsed with the overlapping peptide pool comprising peptides 40 and 41 (see Table 3); T2 cells pulsed with the MelanA/MART1 pool as a negative control (T2 MelanA/MART1 pool); or K562 cells genetically modified in order to express both the HLA-A*0201 allele and to overexpress the WT1 protein (K562 HLA-A*0201 WT1).

After 6 hours of co-culture, expression of CD107a was established by FACS. Results for HD1 indicate the expression of CD107a in >60% of CD8⁺ T-cells following co-culture with T2 cells pulsed with the WT1 pool (FIG. 4a). Similarly, co-culture of WT1 specific T-cells with genetically modified K562 cells (expressing HLA-A*0201 allele and the WT1 protein) resulted in CD107a expression by >60% of CD8⁺ T-cells (FIG. 4a).

In contrast, CD107a expression by CD8⁺ T-cells co-cultured with T2 cells pulsed with the negative control MelanA/MART1 pool was minimal (FIG. 4a).

These results demonstrate that isolated HD1-derived T-cells specifically recognize WT1 peptide comprising the sequence APVLDFAPPGA (SEQ ID NO: 117) when presented by MHC molecules encoded by the HLA-A*0201 allele. Moreover, the results show that HD1-derived T-cells are able to specifically target cells overexpressing the WT1 protein. Accordingly, these experimental data demonstrate that TCRs expressed by HD1-derived T-cells specifically bind to the peptides comprising the APVLDFAPPGA (SEQ ID NO: 117) amino acid sequence and that such TCRs are HLA-A*0201 restricted.

HD3-Derived T-Cells

Being aware that HD3 harbors the HLA-A*0201 allele, we co-cultured enriched WT1-specific T-cells with different target cells: T2 cells pulsed with subpool 16 which was previously determined to contain the immunogenic peptide eliciting immune response (T2-SP16); T2 cells pulsed with the MelanA/MART1 pool as a negative control (T2-Melan A); wild type K562 cells (K562) as negative control; or K562 cells genetically modified to express both the HLA-A*0201 allele and to overexpress the WT1 protein (K562 A2+WT1+).

After 4 days of co-culture, the ability of the HD3 derived T-cells to kill target cells was expressed as elimination index—calculated as the total number of target cells still present after co-culture with the WT1-specific T-cells divided by the total number of target cells alone.

The results demonstrate the ability of WT1-specific T-cells to eliminate target cells expressing the identified specific WT1 epitope (FIG. 4b). In particular, the HD3-derived T-cells eliminated about 95% of the T2 cells pulsed with WT1 peptides comprising APVLDFAPPGA (SEQ ID NO: 117) amino acid sequence (subpool 16; SP16). Furthermore, the HD3-derived T-cells eliminated about 78% of the K562 cells expressing MHC molecules encoded by the HLA-A*0201 allele and overexpressing WT1 protein. In contrast, none of the negative control MelanA/MART1 pool-pulsed T2 cells were eliminated by the HD3-derived T-cells. Similarly, there was minimal elimination of control wild-type K562 cells (FIG. 4b).

These results demonstrate that isolated HD3-derived T-cells specifically recognize WT1 peptide comprising the amino acid sequence APVLDFAPPGA (SEQ ID NO: 117). Moreover, the results show that HD3-derived T-cells are able to specifically target and kill cells overexpressing the WT1 protein via peptide presentation by HLA-A*0201 encoded MHC. Accordingly, these experimental data demonstrate WT1 peptide specificity for TCRs expressed by HD3-derived T-cells and that HD3-derived TCRs are HLA-A*0201 restricted.

HD4-Derived T-Cells

The ability of HD4-derived T-cells to eliminate target cells was assessed by co-culturing the T-cells with primary leukemic blasts (CD33⁺ cells) isolated from an acute myeloid leukemia (AML) patient who was selected on the basis of high expression of the WT1 antigen and HLA typing (HLA-B*3502). As a negative control, HD4-derived T-cells were co-cultured with leukemic blasts from an AML patient who did not express the HLA-B*3502 allele.

After three days of co-culture at an effector to target ratio of 10 to 1, FACS analysis showed the nearly complete clearance of the leukemic blasts (CD33+) harvested from the AML patient expressing the HLA-B*3502 allele following the co-culture with HD4 WT1-specific T-cells (CD3⁺ cells) (FIG. 4c, upper panel). Indeed, only 0.54% of the remaining total cell population was positive for CD33 expression.

In contrast, no clearance of CD33⁺ cells was seen following co-culture of WT1-specific T-cells with the unrelated control AML blasts (FIG. 4c, lower panel). Indeed, in the control sample 7.9% of the total cell population was positive for CD33 expression following co-culture with the HD4-derived WT1-specific T-cells.

Importantly, these results demonstrate the ability of the WT1-specific T-cells derived from HD4 to specifically target and kill leukemic blasts (AML cancer cells) overexpressing WT1 and MHC encoded by the HLA-B*3502. Thus, TCRs derived from HD4 are able to specifically target and kill cancer cells in HLA-B*3502 restricted manner.

### Example 4—Immunoprofiling of Vβ Sequences for WT1 Specific T-Cells

To better identify the TCRs involved in antigenic recognition by the WT1 specific T-cells from HD1-6 and 10 (for HD7, HD8 and HD9, it was not possible to perform the Vβ Immunoprofiling analysis due to a reduced cell fitness), we first performed a multi-parametric FACS analysis to quantitatively determine the TCR Vβ repertoire. Thus, in order to determine the clonality of the expanded WT1-specific T-cells, we used the 10 Test Beta Mark TCR V beta repertoire kit according to manufacturer's recommendations. This kit allows the detection of the expression of 24 different V beta genes in eight individual tubes. In particular, coverage of 75% of the complete repertoire of V beta is guaranteed by using this approach. Results of FACS staining indicated the great prevalence of a specific Vβ for HD1, HD2, HD3, HD5—see FIG. 5. For HD4, HD6 and HD10, an exhaustive determination of the predominant Vβ was not possible, likely due to the intrinsic limitation of the kit which includes antibodies covering 75% of the existing Vβ proteins.

### Example 5— High Throughput Sequencing of TCR α and β Chains Isolated from WT1-Specific T Cells

WT1-specific T-cells were collected at different time points over the co-culture time frame and their RNA was extracted by using the Arcturus Pico Pure RNA extraction kit. CDR3 sequences of the WT1-specific T-cells were amplified by using a modified RACE approach in which a magnetic capture was included after the cDNA synthesis in order to increase the specificity of the reaction and eliminate unwanted templates (Ruggiero et al. Nat. Commun. 6, 8081 (2015)). Samples were sequenced using an Illumina MiSeq sequencer and the CDR3 clonotypes were identified using the MiXTCR software (Bolotin, D A et al. Nature Methods 12, 380-381 (2015)) Additionally, CDR1, CDR2 and CDR3 were further determined using the IMGT V-quest tool (Brochet, X. et al., Nucl. Acids Res. 36, W503-508 (2008). PMID: 18503082; Giudicelli, V., Brochet, X., Lefranc, M.-P., Cold Spring Harb Protoc. 2011 Jun. 1; 2011(6). pii: pdb.prot5633. doi: 10.1101/pdb.prot5633. PMID: 21632778 Abstract also in IMGT booklet with generous provision from Cold Spring Harbor (CSH) Protocol).

Sequencing results demonstrated the increasing predominance of a specific CDR3 clonotype in the WT1-specific T cell population over time for both TCR chains in HD1-10. Predominant α and β chain genotypes, and CDR3 sequences are provided in FIGS. 6A-6J.

In addition, the full length a and β chain amino acid sequences for TCRs derived from HD1-10 were determined—see Table 1. The corresponding nucleotide sequences were also determined—see Table 2.

### Example 6— Functional Validation of the Newly Identified TCRs

TCRs α and β sequences isolated from HD1 and HD3 and recognizing the WT1 VLDFAPPGA (SEQ ID NO: 157) peptide when presented by the HLA-A*0201 allele were cloned into a lentiviral vector under the control of a bidirectional promoter to promote robust and coordinate expression of both TCR chains in transduced lymphocytes. T cells from a healthy individual were transduced with the viral vector encoding either the HD1 TCR or the HD3 TCR. As control, we also transduced cells with the WT1 126-134 TCR. Transduced T cells were functionally validated by co-culture with different target cells represented by the T2 cells pulsed with one of the 2 recognized peptides (VLDFAPPGA (SEQ ID NO: 157) for HD1 and HD3 TCR; RMFPNAPYL (SEQ ID NO: 255) for WT1 126-134 TCR) (FIG. 7a), K562 cells either wild type or engineered in order to express the HLA-A*0201 allele (FIG. 7b), primary AML blasts derived from 3 AML patients and selected according to the expression of the HLA-A*0201 allele and the WT1 expression (FIG. 7c). Upon 3 days of co-culture we observed the ability of each transduced T cell population in specifically recognizing the target peptide when presented by the HLA-A*0201 allele (FIG. 7a) and the greater potential of HD1 T cells in mediating a near complete elimination of the engineered K562 cells. The higher potency of HD1 TCR in recognizing the target antigen was further confirmed by the results of the co-culture with pAML blasts. Here, we observed a greater elimination of both pAML blasts harbouring the HLA-A*0201 allele upon co-culture with HD1 TR T cells compared to the conditions in which HD3 TR and WT1 126-134 TR T lymphocytes were used as effector cells.

Materials and Methods

The WT1 protein sequence previously published by Gessler et al. (Gessler, M. et al. (1990) Nature 343: 774-778) was used to design the peptides used for the stimulation and isolation of WT1-specific T cells. This sequence contains 575 amino acids and includes the first 126 amino acids in the N-terminus missing in the (exon 5+, KTS+) isoform of WT1. We designed 141 pentadecapeptides spanning the whole sequence of the WT1 protein, each overlapping the next one by 11 amino acids.

Peptides were synthesized by PRIMM to specifications of validated sequence, 70% purity, sterility and absence of endotoxin. These peptides were mixed in equal amounts in the WT1 pool at a concentration of 1 μg/ml per peptide. Additionally, 24 subpools were generated, each containing up to 12 peptides (4.17 μg/ml/per peptide) according to a specific mapping matrix in order to have each peptide included in only two overlapping subpools as shown in Table 4 (see mapping grid strategy in Doubrovina, E. et al. Blood 120, 1633-1646 (2012)).

TABLE 4

| | SP1 | SP2 | SP3 | SP4 | SP5 | SP6 | SP7 | SP8 | SP9 | SP10 | SP11 | SP12 |
|---|---|---|---|---|---|---|---|---|---|---|---|---|
| SP13 | 1 | 2 | 3 | 4 | 5 | 6 | 7 | 8 | 9 | 10 | 11 | 12 |
| SP14 | 13 | 14 | 15 | 16 | 17 | 18 | 19 | 20 | 21 | 22 | 23 | 24 |
| SP15 | 25 | 26 | 27 | 28 | 29 | 30 | 31 | 32 | 33 | 34 | 35 | 36 |
| SP16 | 37 | 38 | 39 | 40 | 41 | 42 | 43 | 44 | 45 | 46 | 47 | 48 |
| SP17 | 49 | 50 | 51 | 52 | 53 | 54 | 55 | 56 | 57 | 58 | 59 | 60 |
| SP18 | 61 | 62 | 63 | 64 | 65 | 66 | 67 | 68 | 69 | 70 | 71 | 72 |
| SP19 | 73 | 74 | 75 | 76 | 77 | 78 | 79 | 80 | 81 | 82 | 83 | 84 |
| SP20 | 85 | 86 | 87 | 88 | 89 | 90 | 91 | 92 | 93 | 94 | 95 | 96 |
| SP21 | 97 | 98 | 99 | 100 | 101 | 102 | 103 | 104 | 105 | 106 | 107 | 108 |
| SP22 | 109 | 110 | 111 | 112 | 113 | 114 | 115 | 116 | 117 | 118 | 119 | 120 |
| SP23 | 121 | 122 | 123 | 124 | 125 | 126 | 127 | 128 | 129 | 130 | 131 | 132 |
| SP24 | 133 | 134 | 135 | 136 | 137 | 138 | 139 | 140 | 141 | | | |

Isolation of Peripheral Blood Mononuclear Cells

Peripheral blood was obtained from ten healthy donors at San Raffaele Hospital upon informed consent. Peripheral blood mononuclear cells were isolated using Ficoll-Hypaque density gradient centrifugation.

Immortalized B Cells

Autologous B cells were isolated from PBMCs of healthy donors using the CD19 Microbeads (Miltenyi Biotech). Cells were transduced with a lentiviral vector harbouring the BCL-6/BCL-XL transgene (Kwakkenbos, M. J. et al. Nat. Med. 16, 123 (2009)) and the H/F pseudotype (Lévy, C. et al. Molecular Therapy20 9, 1699-1712, (2012)) and cultured in IMDM supplemented with 10% fetal bovine serum (FBS), penicillin-streptomycin, and 10 ng/ml of IL21 (Miltenyi Biotech). B-cells were re-stimulated every 5 days by co-culture with irradiated (50 Gy) mouse L-cell fibroblasts expressing CD40L (3T3-CD40L) at a B-cell:3T3-CD40L ratio of 10:1.

Cell Lines

We cultured the T2 and K562 cell lines in RPMI 1640 (GIBCO-BRL) supplemented with penicillin, streptomycin, glutamine and 10% FBS (BioWhittaker).

Leukemic Cells

Primary AML cells were obtained from San Raffaele Hospital (OSR) Leukemia biobank and selected according to the expression of WT1 by quantitative PCR and to the HLA typing. All EBV-BLCLs and primary leukemia cells were typed for HLA-A, HLA-B, HLA-C, HLA-DR and HLA-DQ alleles at high resolution at the HLA laboratory of the OSR.

Flow Cytometry

We used FITC-, PE-, PerCP-, APC-, PE-Cychrome 7-, APC Cychrome 7-, Pacific Blue and Brillant Violet-conjugated antibodies directed to human CD3, CD4, CD8, CD107a, IFNγ, TNFα, CD33, CD117, CD34, CD14, Vβ21.3, Vβ8, Vβ7.2 and HLA-A2. APC fluorescently-labelled WT1 VLDFAPPGA (SEQ ID NO: 157) and PE fluorescently-labelled WT1 RMFPNAPYL (SEQ ID NO: 255) dextramers were used following the manufacturer's instructions. Cells were incubated with antibodies for 15 minutes at 4° C. and washed with phosphate-buffered saline containing 1% FBS. Samples were run through a fluorescence-activated cell sorter (FACS) Canto II flow cytometer (BD Biosciences), and data were analyzed by Flow Jo software (Tree star Inc). For intracellular evaluation of cytokine secretion and expression of degranulation markers, the Fix/Perm buffer set (Biolegend) was used according to manufacturer instructions.

Stimulation, Isolation and Expansion of WT1-Specific T-Cells

Freshly isolated PBMCs were resuspended in X-VIVO supplemented with 5% human AB serum, 2 mM glutamine and 1 μg/ml CD28 monoclonal antibody, seeded at a density of 10⁷ cells/ml and stimulated with the WT1 overlapping peptide pool, each peptide present at a concentration of 1 μg/ml.

Antigen-specific T-cells were isolated after 26-30 hours by CD137 expression. More specifically, cells were stained with the PE-conjugated CD137 antibody and sorted using anti-PE microbeads (Miltenyi Biotech). The CD137⁻ fraction was depleted of the CD3 cells using CD3-Microbeads (Miltenyi Biotech), irradiated 30 Gy and used as peptide-loaded APCs in a co-culture with the CD137⁺ fraction at a ratio of 100:1 when possible or at least 20:1 and a final density of 5×10⁶ cells/ml. X-VIVO supplemented with 5% human AB serum, 5 ng/ml IL7, 5 ng/ml IL15 and 10 ng/ml IL21 was used as the medium. Media, including cytokines, was replaced every 2-3 days.

Re-Stimulation of Expanded Antigen-Specific T-Cells

Cells were re-stimulated every 7-14 days with WT1-pulsed autologous APCs (PBMC CD3-depleted cells; immortalized B cells). In the initial re-stimulations, cells were washed 2 days before and plated in cytokine-free medium. APCs were irradiated with 30 Gy, pulsed with the peptide pool overnight and co-cultured with effector cells in X-VIVO supplemented with 5% human AB serum, 1 μg/ml CD28 monoclonal antibody and IL7 (5 ng/ml), IL15 (5 ng/ml), IL21 (10 ng/ml).

Assessment of T Cell Response

The percentage of T-cells responding to the WT1 peptide pool was measured by performing a 6 hours co-culture of the effector cells with autologous APCs (ratio of at least 1:1) pulsed with the desired antigen (WT1 peptide pool, WT1 subpools, WT1 individual peptides, unrelated peptide pool as control). Co-cultures were seeded in X-VIVO supplemented with 5% human

AB serum and supplemented with the CD28 monoclonal antibody (1 μg/ml), Golgi Stop (BD) and CD107a-FITC antibody for assessment of degranulation. Cells were then fixed, permeabilized and stained intracellularly to determine the percentage of CD3⁺CD8⁺ or CD3⁺CD4⁺ cells expressing IFNγ and CD107a.

Mapping of Immunogenic Peptides

T-cells stimulated with the WT1 pool were seeded in different wells and co-cultured with autologous APCs loaded with one of each of the WT1 subpools at a ratio of at least 1:1. T-cell responses to each subpool were measured as previously described by FACS analysis. Deconvolution of the mapping grid was essential to determine which shared peptides were eliciting a T cell response. Once determined the immunogenic peptides, T-cells were further stimulated with APCs loaded with the individual peptides to confirm their immunogenicity.

Evaluation of T Cell Ability to Recognize WT1-Expressing Cells

WT1-specificity and HLA-restricted ability of T-cells to recognize target cells was measured with different experimental procedures. For T-cells derived from HD1, secretion of CD107a was determined by FACS analysis after 6 hours co-culture with target cells; for T cells derived from HD3, elimination index was calculated as the total number of target cells still present after 4 days co-culture with the WT1-specific T-cells divided by the total number of target cells alone; for T-cells derived from HD4, the percentage of CD33⁺ target cells (AML primary cells harbouring the HLA alleles of interest and as control, of AML primary cells not harbouring the specific HLA allele) still present after 3 days co-culture with CD3+WT1-specific T cells was assessed by cytofluorimetric analysis.

Assessment of T Cell Clonality

In order to determine the clonality of the expanded WT1-specific T cells, the 10 Test Beta Mark TCR V beta repertoire kit was used according to manufacturer's recommendations.

TCR Repertoire Sequencing

WT1-specific T cells were collected at different time points over the co-culture time frame and RNA was extracted by using the Arcturus Pico Pure RNA extraction kit. Complementarity determining region (CDR) 3 sequences of the WT1-specific T cells were amplified by using a modified RACE approach (Ruggiero, E. et al. Nat. Commun. 6, 8081 (2015)). Samples were sequenced by using an IlluminaMiSeq sequencer and CDR3 clonotypes identified using the MiXCR software (Bolotin, D A et al. Nature Methods 12, 380-381 (2015)).

Lentiviral Vectors

TCR α and β chain genes isolated from HD1 and HD3 were codon-optimized, cysteine-modified and cloned in a lentiviral vector (LV) under a bidirectional promoter. The amino acid (aa) and nucleotide (nt) sequences were:

| | | SEQ |
|---|---|---|
| | | ID |
| | | NO: |
| HD1 | ATGGAAACCCTGCTGAAGGTGCTGAGCGGCACACTGCTGTGGCAGC | 256 |
| α chain- | TGACATGGGTCCGATCTCAGCAGCCTGTGCAGTCTCCTCAGGCCGT | |
| seq nt | GATTCTGAGAGAAGGCGAGGACGCCGTGATCAACTGCAGCAGCTCT | |
| | AAGGCCCTGTACAGCGTGCACTGGTACAGACAGAAGCACGGCGAGG | |
| | CCCCTGTGTTCCTGATGATCCTGCTGAAAGGCGGCGAGCAGAAGGG | |
| | CCACGAGAAGATCAGCGCCAGCTTCAACGAGAAGAAGCAGCAGTCC | |
| | AGCCTGTACCTGACAGCCAGCCAGCTGAGCTACAGCGGCACCTACT | |
| | TTTGTGGCACCGCCTGGATCAACGACTACAAGCTGTCTTTCGGAGCC | |
| | GGCACCACAGTGACAGTGCGGGCCAATATTCAGAACCCCGATCCTG | |
| | CCGTGTACCAGCTGAGAGACAGCAAGAGCAGCGACAAGAGCGTGTG | |
| | CCTGTTCACCGACTTCGACAGCCAGACCAACGTGTCCCAGAGCAAG | |
| | GACAGCGACGTGTACATCACCGATAAGTGCGTGCTGGACATGCGGA | |
| | GCATGGACTTCAAGAGCAACAGCGCCGTGGCCTGGTCCAACAAGAG | |
| | CGATTTCGCCTGCGCCAACGCCTTCAACAACAGCATTATCCCCGAGG | |
| | ACACATTCTTCCCAAGTCCTGAGAGCAGCTGCGACGTGAAGCTGGT | |
| | GGAAAAGAGCTTCGAGACAGACACCAACCTGAACTTCCAGAACCTGA | |
| | GCGTGATCGGCTTCAGAATCCTGCTGCTCAAGGTGGCCGGCTTCAA | |
| | CCTGCTGATGACCCTGAGACTGTGGTCCAGC | |
| HD1 | METLLKVLSGTLLWQLTWVRSQQPVQSPQAVILREGEDAVINCSSSKAL | 257 |
| α chain- | YSVHWYRQKHGEAPVFLMILLKGGEQKGHEKISASFNEKKQQSSLYLTA | |
| seq aa | SQLSYSGTYFCGTAWINDYKLSFGAGTTVTVRANIQNPDPAVYQLRDSK | |
| | SSDKSVCLFTDFDSQTNVSQSKDSDVYITDKCVLDMRSMDFKSNSAVA | |
| | WSNKSDFACANAFNNSIIPEDTFFPSPESSCDVKLVEKSFETDTNLNFQ | |
| | NLSVIGFRILLLKVAGFNLLMTLRLWSS | |
| HD1 | ATGGGATCTTGGACACTGTGTTGCGTGTCCCTGTGCATCCTGGTGGC | 258 |
| β chain- | CAAGCACACAGATGCCGGCGTGATCCAGTCTCCTAGACACGAAGTG | |
| seq nt | ACCGAGATGGGCCAAGAAGTGACCCTGCGCTGCAAGCCTATCAGCG | |
| | GCCACGATTACCTGTTCTGGTACAGACAGACCATGATGAGAGGCCTG | |
| | GAACTGCTGATCTACTTCAACAACAACGTGCCCATCGACGACAGCGG | |
| | CATGCCCGAGGATAGATTCAGCGCCAAGATGCCCAACGCCAGCTTC | |
| | AGCACCCTGAAGATCCAGCCTAGCGAGCCCAGAGATAGCGCCGTGT | |
| | ACTTCTGCGCCAGCAGAAAGACAGGCGGCTACAGCAATCAGCCCCA | |
| | GCACTTTGGAGATGGCACCCGGCTGAGCATCCTGGAAGATCTGAAG | |
| | AACGTGTTCCCACCTGAGGTGGCCGTGTTCGAGCCTTCTGAGGCCG | |
| | AGATCAGCCACACACAGAAAGCCACACTCGTGTGTCTGGCCACCGG | |
| | CTTCTATCCCGATCACGTGGAACTGTCTTGGTGGGTCAACGGCAAAG | |
| | AGGTGCACAGCGGCGTCTGTACCGATCCTCAGCCTCTGAAAGAGCA | |
| | GCCCGCTCTGAACGACAGCAGATACTGCCTGAGCAGCAGACTGAGA | |
| | GTGTCCGCCACCTTCTGGCAGAACCCCAGAAACCACTTCAGATGCCA | |
| | GGTGCAGTTCTACGGCCTGAGCGAGAACGATGAGTGGACCCAGGAT | |
| | AGAGCCAAGCCTGTGACACAGATCGTGTCTGCCGAAGCCTGGGGCA | |
| | GAGCCGATTGTGGCTTTACCAGCGAGAGCTACCAGCAGGGCGTGCT | |
| | GTCTGCCACAATCCTGTACGAGATCCTGCTGGGCAAAGCCACTCTGT | |
| | ACGCCGTGCTGGTGTCTGCCCTGGTGCTGATGGCCATGGTCAAGCG | |
| | GAAGGATAGCAGGGGC | |
| HD1 | MGSWTLCCVSLCILVAKHTDAGVIQSPRHEVTEMGQEVTLRCKPISGHD | 259 |
| β chain- | YLFWYRQTMMRGLELLIYFNNNVPIDDSGMPEDRFSAKMPNASFSTLKI | |
| seq aa | QPSEPRDSAVYFCASRKTGGYSNQPQHFGDGTRLSILEDLKNVFPPEV | |
| | AVFEPSEAEISHTQKATLVCLATGFYPDHVELSWWVNGKEVHSGVCTD | |
| | PQPLKEQPALNDSRYCLSSRLRVSATFWQNPRNHFRCQVQFYGLSEN | |
| | DEWTQDRAKPVTQIVSAEAWG RADCGFTSESYQQGVLSATILYEILLGK | |
| | ATLYAVLVSALVLMAMVKRKDSRG | |
| HD3 | ATGATCAGCCTGAGAGTGCTGCTGGTCATCCTGTGGCTGCAGCTGT | 260 |
| α chain- | CTTGGGTCTGGTCCCAGCGGAAAGAGGTGGAACAGGACCCCGGAC | |
| seq nt | CTTTCAATGTGCCTGAAGGCGCCACCGTGGCCTTCAACTGCACCTAC | |
| | AGCAATAGCGCCAGCCAGAGCTTCTTCTGGTACAGACAGGACTGCC | |
| | GGAAAGAACCCAAGCTGCTGATGAGCGTGTACAGCAGCGGCAACGA | |
| | GGACGGCAGATTCACAGCCCAGCTGAACAGAGCCAGCCAGTACATC | |
| | AGCCTGCTGATCCGGGATAGCAAGCTGAGCGATAGCGCCACCTACC | |
| | TGTGCGTGGTCAACCTGCTGTCTAATCAAGGCGGCAAGCTGATCTTC | |
| | GGCCAGGGCACAGAGCTGAGCGTGAAGCCCAACATTCAGAACCCCG | |
| | ATCCTGCCGTGTACCAGCTGAGAGACAGCAAGAGCAGCGACAAGAG | |
| | CGTGTGCCTGTTCACCGACTTCGACAGCCAGACCAACGTGTCCCAG | |
| | AGCAAGGACAGCGACGTGTACATCACCGATAAGTGCGTGCTGGACA | |
| | TGCGGAGCATGGACTTCAAGAGCAACAGCGCCGTGGCCTGGTCCAA | |
| | CAAGAGCGATTTCGCCTGCGCCAACGCCTTCAACAACAGCATTATCC | |
| | CCGAGGACACATTCTTCCCAAGTCCTGAGAGCAGCTGCGACGTGAA | |
| | GCTGGTGGAAAAGAGCTTCGAGACAGACACCAACCTGAACTTCCAG | |
| | AACCTGTCCGTGATCGGCTTCCGGATCCTGCTGCTGAAAGTGGCCG | |
| | GCTTCAACCTCCTGATGACCCTGAGACTGTGGTCCAGC | |
| HD3 | MISLRVLLVILWLQLSWVWSQRKEVEQDPGPFNVPEGATVAFNCTYSN | 261 |
| α chain- | SASQSFFWYRQDCRKEPKLLMSVYSSGNEDGRFTAQLNRASQYISLLIR | |
| seq aa | DSKLSDSATYLCVVNLLSNQGGKLIFGQGTELSVKPNIQNPDPAVYQLR | |
| | DSKSSDKSVCLFTDFDSQTNVSQSKDSDVYITDKCVLDMRSMDFKSNS | |
| | AVAWSNKSDFACANAFNNSIIPEDTFFPSPESSCDVKLVEKSFETDTNLN | |
| | FQNLSVIGFRILLLKVAGFNLLMTLRLWSS | |
| HD3 | ATGGGATGTAGACTTCTGTGTTGCGCCGTGCTGTGTCTGCTTGGAGC | 262 |
| β chain- | TGGCGAACTGGTGCCTATGGAAACCGGCGTGACCCAGACACCTAGA | |
| seq nt | CACCTGGTCATGGGCATGACAAACAAGAAAAGCCTGAAGTGCGAGC | |
| | AGCACCTGGGCCACAATGCCATGTACTGGTACAAGCAGAGCGCCAA | |
| | GAAACCCCTGGAACTGATGTTCGTGTACAGCCTGGAAGAGAGGGTC | |
| | GAGAACAACAGCGTGCCCAGCAGATTCAGCCCTGAGTGCCCTAATA | |
| | GCAGCCACCTGTTTCTGCATCTGCACACCCTGCAGCCTGAGGACTCT | |
| | GCCCTGTATCTGTGTGCCAGCAGCCAGGACTACCTGGTGTCCAACG | |
| | AGAAGCTGTTCTTCGGCAGCGGCACACAGCTGAGCGTGCTGGAAGA | |
| | TCTGAAGAACGTGTTCCCACCTGAGGTGGCCGTGTTCGAGCCTTCTG | |
| | AGGCCGAGATCAGCCACACACAGAAAGCCACACTCGTGTGTCTGGC | |
| | CACCGGCTTCTATCCCGATCACGTGGAACTGTCTTGGTGGGTCAACG | |
| | GCAAAGAGGTGCACAGCGGCGTCTGTACCGATCCTCAGCCTCTGAA | |
| | AGAGCAGCCCGCTCTGAACGACAGCAGATACTGCCTGAGCAGCAGA | |
| | CTGAGAGTGTCCGCCACCTTCTGGCAGAACCCCAGAAACCACTTCA | |
| | GATGCCAGGTGCAGTTCTACGGCCTGAGCGAGAACGATGAGTGGAC | |
| | CCAGGATAGAGCCAAGCCTGTGACACAGATCGTGTCTGCCGAAGCC | |
| | TGGGGCAGAGCCGATTGTGGCTTTACCAGCGAGAGCTACCAGCAGG | |
| | GCGTGCTGTCTGCCACAATCCTGTACGAGATCCTGCTGGGAAAAGC | |
| | CACTCTGTACGCTGTGCTGGTGTCCGCTCTGGTGCTGATGGCCATG | |
| | GTCAAGCGGAAGGATAGCAGGGGC | |
| HD3 | MGCRLLCCAVLCLLGAGELVPMETGVTQTPRHLVMGMTNKKSLKCEQH | 263 |
| β chain- | LGHNAMYWYKQSAKKPLELMFVYSLEERVENNSVPSRFSPECPNSSHL | |
| seq aa | FLHLHTLQPEDSALYLCASSQDYLVSNEKLFFGSGTQLSVLEDLKNVFPP | |
| | EVAVFEPSEAEISHTQKATLVCLATGFYPDHVELSWWVNGKEVHSGVC | |
| | TDPQPLKEQPALNDSRYCLSSRLRVSATFWQNPRNHFRCQVQFYGLSE | |
| | NDEWTQDRAKPVTQIVSAEAWGRADCGFTSESYQQGVLSATILYEILLG | |
| | KATLYAVLVSALVLMAMVKRKDSRG | |

LVs were packaged by an integrase-competent third-generation construct and pseudotyped by the vescicular stomatitis virus (VSV) envelope. As control, we included the LV encoding for a WT1 126-134 TCR recognizing the RMFPNAPYL (SEQ ID NO: 255) peptide.

Vector Transductions

For transduction with WT1-TCR lentiviral vector, T lymphocytes isolated from a healthy individual were activated and sorted using magnetic beads conjugated to antibodies to CD3 and CD28 (ClinExVivo CD3/CD28; Invitrogen), following the manufacturer instructions, and cultured in Iscove's Modified Dulbecco's Media (IMDM) (GIBCO-BRL) supplemented with penicillin, streptomycin, 10% FBS and 5 ng ml⁻¹ of each IL-7 and IL-15 (PeproTech). For transduction, T lymphocytes were plated at 2.5×10⁶ cells ml⁻¹ and infected with the LV for 24 h. Afterwards, T cells were cultured at 10⁶ cells ml⁻¹ and expanded. Transduction efficiency was determined by measuring the percentage of the CD3 T cells expressing the specific dextramers. Cells were sorted using APC or PE-fluorescently-labelled HLA-A*0201 dextramer specific for the VLDFAPPGA (SEQ ID NO: 157) or RMFPNAPYL (SEQ ID NO: 255) peptide (Immudex) using anti-APC or anti-PE microbeads (Miltenyi Biotec) following the manufacturer instructions.

Functional Assays

The ability of HD1, HD3 and WT1 126-134 TCR-transferred T-cells to recognize WT1-expressing target cells was measured upon co-culture with (a) T2 cells either pulsed with the WT1 126-134 peptide or with the VLDFAPPGA (SEQ ID NO: 157) peptide (effector:target ratio=1:1); (b) K562 cells either wild type (K562) or genetically modified in order to express the HLA-A*0201 allele (effector:target ratio=1:1); (c) 3 different primary AML blasts selected according to the expression of the HLA-A*0201 allele and of the WT1 antigen (effector:target ratio=5:1). For the co-culture with T2 and K562 cell lines, we included untransduced T cells as control. After 3 days of culture, the percentage of target cells was assessed by cytofluorimetric analysis. The elimination index was calculated as follows: 1-(total number of target cells still present after 3 days co-culture with the WT1-specific T-cells/total number of target cells alone).

All publications mentioned in the above specification are herein incorporated by reference. Various modifications and variations of the described present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in cell biology, immunology, immunotherapy, molecular biology, oncology, or related fields are intended to be within the scope of the following claims.

1. A T-cell receptor (TCR), which binds to a Wilms tumour 1 protein (WT1) peptide when presented by a major histocompatibility complex (MHC), wherein the TCR:
(i) comprises a CDR3α comprising the amino acid sequence of CGTAWINDYKLSF (SEQ ID NO: 3) or a variant thereof having up to three amino acid substitutions, additions or deletions, and a CDR3β comprising the amino acid sequence of CASRKTGGYSNQPQHF (SEQ ID NO: 8) or a variant thereof having up to three amino acid substitutions, additions or deletions;
(ii) comprises a CDR3α comprising the amino acid sequence of CVVNLLSNQGGKLIF (SEQ ID NO: 36) or a variant thereof having up to three amino acid substitutions, additions or deletions, and a CDR3β comprising the amino acid sequence of CASSQDYLVSNEKLFF (SEQ ID NO: 41) or a variant thereof having up to three amino acid substitutions, additions or deletions;
(iii) comprises a CDR3α comprising the amino acid sequence of CAVRLSGSARQLTF (SEQ ID NO: 14) or a variant thereof having up to three amino acid substitutions, additions or deletions, and a CDR3β comprising the amino acid sequence of CASSLLGDEQYF (SEQ ID NO: 24) or a variant thereof having up to three amino acid substitutions, additions or deletions;
(iv) comprises a CDR3α comprising the amino acid sequence of CAVRLSGSARQLTF (SEQ ID NO: 14) or a variant thereof having up to three amino acid substitutions, additions or deletions, and a CDR3β comprising the amino acid sequence of CASSLVALQGAGEQYF (SEQ ID NO: 30) or a variant thereof having up to three amino acid substitutions, additions or deletions;
(v) comprises a CDR3α comprising the amino acid sequence of CAYRSLKYGNKLVF (SEQ ID NO: 19) or a variant thereof having up to three amino acid substitutions, additions or deletions, and a CDR3β comprising the amino acid sequence of CASSLLGDEQYF (SEQ ID NO: 24) or a variant thereof having up to three amino acid substitutions, additions or deletions;
(vi) comprises a CDR3α comprising the amino acid sequence of CAYRSLKYGNKLVF (SEQ ID NO: 19) or a variant thereof having up to three amino acid substitutions, additions or deletions, and a CDR3β comprising the amino acid sequence of CASSLVALQGAGEQYF (SEQ ID NO: 30) or a variant thereof having up to three amino acid substitutions, additions or deletions;
(vii) comprises a CDR3α comprising the amino acid sequence of CATDAYSGNTPLVF (SEQ ID NO: 47) or a variant thereof having up to three amino acid substitutions, additions or deletions, and a CDR3β comprising the amino acid sequence of CASRAAGLDTEAFF (SEQ ID NO: 57) or a variant thereof having up to three amino acid substitutions, additions or deletions;
(viii) comprises a CDR3α comprising the amino acid sequence of CATDAYSGNTPLVF (SEQ ID NO: 47) or a variant thereof having up to three amino acid substitutions, additions or deletions, and a CDR3β comprising the amino acid sequence of CASTQTPYEQYF (SEQ ID NO: 63) or a variant thereof having up to three amino acid substitutions, additions or deletions;
(ix) comprises a CDR3α comprising the amino acid sequence of CATDAYSGNTPLVF (SEQ ID NO: 47) or a variant thereof having up to three amino acid substitutions, additions or deletions, and a CDR3β comprising the amino acid sequence of CASSTVGGEDYGYTF (SEQ ID NO: 69) or a variant thereof having up to three amino acid substitutions, additions or deletions;
(x) comprises a CDR3α comprising the amino acid sequence of CAVRAEIYNQGGKLIF (SEQ ID NO: 52) or a variant thereof having up to three amino acid substitutions, additions or deletions, and a CDR3β comprising the amino acid sequence of CASRAAGLDTEAFF (SEQ ID NO:57) or a variant thereof having up to three amino acid substitutions, additions or deletions;
(xi) comprises a CDR3α comprising the amino acid sequence of CAVRAEIYNQGGKLIF (SEQ ID NO: 52) or a variant thereof having up to three amino acid substitutions, additions or deletions, and a CDR3β comprising the amino acid sequence of CASTQTPYEQYF (SEQ ID NO: 63) or a variant thereof having up to three amino acid substitutions, additions or deletions;
(xii) comprises a CDR3α comprising the amino acid sequence of CAVRAEIYNQGGKLIF (SEQ ID NO: 52) or a variant thereof having up to three amino acid substitutions, additions or deletions, and a CDR3β comprising the amino acid sequence of CASSTVGGEDYGYTF (SEQ ID NO: 69) or a variant thereof having up to three amino acid substitutions, additions or deletions;
(xiii) comprises a CDR3α comprising the amino acid sequence of CAASMAGAGSYQLTF (SEQ ID NO: 75) or a variant thereof having up to three amino acid substitutions, additions or deletions, and a CDR3β comprising the amino acid sequence of CAISVGQGALYEQYF (SEQ ID NO: 80) or a variant thereof having up to three amino acid substitutions, additions or deletions;
(xiv) comprises a CDR3α comprising the amino acid sequence of CAASMAGAGSYQLTF (SEQ ID NO: 75) or a variant thereof having up to three amino acid substitutions, additions or deletions, and a CDR3β comprising the amino acid sequence of CASSVARDRRNYGYTF (SEQ ID NO: 86) or a variant thereof having up to three amino acid substitutions, additions or deletions;
(xv) comprises a CDR3α comprising the amino acid sequence of CAANNARLMF (SEQ ID NO: 92) or a variant thereof having up to three amino acid substitutions, additions or deletions, and a CDR3β comprising the amino acid sequence of CASSDTRAREQFF (SEQ ID NO: 97) or a variant thereof having up to three amino acid substitutions, additions or deletions;
(xvi) comprises a CDR3α comprising the amino acid sequence of CAERLNTDKLIF (SEQ ID NO: 103) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions;
(xvii) comprises a CDR3α comprising the amino acid sequence of CAERLNTDKLIF (SEQ ID NO: 103) or a variant thereof having up to three amino acid substitutions, additions or deletions, and a CDR3β comprising the amino acid sequence of CSVGGSGSYNEQFF (SEQ ID NO: 169) or a variant thereof having up to three amino acid substitutions, additions or deletions;
(xviii) comprises a CDR3α comprising the amino acid sequence of CAVEATDSWGKLQF (SEQ ID NO: 108) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions;
(xix) comprises a CDR3α comprising the amino acid sequence of CAVEATDSWGKLQF (SEQ ID NO: 108) or a variant thereof having up to three amino acid substitutions, additions or deletions, and a CDR3β comprising the amino acid sequence of CSVGGSGSYNEQFF (SEQ ID NO: 169) or a variant thereof having up to three amino acid substitutions, additions or deletions;
(xx) comprises a CDR3α comprising the amino acid sequence of CAVRTSYDKVIF (SEQ ID NO: 113) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions;
(xxi) comprises a CDR3α comprising the amino acid sequence of CAVRTSYDKVIF (SEQ ID NO: 113) or a variant thereof having up to three amino acid substitutions, additions or deletions, and a CDR3β comprising the amino acid sequence of CSVGGSGSYNEQFF (SEQ ID NO: 169) or a variant thereof having up to three amino acid substitutions, additions or deletions;
(xxii) comprises a CDR3α comprising the amino acid sequence of CAVTVGNKLVF (SEQ ID NO: 175) or a variant thereof having up to three amino acid substitutions, additions or deletions, and a CDR3β comprising the amino acid sequence of CASRGWREQFF (SEQ ID NO: 180) or a variant thereof having up to three amino acid substitutions, additions or deletions;
(xxiii) comprises a CDR3α comprising the amino acid sequence of CAARSYNTDKLIF (SEQ ID NO: 186) or a variant thereof having up to three amino acid substitutions, additions or deletions, and a CDR3β comprising the amino acid sequence of CASSWGYQETQYF (SEQ ID NO: 196) or a variant thereof having up to three amino acid substitutions, additions or deletions;
(xxiv) comprises a CDR3α comprising the amino acid sequence of CAARSYNTDKLIF (SEQ ID NO: 186) or a variant thereof having up to three amino acid substitutions, additions or deletions, and a CDR3β comprising the amino acid sequence of CASSPTGGEYYGYTF (SEQ ID NO: 202) or a variant thereof having up to three amino acid substitutions, additions or deletions;
(xxv) comprises a CDR3α comprising the amino acid sequence of CAARSYNTDKLIF (SEQ ID NO: 186) or a variant thereof having up to three amino acid substitutions, additions or deletions, and a CDR3β comprising the amino acid sequence of CASSSYPLRTGRYNSYNSPLHF (SEQ ID NO: 208) or a variant thereof having up to three amino acid substitutions, additions or deletions;
(xxvi) comprises a CDR3α comprising the amino acid sequence of CAASYNNARLMF (SEQ ID NO: 191) or a variant thereof having up to three amino acid substitutions, additions or deletions, and a CDR3β comprising the amino acid sequence of CASSWGYQETQYF (SEQ ID NO: 196) or a variant thereof having up to three amino acid substitutions, additions or deletions;
(xxvii) comprises a CDR3α comprising the amino acid sequence of CAASYNNARLMF (SEQ ID NO: 191) or a variant thereof having up to three amino acid substitutions, additions or deletions, and a CDR3β comprising the amino acid sequence of CASSPTGGEYYGYTF (SEQ ID NO: 202) or a variant thereof having up to three amino acid substitutions, additions or deletions;
(xxviii) comprises a CDR3α comprising the amino acid sequence of CAASYNNARLMF (SEQ ID NO: 191) or a variant thereof having up to three amino acid substitutions, additions or deletions, and a CDR3β comprising the amino acid sequence of CASSSYPLRTGRYNSYNSPLHF (SEQ ID NO: 208) or a variant thereof having up to three amino acid substitutions, additions or deletions;
(xxix) comprises a CDR3α comprising the amino acid sequence of CAASGGRDDKIIF (SEQ ID NO: 214) or a variant thereof having up to three amino acid substitutions, additions or deletions, and a CDR3β comprising the amino acid sequence of CASSYSRTESTDTQYF (SEQ ID NO: 219) or a variant thereof having up to three amino acid substitutions, additions or deletions;
(xxx) comprises a CDR3α comprising the amino acid sequence of CAANNARLMF (SEQ ID NO: 92) or a variant thereof having up to three amino acid substitutions, additions or deletions, and a CDR3β comprising the amino acid sequence of CASSPGQHGELFF (SEQ ID NO: 271) or a variant thereof having up to three amino acid substitutions, additions or deletions;
(xxxi) comprises a CDR3α comprising the amino acid sequence of CAASATGNQFYF (SEQ ID NO: 266) or a variant thereof having up to three amino acid substitutions, additions or deletions, and a CDR3β comprising the amino acid sequence of CASSDTRAREQFF (SEQ ID NO: 97) or a variant thereof having up to three amino acid substitutions, additions or deletions;
(xxxii) comprises a CDR3α comprising the amino acid sequence of CAASATGNQFYF (SEQ ID NO: 266) or a variant thereof having up to three amino acid substitutions, additions or deletions, and a CDR3β comprising the amino acid sequence of CASSPGQHGELFF (SEQ ID NO: 271) or a variant thereof having up to three amino acid substitutions, additions or deletions;
(xxxiii) comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions;
(xxxiv) comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSVGGSGSYNEQFF (SEQ ID NO: 169) or a variant thereof having up to three amino acid substitutions, additions or deletions;
(xxxv) comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions;
(xxxvi) comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CASSLGLSISQETQYF (SEQ ID NO: 288) or a variant thereof having up to three amino acid substitutions, additions or deletions;
(xxxvii) comprises a CDR3α comprising the amino acid sequence of CAERLNTDKLIF (SEQ ID NO: 103) or a variant thereof having up to three amino acid substitutions, additions or deletions, and a CDR3β comprising the amino acid sequence of CASSLGLSISQETQYF (SEQ ID NO: 288) or a variant thereof having up to three amino acid substitutions, additions or deletions;
(xxxviii) comprises a CDR3α comprising the amino acid sequence of CAVEATDSWGKLQF (SEQ ID NO: 108) or a variant thereof having up to three amino acid substitutions, additions or deletions, and a CDR3β comprising the amino acid sequence of CASSLGLSISQETQYF (SEQ ID NO: 288) or a variant thereof having up to three amino acid substitutions, additions or deletions;
(xxxix) comprises a CDR3α comprising the amino acid sequence of CAVRTSYDKVIF (SEQ ID NO: 113) or a variant thereof having up to three amino acid substitutions, additions or deletions, and a CDR3β comprising the amino acid sequence of CASSLGLSISQETQYF (SEQ ID NO: 288) or a variant thereof having up to three amino acid substitutions, additions or deletions;
(xxxx) comprises a CDR3α comprising the amino acid sequence of CAERLNTDKLIF (SEQ ID NO: 103) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions;
(xxxxi) comprises a CDR3α comprising the amino acid sequence of CAVEATDSWGKLQF (SEQ ID NO: 108) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions; or
(xxxxii) comprises a CDR3α comprising the amino acid sequence of CAVRTSYDKVIF (SEQ ID NO: 113) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions.

2. A TCR according to claim 1 comprising the following CDR sequences:
(i)
(SEQ ID NO: 1)
CDR1α - KALYS,
(SEQ ID NO: 2)
CDR2α - LLKGGEQ,
(SEQ ID NO: 3)
CDR3α - CGTAWINDYKLSF,
(SEQ ID NO: 6)
CDR1β - SGHDY,
(SEQ ID NO: 7)
CDR2β - FNNNVP,
and
(SEQ ID NO: 8)
CDR3β - CASRKTGGYSNQPQHF,
or variants thereof each having up to three amino acid substitutions, additions or deletions;
(ii)
(SEQ ID NO: 34)
CDR1α - NSASQS,
(SEQ ID NO: 35)
CDR2α - VYSSGN,
(SEQ ID NO: 36)
CDR3α - CVVNLLSNQGGKLIF,
(SEQ ID NO: 39)
CDR1β - LGHNA,
(SEQ ID NO: 40)
CDR2β - YSLEER,
and
(SEQ ID NO: 41)
CDR3β - CASSQDYLVSNEKLFF,
or variants thereof each having up to three amino acid substitutions, additions or deletions;
(iii)
(SEQ ID NO: 12)
CDR1α - SSVPPY,
(SEQ ID NO: 13)
CDR2α - YTSAATLV,
(SEQ ID NO: 14)
CDR3α - CAVRLSGSARQLTF,
(SEQ ID NO: 22)
CDR1β - SGHAT,
(SEQ ID NO: 23)
CDR2β - FQNNGV,
and
(SEQ ID NO: 24)
CDR3β - CASSLLGDEQYF,
or variants thereof each having up to three amino acid substitutions, additions or deletions;
(iv)
(SEQ ID NO: 12)
CDR1α - SSVPPY,
(SEQ ID NO: 13)
CDR2α - YTSAATLV,
(SEQ ID NO: 14)
CDR3α - CAVRLSGSARQLTF,
(SEQ ID NO: 28)
CDR1β - SGHTA,
(SEQ ID NO: 29)
CDR2β - FQGNSA,
and
(SEQ ID NO: 30)
CDR3β - CASSLVALQGAGEQYF,
or variants thereof each having up to three amino acid substitutions, additions or deletions;
(v)
(SEQ ID NO: 17)
CDR1α - TSESDYY,
(SEQ ID NO: 18)
CDR2α - QEAYKQQN,
(SEQ ID NO: 19)
CDR3α - CAYRSLKYGNKLVF,
(SEQ ID NO: 22)
CDR1β - SGHAT,
(SEQ ID NO: 23)
CDR2β - FQNNGV,
and
(SEQ ID NO: 24)
CDR3β - CASSLLGDEQYF,
or variants thereof each having up to three amino acid substitutions, additions or deletions;
(vi)
(SEQ ID NO: 17)
CDR1α - TSESDYY,
(SEQ ID NO: 18)
CDR2α - QEAYKQQN,
(SEQ ID NO: 19)
CDR3α - CAYRSLKYGNKLVF,
(SEQ ID NO: 28)
CDR1β - SGHTA,
(SEQ ID NO: 29)
CDR2β - FQGNSA,
and
(SEQ ID NO: 30)
CDR3β - CASSLVALQGAGEQYF,
or variants thereof each having up to three amino acid substitutions, additions or deletions;
(vii)
(SEQ ID NO: 45)
CDR1α - TSINN,
(SEQ ID NO: 46)
CDR2α - IRSNERE,
(SEQ ID NO: 47)
CDR3α - CATDAYSGNTPLVF,
(SEQ ID NO: 55)
CDR1β - MNHNS,
(SEQ ID NO: 56)
CDR2β - SASEGT,
and
(SEQ ID NO: 57)
CDR3β - CASRAAGLDTEAFF,
or variants thereof each having up to three amino acid substitutions, additions or deletions;
(viii)
(SEQ ID NO: 45)
CDR1α - TSINN,
(SEQ ID NO: 46)
CDR2α - IRSNERE,
(SEQ ID NO: 47)
CDR3α - CATDAYSGNTPLVF,
(SEQ ID NO: 61)
CDR1β - MNHNY,
(SEQ ID NO: 62)
CDR2β - SVGAGI,
and
(SEQ ID NO: 63)
CDR3β - CASTQTPYEQYF,
or variants thereof each having up to three amino acid substitutions, additions or deletions;
(ix)
(SEQ ID NO: 45)
CDR1α - TSINN,
(SEQ ID NO: 46)
CDR2α - IRSNERE,
(SEQ ID NO: 47)
CDR3α - CATDAYSGNTPLVF,
(SEQ ID NO: 67)
CDR1β - SGHNS,
(SEQ ID NO: 68)
CDR2β - FNNNVP,
and
(SEQ ID NO: 69)
CDR3β - CASSTVGGEDYGYTF,
or variants thereof each having up to three amino acid substitutions, additions or deletions;
(x)
(SEQ ID NO: 50)
CDR1α - DSAIYN,
(SEQ ID NO: 51)
CDR2α - IQSSQRE,
(SEQ ID NO: 52)
CDR3α - CAVRAEIYNQGGKLIF,
(SEQ ID NO: 55)
CDR1β - MNHNS,
(SEQ ID NO: 56)
CDR2β - SASEGT,
and
(SEQ ID NO: 57)
CDR3β - CASRAAGLDTEAFF,
or variants thereof each having up to three amino acid substitutions, additions or deletions;
(xi)
(SEQ ID NO: 50)
CDR1α - DSAIYN,
(SEQ ID NO: 51)
CDR2α - IQSSQRE,
(SEQ ID NO: 52)
CDR3α - CAVRAEIYNQGGKLIF,
(SEQ ID NO: 61)
CDR1β - MNHNY,
(SEQ ID NO: 62)
CDR2β - SVGAGI,
and
(SEQ ID NO: 63)
CDR3β - CASTQTPYEQYF,
or variants thereof each having up to three amino acid substitutions, additions or deletions;
(xii)
(SEQ ID NO: 50)
CDR1α - DSAIYN,
(SEQ ID NO: 51)
CDR2α - IQSSQRE,
(SEQ ID NO: 52)
CDR3α - CAVRAEIYNQGGKLIF,
(SEQ ID NO: 67)
CDR1β - SGHNS,
(SEQ ID NO: 68)
CDR2β - FNNNVP,
and
(SEQ ID NO: 69)
CDR3β - CASSTVGGEDYGYTF,
or variants thereof each having up to three amino acid substitutions, additions or deletions;
(xiii)
(SEQ ID NO: 73)
CDR1α - DSASNY,
(SEQ ID NO: 74)
CDR2α - IRSNVGE,
(SEQ ID NO: 75)
CDR3α - CAASMAGAGSYQLTF,
(SEQ ID NO: 78)
CDR1β - ENHRY,
(SEQ ID NO: 79)
CDR2β - SYGVKD,
and
(SEQ ID NO: 80)
CDR3β - CAISVGQGALYEQYF,
or variants thereof each having up to three amino acid substitutions, additions or deletions;
(xiv)
(SEQ ID NO: 73)
CDR1α - DSASNY,
(SEQ ID NO: 74)
CDR2α - IRSNVGE,
(SEQ ID NO: 75)
CDR3α - CAASMAGAGSYQLTF,
(SEQ ID NO: 84)
CDR1β - SGDLS,
(SEQ ID NO: 85)
CDR2β - YYNGEE,
and
(SEQ ID NO: 86)
CDR3β - CASSVARDRRNYGYTF,
or variants thereof each having up to three amino acid substitutions, additions or deletions;
(xv)
(SEQ ID NO: 90)
CDR1α - NSMFDY,
(SEQ ID NO: 91)
CDR2α - ISSIKDK,
(SEQ ID NO: 92)
CDR3α - CAANNARLMF,
(SEQ ID NO: 95)
CDR1β - SGHNS,
(SEQ ID NO: 96)
CDR2β - FNNNVP,
and
(SEQ ID NO: 97)
CDR3β - CASSDTRAREQFF,
or variants thereof each having up to three amino acid substitutions, additions or deletions;
(xvi)
(SEQ ID NO: 101)
CDR1α - DSSSTY,
(SEQ ID NO: 102)
CDR2α - IFSNMDM,
(SEQ ID NO: 103)
CDR3α - CAERLNTDKLIF,
(SEQ ID NO: 161)
CDR1β - DFQATT,
(SEQ ID NO: 162)
CDR2β - SNEGSKA,
and
(SEQ ID NO: 163)
CDR3β - CSARDSVSGNTIYF,
or variants thereof each having up to three amino acid substitutions, additions or deletions;
(xvii)
(SEQ ID NO: 101)
CDR1α - DSSSTY,
(SEQ ID NO: 102)
CDR2α - IFSNMDM,
(SEQ ID NO: 103)
CDR3α - CAERLNTDKLIF,
(SEQ ID NO: 167)
CDR1β - SQVTM,
(SEQ ID NO: 168)
CDR2β - ANQGSEA,
and
(SEQ ID NO: 169)
CDR3β - CSVGGSGSYNEQFF,
or variants thereof each having up to three amino acid substitutions, additions or deletions;
(xviii)
(SEQ ID NO: 106)
CDR1α - DSVNN,
(SEQ ID NO: 107)
CDR2α - IPSGT,
(SEQ ID NO: 108)
CDR3α - CAVEATDSWGKLQF,
(SEQ ID NO: 161)
CDR1β - DFQATT,
(SEQ ID NO: 162)
CDR2β - SNEGSKA,
and
(SEQ ID NO: 163)
CDR3β - CSARDSVSGNTIYF,
or variants thereof each having up to three amino acid substitutions, additions or deletions;
(xix)
(SEQ ID NO: 106)
CDR1α - DSVNN,
(SEQ ID NO: 107)
CDR2α - IPSGT,
(SEQ ID NO: 108)
CDR3α - CAVEATDSWGKLQF,
(SEQ ID NO: 167)
CDR1β - SQVTM,
(SEQ ID NO: 168)
CDR2β - ANQGSEA,
and
(SEQ ID NO: 169)
CDR3β - CSVGGSGSYNEQFF,
or variants thereof each having up to three amino acid substitutions, additions or deletions;
(xx)
(SEQ ID NO: 111)
CDR1α - DSASNY
(SEQ ID NO: 112)
CDR2α - IRSNVGE,
(SEQ ID NO: 113)
CDR3α - CAVRTSYDKVIF,
(SEQ ID NO: 161)
CDR1β - DFQATT,
(SEQ ID NO: 162)
CDR2β - SNEGSKA,
and
(SEQ ID NO: 163)
CDR3β - CSARDSVSGNTIYF,
or variants thereof each having up to three amino acid substitutions, additions or deletions;
(xxi)
(SEQ ID NO: 111)
CDR1α - DSASNY,
(SEQ ID NO: 112)
CDR2α - IRSNVGE,
(SEQ ID NO: 113)
CDR3α - CAVRTSYDKVIF,
(SEQ ID NO: 167)
CDR1β - SQVTM,
(SEQ ID NO: 168)
CDR2β - ANQGSEA,
and
(SEQ ID NO: 169)
CDR3β - CSVGGSGSYNEQFF,
or variants thereof each having up to three amino acid substitutions, additions or deletions;
(xxii)
(SEQ ID NO: 173)
CDR1α - VGISA,
(SEQ ID NO: 174)
CDR2α - LSSGK,
(SEQ ID NO: 175)
CDR3α - CAVTVGNKLVF,
(SEQ ID NO: 178)
CDR1β - MNHNS,
(SEQ ID NO: 179)
CDR2β - SASEGT,
and
(SEQ ID NO: 180)
CDR3β - CASRGWREQFF,
or variants thereof each having up to three amino acid substitutions, additions or deletions;
(xxiii)
(SEQ ID NO: 184)
CDR1α - VGISA,
(SEQ ID NO: 185)
CDR2α - LSSGK,
(SEQ ID NO: 186)
CDR3α - CAARSYNTDKLIF,
(SEQ ID NO: 194)
CDR1β - SGHTS,
(SEQ ID NO: 195)
CDR2β - YDEGEE,
and
(SEQ ID NO: 196)
CDR3β - CASSWGYQETQYF,
or variants thereof each having up to three amino acid substitutions, additions or deletions;
(xxiv)
(SEQ ID NO: 184)
CDR1α - VGISA,
(SEQ ID NO: 185)
CDR2α - LSSGK,
(SEQ ID NO: 186)
CDR3α - CAARSYNTDKLIF,
(SEQ ID NO: 200)
CDR1β - KGHSH,
(SEQ ID NO: 201)
CDR2β - LQKENI,
and
(SEQ ID NO: 202)
CDR3β - CASSPTGGEYYGYTF,
or variants thereof each having up to three amino acid substitutions, additions or deletions;
(xxv)
(SEQ ID NO: 184)
CDR1α - VGISA,
(SEQ ID NO: 185)
CDR2α - LSSGK,
(SEQ ID NO: 186)
CDR3α - CAARSYNTDKLIF,
(SEQ ID NO: 206)
CDR1β - MNHEY,
(SEQ ID NO: 207)
CDR2β - SVGAGI,
and
(SEQ ID NO: 208)
CDR3β - CASSSYPLRTGRYNSYNSPLHF,
or variants thereof each having up to three amino acid substitutions, additions or deletions;
(xxvi)
(SEQ ID NO: 189)
CDR1α - NSMFDY,
(SEQ ID NO: 190)
CDR2α - ISSIKDK,
(SEQ ID NO: 191)
CDR3α - CAASYNNARLMF,
(SEQ ID NO: 194)
CDR1β - SGHTS,
(SEQ ID NO: 195)
CDR2β - YDEGEE,
and
(SEQ ID NO: 196)
CDR3β - CASSWGYQETQYF,
or variants thereof each having up to three amino acid substitutions, additions or deletions;
(xxvii)
(SEQ ID NO: 189)
CDR1α - NSMFDY,
(SEQ ID NO: 190)
CDR2α - ISSIKDK,
(SEQ ID NO: 191)
CDR3α - CAASYNNARLMF,
(SEQ ID NO: 200)
CDR1β - KGHSH,
(SEQ ID NO: 201)
CDR2β - LQKENI,
and
(SEQ ID NO: 202)
CDR3β - CASSPTGGEYYGYTF,
or variants thereof each having up to three amino acid substitutions, additions or deletions;
(xxviii)
(SEQ ID NO: 189)
CDR1α-NSMFDY,
(SEQ ID NO: 190)
CDR2α-ISSIKDK,
(SEQ ID NO: 191)
CDR3α-CAASYNNARLMF,
(SEQ ID NO: 206)
CDR1β-MNHEY,
(SEQ ID NO: 207)
CDR2β-SVGAGI,
and
(SEQ ID NO: 208)
CDR3β-CASSSYPLRTGRYNSYNSPLHF,
or variants thereof each having up to three amino acid substitutions, additions or deletions;
(xxix)
(SEQ ID NO: 212)
CDR1α-NSMFDY,
(SEQ ID NO: 213)
CDR2α-ISSIKDK,
(SEQ ID NO: 214)
CDR3α-CAASGGRDDKIIF,
(SEQ ID NO: 217)
CDR1β-MNHEY,
(SEQ ID NO: 218)
CDR2β-SVGAGI,
and
(SEQ ID NO: 219)
CDR3β-CASSYSRTESTDTQYF,
or variants thereof each having up to three amino acid substitutions, additions or deletions;
(xxx)
(SEQ ID NO: 90)
CDR1α-NSMFDY,
(SEQ ID NO: 91)
CDR2α-ISSIKDK,
(SEQ ID NO: 92)
CDR3α-CAANNARLMF,
(SEQ ID NO: 269)
CDR1β-SGHRS,
(SEQ ID NO: 270)
CDR2β-YFSETQ,
and
(SEQ ID NO: 271)
CDR3β-CASSPGQHGELFF,
or variants thereof each having up to three amino acid substitutions, additions or deletions;
(xxxi)
(SEQ ID NO: 264)
CDR1α-NSMFDY,
(SEQ ID NO: 265)
CDR2α-ISSIKDK,
(SEQ ID NO: 266)
CDR3α-CAASATGNQFYF,
(SEQ ID NO: 95)
CDR1β-SGHNS,
(SEQ ID NO: 96)
CDR2β-FNNNVP,
and
(SEQ ID NO: 97)
CDR3β-CASSDTRAREQFF,
or variants thereof each having up to three amino acid substitutions, additions or deletions;
(xxxii)
(SEQ ID NO: 264)
CDR1α-NSMFDY,
(SEQ ID NO: 265)
CDR2α-ISSIKDK,
(SEQ ID NO: 266)
CDR3α-CAASATGNQFYF,
(SEQ ID NO: 269)
CDR1β-SGHRS,
(SEQ ID NO: 270)
CDR2β-YFSETQ,
and
(SEQ ID NO: 271)
CDR3β-CASSPGQHGELFF,
or variants thereof each having up to three amino acid substitutions, additions or deletions;
(xxxiii)
(SEQ ID NO: 275)
CDR1α-TSINN,
(SEQ ID NO: 276)
CDR2α-IRSNERE,
(SEQ ID NO: 277)
CDR3α-CATDGDSSYKLIF,
(SEQ ID NO: 161)
CDR1β-DFQATT,
(SEQ ID NO: 162)
CDR2β-SNEGSKA,
and
(SEQ ID NO: 163)
CDR3β-CSARDSVSGNTIYF,
or variants thereof each having up to three amino acid substitutions, additions or deletions;
(xxxiv)
(SEQ ID NO: 275)
CDR1α-TSINN,
(SEQ ID NO: 276)
CDR2α-IRSNERE,
(SEQ ID NO: 277)
CDR3α-CATDGDSSYKLIF,
(SEQ ID NO: 167)
CDR1β-SQVTM,
(SEQ ID NO: 168)
CDR2β-ANQGSEA,
and
(SEQ ID NO: 169)
CDR3β-CSVGGSGSYNEQFF,
or variants thereof each having up to three amino acid substitutions, additions or deletions;
(xxxv)
(SEQ ID NO: 275)
CDR1α-TSINN,
(SEQ ID NO: 276)
CDR2α-IRSNERE,
(SEQ ID NO: 277)
CDR3α-CATDGDSSYKLIF,
(SEQ ID NO: 280)
CDR1β-DFQATT,
(SEQ ID NO: 281)
CDR2β-SNEGSKA,
and
(SEQ ID NO: 282)
CDR3β-CSARDVLTGDYGYTF,
or variants thereof each having up to three amino acid substitutions, additions or deletions;
(xxxvi)
(SEQ ID NO: 275)
CDR1α-TSINN,
(SEQ ID NO: 276)
CDR2α-IRSNERE,
(SEQ ID NO: 277)
CDR3α-CATDGDSSYKLIF,
(SEQ ID NO: 286)
CDR1β-SGHDY,
(SEQ ID NO: 287)
CDR2β-FNNNVP,
and
(SEQ ID NO: 288)
CDR3β-CASSLGLSISQETQYF,
or variants thereof each having up to three amino acid substitutions, additions or deletions;
(xxxvii)
(SEQ ID NO: 101)
CDR1α-DSSSTY,
(SEQ ID NO: 102)
CDR2α-IFSNMDM,
(SEQ ID NO: 103)
CDR3α-CAERLNTDKLIF,
(SEQ ID NO: 286)
CDR1β-SGHDY,
(SEQ ID NO: 287)
CDR2β-FNNNVP,
and
(SEQ ID NO: 288)
CDR3β-CASSLGLSISQETQYF,
or variants thereof each having up to three amino acid substitutions, additions or deletions;
(xxxviii)
(SEQ ID NO: 106)
CDR1α-DSVNN,
(SEQ ID NO: 107)
CDR2α-IPSGT,
(SEQ ID NO: 108)
CDR3α-CAVEATDSWGKLQF,
(SEQ ID NO: 286)
CDR1β-SGHDY,
(SEQ ID NO: 287)
CDR2β-FNNNVP,
and
(SEQ ID NO: 288)
CDR3β-CASSLGLSISQETQYF,
or variants thereof each having up to three amino acid substitutions, additions or deletions;
(xxxix)
(SEQ ID NO: 111)
CDR1α-DSASNY,
(SEQ ID NO: 112)
CDR2α-IRSNVGE,
(SEQ ID NO: 113)
CDR3α-CAVRTSYDKVIF,
(SEQ ID NO: 286)
CDR1β-SGHDY,
(SEQ ID NO: 287)
CDR2β-FNNNVP,
and
(SEQ ID NO: 288)
CDR3β-CASSLGLSISQETQYF,
or variants thereof each having up to three amino acid substitutions, additions or deletions;
(xxxx)
(SEQ ID NO: 101)
CDR1α-DSSSTY,
(SEQ ID NO: 102)
CDR2α-IFSNMDM,
(SEQ ID NO: 103)
CDR3α-CAERLNTDKLIF,
(SEQ ID NO: 280)
CDR1β-DFQATT,
(SEQ ID NO: 281)
CDR2β-SNEGSKA,
and
(SEQ ID NO: 282)
CDR3β-CSARDVLTGDYGYTF,
or variants thereof each having up to three amino acid substitutions, additions or deletions;
(xxxxi)
(SEQ ID NO: 106)
CDR1α-DSVNN,
(SEQ ID NO: 107)
CDR2α-IPSGT,
(SEQ ID NO: 108)
CDR3α-CAVEATDSWGKLQF,
(SEQ ID NO: 280)
CDR1β-DFQATT,
(SEQ ID NO: 281)
CDR2β-SNEGSKA,
and
(SEQ ID NO: 282)
CDR3β-CSARDVLTGDYGYTF,
or variants thereof each having up to three amino acid substitutions, additions or deletions; or
(xxxxii)
(SEQ ID NO: 111)
CDR1α-DSASNY,
(SEQ ID NO: 112)
CDR2α-IRSNVGE,
(SEQ ID NO: 113)
CDR3α-CAVRTSYDKVIF,
(SEQ ID NO: 280)
CDR1β-DFQATT,
(SEQ ID NO: 281)
CDR2β-SNEGSKA,
and
(SEQ ID NO: 282)
CDR3β-CSARDVLTGDYGYTF,
or variants thereof each having up to three amino acid substitutions, additions or deletions.

3. A TCR according to claim 1 comprising:
(i) an α chain variable domain comprising the amino acid sequence of SEQ ID NO: 4 or a variant thereof having at least 75% sequence identity thereto; and a β chain variable domain comprising the amino acid sequence of SEQ ID NO: 9 or a variant thereof having at least 75% sequence identity thereto;
(ii) an α chain variable domain comprising the amino acid sequence of SEQ ID NO: 37 or a variant thereof having at least 75% sequence identity thereto; and a β chain variable domain comprising the amino acid sequence of SEQ ID NO: 42 or a variant thereof having at least 75% sequence identity thereto;
(iii) an α chain variable domain comprising the amino acid sequence of SEQ ID NO: 15 or a variant thereof having at least 75% sequence identity thereto; and a β chain variable domain comprising the amino acid sequence of SEQ ID NO: 25 or a variant thereof having at least 75% sequence identity thereto;
(iv) an α chain variable domain comprising the amino acid sequence of SEQ ID NO: 15 or a variant thereof having at least 75% sequence identity thereto; and a β chain variable domain comprising the amino acid sequence of SEQ ID NO: 31 or a variant thereof having at least 75% sequence identity thereto;
(v) an α chain variable domain comprising the amino acid sequence of SEQ ID NO: 20 or a variant thereof having at least 75% sequence identity thereto; and a β chain variable domain comprising the amino acid sequence of SEQ ID NO: 25 or a variant thereof having at least 75% sequence identity thereto;
(vi) an α chain variable domain comprising the amino acid sequence of SEQ ID NO: 20 or a variant thereof having at least 75% sequence identity thereto; and a β chain variable domain comprising the amino acid sequence of SEQ ID NO: 31 or variants thereof having at least 75% sequence identity thereto;
(vii) an α chain variable domain comprising the amino acid sequence of SEQ ID NO: 48 or a variant thereof having at least 75% sequence identity thereto; and a β chain variable domain comprising the amino acid sequence of SEQ ID NO: 58 or a variant thereof having at least 75% sequence identity thereto;
(viii) an α chain variable domain comprising the amino acid sequence of SEQ ID NO: 48 or a variant thereof having at least 75% sequence identity thereto; and a β chain variable domain comprising the amino acid sequence of SEQ ID NO: 64 or a variant thereof having at least 75% sequence identity thereto;
(ix) an α chain variable domain comprising the amino acid sequence of SEQ ID NO: 48 or a variant thereof having at least 75% sequence identity thereto; and a β chain variable domain comprising the amino acid sequence of SEQ ID NO: 70 or a variant thereof having at least 75% sequence identity thereto;
(x) an α chain variable domain comprising the amino acid sequence of SEQ ID NO: 53 or a variant thereof having at least 75% sequence identity thereto; and a β chain variable domain comprising the amino acid sequence of SEQ ID NO: 58 or a variant thereof having at least 75% sequence identity thereto;
(xi) an α chain variable domain comprising the amino acid sequence of SEQ ID NO: 53 or a variant thereof having at least 75% sequence identity thereto; and a β chain variable domain comprising the amino acid sequence of SEQ ID NO: 64 or a variant thereof having at least 75% sequence identity thereto;
(xii) an α chain variable domain comprising the amino acid sequence of SEQ ID NO: 53 or a variant thereof having at least 75% sequence identity thereto; and a β chain variable domain comprising the amino acid sequence of SEQ ID NO: 70 or a variant thereof having at least 75% sequence identity thereto;
(xiii) an α chain variable domain comprising the amino acid sequence of SEQ ID NO: 76 or a variant thereof having at least 75% sequence identity thereto; and a β chain variable domain comprising the amino acid sequence of SEQ ID NO: 81 or a variant thereof having at least 75% sequence identity thereto;
(xiv) an α chain variable domain comprising the amino acid sequence of SEQ ID NO: 76 or a variant thereof having at least 75% sequence identity thereto; and a β chain variable domain comprising the amino acid sequence of SEQ ID NO: 87 or a variant thereof having at least 75% sequence identity thereto;
(xv) an α chain variable domain comprising the amino acid sequence of SEQ ID NO: 93 or a variant thereof having at least 75% sequence identity thereto; and a β chain variable domain comprising the amino acid sequence of SEQ ID NO: 98 or a variant thereof having at least 75% sequence identity thereto;
(xvi) an α chain variable domain comprising the amino acid sequence of SEQ ID NO: 104 or a variant thereof having at least 75% sequence identity thereto; and a β chain variable domain comprising the amino acid sequence of SEQ ID NO: 164 or a variant thereof having at least 75% sequence identity thereto;
(xvii) an α chain variable domain comprising the amino acid sequence of SEQ ID NO: 104 or a variant thereof having at least 75% sequence identity thereto; and a β chain variable domain comprising the amino acid sequence of SEQ ID NO: 170 or a variant thereof having at least 75% sequence identity thereto;
(xviii) an α chain variable domain comprising the amino acid sequence of SEQ ID NO: 109 or a variant thereof having at least 75% sequence identity thereto; and a β chain variable domain comprising the amino acid sequence of SEQ ID NO: 164 or a variant thereof having at least 75% sequence identity thereto;
(xix) an α chain variable domain comprising the amino acid sequence of SEQ ID NO: 109 or a variant thereof having at least 75% sequence identity thereto; and a β chain variable domain comprising the amino acid sequence of SEQ ID NO: 170 or a variant thereof having at least 75% sequence identity thereto;
(xx) an α chain variable domain comprising the amino acid sequence of SEQ ID NO: 114 or a variant thereof having at least 75% sequence identity thereto; and a β chain variable domain comprising the amino acid sequence of SEQ ID NO: 164 or a variant thereof having at least 75% sequence identity thereto;
(xxi) an α chain variable domain comprising the amino acid sequence of SEQ ID NO: 114 or a variant thereof having at least 75% sequence identity thereto; and a β chain variable domain comprising the amino acid sequence of SEQ ID NO: 170 or a variant thereof having at least 75% sequence identity thereto;
(xxii) an α chain variable domain comprising the amino acid sequence of SEQ ID NO: 176 or a variant thereof having at least 75% sequence identity thereto; and a β chain variable domain comprising the amino acid sequence of SEQ ID NO: 181 or a variant thereof having at least 75% sequence identity thereto;
(xxiii) an α chain variable domain comprising the amino acid sequence of SEQ ID NO: 187 or a variant thereof having at least 75% sequence identity thereto; and a β chain variable domain comprising the amino acid sequence of SEQ ID NO: 197 or a variant thereof having at least 75% sequence identity thereto;
(xxiv) an α chain variable domain comprising the amino acid sequence of SEQ ID NO: 187 or a variant thereof having at least 75% sequence identity thereto; and a β chain variable domain comprising the amino acid sequence of SEQ ID NO: 203 or a variant thereof having at least 75% sequence identity thereto;
(xxv) an α chain variable domain comprising the amino acid sequence of SEQ ID NO: 187 or a variant thereof having at least 75% sequence identity thereto; and a β chain variable domain comprising the amino acid sequence of SEQ ID NO: 209 or a variant thereof having at least 75% sequence identity thereto;
(xxvi) an α chain variable domain comprising the amino acid sequence of SEQ ID NO: 192 or a variant thereof having at least 75% sequence identity thereto; and a β chain variable domain comprising the amino acid sequence of SEQ ID NO: 197 or a variant thereof having at least 75% sequence identity thereto;
(xxvii) an α chain variable domain comprising the amino acid sequence of SEQ ID NO: 192 or a variant thereof having at least 75% sequence identity thereto; and a β chain variable domain comprising the amino acid sequence of SEQ ID NO: 203 or a variant thereof having at least 75% sequence identity thereto;
(xxviii) an α chain variable domain comprising the amino acid sequence of SEQ ID NO: 192 or a variant thereof having at least 75% sequence identity thereto; and a β chain variable domain comprising the amino acid sequence of SEQ ID NO: 209 or a variant thereof having at least 75% sequence identity thereto;
(xxix) an α chain variable domain comprising the amino acid sequence of SEQ ID NO: 215 or a variant thereof having at least 75% sequence identity thereto; and a β chain variable domain comprising the amino acid sequence of SEQ ID NO: 220 or a variant thereof having at least 75% sequence identity thereto;
(xxx) an α chain variable domain comprising the amino acid sequence of SEQ ID NO: 93 or a variant thereof having at least 75% sequence identity thereto; and a β chain variable domain comprising the amino acid sequence of SEQ ID NO: 272 or a variant thereof having at least 75% sequence identity thereto;
(xxxi) an α chain variable domain comprising the amino acid sequence of SEQ ID NO: 267 or a variant thereof having at least 75% sequence identity thereto; and a β chain variable domain comprising the amino acid sequence of SEQ ID NO: 98 or a variant thereof having at least 75% sequence identity thereto;
(xxxii) an α chain variable domain comprising the amino acid sequence of SEQ ID NO: 267 or a variant thereof having at least 75% sequence identity thereto; and a β chain variable domain comprising the amino acid sequence of SEQ ID NO: 272 or a variant thereof having at least 75% sequence identity thereto;
(xxxiii) an α chain variable domain comprising the amino acid sequence of SEQ ID NO: 278 or a variant thereof having at least 75% sequence identity thereto; and a β chain variable domain comprising the amino acid sequence of SEQ ID NO: 164 or a variant thereof having at least 75% sequence identity thereto;
(xxxiv) an α chain variable domain comprising the amino acid sequence of SEQ ID NO: 278 or a variant thereof having at least 75% sequence identity thereto; and a β chain variable domain comprising the amino acid sequence of SEQ ID NO: 170 or a variant thereof having at least 75% sequence identity thereto;
(xxxv) an α chain variable domain comprising the amino acid sequence of SEQ ID NO: 278 or a variant thereof having at least 75% sequence identity thereto; and a β chain variable domain comprising the amino acid sequence of SEQ ID NO: 283 or a variant thereof having at least 75% sequence identity thereto;
(xxxvi) an α chain variable domain comprising the amino acid sequence of SEQ ID NO: 278 or a variant thereof having at least 75% sequence identity thereto; and a β chain variable domain comprising the amino acid sequence of SEQ ID NO: 289 or a variant thereof having at least 75% sequence identity thereto;
(xxxvii) an α chain variable domain comprising the amino acid sequence of SEQ ID NO: 104 or a variant thereof having at least 75% sequence identity thereto; and a β chain variable domain comprising the amino acid sequence of SEQ ID NO: 289 or a variant thereof having at least 75% sequence identity thereto;
(xxxviii) an α chain variable domain comprising the amino acid sequence of SEQ ID NO: 109 or a variant thereof having at least 75% sequence identity thereto; and a β chain variable domain comprising the amino acid sequence of SEQ ID NO: 289 or a variant thereof having at least 75% sequence identity thereto;
(xxxix) an α chain variable domain comprising the amino acid sequence of SEQ ID NO: 114 or a variant thereof having at least 75% sequence identity thereto; and a β chain variable domain comprising the amino acid sequence of SEQ ID NO: 289 or a variant thereof having at least 75% sequence identity thereto;
(xxxx) an α chain variable domain comprising the amino acid sequence of SEQ ID NO: 104 or a variant thereof having at least 75% sequence identity thereto; and a β chain variable domain comprising the amino acid sequence of SEQ ID NO: 283 or a variant thereof having at least 75% sequence identity thereto;
(xxxxi) an α chain variable domain comprising the amino acid sequence of SEQ ID NO: 109 or a variant thereof having at least 75% sequence identity thereto; and a β chain variable domain comprising the amino acid sequence of SEQ ID NO: 283 or a variant thereof having at least 75% sequence identity thereto; or
(xxxxii) an α chain variable domain comprising the amino acid sequence of SEQ ID NO: 114 or a variant thereof having at least 75% sequence identity thereto; and a β chain variable domain comprising the amino acid sequence of SEQ ID NO: 283 or a variant thereof having at least 75% sequence identity thereto.

4. A TCR according to claim 1 comprising:
(i) an α chain comprising the amino acid sequence of SEQ ID NO: 5 or a variant thereof having at least 75% sequence identity thereto; and a β chain comprising an amino acid sequence selected from the group consisting of SEQ ID NO: 10, SEQ ID NO: 11 and variants of SEQ ID NOs: 10 and 11 having at least 75% sequence identity thereto;
(ii) an α chain comprising the amino acid sequence of SEQ ID NO: 38 or a variant thereof having at least 75% sequence identity thereto; and a β chain comprising an amino acid sequence selected from the group consisting of SEQ ID NO: 43, SEQ ID NO: 44 and variants of SEQ ID NOs: 43 and 44 having at least 75% sequence identity thereto;
(iii) an α chain comprising the amino acid sequence of SEQ ID NO: 16 or a variant thereof having at least 75% sequence identity thereto; and a β chain comprising an amino acid sequence selected from the group consisting of SEQ ID NO: 26, SEQ ID NO: 27 and variants of SEQ ID NOs: 26 and 27 having at least 75% sequence identity thereto;
(iv) an α chain comprising the amino acid sequence of SEQ ID NO: 16 or a variant thereof having at least 75% sequence identity thereto; and a β chain comprising an amino acid sequence selected from the group consisting of SEQ ID NO: 32, SEQ ID NO: 33 and variants of SEQ ID NOs: 32 and 33 having at least 75% sequence identity thereto;
(v) an α chain comprising the amino acid sequence of SEQ ID NO: 21 or a variant thereof having at least 75% sequence identity thereto; and a β chain comprising an amino acid sequence selected from the group consisting of SEQ ID NO: 26, SEQ ID NO: 27 and variants of SEQ ID NOs: 26 and 27 having at least 75% sequence identity thereto;
(vi) an α chain comprising the amino acid sequence of SEQ ID NO: 21 or a variant thereof having at least 75% sequence identity thereto; and a β chain comprising an amino acid sequence selected from the group consisting of SEQ ID NO: 32, SEQ ID NO: 33 and variants of SEQ ID NOs: 32 and 33 having at least 75% sequence identity thereto;
(vii) an α chain comprising the amino acid sequence of SEQ ID NO: 49 or a variant thereof having at least 75% sequence identity thereto; and a β chain comprising an amino acid sequence selected from the group consisting of SEQ ID NO: 59, SEQ ID NO: 60 and variants of SEQ ID NOs: 59 and 60 having at least 75% sequence identity thereto;
(viii) an α chain comprising the amino acid sequence of SEQ ID NO: 49 or a variant thereof having at least 75% sequence identity thereto; and a β chain comprising an amino acid sequence selected from the group consisting of SEQ ID NO: 65, SEQ ID NO: 66 and variants of SEQ ID NOs: 65 and 66 having at least 75% sequence identity thereto;
(ix) an α chain comprising the amino acid sequence of SEQ ID NO: 49 or a variant thereof having at least 75% sequence identity thereto; and a β chain comprising an amino acid sequence selected from the group consisting of SEQ ID NO: 71, SEQ ID NO: 72 and variants of SEQ ID NOs: 71 and 72 having at least 75% sequence identity thereto;
(x) an α chain comprising the amino acid sequence of SEQ ID NO: 54 or a variant thereof having at least 75% sequence identity thereto; and a β chain comprising an amino acid sequence selected from the group consisting of SEQ ID NO: 59, SEQ ID NO: 60 and variants of SEQ ID NOs: 59 and 60 having at least 75% sequence identity thereto;
(xi) an α chain comprising the amino acid sequence of SEQ ID NO: 54 or a variant thereof having at least 75% sequence identity thereto; and a β chain comprising an amino acid sequence selected from the group consisting of SEQ ID NO: 65, SEQ ID NO: 66 and variants of SEQ ID NOs: 65 and 66 having at least 75% sequence identity thereto;
(xii) an α chain comprising the amino acid sequence of SEQ ID NO: 54 or a variant thereof having at least 75% sequence identity thereto; and a β chain comprising an amino acid sequence selected from the group consisting of SEQ ID NO: 71, SEQ ID NO: 72 and variants of SEQ ID NOs: 71 and 72 having at least 75% sequence identity thereto;
(xiii) an α chain comprising the amino acid sequence of SEQ ID NO: 77 or a variant thereof having at least 75% sequence identity thereto; and a β chain comprising an amino acid sequence selected from the group consisting of SEQ ID NO: 82, SEQ ID NO: 83 and variants of SEQ ID NOs: 82 and 83 having at least 75% sequence identity thereto;
(xiv) an α chain comprising the amino acid sequence of SEQ ID NO: 77 or a variant thereof having at least 75% sequence identity thereto; and a β chain comprising an amino acid sequence selected from the group consisting of SEQ ID NO: 88, SEQ ID NO: 89 and variants of SEQ ID NOs: 88 and 89 having at least 75% sequence identity thereto;
(xv) an α chain comprising the amino acid sequence of SEQ ID NO: 94 or a variant thereof having at least 75% sequence identity thereto; and a β chain comprising an amino acid sequence selected from the group consisting of SEQ ID NO: 99, SEQ ID NO: 100 and variants of SEQ ID NO: 99, SEQ ID NO: 100 having at least 75% sequence identity thereto;
(xvi) an α chain comprising the amino acid sequence of SEQ ID NO: 105 or a variant thereof having at least 75% sequence identity thereto; and a β chain comprising an amino acid sequence selected from the group consisting of SEQ ID NO: 165, SEQ ID NO: 166 and variants of SEQ ID NOs: 165 and 166 having at least 75% sequence identity thereto;
(xvii) an α chain comprising the amino acid sequence of SEQ ID NO: 105 or a variant thereof having at least 75% sequence identity thereto; and a β chain comprising an amino acid sequence selected from the group consisting of SEQ ID NO: 171, SEQ ID NO: 172 and variants of SEQ ID NOs: 171 and 172 having at least 75% sequence identity thereto;
(xviii) an α chain comprising the amino acid sequence of SEQ ID NO: 110 or a variant thereof having at least 75% sequence identity thereto; and a β chain comprising an amino acid sequence selected from the group consisting of SEQ ID NO: 165, SEQ ID NO: 166 and variants of SEQ ID NOs: 165 and 166 having at least 75% sequence identity thereto;
(xix) an α chain comprising the amino acid sequence of SEQ ID NO: 110 or a variant thereof having at least 75% sequence identity thereto; and a β chain comprising an amino acid sequence selected from the group consisting of SEQ ID NO: 171, SEQ ID NO: 172 and variants of SEQ ID NOs: 171 and 172 having at least 75% sequence identity thereto;
(xx) an α chain comprising the amino acid sequence of SEQ ID NO: 160 or a variant thereof having at least 75% sequence identity thereto; and a β chain comprising an amino acid sequence selected from the group consisting of SEQ ID NO: 165, SEQ ID NO: 166 and variants of SEQ ID NOs: 165 and 166 having at least 75% sequence identity thereto;
(xxi) an α chain comprising the amino acid sequence of SEQ ID NO: 160 or a variant thereof having at least 75% sequence identity thereto; and a β chain comprising an amino acid sequence selected from the group consisting of SEQ ID NO: 171, SEQ ID NO: 172 and variants of SEQ ID NOs: 171 and 172 having at least 75% sequence identity thereto;
(xxii) an α chain comprising the amino acid sequence of SEQ ID NO: 177 or a variant thereof having at least 75% sequence identity thereto; and a β chain comprising an amino acid sequence selected from the group consisting of SEQ ID NO: 182, SEQ ID NO: 183 and variants of SEQ ID NOs: 182 and 183 having at least 75% sequence identity thereto;
(xxiii) an α chain comprising the amino acid sequence of SEQ ID NO: 188 or a variant thereof having at least 75% sequence identity thereto; and a β chain comprising an amino acid sequence selected from the group consisting of SEQ ID NO: 198, SEQ ID NO: 199 and variants of SEQ ID NOs: 198 and 199 having at least 75% sequence identity thereto;
(xxiv) an α chain comprising the amino acid sequence of SEQ ID NO: 188 or a variant thereof having at least 75% sequence identity thereto; and a β chain comprising an amino acid sequence selected from the group consisting of SEQ ID NO: 204, SEQ ID NO: 205 and variants of SEQ ID NOs: 204 and 205 having at least 75% sequence identity thereto;
(xxv) an α chain comprising the amino acid sequence of SEQ ID NO: 188 or a variant thereof having at least 75% sequence identity thereto; and a β chain comprising an amino acid sequence selected from the group consisting of SEQ ID NO: 210, SEQ ID NO: 211 and variants of SEQ ID NOs: 210 and 211 having at least 75% sequence identity thereto;
(xxvi) an α chain comprising the amino acid sequence of SEQ ID NO: 193 or a variant thereof having at least 75% sequence identity thereto; and a β chain comprising an amino acid sequence selected from the group consisting of SEQ ID NO: 198, SEQ ID NO: 199 and variants of SEQ ID NOs: 198 and 199 having at least 75% sequence identity thereto;
(xxvii) an α chain comprising the amino acid sequence of SEQ ID NO: 193 or a variant thereof having at least 75% sequence identity thereto; and a β chain comprising an amino acid sequence selected from the group consisting of SEQ ID NO: 204, SEQ ID NO: 205 and variants of SEQ ID NOs: 204 and 205 having at least 75% sequence identity thereto;
(xxviii) an α chain comprising the amino acid sequence of SEQ ID NO: 193 or a variant thereof having at least 75% sequence identity thereto; and a β chain comprising an amino acid sequence selected from the group consisting of SEQ ID NO: 210, SEQ ID NO: 211 and variants of SEQ ID NOs: 210 and 211 having at least 75% sequence identity thereto;
(xxix) an α chain comprising the amino acid sequence of SEQ ID NO: 216 or a variant thereof having at least 75% sequence identity thereto; and a β chain comprising an amino acid sequence selected from the group consisting of SEQ ID NO: 221, SEQ ID NO: 222 and variants of SEQ ID NOs: 221 and 222 having at least 75% sequence identity thereto;
(xxx) an α chain comprising the amino acid sequence of SEQ ID NO: 94 or a variant thereof having at least 75% sequence identity thereto; and a β chain comprising an amino acid sequence selected from the group consisting of SEQ ID NO: 273, SEQ ID NO: 274 and variants of SEQ ID NOs: 273 and 274 having at least 75% sequence identity thereto;
(xxxi) an α chain comprising the amino acid sequence of SEQ ID NO: 268 or a variant thereof having at least 75% sequence identity thereto; and a β chain comprising an amino acid sequence selected from the group consisting of SEQ ID NO: 99, SEQ ID NO: 100 and variants of SEQ ID NOs: 99 and 100 having at least 75% sequence identity thereto;
(xxxii) an α chain comprising the amino acid sequence of SEQ ID NO: 268 or a variant thereof having at least 75% sequence identity thereto; and a β chain comprising an amino acid sequence selected from the group consisting of SEQ ID NO: 273, SEQ ID NO: 274 and variants of SEQ ID NOs: 273 and 274 having at least 75% sequence identity thereto;
(xxxiii) an α chain comprising the amino acid sequence of SEQ ID NO: 279 or a variant thereof having at least 75% sequence identity thereto; and a β chain comprising an amino acid sequence selected from the group consisting of SEQ ID NO: 165, SEQ ID NO: 166 and variants of SEQ ID NOs: 165 and 166 having at least 75% sequence identity thereto;
(xxxiv) an α chain comprising the amino acid sequence of SEQ ID NO: 279 or a variant thereof having at least 75% sequence identity thereto; and a β chain comprising an amino acid sequence selected from the group consisting of SEQ ID NO: 171, SEQ ID NO: 172 and variants of SEQ ID NOs: 171 and 172 having at least 75% sequence identity thereto;
(xxxv) an α chain comprising the amino acid sequence of SEQ ID NO: 279 or a variant thereof having at least 75% sequence identity thereto; and a β chain comprising an amino acid sequence selected from the group consisting of SEQ ID NO: 284, SEQ ID NO: 285 and variants of SEQ ID NOs: 284 and 285 having at least 75% sequence identity thereto;
(xxxvi) an α chain comprising the amino acid sequence of SEQ ID NO: 279 or a variant thereof having at least 75% sequence identity thereto; and a β chain comprising an amino acid sequence selected from the group consisting of SEQ ID NO: 290, SEQ ID NO: 291 and variants of SEQ ID NOs: 290 and 291 having at least 75% sequence identity thereto;
(xxxvii) an α chain comprising the amino acid sequence of SEQ ID NO: 105 or a variant thereof having at least 75% sequence identity thereto; and a β chain comprising an amino acid sequence selected from the group consisting of SEQ ID NO: 290, SEQ ID NO: 291 and variants of SEQ ID NOs: 290 and 291 having at least 75% sequence identity thereto;
(xxxviii) an α chain comprising the amino acid sequence of SEQ ID NO: 110 or a variant thereof having at least 75% sequence identity thereto; and a β chain comprising an amino acid sequence selected from the group consisting of SEQ ID NO: 290, SEQ ID NO: 291 and variants of SEQ ID NOs: 290 and 291 having at least 75% sequence identity thereto;
(xxxix) an α chain comprising the amino acid sequence of SEQ ID NO: 160 or a variant thereof having at least 75% sequence identity thereto; and a β chain comprising an amino acid sequence selected from the group consisting of SEQ ID NO: 290, SEQ ID NO: 291 and variants of SEQ ID NOs: 290 and 291 having at least 75% sequence identity thereto;
(xxxx) an α chain comprising the amino acid sequence of SEQ ID NO: 105 or a variant thereof having at least 75% sequence identity thereto; and a β chain comprising an amino acid sequence selected from the group consisting of SEQ ID NO: 284, SEQ ID NO: 285 and variants of SEQ ID NOs: 284 and 285 having at least 75% sequence identity thereto;
(xxxxi) an α chain comprising the amino acid sequence of SEQ ID NO: 110 or a variant thereof having at least 75% sequence identity thereto; and a β chain comprising an amino acid sequence selected from the group consisting of SEQ ID NO: 284, SEQ ID NO: 285 and variants of SEQ ID NOs: 284 and 285 having at least 75% sequence identity thereto; or
(xxxxii) an α chain comprising the amino acid sequence of SEQ ID NO: 160 or a variant thereof having at least 75% sequence identity thereto; and a β chain comprising an amino acid sequence selected from the group consisting of SEQ ID NO: 284, SEQ ID NO: 285 and variants of SEQ ID NOs: 284 and 285 having at least 75% sequence identity thereto.

5. (canceled)

6. A TCR according to claim 1 which binds to an MHC I and/or MHC II peptide complex.

7. A TCR according to claim 1, which is restricted to a human leukocyte antigen (HLA) allele.

8. A TCR according to:
a. (i), (ii), (xv), (xvi), (xxix), (xxx)-(xxxiii), (xxxv) or (xxxx) of claim 1, which is restricted to HLA-A*0201;
b. (vii) of claim 1, which is restricted to HLA-B*3502; or
c. any of (xiii)-(xiv) of claim 1, which is restricted to HLA-B*3501.

9. A TCR according to claim 1 comprising one or more mutations at the α chain/β chain interface, such that when the α chain and the β chain are expressed in a T-cell, the frequency of mispairing between said chains and endogenous TCR α and β chains is reduced.

10. A TCR according to claim 9, wherein the one or more mutations introduce a cysteine residue into the constant region domain of each of the α chain and the β chain, wherein the cysteine residues are capable of forming a disulphide bond between the α chain and the β chain.

11. A TCR according to claim 1, which comprises a murinized constant region.

12. A TCR according to claim 1, wherein the TCR is a soluble TCR.

13. An isolated polynucleotide encoding an α chain of a T-cell receptor (TCR) and/or a β chain of a TCR, wherein the TCR binds to a Wilms tumour 1 protein (WT1) peptide when presented by a major histocompatibility complex (MHC), and wherein the TCR:
(i) comprises a CDR3α comprising the amino acid sequence of CGTAWINDYKLSF (SEQ ID NO: 3) or a variant thereof having up to three amino acid substitutions, additions or deletions, and a CDR3β comprising the amino acid sequence of CASRKTGGYSNQPQHF (SEQ ID NO: 8) or a variant thereof having up to three amino acid substitutions, additions or deletions;
(ii) comprises a CDR3α comprising the amino acid sequence of CVVNLLSNQGGKLIF (SEQ ID NO: 36) or a variant thereof having up to three amino acid substitutions, additions or deletions, and a CDR3β comprising the amino acid sequence of CASSQDYLVSNEKLFF (SEQ ID NO: 41) or a variant thereof having up to three amino acid substitutions, additions or deletions;
(iii) comprises a CDR3α comprising the amino acid sequence of CAVRLSGSARQLTF (SEQ ID NO: 14) or a variant thereof having up to three amino acid substitutions, additions or deletions, and a CDR3β comprising the amino acid sequence of CASSLLGDEQYF (SEQ ID NO: 24) or a variant thereof having up to three amino acid substitutions, additions or deletions;
(iv) comprises a CDR3α comprising the amino acid sequence of CAVRLSGSARQLTF (SEQ ID NO: 14) or a variant thereof having up to three amino acid substitutions, additions or deletions, and a CDR3β comprising the amino acid sequence of CASSLVALQGAGEQYF (SEQ ID NO: 30) or a variant thereof having up to three amino acid substitutions, additions or deletions;
(v) comprises a CDR3α comprising the amino acid sequence of CAYRSLKYGNKLVF (SEQ ID NO: 19) or a variant thereof having up to three amino acid substitutions, additions or deletions, and a CDR3β comprising the amino acid sequence of CASSLLGDEQYF (SEQ ID NO: 24) or a variant thereof having up to three amino acid substitutions, additions or deletions;
(vi) comprises a CDR3α comprising the amino acid sequence of CAYRSLKYGNKLVF (SEQ ID NO: 19) or a variant thereof having up to three amino acid substitutions, additions or deletions, and a CDR3β comprising the amino acid sequence of CASSLVALQGAGEQYF (SEQ ID NO: 30) or a variant thereof having up to three amino acid substitutions, additions or deletions;
(vii) comprises a CDR3α comprising the amino acid sequence of CATDAYSGNTPLVF (SEQ ID NO: 47) or a variant thereof having up to three amino acid substitutions, additions or deletions, and a CDR3β comprising the amino acid sequence of CASRAAGLDTEAFF (SEQ ID NO: 57) or a variant thereof having up to three amino acid substitutions, additions or deletions;
(viii) comprises a CDR3α comprising the amino acid sequence of CATDAYSGNTPLVF (SEQ ID NO: 47) or a variant thereof having up to three amino acid substitutions, additions or deletions, and a CDR3β comprising the amino acid sequence of CASTQTPYEQYF (SEQ ID NO: 63) or a variant thereof having up to three amino acid substitutions, additions or deletions;
(ix) comprises a CDR3α comprising the amino acid sequence of CATDAYSGNTPLVF (SEQ ID NO: 47) or a variant thereof having up to three amino acid substitutions, additions or deletions, and a CDR3β comprising the amino acid sequence of CASSTVGGEDYGYTF (SEQ ID NO: 69) or a variant thereof having up to three amino acid substitutions, additions or deletions;
(x) comprises a CDR3α comprising the amino acid sequence of CAVRAEIYNQGGKLIF (SEQ ID NO: 52) or a variant thereof having up to three amino acid substitutions, additions or deletions, and a CDR3β comprising the amino acid sequence of CASRAAGLDTEAFF (SEQ ID NO:57) or a variant thereof having up to three amino acid substitutions, additions or deletions;
(xi) comprises a CDR3α comprising the amino acid sequence of CAVRAEIYNQGGKLIF (SEQ ID NO: 52) or a variant thereof having up to three amino acid substitutions, additions or deletions, and a CDR3β comprising the amino acid sequence of CASTQTPYEQYF (SEQ ID NO: 63) or a variant thereof having up to three amino acid substitutions, additions or deletions;
(xii) comprises a CDR3α comprising the amino acid sequence of CAVRAEIYNQGGKLIF (SEQ ID NO: 52) or a variant thereof having up to three amino acid substitutions, additions or deletions, and a CDR3β comprising the amino acid sequence of CASSTVGGEDYGYTF (SEQ ID NO: 69) or a variant thereof having up to three amino acid substitutions, additions or deletions;
(xiii) comprises a CDR3α comprising the amino acid sequence of CAASMAGAGSYQLTF (SEQ ID NO: 75) or a variant thereof having up to three amino acid substitutions, additions or deletions, and a CDR3β comprising the amino acid sequence of CAISVGQGALYEQYF (SEQ ID NO: 80) or a variant thereof having up to three amino acid substitutions, additions or deletions;
(xiv) comprises a CDR3α comprising the amino acid sequence of CAASMAGAGSYQLTF (SEQ ID NO: 75) or a variant thereof having up to three amino acid substitutions, additions or deletions, and a CDR3β comprising the amino acid sequence of CASSVARDRRNYGYTF (SEQ ID NO: 86) or a variant thereof having up to three amino acid substitutions, additions or deletions;
(xv) comprises a CDR3α comprising the amino acid sequence of CAANNARLMF (SEQ ID NO: 92) or a variant thereof having up to three amino acid substitutions, additions or deletions, and a CDR3β comprising the amino acid sequence of CASSDTRAREQFF (SEQ ID NO: 97) or a variant thereof having up to three amino acid substitutions, additions or deletions;
(xvi) comprises a CDR3α comprising the amino acid sequence of CAERLNTDKLIF (SEQ ID NO: 103) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions;
(xvii) comprises a CDR3α comprising the amino acid sequence of CAERLNTDKLIF (SEQ ID NO: 103) or a variant thereof having up to three amino acid substitutions, additions or deletions, and a CDR3β comprising the amino acid sequence of CSVGGSGSYNEQFF (SEQ ID NO: 169) or a variant thereof having up to three amino acid substitutions, additions or deletions;
(xviii) comprises a CDR3α comprising the amino acid sequence of CAVEATDSWGKLQF (SEQ ID NO: 108) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions;
(xix) comprises a CDR3α comprising the amino acid sequence of CAVEATDSWGKLQF (SEQ ID NO: 108) or a variant thereof having up to three amino acid substitutions, additions or deletions, and a CDR3β comprising the amino acid sequence of CSVGGSGSYNEQFF (SEQ ID NO: 169) or a variant thereof having up to three amino acid substitutions, additions or deletions;
(xx) comprises a CDR3α comprising the amino acid sequence of CAVRTSYDKVIF (SEQ ID NO: 113) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions;
(xxi) comprises a CDR3α comprising the amino acid sequence of CAVRTSYDKVIF (SEQ ID NO: 113) or a variant thereof having up to three amino acid substitutions, additions or deletions, and a CDR3β comprising the amino acid sequence of CSVGGSGSYNEQFF (SEQ ID NO: 169) or a variant thereof having up to three amino acid substitutions, additions or deletions;
(xxii) comprises a CDR3α comprising the amino acid sequence of CAVTVGNKLVF (SEQ ID NO: 175) or a variant thereof having up to three amino acid substitutions, additions or deletions, and a CDR3β comprising the amino acid sequence of CASRGWREQFF (SEQ ID NO: 180) or a variant thereof having up to three amino acid substitutions, additions or deletions;
(xxiii) comprises a CDR3α comprising the amino acid sequence of CAARSYNTDKLIF (SEQ ID NO: 186) or a variant thereof having up to three amino acid substitutions, additions or deletions, and a CDR3β comprising the amino acid sequence of CASSWGYQETQYF (SEQ ID NO: 196) or a variant thereof having up to three amino acid substitutions, additions or deletions;
(xxiv) comprises a CDR3α comprising the amino acid sequence of CAARSYNTDKLIF (SEQ ID NO: 186) or a variant thereof having up to three amino acid substitutions, additions or deletions, and a CDR3β comprising the amino acid sequence of CASSPTGGEYYGYTF (SEQ ID NO: 202) or a variant thereof having up to three amino acid substitutions, additions or deletions;
(xxv) comprises a CDR3α comprising the amino acid sequence of CAARSYNTDKLIF (SEQ ID NO: 186) or a variant thereof having up to three amino acid substitutions, additions or deletions, and a CDR3β comprising the amino acid sequence of CASSSYPLRTGRYNSYNSPLHF (SEQ ID NO: 208) or a variant thereof having up to three amino acid substitutions, additions or deletions;
(xxvi) comprises a CDR3α comprising the amino acid sequence of CAASYNNARLMF (SEQ ID NO: 191) or a variant thereof having up to three amino acid substitutions, additions or deletions, and a CDR3β comprising the amino acid sequence of CASSWGYQETQYF (SEQ ID NO: 196) or a variant thereof having up to three amino acid substitutions, additions or deletions;
(xxvii) comprises a CDR3α comprising the amino acid sequence of CAASYNNARLMF (SEQ ID NO: 191) or a variant thereof having up to three amino acid substitutions, additions or deletions, and a CDR3β comprising the amino acid sequence of CASSPTGGEYYGYTF (SEQ ID NO: 202) or a variant thereof having up to three amino acid substitutions, additions or deletions;
(xxviii) comprises a CDR3α comprising the amino acid sequence of CAASYNNARLMF (SEQ ID NO: 191) or a variant thereof having up to three amino acid substitutions, additions or deletions, and a CDR3β comprising the amino acid sequence of CASSSYPLRTGRYNSYNSPLHF (SEQ ID NO: 208) or a variant thereof having up to three amino acid substitutions, additions or deletions;
(xxix) comprises a CDR3α comprising the amino acid sequence of CAASGGRDDKIIF (SEQ ID NO: 214) or a variant thereof having up to three amino acid substitutions, additions or deletions, and a CDR3β comprising the amino acid sequence of CASSYSRTESTDTQYF (SEQ ID NO: 219) or a variant thereof having up to three amino acid substitutions, additions or deletions;
(xxx) comprises a CDR3α comprising the amino acid sequence of CAANNARLMF (SEQ ID NO: 92) or a variant thereof having up to three amino acid substitutions, additions or deletions, and a CDR3β comprising the amino acid sequence of CASSPGQHGELFF (SEQ ID NO: 271) or a variant thereof having up to three amino acid substitutions, additions or deletions;
(xxxi) comprises a CDR3α comprising the amino acid sequence of CAASATGNQFYF (SEQ ID NO: 266) or a variant thereof having up to three amino acid substitutions, additions or deletions, and a CDR3β comprising the amino acid sequence of CASSDTRAREQFF (SEQ ID NO: 97) or a variant thereof having up to three amino acid substitutions, additions or deletions;
(xxxii) comprises a CDR3α comprising the amino acid sequence of CAASATGNQFYF (SEQ ID NO: 266) or a variant thereof having up to three amino acid substitutions, additions or deletions, and a CDR3β comprising the amino acid sequence of CASSPGQHGELFF (SEQ ID NO: 271) or a variant thereof having up to three amino acid substitutions, additions or deletions;
(xxxiii) comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSARDSVSGNTIYF (SEQ ID NO: 163) or a variant thereof having up to three amino acid substitutions, additions or deletions;
(xxxiv) comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSVGGSGSYNEQFF (SEQ ID NO: 169) or a variant thereof having up to three amino acid substitutions, additions or deletions;
(xxxv) comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions;
(xxxvi) comprises a CDR3α comprising the amino acid sequence of CATDGDSSYKLIF (SEQ ID NO: 277) or a variant thereof having up to three amino acid substitutions, additions or deletions, and a CDR3β comprising the amino acid sequence of CASSLGLSISQETQYF (SEQ ID NO: 288) or a variant thereof having up to three amino acid substitutions, additions or deletions;
(xxxvii) comprises a CDR3α comprising the amino acid sequence of CAERLNTDKLIF (SEQ ID NO: 103) or a variant thereof having up to three amino acid substitutions, additions or deletions, and a CDR3β comprising the amino acid sequence of CASSLGLSISQETQYF (SEQ ID NO: 288) or a variant thereof having up to three amino acid substitutions, additions or deletions;
(xxxviii) comprises a CDR3α comprising the amino acid sequence of CAVEATDSWGKLQF (SEQ ID NO: 108) or a variant thereof having up to three amino acid substitutions, additions or deletions, and a CDR3β comprising the amino acid sequence of CASSLGLSISQETQYF (SEQ ID NO: 288) or a variant thereof having up to three amino acid substitutions, additions or deletions;
(xxxix) comprises a CDR3α comprising the amino acid sequence of CAVRTSYDKVIF (SEQ ID NO: 113) or a variant thereof having up to three amino acid substitutions, additions or deletions, and a CDR3β comprising the amino acid sequence of CASSLGLSISQETQYF (SEQ ID NO: 288) or a variant thereof having up to three amino acid substitutions, additions or deletions;
(xxxx) comprises a CDR3α comprising the amino acid sequence of CAERLNTDKLIF (SEQ ID NO: 103) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions;
(xxxxi) comprises a CDR3α comprising the amino acid sequence of CAVEATDSWGKLQF (SEQ ID NO: 108) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions; or
(xxxxii) comprises a CDR3α comprising the amino acid sequence of CAVRTSYDKVIF (SEQ ID NO: 113) or a variant thereof having up to three amino acid substitutions, additions or deletions, and a CDR3β comprising the amino acid sequence of CSARDVLTGDYGYTF (SEQ ID NO: 282) or a variant thereof having up to three amino acid substitutions, additions or deletions.

14. An isolated polynucleotide according to claim 13, wherein the polynucleotide encodes the α chain linked to the β chain.

15. An isolated polynucleotide according to claim 13, which further encodes one or more short interfering RNA (siRNA) or other agents capable of reducing or preventing expression of one or more endogenous TCR gene.

16. A vector comprising a polynucleotide according to claim 13.

17. A vector according to claim 16 comprising a polynucleotide that encodes one or more CD3 chains, CD8, a suicide gene, and/or a selectable marker.

18. A cell comprising a TCR according to claim 1 or a polynucleotide endocind the α chain and/or the β chain thereof,
optionally wherein the cell further comprises a vector which encodes one or more CD3 chains, CD8, a suicide gene and/or a selectable marker.

19. A cell according to claim 18, wherein the cell is a T-cell, a lymphocyte, or a stem cell, optionally wherein the T-cell, the lymphocyte, or the stem cell is selected from the group consisting of CD4 cells, CD8 cells, naive T-cells, memory stem T-cells, central memory T-cells, double negative T-cells, effector memory T-cells, effector T-cells, Th0 cells, Tc0 cells, Th1 cells, Tc1 cells, Th2 cells, Tc2 cells, Th17 cells, Th22 cells, gamma/delta T-cells, natural killer (NK) cells, natural killer T (NKT) cells, hematopoietic stem cells and pluripotent stem cells.

20. A cell according to claim 19, wherein the cell is a T-cell which has been isolated from a subject.

21. A cell according to claim 18, wherein an endogenous gene encoding a TCR α chain and/or an endogenous gene encoding a TCR β chain is disrupted,
optionally wherein the endogenous gene encoding a TCR α chain and/or the endogenous gene encoding a TCR β chain is disrupted by insertion of an expression cassette comprising a polynucleotide sequence encoding the TCR,
further optionally wherein one or more endogenous genes encoding an MHC is disrupted,
further optionally wherein an endogenous gene involved in persistence, expansion, activity, resistance to exhaustion/senescence/inhibitory signals, homing capacity, or other T-cell functions is disrupted.

22. A method of preparing a cell, which comprises the step of introducing a vector according to claim 16 into a cell in vitro, ex vivo or in vivo, e.g. by transfection or transduction.

23. A method of preparing a cell according to claim 22, which comprises the step of T-cell editing, which comprises disrupting an endogenous gene encoding a TCR α chain and/or an endogenous gene encoding a TCR β chain with an artificial nuclease.

24. A method of preparing a cell, which comprises the step of targeted integration of an expression cassette into the endogenous gene encoding the TCR α chain and/or the endogenous gene encoding the TCR β chain disrupted by the artificial nuclease, wherein the expression cassette comprises a polynucleotide sequence comprising a nucleotide sequence encoding a TCR of claim 1.

25. A method of preparing a cell according to claim 22, which comprises the step of disrupting one or more endogenous genes encoding an MHC.

26. A method of preparing a cell according to claim 22, which comprises the step of disrupting one or more endogenous genes to modify the persistence, expansion, activity, resistance to exhaustion/senescence/inhibitory signals, homing capacity, or other T-cell functions.

27. (canceled)

28. A chimeric molecule comprising a TCR of claim 1, or a portion thereof, conjugated to a non-cellular substrate, a toxin and/or an antibody, optionally wherein the non-cellular substrate is selected from the group consisting of nanoparticles, exosomes and other non-cellular substrates.

29-30. (canceled)

31. A method for treating and/or preventing a disease associated with expression of WT1, which comprises the step of administering a TCR according to claim 1, at least one polynucleotide encoding said TCR, or a cell comprising said TCR, to a subject in need thereof.

32. The method of claim 31, wherein the disease associated with expression of WT1 is a proliferative disorder.

33. (canceled)

34. A method of adoptive cell transfer comprising administering a cell according to claim 18 to a subject in need thereof.

35. The method of claim 32, wherein the proliferative disorder is a hematological malignancy or a solid tumor.

36. The method of claim 35, wherein the hematological malignancy is acute myeloid leukemia.

37. The method of claim 35, wherein the proliferative disorder is a solid tumor.

38. The isolated polynucleotide according to claim 14, wherein a linker is present in between the α chain and β chain.

39. The isolated polynucleotide according to claim 38, wherein the linker is a 2A self-cleaving peptide.

40. The isolated polynucleotide according to claim 14, wherein the nucleotide sequence encoding the β chain is 5′ of the nucleotide sequence encoding the α chain.

41. The isolated polynucleotide according to claim 14, wherein the nucleotide sequence encoding the α chain is 5′ of the nucleotide sequence encoding the β chain.

42. The vector according to claim 16, wherein the vector is an AAV.

43. The cell according to claim 21, wherein the endogenous gene encoding the TCR α chain is disrupted by insertion of an expression cassette comprising a polynucleotide sequence encoding the TCR.

44. The cell according to claim 43, wherein the polynucleotide sequence encoding the TCR is operably linked to an expression control sequence.

45. The cell according to claim 44, wherein the expression control sequence is a promoter.
